SEQUENCE LISTING (336 sequences)

SEQ ID NO: 1 - protein, 1345 aa, Streptococcus mutans
  REGION 10..21: note = misc_feature - N-terminal RuvC-like domain
  REGION 759..766: note = misc_feature - RuvC-like domain
  REGION 837..863: note = misc_feature - HNH-like domain
  REGION 982..989: note = misc_feature - RuvC-like domain
MKKPYSIGLDIGTNSVGWAVVTDDYKVPAKKMKVLGNTDKSHIEKNLLGALLFDSGNTAEDRRLKRTARRRYTRRRNRILYLQEIFSEEMGKVDDSFFHRLEDSFLVTEDKRGERHPIFGNLEEEVKYHENFPTIYHLRQYLADNPEKVDLRLVYLALAHIIKFRGHFLIEGKFDTRNNDVQRLFQEFLAVYDNTFENSSLQEQNVQVEEILTDKISKSAKKDRVLKLFPNEKSNGRFAEFLKLIVGNQADFKKHFELEEKAPLQFSKDTYEEELEVLLAQIGDNYAELFLSAKKLYDSILLSGILTVTDVGTKAPLSASMIQRYNEHQMDLAQLKQFIRQKLSDKYNEVFSDVSKDGYAGYIDGKTNQEAFYKYLKGLLNKIEGSGYFLDKIEREDFLRKQRTFDNGSIPHQIHLQEMRAIIRRQAEFYPFLADNQDRIEKLLTFRIPYYVGPLARGKSDFAWLSRKSADKITPWNFDEIVDKESSAEAFINRMTNYDLYLPNQKVLPKHSLLYEKFTVYNELTKVKYKTEQGKTAFFDANMKQEIFDGVFKVYRKVTKDKLMDFLEKEFDEFRIVDLTGLDKENKVFNASYGTYHDLCKILDKDFLDNSKNEKILEDIVLTLTLFEDREMIRKRLENYSDLLTKEQVKKLERRHYTGWGRLSAELIHGIRNKESRKTILDYLIDDGNSNRNFMQLINDDALSFKEEIAKAQVIGETDNLNQVVSDIAGSPAIKKGILQSLKIVDELVKIMGHQPENIVVEMARENQFTNQGRRNSQQRLKGLTDSIKEFGSQILKEHPVENSQLQNDRLFLYYLQNGRDMYTGEELDIDYLSQYDIDHIIPQAFIKDNSIDNRVLTSSKENRGKSDDVPSKDVVRKMKSYWSKLLSAKLITQRKFDNLTKAERGGLTDDDKAGFIKRQLVETRQITKHVARILDERFNTETDENNKKIRQVKIVTLKSNLVSNFRKEFELYKVREINDYHHAHDAYLNAVIGKALLGVYPQLEPEFVYGDYPHFHGHKENKATAKKFFYSNIMNFFKKDDVRTDKNGEIIWKKDEHISNIKKVLSYPQVNIVKKVEEQTGGFSKESILPKGNSDKLIPRKTKKFYWDTKKYGGFDSPIVAYSILVIADIEKGKSKKLKTVKALVGVTIMEKMTFERDPVAFLERKGYRNVQEENIIKLPKYSLFKLENGRKRLLASARELQKGNEIVLPNHLGTLLYHAKNIHKVDEPKHLDYVDKHKDEFKELLDVVSNFSKKYTLAEGNLEKIKELYAQNNGEDLKELASSFINLLTFTAIGAPATFKFFDKNIDRKRYTSTTEILNATLIHQSITGLYETRIDLNKLGGD

SEQ ID NO: 2 - protein, 1368 aa, Streptococcus pyogenes [sequence omitted]

SEQ ID NO: 3 - protein, 1388 aa, Streptococcus thermophilus
  REGION 10..21: note = misc_feature - N-terminal RuvC-like domain
  REGION 760..767: note = misc_feature - RuvC-like domain
  REGION 844..870: note = misc_feature - HNH-like domain
  REGION 989..996: note = misc_feature - RuvC-like domain
MTKPYSIGLDIGTNSVGWAVTTDNYKVPSKKMKVLGNTSKKYIKKNLLGVLLFDSGITAEGRRLKRTARRRYTRRRNRILYLQEIFSTEMATLDDAFFQRLDDSFLVPDDKRDSKYPIFGNLVEEKAYHDEFPTIYHLRKYLADSTKKADLRLVYLALAHMIKYRGHFLIEGEFNSKNNDIQKNFQDFLDTYNAIFESDLSLENSKQLEEIVKDKISKLEKKDRILKLFPGEKNSGIFSEFLKLIVGNQADFRKCFNLDEKASLHFSKESYDEDLETLLGYIGDDYSDVFLKAKKLYDAILLSGFLTVTDNETEAPLSSAMIKRYNEHKEDLALLKEYIRNISLKTYNEVFKDDTKNGYAGYIDGKTNQEDFYVYLKKLLAEFEGADYFLEKIDREDFLRKQRTFDNGSIPYQIHLQEMRAILDKQAKFYPFLAKNKERIEKILTFRIPYYVGPLARGNSDFAWSIRKRNEKITPWNFEDVIDKESSAEAFINRMTSFDLYLPEEKVLPKHSLLYETFNVYNELTKVRFIAESMRDYQFLDSKQKKDIVRLYFKDKRKVTDKDIIEYLHAIYGYDGIELKGIEKQFNSSLSTYHDLLNIINDKEFLDDSSNEAIIEEIIHTLTIFEDREMIKQRLSKFENIFDKSVLKKLSRRHYTGWGKLSAKLINGIRDEKSGNTILDYLIDDGISNRNFMQLIHDDALSFKKKIQKAQIIGDEDKGNIKEVVKSLPGSPAIKKGILQSIKIVDELVKVMGGRKPESIVVEMARENQYTNQGKSNSQQRLKRLEKSLKELGSKILKENIPAKLSKIDNNALQNDRLYLYYLQNGKDMYTGDDLDIDRLSNYDIDHIIPQAFLKDNSIDNKVLVSSASNRGKSDDVPSLEVVKKRKTFWYQLLKSKLISQRKFDNLTKAERGGLSPEDKAGFIQRQLVETRQITKHVARLLDEKFNNKKDENNRAVRTVKIITLKSTLVSQFRKDFELYKVREINDFHHAHDAYLNAVVASALLKKYPKLEPEFVYGDYPKYNSFRERKSATEKVYFYSNIMNIFKKSISLADGRVIERPLIEVNEETGESVWNKESDLATVRRVLSYPQVNVVKKVEEQNHGLDRGKPKGLFNANLSSKPKPNSNENLVGAKEYLDPKKYGGYAGISNSFTVLVKGTIEKGAKKKITNVLEFQGISILDRINYRKDKLNFLLEKGYKDIELIIELPKYSLFELSDGSRRMLASILSTNNKRGEIHKGNQIFLSQKFVKLLYHAKRISNTINENHRKYVENHKKEFEELFYYILEFNENYVGAKKNGKLLNSAFQSWQNHSIDELCSSFIGPTGSERKGLFELTSRGSAADFEFLGVKIPRYRDYTPSSLLKDATLIHQSVTGLYETRIDLAKLGEG

SEQ ID NO: 4 - protein, 1053 aa, Staphylococcus aureus
MKRNYILGLDIGITSVGYGIIDYETRDVIDAGVRLFKEANVENNEGRRSKRGARRLKRRRRHRIQRVKKLLFDYNLLTDHSELSGINPYEARVKGLSQKLSEEEFSAALLHLAKRRGVHNVNEVEEDTGNELSTKEQISRNSKALEEKYVAELQLERLKKDGEVRGSINRFKTSDYVKEAKQLLKVQKAYHQLDQSFIDTYIDLLETRRTYYEGPGEGSPFGWKDIKEWYEMLMGHCTYFPEELRSVKYAYNADLYNALNDLNNLVITRDENEKLEYYEKFQIIENVFKQKKKPTLKQIAKEILVNEEDIKGYRVTSTGKPEFTNLKVYHDIKDITARKEIIENAELLDQIAKILTIYQSSEDIQEELTNLNSELTQEEIEQISNLKGYTGTHNLSLKAINLILDELWHTNDNQIAIFNRLKLVPKKVDLSQQKEIPTTLVDDFILSPVVKRSFIQSIKVINAIIKKYGLPNDIIIELAREKNSKDAQKMINEMQKRNRQTNERIEEIIRTTGKENAKYLIEKIKLHDMQEGKCLYSLEAIPLEDLLNNPFNYEVDHIIPRSVSFDNSFNNKVLVKQEENSKKGNRTPFQYLSSSDSKISYETFKKHILNLAKGKGRISKTKKEYLLEERDINRFSVQKDFINRNLVDTRYATRGLMNLLRSYFRVNNLDVKVKSINGGFTSFLRRKWKFKKERNKGYKHHAEDALIIANADFIFKEWKKLDKAKKVMENQMFEEKQAESMPEIETEQEYKEIFITPHQIKHIKDFKDYKYSHRVDKKPNRELINDTLYSTRKDDKGNTLIVNNLNGLYDKDNDKLKKLINKSPEKLLMYHHDPQTYQKLKLIMEQYGDEKNPLYKYYEETGNYLTKYSKKDNGPVIKKIKYYGNKLNAHLDITDDYPNSRNKVVKLSLKPYRFDVYLDNGVYKFVTVKNLDVIKKENYYEVNSKCYEEAKKLKKISNQAEFIASFYNNDLIKINGELYRVIGVNNDLLNRIEVNMIDITYREYLENMNDKRPPRIIKTIASKTQSIKKYSTDILGNLYEVKSKKHPQIIKKG

SEQ ID NO: 5 - protein, 1082 aa, Neisseria meningitidis
MAAFKPNPINYILGLDIGIASVGWAMVEIDEDENPICLIDLGVRVFERAEVPKTGDSLAMARRLARSVRRLTRRRAHRLLRARRLLKREGVLQAADFDENGLIKSLPNTPWQLRAAALDRKLTPLEWSAVLLHLIKHRGYLSQRKNEGETADKELGALLKGVADNAHALQTGDFRTPAELALNKFEKESGHIRNQRGDYSHTFSRKDLQAELILLFEKQKEFGNPHVSGGLKEGIETLLMTQRPALSGDAVQKMLGHCTFEPAEPKAAKNTYTAERFIWLTKLNNLRILEQGSERPLTDTERATLMDEPYRKSKLTYAQARKLLGLEDTAFFKGLRYGKDNAEASTLMEMKAYHAISRALEKEGLKDKKSPLNLSPELQDEIGTAFSLFKTDEDITGRLKDRIQPEILEALLKHISFDKFVQISLKALRRIVPLMEQGKRYDEACAEIYGDHYGKKNTEEKIYLPPIPADEIRNPVVLRALSQARKVINGVVRRYGSPARIHIETAREVGKSFKDRKEIEKRQEENRKDREKAAAKFREYFPNFVGEPKSKDILKLRLYEQQHGKCLYSGKEINLGRLNEKGYVEIDHALPFSRTWDDSFNNKVLVLGSENQNKGNQTPYEYFNGKDNSREWQEFKARVETSRFPRSKKQRILLQKFDEDGFKERNLNDTRYVNRFLCQFVADRMRLTGKGKKRVFASNGQITNLLRGFWGLRKVRAENDRHHALDAVVVACSTVAMQQKITRFVRYKEMNAFDGKTIDKETGEVLHQKTHFPQPWEFFAQEVMIRVFGKPDGKPEFEEADTPEKLRTLLAEKLSSRPEAVHEYVTPLFVSRAPNRKMSGQGHMETVKSAKRLDEGVSVLRVPLTQLKLKDLEKMVNREREPKLYEALKARLEAHKDDPAKAFAEPFYKYDKAGNRTQQVKAVRVEQVQKTGVWVRNHNGIADNATMVRVDVFEKGDKYYLVPIYSWQVAKGILPDRAVVQGKDEEDWQLIDDSFNFKFSLHPNDLVEVITKKARMFGYFASCHRGTGNINIRIHDLDHKIGKNGILEGIGVKTALSFQKYQIDELGKEIRPCRLKKRPPVR

SEQ ID NO: 6 - protein, 859 aa, synthetic construct
  REGION 1..859: note = Synthetic Cas9 consensus sequence derived from Sm, Sp, St, and Li Cas9 sequences
  VARIANT 4: note = Xaa can be any naturally occurring amino acid
  REGION 8..18: note = misc_feature - N-terminal RuvC-like domain
  VARIANT 21: note = Xaa can be any naturally occurring amino acid
  VARIANT 23: note = Xaa can be any naturally occurring amino acid
  VARIANT 24: note = Xaa can be any naturally occurring amino acid
  VARIANT 26: note = Xaa can be any naturally occurring amino acid
  VARIANT 29..31: note = Each Xaa can independently be any naturally occurring amino acid
  VARIANT 33: note = Xaa can be any naturally occurring amino acid
  VARIANT 36: note = Xaa can be any naturally occurring amino acid
  VARIANT 45: note = Xaa can be any naturally occurring amino acid
  VARIANT 54: note = Xaa can be any naturally occurring amino acid
  VARIANT 63: note = Xaa can be any naturally occurring amino acid
  VARIANT 71: note = Xaa can be any naturally occurring amino acid
  VARIANT 75: note = Xaa can be any naturally occurring amino acid
  VARIANT 76: note = Xaa can be any naturally occurring amino acid
  VARIANT 78..80: note = Each Xaa can independently be any naturally occurring amino acid
  VARIANT 82: note = Xaa can be any naturally occurring amino acid
  VARIANT 84: note = Xaa can be any naturally occurring amino acid
  VARIANT 85: note = Xaa can be any naturally occurring amino acid
  VARIANT 89: note = Xaa can be any naturally occurring amino acid
  VARIANT 90: note = Xaa can be any naturally occurring amino acid
  VARIANT 98: note = Xaa can be any naturally occurring amino acid
  VARIANT 100: note = Xaa can be any naturally occurring amino acid
  VARIANT 106: note = Xaa can be any naturally occurring amino acid
  VARIANT 113: note = Xaa can be any naturally occurring amino acid
  VARIANT 116: note = Xaa can be any naturally occurring amino acid
  VARIANT 125: note = Xaa can be any naturally occurring amino acid
  VARIANT 126: note = Xaa can be any naturally occurring amino acid
  VARIANT 128..133: note = Each Xaa can independently be any naturally occurring amino acid
  VARIANT 135: note = Xaa can be any naturally occurring amino acid
  VARIANT 137: note = Xaa can be any naturally occurring amino acid
  VARIANT 139..147: note = Each Xaa can independently be any naturally occurring amino acid
  VARIANT 153..155: note = Each Xaa can independently be any naturally occurring amino acid
  VARIANT 157: note = Xaa can be any naturally occurring amino acid
  VARIANT 159: note = Xaa can be any naturally occurring amino acid
  VARIANT 161: note = Xaa can be any naturally occurring amino acid
  VARIANT 163: note = Xaa can be any naturally occurring amino acid
  VARIANT 166..168: note = Each Xaa can independently be any naturally occurring amino acid
  VARIANT 170: note = Xaa can be any naturally occurring amino acid
  VARIANT 171: note = Xaa can be any naturally occurring amino acid
  VARIANT 173..175: note = Each Xaa can independently be any naturally occurring amino acid
  VARIANT 177: note = Xaa can be any naturally occurring amino acid
  VARIANT 183: note = Xaa can be any naturally occurring amino acid
  VARIANT 185..187: note = Each Xaa can independently be any naturally occurring amino acid
  VARIANT 189: note = Xaa can be any naturally occurring amino acid
  VARIANT 192..195: note = Each Xaa can independently be any naturally occurring amino acid
  VARIANT 198: note = Xaa can be any naturally occurring amino acid
  VARIANT 199: note = Xaa can be any naturally occurring amino acid
  VARIANT 202: note = Xaa can be any naturally occurring amino acid
  VARIANT 206: note = Xaa can be any naturally occurring amino acid
  VARIANT 207: note = Xaa can be any naturally occurring amino acid
  VARIANT 210: note = Xaa can be any naturally occurring amino acid
  VARIANT 212: note = Xaa can be any naturally occurring amino acid
  VARIANT 213: note = Xaa can be any naturally occurring amino acid
  VARIANT 219: note = Xaa can be any naturally occurring amino acid
  VARIANT 220: note = Xaa can be any naturally occurring amino acid
  VARIANT 222: note = Xaa can be any naturally occurring amino acid
  VARIANT 224: note = Xaa can be any naturally occurring amino acid
  VARIANT 226: note = Xaa can be any naturally occurring amino acid
  VARIANT 236: note = Xaa can be any naturally occurring amino acid
  VARIANT 240: note = Xaa can be any naturally occurring amino acid
  VARIANT 241: note = Xaa can be any naturally occurring amino acid
  VARIANT 244..246: note = Each Xaa can independently be any naturally occurring amino acid
  VARIANT 248: note = Xaa can be any naturally occurring amino acid
  VARIANT 249: note = Xaa can be any naturally occurring amino acid
  VARIANT 250: note = Xaa can be any naturally occurring amino acid
  VARIANT 252..254: note = Each Xaa can independently be any naturally occurring amino acid
  VARIANT 256: note = Xaa can be any naturally occurring amino acid
  VARIANT 257: note = Xaa can be any naturally occurring amino acid
  VARIANT 268: note = Xaa can be any naturally occurring amino acid
  VARIANT 271: note = Xaa can be any naturally occurring amino acid
  VARIANT 273: note = Xaa can be any naturally occurring amino acid
  VARIANT 277: note = Xaa can be any naturally occurring amino acid
  VARIANT 280: note = Xaa can be any naturally occurring amino acid
  VARIANT 281: note = Xaa can be any naturally occurring amino acid
  VARIANT 283: note = Xaa can be any naturally occurring amino acid
  VARIANT 289: note = Xaa can be any naturally occurring amino acid
  VARIANT 290: note = Xaa can be any naturally occurring amino acid
  VARIANT 292..294: note = Each Xaa can independently be any naturally occurring amino acid
  VARIANT 301: note = Xaa can be any naturally occurring amino acid
  VARIANT 308: note = Xaa can be any naturally occurring amino acid
  VARIANT 319..322: note = Each Xaa can independently be any naturally occurring amino acid
  VARIANT 328: note = Xaa can be any naturally occurring amino acid
  VARIANT 329: note = Xaa can be any naturally occurring amino acid
  VARIANT 335..337: note = Each Xaa can independently be any naturally occurring amino acid
  VARIANT 346: note = Xaa can be any naturally occurring amino acid
  VARIANT 347: note = Xaa can be any naturally occurring amino acid
  VARIANT 356..361: note = Each Xaa can independently be any naturally occurring amino acid
  VARIANT 363: note = Xaa can be any naturally occurring amino acid
  VARIANT 370..373: note = Each Xaa can independently be any naturally occurring amino acid
  VARIANT 375: note = Xaa can be any naturally occurring amino acid
  VARIANT 376: note = Xaa can be any naturally occurring amino acid
  VARIANT 379: note = Xaa can be any naturally occurring amino acid
  VARIANT 386..390: note = Each Xaa can independently be any naturally occurring amino acid
  VARIANT 393: note = Xaa can be any naturally occurring amino acid
  VARIANT 395: note = Xaa can be any naturally occurring amino acid
  VARIANT 396: note = Xaa can be any naturally occurring amino acid
  VARIANT 398: note = Xaa can be any naturally occurring amino acid
  VARIANT 400: note = Xaa can be any naturally occurring amino acid
  VARIANT 403: note = Xaa can be any naturally occurring amino acid
  VARIANT 407: note = Xaa can be any naturally occurring amino acid
  VARIANT 410: note = Xaa can be any naturally occurring amino acid
  VARIANT 411: note = Xaa can be any naturally occurring amino acid
  VARIANT 413..416: note = Each Xaa can independently be any naturally occurring amino acid
  VARIANT 418: note = Xaa can be any naturally occurring amino acid
  VARIANT 422: note = Xaa can be any naturally occurring amino acid
  VARIANT 428: note = Xaa can be any naturally occurring amino acid
  VARIANT 431: note = Xaa can be any naturally occurring amino acid
  VARIANT 433: note = Xaa can be any naturally occurring amino acid
  VARIANT 437..439: note = Each Xaa can independently be any naturally occurring amino acid
  VARIANT 445: note = Xaa can be any naturally occurring amino acid
  VARIANT 451: note = Xaa can be any naturally occurring amino acid
  VARIANT 456: note = Xaa can be any naturally occurring amino acid
  VARIANT 459: note = Xaa can be any naturally occurring amino acid
  VARIANT 465..469: note = Each Xaa can independently be any naturally occurring amino acid
  VARIANT 481: note = Xaa can be any naturally occurring amino acid
  VARIANT 482: note = Xaa can be any naturally occurring amino acid
  VARIANT 484: note = Xaa can be any naturally occurring amino acid
  VARIANT 490: note = Xaa can be any naturally occurring amino acid
  VARIANT 494: note = Xaa can be any naturally occurring amino acid
  REGION 495..502: note = misc_feature - RuvC-like domain
  VARIANT 497: note = Xaa can be any naturally occurring amino acid
  VARIANT 506: note = Xaa can be any naturally occurring amino acid
  VARIANT 510: note = Xaa can be any naturally occurring amino acid
  VARIANT 513: note = Xaa can be any naturally occurring amino acid
  VARIANT 514: note = Xaa can be any naturally occurring amino acid
  VARIANT 517: note = Xaa can be any naturally occurring amino acid
  VARIANT 520: note = Xaa can be any naturally occurring amino acid
  VARIANT 525: note = Xaa can be any naturally occurring amino acid
  VARIANT 526: note = Xaa can be any naturally occurring amino acid
  VARIANT 529: note = Xaa can be any naturally occurring amino acid
  VARIANT 531: note = Xaa can be any naturally occurring amino acid
  VARIANT 532: note = Xaa can be any naturally occurring amino acid
  VARIANT 534: note = Xaa can be any naturally occurring amino acid
  VARIANT 542: note = Xaa can be any naturally occurring amino acid
  VARIANT 546: note = Xaa can be any naturally occurring amino acid
  VARIANT 547: note = Xaa can be any naturally occurring amino acid
  VARIANT 553: note = Xaa can be any naturally occurring amino acid
  REGION 555..575: note = misc_feature - HNH-like domain
  VARIANT 556: note = Xaa can be any naturally occurring amino acid
  VARIANT 560: note = Xaa can be any naturally occurring amino acid
  VARIANT 563: note = Xaa can be any naturally occurring amino acid
  VARIANT 565: note = Xaa can be any naturally occurring amino acid
  VARIANT 567: note = Xaa can be any naturally occurring amino acid
  VARIANT 579: note = Xaa can be any naturally occurring amino acid
  VARIANT 582: note = Xaa can be any naturally occurring amino acid
  VARIANT 583: note = Xaa can be any naturally occurring amino acid
  VARIANT 585: note = Xaa can be any naturally occurring amino acid
  VARIANT 588: note = Xaa can be any naturally occurring amino acid
  VARIANT 590: note = Xaa can be any naturally occurring amino acid
  VARIANT 592: note = Xaa can be any naturally occurring amino acid
  VARIANT 594..596: note = Each Xaa can independently be any naturally occurring amino acid
  VARIANT 610: note = Xaa can be any naturally occurring amino acid
  VARIANT 616: note = Xaa can be any naturally occurring amino acid
  VARIANT 628: note = Xaa can be any naturally occurring amino acid
  VARIANT 631: note = Xaa can be any naturally occurring amino acid
  VARIANT 633: note = Xaa can be any naturally occurring amino acid
  VARIANT 634: note = Xaa can be any naturally occurring amino acid
  VARIANT 636: note = Xaa can be any naturally occurring amino acid
  VARIANT 638..641: note = Each Xaa can independently be any naturally occurring amino acid
  VARIANT 643..645: note = Each Xaa can independently be any naturally occurring amino acid
  VARIANT 653: note = Xaa can be any naturally occurring amino acid
  VARIANT 657: note = Xaa can be any naturally occurring amino acid
  VARIANT 659: note = Xaa can be any naturally occurring amino acid
  VARIANT 660: note = Xaa can be any naturally occurring amino acid
  VARIANT 665: note = Xaa can be any naturally occurring amino acid
  VARIANT 666: note = Xaa can be any naturally occurring amino acid
  VARIANT 668: note = Xaa can be any naturally occurring amino acid
  VARIANT 669: note = Xaa can be any naturally occurring amino acid
  REGION 670..677: note = misc_feature - RuvC-like domain
  VARIANT 680: note = Xaa can be any naturally occurring amino acid
  VARIANT 681: note = Xaa can be any naturally occurring amino acid
  VARIANT 683: note = Xaa can be any naturally occurring amino acid
  VARIANT 686: note = Xaa can be any naturally occurring amino acid
  VARIANT 696: note = Xaa can be any naturally occurring amino acid
  VARIANT 697: note = Xaa can be any naturally occurring amino acid
  VARIANT 704: note = Xaa can be any naturally occurring amino acid
  VARIANT 708: note = Xaa can be any naturally occurring amino acid
  VARIANT 710: note = Xaa can be any naturally occurring amino acid
  VARIANT 711: note = Xaa can be any naturally occurring amino acid
  VARIANT 714: note = Xaa can be any naturally occurring amino acid
  VARIANT 717..720: note = Each Xaa can independently be any naturally occurring amino acid
  VARIANT 722: note = Xaa can be any naturally occurring amino acid
  VARIANT 725: note = Xaa can be any naturally occurring amino acid
  VARIANT 727: note = Xaa can be any naturally occurring amino acid
  VARIANT 733..735: note = Each Xaa can independently be any naturally occurring amino acid
  VARIANT 740: note = Xaa can be any naturally occurring amino acid
  VARIANT 742: note = Xaa can be any naturally occurring amino acid
  VARIANT 749..754: note = Each Xaa can independently be any naturally occurring amino acid
  VARIANT 758..761: note = Each Xaa can independently be any naturally occurring amino acid
  VARIANT 763..768: note = Each Xaa can independently be any naturally occurring amino acid
  VARIANT 771: note = Xaa can be any naturally occurring amino acid
  VARIANT 774..777: note = Each Xaa can independently be any naturally occurring amino acid
  VARIANT 782: note = Xaa can be any naturally occurring amino acid
  VARIANT 784..786: note = Each Xaa can independently be any naturally occurring amino acid
  VARIANT 788: note = Xaa can be any naturally occurring amino acid
  VARIANT 790: note = Xaa can be any naturally occurring amino acid
  VARIANT 795: note = Xaa can be any naturally occurring amino acid
  VARIANT 799: note = Xaa can be any naturally occurring amino acid
  VARIANT 801: note = Xaa can be any naturally occurring amino acid
  VARIANT 802: note = Xaa can be any naturally occurring amino acid
  VARIANT 804: note = Xaa can be any naturally occurring amino acid
  VARIANT 806..813: note = Each Xaa can independently be any naturally occurring amino acid
  VARIANT 815..818: note = Each Xaa can independently be any naturally occurring amino acid
  VARIANT 820: note = Xaa can be any naturally occurring amino acid
  VARIANT 823..827: note = Each Xaa can independently be any naturally occurring amino acid
  VARIANT 829: note = Xaa can be any naturally occurring amino acid
  VARIANT 830: note = Xaa can be any naturally occurring amino acid
  VARIANT 832: note = Xaa can be any naturally occurring amino acid
  VARIANT 835..837: note = Each Xaa can independently be any naturally occurring amino acid
  VARIANT 842..844: note = Each Xaa can independently be any naturally occurring amino acid
  VARIANT 846: note = Xaa can be any naturally occurring amino acid
  VARIANT 848: note = Xaa can be any naturally occurring amino acid
  VARIANT 851: note = Xaa can be any naturally occurring amino acid
  VARIANT 857: note = Xaa can be any naturally occurring amino acid
MKYXIGLDIGTNSVGWAVTDXYXXKXKGXXXIXKNXGLFDGTARXRTARRRRRXNRIYLQIFXEMDFFRLXSFVXXKXXXPXFXXEYHXXPTIYHLRXLXKDLRLXYLALAHXIKXRGNFLIEGXXNXXXXXXYXFXIXXXXXXXXXPEKGFXXXLXGXFXFXLEXXXKXXYXXXLXLLIGDXYXXXFXAKXXXXLSXXVTXALSXXMIXRXXHDLLKXXYXEXFXKGYAGYIDGXQFYXXKLXXXGXXXKXXXEXXLRKQRTFDNGXIPXQXHLEXAIXXQXYPFLNXXIXXXTFRIPYXVGPLAGXSFAWRKIPWNXXXXDSAFIXXMTDLPXXXVLPKHSLYXXVYNELTKVXXXXXXKXIFKRKVXXXXGXXFNXSTYHDLXXXXXLDXNXXEXIXLTXFEDXMIXXLXXXXKXLRRXYTGWGXLSXLXGIRXXXSTILDXLDNRNXMQLIXDLXFKIKQXXXXXGSPAIKKGILQXXKXVDELVXMGPXIVXEMARENQTXGNSXRKXXKEXGSXILKEXXNLXNXXLXLYYLQNGXDMYXXLDILSXYDXDHIXPQXFXDXSIDNVLSNRKDXVPXXVXKKXWXLXLXXXRKFDLTKAERGGLXDKAFIXRQLVETRQITKXVAXLXXNXDXXXXVXXXTLKSLVSXFRKXFXXLYKVXXNXXHHAHDAYLNVXXLXYPXLEEFVYGDYXXKATKFYXNIMXFXXGEXWKXXXXVXMQXNXVKKEQXXXPKNSXLXKDKYGGXXXXXXKGKXXXXIXXXXXXFLXGYXXXXLPKYXLXXXGXRXLASEXKGNXLXXLXAXXXXXXXXEXXXXFXANXXXXXLXXGXAFXXXIRRYXXXTXIXQSXTGLYEXRL

SEQ ID NO: 7 - protein, 8 aa, synthetic construct
  REGION 1..8: note = RuvC-like domain
  VARIANT 2: note = Xaa is Val or His
  VARIANT 3: note = Xaa is Ile, Leu, or Val
  VARIANT 5: note = Xaa is Met or Thr
IXXEXARE

SEQ ID NO: 8 - protein, 8 aa, synthetic construct
  REGION 1..8: note = RuvC-like domain
  VARIANT 3: note = Xaa is Ile, Leu, or Val
IVXEMARE

SEQ ID NO: 9 - protein, 8 aa, synthetic construct
  REGION 1..8: note = RuvC-like domain
  VARIANT 4: note = Xaa is His or Leu
  VARIANT 7: note = Xaa is Arg or Val
  VARIANT 8: note = Xaa is Glu or Val
HHAXDAXX

SEQ ID NO: 10 - protein, 8 aa, synthetic construct
  REGION 1..8: note = RuvC-like domain
HHAHDAYL

SEQ ID NO: 11 - protein, 30 aa, synthetic construct
  REGION 1..30: note = N-terminal RuvC-like domain
  VARIANT 2: note = Xaa is Lys or Pro
  VARIANT 4: note = Xaa is Val, Leu, Ile, or Phe
  VARIANT 5: note = Xaa is Gly, Ala, or Ser
  VARIANT 6: note = Xaa is Leu, Ile, Val, or Phe
  REGION 7..26: note = N-terminal RuvC-like domain, each Xaa can be any amino acid or absent, region may encompass 5-20 residues
  VARIANT 29: note = Xaa is Asp, Glu, Asn, or Gln
KXYXXXXXXXXXXXXXXXXXXXXXXXTDXY

SEQ ID NO: 12 - skipped

SEQ ID NO: 13 - protein, 12 aa, synthetic construct
  REGION 1..12: note = N-terminal RuvC-like domain
  VARIANT 2: note = Xaa is Ile, Val, Met, Leu, or Thr
  VARIANT 4: note = Xaa is Thr, Ile, Val, Ser, Asn, Tyr, Glu, or Leu
  VARIANT 5: note = Xaa is Asn, Ser, Gly, Ala, Asp, Thr, Arg, Met, or Phe
  VARIANT 7: note = Xaa is Val, Ile, Leu, Cys, Thr, or Phe
  VARIANT 9: note = Xaa is Trp, Phe, Val, Tyr, Ser, or Leu
  VARIANT 10: note = Xaa is Ala, Ser, Cys, Val, or Gly
  VARIANT 11: note = Xaa is Val, Ile, Leu, Ala, Met, or His
  VARIANT 12: note = Any amino acid or absent
DXGXXSXGXXXX

SEQ ID NO: 14 - protein, 12 aa, synthetic construct
  REGION 1..12: note = N-terminal RuvC-like domain
  VARIANT 4: note = Xaa is Thr, Ile, Val, Ser, Asn, Tyr, Glu, or Leu
  VARIANT 5: note = Xaa is Asn, Ser, Gly, Ala, Asp, Thr, Arg, Met, or Phe
  VARIANT 11: note = Xaa is Val, Ile, Leu, Ala, Met, or His
  VARIANT 12: note = Any amino acid or absent
DIGXXSVGWAXX

SEQ ID NO: 15 - protein, 12 aa, synthetic construct
  REGION 1..12: note = N-terminal RuvC-like domain
  VARIANT 12: note = Any non-polar alkyl amino acid or a hydroxyl amino acid
DIGTNSVGWAVX

SEQ ID NO: 16 - protein, 73 aa, synthetic construct
  REGION 1..73: note = HNH-like domain
  VARIANT 8: note = Xaa is Lys or Arg
  VARIANT 12: note = Xaa is Val or Thr
  VARIANT 13: note = Xaa is Gly or Asp
  VARIANT 14: note = Xaa is Glu, Gln, or Asp
  VARIANT 15: note = Xaa is Glu or Asp
  VARIANT 19: note = Xaa is Asp, Asn, or His
  VARIANT 20: note = Xaa is Tyr, Arg, or Asn
  VARIANT 23: note = Xaa is Gln, Asp, or Asn
  MOD_RES 25..64: note = HNH-like domain, each Xaa can be any amino acid or absent, region may encompass 15-40 residues
  VARIANT 67: note = Xaa is Gly or Glu
  VARIANT 69: note = Xaa is Ser or Gly
  VARIANT 71: note = Xaa is Asp or Asn
LYYLQNGXDMYXXXXLDIXXLSXYXXXXXXXXXXXXXXXXXXXXXXXXXXXXXXXXXXXXXXXXNRXKXDXVP

SEQ ID NO: 17 - protein, 27 aa, synthetic construct
  REGION 1..27: note = HNH-like domain
  VARIANT 1: note = Xaa is Asp, Glu, Gln, or Asn
  VARIANT 2: note = Xaa is Leu, Ile, Arg, Gln, Val, Met, or Lys
  VARIANT 3: note = Xaa is Asp or Glu
  VARIANT 5: note = Xaa is Ile, Val, Thr, Ala, or Leu
  VARIANT 6: note = Xaa is Val, Tyr, Ile, Leu, Phe, or Trp
  VARIANT 8: note = Xaa is Gln, His, Arg, Lys, Tyr, Ile, Leu, Phe, or Trp
  VARIANT 9: note = Xaa is Ser, Ala, Asp, Thr, or Lys
  VARIANT 10: note = Xaa is Phe, Leu, Val, Lys, Tyr, Met, Ile, Arg, Ala, Glu, Asp, or Gln
  VARIANT 11: note = Xaa is Leu, Arg, Thr, Ile, Val, Ser, Cys, Tyr, Lys, Phe, or Gly
  VARIANT 12: note = Xaa is Lys, Gln, Tyr, Thr, Phe, Leu, Trp, Met, Ala, Glu, Gly, or Ser
  VARIANT 13: note = Xaa is Asp, Ser, Asn, Arg, Leu, or Thr
  VARIANT 14: note = Xaa is Asp, Asn, or Ser
  VARIANT 15: note = Xaa is Ser, Ala, Thr, Gly, or Arg
  VARIANT 16: note = Xaa is Ile, Leu, Phe, Ser, Arg, Tyr, Gln, Trp, Asp, Lys, or His
  VARIANT 17: note = Xaa is Asp, Ser, Ile, Asn, Glu, Ala, His, Phe, Leu, Gln, Met, Gly, Tyr, or Val
  VARIANT 19: note = Xaa is Lys, Leu, Arg, Met, Thr, or Phe
  VARIANT 20: note = Xaa is Val, Leu, Ile, Ala, or Thr
  VARIANT 21: note = Xaa is Leu, Ile, Val, or Ala
  VARIANT 22: note = Xaa is Thr, Val, Cys, Glu, Ser, or Ala
  VARIANT 23: note = Xaa is Arg, Phe, Thr, Trp, Glu, Leu, Asn, Cys, Lys, Val, Ser, Gln, Ile, Tyr, His, or Ala
  VARIANT 24: note = Xaa is Ser, Pro, Arg, Lys, Asn, Ala, His, Gln, Gly, or Leu
  VARIANT 25: note = Xaa is Asp, Gly, Thr, Asn, Ser, Lys, Ala, Ile, Glu, Leu, Gln, Arg, or Tyr
  VARIANT 26: note = Xaa is Lys, Val, Ala, Glu, Tyr, Ile, Cys, Leu, Ser, Thr, Gly, Lys, Met, Asp, or Phe
XXXHXXPXXXXXXXXXXNXXXXXXXXN

SEQ ID NO: 18 - protein, 27 aa, synthetic construct
  REGION 1..27: note = HNH-like domain
  VARIANT 1: note = Xaa is Asp or Glu
  VARIANT 2: note = Xaa is Leu, Ile, Arg, Gln, Val, Met, or Lys
  VARIANT 3: note = Xaa is Asp or Glu
  VARIANT 5: note = Xaa is Ile, Val, Thr, Ala, or Leu
  VARIANT 6: note = Xaa is Val, Tyr, Ile, Leu, Phe, or Trp
  VARIANT 8: note = Xaa is Gln, His, Arg, Lys, Tyr, Ile, Leu, Phe, or Trp
  VARIANT 10: note = Xaa is Phe, Leu, Val, Lys, Tyr, Met, Ile, Arg, Ala, Glu, Asp, or Gln
  VARIANT 11: note = Xaa is Leu, Arg, Thr, Ile, Val, Ser, Cys, Tyr, Lys, Phe, or Gly
  VARIANT 12: note = Xaa is Lys, Gln, Tyr, Thr, Phe, Leu, Trp, Met, Ala, Glu, Gly, or Ser
  VARIANT 16: note = Xaa is Ile, Leu, Phe, Ser, Arg, Tyr, Gln, Trp, Asp, Lys, or His
  VARIANT 17: note = Xaa is Asp, Ser, Ile, Asn, Glu, Ala, His, Phe, Leu, Gln, Met, Gly, Tyr, or Val
  VARIANT 22: note = Xaa is Thr, Val, Cys, Glu, Ser, or Ala
  VARIANT 23: note = Xaa is Arg, Phe, Thr, Trp, Glu, Leu, Asn, Cys, Lys, Val, Ser, Gln, Ile, Tyr, His, or Ala
  VARIANT 24: note = Xaa is Ser, Pro, Arg, Lys, Asn, Ala, His, Gln, Gly, or Leu
  VARIANT 25: note = Xaa is Asp, Gly, Thr, Asn, Ser, Lys, Ala, Ile, Glu, Leu, Gln, Arg, or Tyr
  VARIANT 26: note = Xaa is Lys, Val, Ala, Glu, Tyr, Ile, Cys, Leu, Ser, Thr, Gly, Lys, Met, Asp, or Phe
XXXHXXPXSXXXDDSXXNKVLXXXXXN

SEQ ID NO: 19 - protein, 27 aa, synthetic construct
  REGION 1..27: note = HNH-like domain
  VARIANT 1: note = Xaa is Asp or Glu
  VARIANT 3: note = Xaa is Asp or Glu
  VARIANT 8: note = Xaa is Gln, His, Arg, Lys, Tyr, Ile, Leu, or Trp
  VARIANT 10: note = Xaa is Phe, Leu, Val, Lys, Tyr, Met, Ile, Arg, Ala, Glu, Asp, or Gln
  VARIANT 11: note = Xaa is Leu, Arg, Thr, Ile, Val, Ser, Cys, Tyr, Lys, Phe, or Gly
  VARIANT 12: note = Xaa is Lys, Gln, Tyr, Thr, Phe, Leu, Trp, Met, Ala, Glu, Gly, or Ser
  VARIANT 16: note = Xaa is Ile, Leu, Phe, Ser, Arg, Tyr, Gln, Trp, Asp, Lys, or His
  VARIANT 17: note = Xaa is Asp, Ser, Ile, Asn, Glu, Ala, His, Phe, Leu, Gln, Met, Gly, Tyr, or Val
  VARIANT 23: note = Xaa is Arg, Phe, Thr, Trp, Glu, Leu, Asn, Cys, Lys, Val, Ser, Gln, Ile, Tyr, His, or Ala
  VARIANT 24: note = Xaa is Ser, Pro, Arg, Lys, Asn, Ala, His, Gln, Gly, or Leu
  VARIANT 25: note = Xaa is Asp, Gly, Thr, Asn, Ser, Lys, Ala, Ile, Glu, Leu, Gln, Arg, or Tyr
  VARIANT 26: note = Xaa is Lys, Val, Ala, Glu, Tyr, Ile, Cys, Leu, Ser, Thr, Gly, Lys, Met, Asp, or Phe
XVXHIVPXSXXXDDSXXNKVLTXXXXN

SEQ ID NO: 20 - protein, 27 aa, synthetic construct
  REGION 1..27: note = HNH-like domain
  VARIANT 2: note = Xaa is Ile or Val
  VARIANT 6: note = Xaa is Ile or Val
  VARIANT 9: note = Xaa is Ala or Ser
  VARIANT 11: note = Xaa is Ile or Leu
  VARIANT 12: note = Xaa is Lys or Thr
  VARIANT 14: note = Xaa is Asp or Asn
  VARIANT 19: note = Xaa is Arg, Lys, or Leu
  VARIANT 22: note = Xaa is Thr or Val
  VARIANT 23: note = Xaa is Ser or Arg
  VARIANT 25: note = Xaa is Lys, Asp, or Ala
  VARIANT 26: note = Xaa is Glu, Lys, Gly, or Asn
DXDHIXPQXFXXDXSIDNXVLXXSXXN

SEQ ID NO: 21 - RNA, 116 nt, synthetic construct
  misc_feature 1..116: note = gRNA
  variation 1..20: note = a, c, u, g, unknown or other
  misc_feature 1..20: note = targeting region
  misc_feature 21..42: note = first complementarity domain
  misc_feature 43..46: note = linking domain
  misc_feature 47..70: note = second complementarity domain
  source 1..116: mol_type = other RNA
nnnnnnnnnnnnnnnnnnnngttttagagctatgctgttttggaaacaaaacagcatagcaagttaaaataaggctagtccgttatcaacttgaaaaagtggcaccgagtcggtgc

SEQ ID NO: 22 - RNA, 116 nt, synthetic construct
  misc_feature 1..116: note = gRNA
  variation 1..20: note = a, c, u, g, unknown or other
  misc_feature 1..20: note = targeting region
  misc_feature 21..42: note = first complementarity domain
  misc_feature 43..46: note = linking domain
  misc_feature 47..70: note = second complementarity domain
  source 1..116: mol_type = other RNA
nnnnnnnnnnnnnnnnnnnngtattagagctatgctgtattggaaacaatacagcatagcaagttaatataaggctagtccgttatcaacttgaaaaagtggcaccgagtcggtgc

SEQ ID NO: 23 - RNA, 96 nt, synthetic construct
  misc_feature 1..96: note = gRNA
  variation 1..20: note = a, c, u, g, unknown or other
  misc_feature 1..20: note = targeting domain
  misc_feature 21..32: note = first complementarity domain
  misc_feature 33..36: note = linking domain
  misc_feature 37..50: note = second complementarity domain
  misc_feature 51..62: note = proximal domain
  source 1..96: mol_type = other RNA
nnnnnnnnnnnnnnnnnnnngtttaagagctagaaatagcaagtttaaataaggctagtccgttatcaacttgaaaaagtggcaccgagtcggtgc

SEQ ID NO: 24 - RNA, 47 nt, synthetic construct
  misc_feature 1..47: note = gRNA proximal and tail domains derived from S. pyogenes
  source 1..47: mol_type = other RNA
aaggctagtccgttatcaacttgaaaaagtggcaccgagtcggtgct

SEQ ID NO: 25 - RNA, 49 nt, synthetic construct
  misc_feature 1..49: note = gRNA proximal and tail domains
  source 1..49: mol_type = other RNA
aaggctagtccgttatcaacttgaaaaagtggcaccgagtcggtggtgc

SEQ ID NO: 26 - RNA, 51 nt, synthetic construct
  misc_feature 1..51: note = gRNA proximal and tail domains
  source 1..51: mol_type = other RNA
aaggctagtccgttatcaacttgaaaaagtggcaccgagtcggtgcggatc

SEQ ID NO: 27 - RNA, 31 nt, synthetic construct
  misc_feature 1..31: note = gRNA proximal and tail domains
  source 1..31: mol_type = other RNA
aaggctagtccgttatcaacttgaaaaagtg

SEQ ID NO: 28 - RNA, 18 nt, synthetic construct
  misc_feature 1..18: note = gRNA proximal and tail domains
  source 1..18: mol_type = other RNA
aaggctagtccgttatca

SEQ ID NO: 29 - RNA, 12 nt, synthetic construct
  misc_feature 1..12: note = gRNA proximal and tail domains
  source 1..12: mol_type = other RNA
aaggctagtccg

SEQ ID NO: 30 - RNA, 102 nt, synthetic construct
  misc_feature 1..102: note = Unimolecular gRNA derived from S. aureus
  variation 1..20: note = n is a, c, g, or u
  misc_feature 1..20: note = Targeting domain
  source 1..102: mol_type = other RNA
nnnnnnnnnnnnnnnnnnnngttttagtactctggaaacagaatctactaaaacaaggcaaaatgccgtgtttatctcgtcaacttgttggcgagatttttt

SEQ ID NO: 31 - RNA, 42 nt, synthetic construct
  misc_feature 1..42: note = Modular gRNA derived from S. pyogenes
  variation 1..20: note = a, c, u, g, unknown or other
  misc_feature 1..20: note = Targeting domain
  misc_feature 21..42: note = First complementarity domain
  source 1..42: mol_type = other RNA
nnnnnnnnnnnnnnnnnnnngttttagagctatgctgttttg

SEQ ID NO: 32 - RNA, 85 nt, synthetic construct
  misc_feature 1..85: note = Modular gRNA derived from S. pyogenes
  misc_feature 1..9: note = 5' extension domain
  misc_feature 10..33: note = Second complementarity domain
  misc_feature 34..45: note = Proximal domain
  misc_feature 46..85: note = Tail domain
  source 1..85: mol_type = other RNA
ggaaccattcaaaacagcatagcaagttaaaataaggctagtccgttatcaacttgaaaaagtggcaccgagtcggtgctttttt

SEQ ID NO: 33 - RNA, 62 nt, synthetic construct
  misc_feature 1..62: note = Unimolecular gRNA derived from S. pyogenes
  variation 1..20: note = a, c, u, g, unknown or other
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  source 1..62: mol_type = other RNA
nnnnnnnnnnnnnnnnnnnngttttagagctagaaatagcaagttaaaataaggctagtccg

SEQ ID NO: 34 - RNA, 102 nt, synthetic construct
  misc_feature 1..102: note = Unimolecular gRNA derived from S. pyogenes
  variation 1..20: note = a, c, u, g, unknown or other
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  misc_feature 63..102: note = Tail domain
  source 1..102: mol_type = other RNA
nnnnnnnnnnnnnnnnnnnngttttagagctagaaatagcaagttaaaataaggctagtccgttatcaacttgaaaaagtggcaccgagtcggtgctttttt

SEQ ID NO: 35 - RNA, 75 nt, synthetic construct
  misc_feature 1..75: note = Unimolecular gRNA derived from S. pyogenes
  misc_feature 1..20: note = Targeting domain
  variation 1..20: note = a, c, u, g, unknown or other
  misc_feature 21..36: note = First complementarity domain
  misc_feature 37..40: note = Linking domain
  misc_feature 41..58: note = Second complementarity domain
  misc_feature 59..70: note = Proximal domain
  misc_feature 71..75: note = Tail domain
  source 1..75: mol_type = other RNA
nnnnnnnnnnnnnnnnnnnngttttagagctatgctgaaaagcatagcaagttaaaataaggctagtccgttatc

SEQ ID NO: 36 - RNA, 87 nt, synthetic construct
  misc_feature 1..87: note = Unimolecular gRNA derived from S. pyogenes
  variation 1..20: note = a, c, u, g, unknown or other
  misc_feature 1..20: note = Targeting domain
  misc_feature 21..32: note = First complementarity domain
  misc_feature 43..46: note = Linking domain
  misc_feature 57..70: note = Second complementarity domain
  misc_feature 71..82: note = Proximal domain
  misc_feature 83..87: note = Tail domain
  source 1..87: mol_type = other RNA
nnnnnnnnnnnnnnnnnnnngttttagagctatgctgttttggaaacaaaacagcatagcaagttaaaataaggctagtccgttatc

SEQ ID NO: 37 - RNA, 42 nt, synthetic construct
  misc_feature 1..42: note = Modular gRNA derived from S. thermophilus
  variation 1..20: note = a, c, u, g, unknown or other
  misc_feature 1..20: note = Targeting domain
  misc_feature 21..42: note = First complementarity domain
  source 1..42: mol_type = other RNA
nnnnnnnnnnnnnnnnnnnngttttagagctgtgttgtttcg

SEQ ID NO: 38 - RNA, 78 nt, synthetic construct
  misc_feature 1..78: note = Modular gRNA derived from S. thermophilus
  misc_feature 1..3: note = 5' extension domain
  misc_feature 4..27: note = Second complementarity domain
  misc_feature 28..40: note = Proximal domain
  misc_feature 41..78: note = Tail domain
  source 1..78: mol_type = other RNA
gggcgaaacaacacagcgagttaaaataaggcttagtccgtactcaacttgaaaaggtggcaccgattcggtgttttt

SEQ ID NO: 39 - RNA, 85 nt, synthetic construct
  misc_feature 1..85: note = Modular gRNA derived from S. pyogenes
  source 1..85: mol_type = other RNA
gaaccattcaaaacagcatagcaagttaaaataaggctagtccgttatcaacttgaaaaagtggcaccgagtcggtgcttttttt

SEQ ID NO: 40 - RNA, 96 nt, synthetic construct
  misc_feature 1..96: note = gRNA from S. pyogenes
  variation 1..20: note = n is a, c, g, or u
  misc_feature 1..20: note = Targeting domain
  source 1..96: mol_type = other RNA
nnnnnnnnnnnnnnnnnnnngttttagagctagaaatagcaagttaaaataaggctagtccgttatcaacttgaaaaagtggcaccgagtcggtgc

SEQ ID NO: 41 - RNA, 96 nt, synthetic construct
  misc_feature 1..96: note = gRNA
  variation 1..20: note = n is a, c, g, or u
  misc_feature 1..20: note = Targeting domain
  misc_feature 21..32: note = First complementarity domain
  misc_feature 33..36: note = Linking domain
  misc_feature 37..50: note = Second complementarity domain
  misc_feature 51..62: note = Proximal domain
  source 1..96: mol_type = other RNA
nnnnnnnnnnnnnnnnnnnngtattagagctagaaatagcaagttaatataaggctagtccgttatcaacttgaaaaagtggcaccgagtcggtgc

SEQ ID NO: 42 - RNA, 104 nt, synthetic construct
  misc_feature 1..104: note = gRNA
  variation 1..20: note = n is a, c, g, or u
  misc_feature 1..20: note = Targeting domain
  misc_feature 21..36: note = First complementarity domain
  misc_feature 37..40: note = Linking domain
  misc_feature 41..58: note = Second complementarity domain
  source 1..104: mol_type = other RNA
nnnnnnnnnnnnnnnnnnnngttttagagctatgctgaaaagcatagcaagttaaaataaggctagtccgttatcaacttgaaaaagtggcaccgagtcggtgc

SEQ ID NO: 43 - RNA, 106 nt, synthetic construct
  misc_feature 1..106: note = gRNA
  variation 1..20: note = n is a, c, g, or u
  misc_feature 1..20: note = Targeting domain
  misc_feature 21..37: note = First complementarity domain
  misc_feature 38..41: note = Linking domain
  misc_feature 42..60: note = Second complementarity domain
  source 1..106: mol_type = other RNA
nnnnnnnnnnnnnnnnnnnngttttagagctatgctggaaacagcatagcaagttaaaataaggctagtccgttatcaacttgaaaaagtggcaccgagtcggtgc

SEQ ID NO: 44 - protein, 12 aa, Peptoniphilus duerdenii
DIGTASVGWAVT

SEQ ID NO: 45 - protein, 12 aa, Treponema denticola
DVGTGSVGWAVT

SEQ ID NO: 46 - protein, 12 aa, Streptococcus mutans
DIGTNSVGWAVV

SEQ ID NO: 47 - protein, 12 aa, Streptococcus pyogenes
DIGTNSVGWAVI

SEQ ID NO: 48 - protein, 12 aa, Listeria innocua
DIGTNSVGWAVL

SEQ ID NO: 49 - protein, 12 aa, Flavobacterium branchiophilum
DLGTNSIGWAVV

SEQ ID NO: 50 - protein, 11 aa, Pedobacter glucosidilyticus
DLGTNSIGWAI

SEQ ID NO: 51 - protein, 12 aa, Bacteroides fragilis
DLGTNSIGWALV

SEQ ID NO: 52 - protein, 12 aa, Fusobacterium nucleatum
DIGTNSVGWCVT

SEQ ID NO: 53 - protein, 12 aa, Acidaminococcus sp.
DIGTNSVGYAVT

SEQ ID NO: 54 - protein, 12 aa, Coprococcus catus
DMGTGSLGWAVT

SEQ ID NO: 55 - protein, 12 aa, Oenococcus kitaharae
DIGTSSVGWAAI

SEQ ID NO: 56 - protein, 12 aa, Catenibacterium mitsuokai
DLGTGSVGWAVV

SEQ ID NO: 57 - protein, 12 aa, Mycoplasma gallisepticum
DLGVGSVGWAIV

SEQ ID NO: 58 - protein, 12 aa, Mycoplasma ovipneumoniae
DLGIASIGWAII

SEQ ID NO: 59 - protein, 12 aa, Mycoplasma canis
DLGIASVGWAIV

SEQ ID NO: 60 - protein, 12 aa, Mycoplasma synoviae
DLGVASVGWSIV

SEQ ID NO: 61 - protein, 12 aa, Eubacterium rectale
DIGIASVGWAIL

SEQ ID NO: 62 - protein, 12 aa, Enterococcus faecalis
DLGISSVGWSVI

SEQ ID NO: 63 - protein, 12 aa, Ilyobacter polytropus
DIGIASVGWSVI

SEQ ID NO: 64 - protein, 12 aa, Ruminococcus albus
DVGIGSIGWAVI

SEQ ID NO: 65 - protein, 12 aa, Elusimicrobium minutum
DLGVGSIGFAIV

SEQ ID NO: 66 - protein, 12 aa, Akkermansia muciniphila
DIGYASIGWAVI

SEQ ID NO: 67 - protein, 12 aa, Prevotella ruminicola
DTGTNSLGWAIV

SEQ ID NO: 68 - protein, 12 aa, synthetic construct
DLGTNSIGWCLL

SEQ ID NO: 69 - protein, 12 aa, Rhodospirillum rubrum
DIGTDSLGWAVF

SEQ ID NO: 70 - protein, 12 aa, Lactobacillus rhamnosus
DIGSNSIGFAVV

SEQ ID NO: 71 - protein, 12 aa, Sphaerochaeta globosa
DLGVGSIGVAVA

SEQ ID NO: 72 - protein, 12 aa, Rhodopseudomonas palustris
DLGIASCGWGVV

SEQ ID NO: 73 - protein, 12 aa, Mycoplasma mobile
DLGIASVGWCLT

SEQ ID NO: 74 - protein, 12 aa, Streptococcus thermophilus
DIGIGSVGVGIL

SEQ ID NO: 75 - protein, 12 aa, Staphylococcus lugdunensis
DIGITSVGYGLI

SEQ ID NO: 76 - protein, 12 aa, Eubacterium dolichum
DIGITSVGFGII

SEQ ID NO: 77 - protein, 12 aa, Lactobacillus coryniformis
DVGITSTGYAVL

SEQ ID NO: 78 - protein, 12 aa, Nitratifractor salsuginis
DLGITSFGYAIL

SEQ ID NO: 79 - protein, 12 aa, Bifidobacterium bifidum
DIGNASVGWSAF

SEQ ID NO: 80 - protein, 12 aa, Lactobacillus gasseri
DVGTNSCGWVAM

SEQ ID NO: 81 - protein, 12 aa, Acidothermus cellulolyticus
DVGERSIGLAAV

SEQ ID NO: 82 - protein, 12 aa, Bifidobacterium longum
DVGLNSVGLAAV

SEQ ID NO: 83 - protein, 12 aa, Bifidobacterium dentium
DVGLMSVGLAAI

SEQ ID NO: 84 - protein, 12 aa, Corynebacterium diphtheriae
DVGTFSVGLAAI

SEQ ID NO: 85 - protein, 12 aa, Staphylococcus pseudintermedius
DIGTGSVGYACM

SEQ ID NO: 86 - protein, 12 aa, Capnocytophaga ochracea
DLGTTSIGFAHI

SEQ ID NO: 87 - protein, 12 aa, Prevotella denticola
DLGTNSIGSSVR

SEQ ID NO: 88 - protein, 12 aa, Ralstonia solanacearum
DIGTNSIGWAVI

SEQ ID NO: 89 - protein, 12 aa, Pasteurella multocida
DLGIASVGWAVV

SEQ ID NO: 90 - protein, 12 aa, Comamonas granuli
DIGIASVGWAVL

SEQ ID NO: 91 - protein, 12 aa, Helicobacter mustelae
DIGIASIGWAVI

SEQ ID NO: 92 - protein, 12 aa, Agathobacter rectalis
DIGIASVGWAII

SEQ ID NO: 93 - protein, 12 aa, Clostridium cellulolyticum
DVGIASVGWAVI

SEQ ID NO: 94 - protein, 11 aa, Methylophilus sp.
DIGIASVGWAL

SEQ ID NO: 95 - protein, 12 aa, Neisseria meningitidis
DIGIASVGWAMV

SEQ ID NO: 96 - protein, 12 aa, Clostridium perfringens
DIGITSVGWAVI

SEQ ID NO: 97 - protein, 12 aa, Wolinella succinogenes
DLGISSLGWAIV

SEQ ID NO: 98 - protein, 12 aa, Azospirillum sp.
DLGTNSIGWGLL

SEQ ID NO: 99 - protein, 12 aa, Verminephrobacter eiseniae
DLGSTSLGWAIF

SEQ ID NO: 100 - protein, 12 aa, Campylobacter jejuni
DIGISSIGWAFS

SEQ ID NO: 101 - protein, 12 aa, Parvibaculum lavamentivorans
DIGTTSIGFSVI

SEQ ID NO: 102 - protein, 12 aa, Dinoroseobacter shibae
DIGTSSIGWWLY

SEQ ID NO: 103 - protein, 12 aa, Nitrobacter hamburgensis
DLGSNSLGWFVT

SEQ ID NO: 104 - protein, 12 aa, Bradyrhizobium sp.
DLGANSLGWFVV

SEQ ID NO: 105 - protein, 15 aa, Bacillus cereus
DIGLRIGITSCGWSI

SEQ ID NO: 106 - protein, 12 aa, Sutterella wadsworthensis
DMGAKYTGVFYA

SEQ ID NO: 107 - protein, 12 aa, Wolinella succinogenes
DLGGKNTGFFSF

SEQ ID NO: 108 - protein, 12 aa, Francisella tularensis
DLGVKNTGVFSA

SEQ ID NO: 109 - protein, 12 aa, unidentified
  source 1..12: note = gamma proteobacterium
DLGAKFTGVALY

SEQ ID NO: 110 - protein, 12 aa, Legionella pneumophila
DLGGKFTGVCLS

SEQ ID NO: 111 - protein, 12 aa, Parasutterella excrementihominis
DLGGTYTGTFIT

SEQ ID NO: 112 - protein, 12 aa, Streptococcus thermophilus
DIGTNSVGWAVT

SEQ ID NO: 113 - protein, 12 aa, Eubacterium yurii
DVGTNSVGWAVT

SEQ ID NO: 114 - protein, 12 aa, Butyrivibrio hungatei
DMGTNSVGWAVT

SEQ ID NO: 115 - protein, 12 aa, Solobacterium moorei
DVGTSSVGWAVT

SEQ ID NO: 116 - protein, 27 aa, Treponema denticola
DIDHIYPQSKIKDDSISNRVLVCSSCN

SEQ ID NO: 117 - protein, 27 aa, Coprococcus catus
DIDHIYPQSKTMDDSLNNRVLVKKNYN

SEQ ID NO: 118 - protein, 27 aa, Peptoniphilus duerdenii
DQDHIYPKSKIYDDSLENRVLVKKNLN

SEQ ID NO: 119 - protein, 27 aa, Catenibacterium mitsuokai
QIDHIVPQSLVKDDSFDNRVLVVPSEN

SEQ ID NO: 120 - protein, 27 aa, Streptococcus mutans
DIDHIIPQAFIKDNSIDNRVLTSSKEN

SEQ ID NO: 121 - protein, 27 aa, Streptococcus thermophilus
DIDHIIPQAFLKDNSIDNKVLVSSASN

SEQ ID NO: 122 - protein, 27 aa, Oenococcus kitaharae
DIDHIIPQAYTKDNSLDNRVLVSNITN

SEQ ID NO: 123 - protein, 27 aa, Listeria innocua
DIDHIVPQSFITDNSIDNLVLTSSAGN

SEQ ID NO: 124 - protein, 27 aa, Streptococcus pyogenes
DVDHIVPQSFLKDDSIDNKVLTRSDKN

SEQ ID NO: 125 - protein, 27 aa, Acidaminococcus sp.
NIDHIYPQSMVKDDSLDNKVLVQSEIN

SEQ ID NO: 126 - protein, 27 aa, Lactobacillus rhamnosus
DIDHILPQSLIKDDSLDNRVLVNATIN

SEQ ID NO: 127 - protein, 27 aa, Lactobacillus gasseri
DIDHILPQSFIKDDSLENRVLVKKAVN

SEQ ID NO: 128 - protein, 27 aa, Staphylococcus pseudintermedius
EVDHIFPRSFIKDDSIDNKVLVIKKMN

SEQ ID NO: 129 - protein, 27 aa, Olsenella uli
EVDHIIPRSYIKDDSFENKVLVYREEN

SEQ ID NO: 130 - protein, 27 aa, Bifidobacterium bifidum
DIDHIIPQAVTQNDSIDNRVLVARAEN

SEQ ID NO: 131 - protein, 27 aa, Mycoplasma gallisepticum
EIDHIIPYSISFDDSSSNKLLVLAESN

SEQ ID NO: 132 - protein, 27 aa, Mycoplasma canis
EIDHIIPYSLCFDDSSANKVLVHKQSN

SEQ ID NO: 133 - protein, 27 aa, Ilyobacter polytropus
DIDHIIPYSRSMDDSYSNKVLVLSGEN

SEQ ID NO: 134 - protein, 27 aa, synthetic construct
DIDHIIPYSKSMDDSFNNKVLCLAEEN

SEQ ID NO: 135 - protein, 27 aa, Campylobacter jejuni
EIDHIYPYSRSFDDSYMNKVLVFTKQN

SEQ ID NO: 136 - protein, 27 aa, Clostridium cellulolyticum
QIDHIYPYSRSMDDSYMNKVLVLTDEN

SEQ ID NO: 137 - protein, 27 aa, Clostridium perfringens
EIDHIIPFSRSFDDSLSNKILVLGSEN

SEQ ID NO: 138 - protein, 27 aa, Neisseria meningitidis
EIDHALPFSRTWDDSFNNKVLVLGSEN

SEQ ID NO: 139 - protein, 27 aa, Pasteurella multocida
EIDHALPFSRTWDDSFNNKVLVLASEN

SEQ ID NO: 140 - protein, 27 aa, Enterococcus faecalis
EIDHIIPISISLDDSINNKVLVLSKAN

SEQ ID NO: 141 - protein, 27 aa, Eubacterium dolichum
EVDHIIPISISLDDSITNKVLVTHREN

SEQ ID NO: 142 - protein, 27 aa, Acidovorax ebreus
QVDHALPYSRSYDDSKNNKVLVLTHEN

SEQ ID NO: 143 - protein, 27 aa, Streptococcus thermophilus
EVDHILPLSITFDDSLANKVLVYATAN

SEQ ID NO: 144 - protein, 27 aa, Eubacterium rectale
EIDHIIPRSISFDDARSNKVLVYRSEN

SEQ ID NO: 145 - protein, 27 aa, Staphylococcus lugdunensis
EVDHIIPRSVSFDNSYHNKVLVKQSEN

SEQ ID NO: 146 - protein, 27 aa, Roseburia intestinalis
DIDHILPYSITFDDSFRNKVLVTSQEN

SEQ ID NO: 147 - protein, 27 aa, Wolinella succinogenes
EIDHILPRSRSADDSFANKVLCLARAN

SEQ ID NO: 148 - protein, 27 aa, synthetic construct
EIEHLLPFSLTLDDSMANKTVCFRQAN

SEQ ID NO: 149 - protein, 27 aa, Azospirillum sp.
DIDHILPFSVSLDDSAANKVVCLREAN

SEQ ID NO: 150 - protein, 27 aa, Bradyrhizobium sp.
DIDHLIPFSISWDDSAANKVVCMRYAN

SEQ ID NO: 151 - protein, 27 aa, Nitrobacter hamburgensis
DIDHILPVAMTLDDSPANKIICMRYAN

SEQ ID NO: 152 - protein, 27 aa, Dinoroseobacter shibae
DVDHILPYSRTLDDSFPNRTLCLREAN

SEQ ID NO: 153 - protein, 27 aa, Verminephrobacter eiseniae
EIEHILPFSRTLDDSLNNRTVAMRRAN

SEQ ID NO: 154 - protein, 27 aa, Lactobacillus coryniformis
EVDHIIPYSISWDDSYTNKVLTSAKCN

SEQ ID NO: 155 - protein, 27 aa, Rhodopseudomonas palustris
QVDHILPWSRFGDDSYLNKTLCTARSN

SEQ ID NO: 156 - protein, 27 aa, Ralstonia syzygii
QVDHILPFSKTLDDSFANKVLAQHDAN

SEQ ID NO: 157 - protein, 27 aa, Helicobacter mustelae
QIDHAFPLSRSLDDSQSNKVLCLTSSN

SEQ ID NO: 158 - protein, 27 aa, Mycoplasma mobile
DIDHIVPRSISFDDSFSNLVIVNKLDN

SEQ ID NO: 159 - protein, 27 aa, Mycoplasma ovipneumoniae
EIEHIIPYSMSYDNSQANKILTEKAEN

SEQ ID NO: 160 - protein, 27 aa, Mycoplasma synoviae
EIDHVIPYSKSADDSWFNKLLVKKSTN

SEQ ID NO: 161 - protein, 27 aa, Aminomonas paucivorans
EMDHILPYSRSLDNGWHNRVLVHGKDN

SEQ ID NO: 162 - protein, 27 aa, Ruminococcus albus
EVDHIVPYSLILDNTINNKALVYAEEN

SEQ ID NO: 163 - protein, 27 aa, Fibrobacter succinogenes
EIEHVIPQSLYFDDSFSNKVICEAEVN

SEQ ID NO: 164 - protein, 27 aa, Bacteroides fragilis
DIEHIIPQARLFDDSFSNKTLEARSVN

SEQ ID NO: 165 - protein, 27 aa, Capnocytophaga sputigena
EIEHIVPKARVFDDSFSNKTLTFHRIN

SEQ ID NO: 166 - protein, 28 aa, Finegoldia magna
DKDHIIPQSMKKDDSIINNLVLVNKNAN

SEQ ID NO: 167 - protein, 27 aa, Parvibaculum lavamentivorans
EVEHIWPRSRSFDNSPRNKTLCRKDVN

SEQ ID NO: 168 - protein, 27 aa, Bacillus cereus
IVNHIIPYNRSFDDTYHNRVLTLTETK

SEQ ID NO: 169 - protein, 27 aa, Prevotella micans
DMEHTIPKSISFDNSDQNLTLCESYYN

SEQ ID NO: 170 - protein, 27 aa, Prevotella ruminicola
DIEHTIPRSAGGDSTKMNLTLCSSRFN

SEQ ID NO: 171 - protein, 27 aa, Flavobacterium columnare
DIEHTIPRSISQDNSQMNKTLCSLKFN

SEQ ID NO: 172 - protein, 27 aa, Rhodospirillum rubrum
DIDHVIPLARGGRDSLDNMVLCQSDAN

SEQ ID NO: 173 - protein, 27 aa, Elusimicrobium minutum
DIEHLFPIAESEDNGRNNLVISHSACN

SEQ ID NO: 174 - protein, 27 aa, Sphaerochaeta globosa
DVDHIFPRDDTADNSYGNKVVAHRQCN

SEQ ID NO: 175 - protein, 27 aa, Nitratifractor salsuginis
DIEHIVPQSLGGLSTDYNTIVTLKSVN

SEQ ID NO: 176 - protein, 27 aa, Acidothermus cellulolyticus
ELDHIVPRTDGGSNRHENLAITCGACN

SEQ ID NO: 177 - protein, 28 aa, Bifidobacterium longum
EMDHIVPRKGVGSTNTRTNFAAVCAECN

SEQ ID NO: 178 - protein, 28 aa, Bifidobacterium dentium
EMDHIVPRKGVGSTNTRVNLAAACAACN

SEQ ID NO: 179 - protein, 28 aa, Corynebacterium diphtheriae
EMDHIVPRAGQGSTNTRENLVAVCHRCN

SEQ ID NO: 180 - protein, 33 aa, Sutterella wadsworthensis
EIDHILPRSLIKDARGIVFNAEPNLIYASSRGN

SEQ ID NO: 181 - protein, 33 aa, unidentified
  source 1..33: note = gamma proteobacterium
EIDHIIPRSLTGRTKKTVFNSEANLIYCSSKGN

SEQ ID NO: 182 - protein, 33 aa, Parasutterella excrementihominis
EIDHIIPRSLTLKKSESIYNSEVNLIFVSAQGN

SEQ ID NO: 183 - protein, 33 aa, Legionella pneumophila
EIDHIYPRSLSKKHFGVIFNSEVNLIYCSSQGN

SEQ ID NO: 184 - protein, 33 aa, Wolinella succinogenes
EIDHILPRSHTLKIYGTVFNPEGNLIYVHQKCN

SEQ ID NO: 185 - protein, 30 aa, Francisella tularensis
ELDHIIPRSHKKYGTLNDEANLICVTRGDN

SEQ ID NO: 186 - protein, 27 aa, Akkermansia muciniphila
ELEHIVPHSFRQSNALSSLVLTWPGVN

SEQ ID NO: 187 - protein, 27 aa, Solobacterium moorei
DIDHIYPRSKIKDDSITNRVLVEKDIN

SEQ ID NO: 188 - protein, 28 aa, Veillonella atypica
YDIDHIYPRSLTKDDSFDNLVLCERTAN

SEQ ID NO: 189 - protein, 28 aa, Fusobacterium nucleatum
DIDHIYPRSKVIKDDSFDNLVLVLKNEN

SEQ ID NO: 190 - protein, 27 aa, Filifactor alocis
DRDHIYPQSKIKDDSIDNLVLVNKTYN

SEQ ID NO: 191-197 - skipped (7 entries)

SEQ ID NO: 198 - DNA, 4758 bp, Homo sapiens
  misc_feature 2716..2719: note = HPFH deletion site (4 bp del -225 to -222)
  misc_feature 2748..2753: note = GATA1 binding motif
  misc_feature 2762..2767: note = GATA1 binding motif
  misc_feature 2791..2799: note = FKLF transcription factor binding motif
  misc_feature 2823..2830: note = CP1/Coup TFII binding motif
  misc_feature 2824..2836: note = HPFH deletion site (13 bp del -114 to -102)
  misc_feature 2991..2993: note = Start codon
  source 1..4758: mol_type = other DNA
tttaggaagtcaaggtttaggcagggatagccattctattttattaggggcaatactatttccaacggcatctggcttttctcagcccttgtgaggctctacagggaggttgaggtgttagagatcagagcaggaaacaggtttttctttccacggtaactacaatgaagtgatccttactttactaaggaacttttcattttaagtgttgacgcatgcctaaagaggtgaaattaatcccatacccttaagtctacagactggtcacagcatttcaaggaggagacctcattgtaagcttctagggaggtggggacttaggtgaaggaaatgagccagcagaagctcacaagtcagcatcagcgtgtcatgtctcagcagcagaacagcacggtcagatgaaaatatagtgtgaagaatttgtataacattaattgagaaggcagattcactggagttcttatataattgaaagttaatgcacgttaataagcaagagtttagtttaatgtgatggtgttatgaacttaacgcttgtgtctccagaaaattcacatgctgaatccccaactcccaattggctccatttgtgggggaggctttggaaaagtaatcaggtttagaggagctcatgagagcagatccccatcatagaattattttcctcatcagaagcagagagattagccatttctcttccttctggtgaggacacagtgggaagtcagccacctgcaacccaggaagagagccctgaccaggaaccagcagaaaagtgagaaaaaatcctgttgttgaagtcacccagtctatgctattttgttatagcaccttgcactaagtaaggcagatgaagaaagagaaaaaaataagcttcggtgttcagtggattagaaaccatgtttatctcaggtttacaaatctccacttgtcctctgtgtttcagaataaaataccaactctactactctcatctgtaagatgcaaatagtaagcctgagcccttctgtctaactttgaattctattttttcttcaacgtactttaggcttgtaatgtgtttatatacagtgaaatgtcaagttctttctttatatttctttctttcttttttttcctcagcctcagagttttccacatgcccttcctactttcaggaacttctttctccaaacgtcttctgcctggctccatcaaatcataaaggacccacttcaaatgccatcactcactaccatttcacaattcgcactttctttctttgtcctttttttttttagtaaaacaagtttataaaaaattgaaggaataaatgaatggctacttcataggcagagtagacgcaagggctactggttgccgatttttattgttatttttcaatagtatgctaaacaaggggtagattatttatgctgcccatttttagaccataaaagataacttcctgatgttgccatggcatttttttccttttaattttatttcatttcattttaatttcgaaggtacatgtgcaggatgtgcaggcttgttacatgggtaaatgtgtgtctttctggccttttagccatctgtatcaatgagcagatataagctttacacaggatcatgaaggatgaaagaatttcaccaatattataataatttcaatcaacctgatagcttaggggataaactaatttgaagatacagcttgcctccgataagccagaattccagagcttctggcattataatctagcaaggttagagatcatggatcactttcagagaaaaacaaaaacaaactaaccaaaagcaaaacagaaccaaaaaaccaccataaatacttcctaccctgttaatggtccaatatgtcagaaacagcactgtgttagaaataaagctgtctaaagtacactaatattcgagttataatagtgtgtggactattagtcaataaaaacaacccttgcctctttagagttgttttccatgtacacgcacatcttatgtcttagagtaagattccctgagaagtgaacctagcatttatacaagataattaattctaatccacagtacctgccaaagaacattctaccatcatctttactgagcatagaagagctacgccaaaaccctgggtcatcagccagcacacacacttatccagtggtaaatacacatcatctggtgtatacatacatacctgaatatggaatcaaatatttttctaagatgaaacagtcatgatttatttcaaataggtacggataagtagatattgaggtaagcattaggtcttatattatgtaacactaatctattactgcgctgaaactgtggctttatagaaattgttttcactgcactattgagaaattaagagataatggcaaaagtcacaaagagtatattcaaaaagaagtatagcactttttccttagaaaccactgctaactgaaagagactaagatttgtcccgtcaaaaatcctggacctatgcctaaaacacatttcacaatccctgaacttttcaaaaattggtacatgctttagctttaaactacaggcctcactggagctagagacaagaaggtaaaaaacggctgacaaaagaagtcctggtatcctctatgatgggagaaggaaactagctaaagggaagaataaattagagaaaaactggaatgactgaatcggaacaaggcaaaggctataaaaaaaattagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccacaaagcacctggatgatctcaagggcacctttgcccagctgagtgaactgcactgtgacaagctgcatgtggatcctgagaacttcaaggtgagtccaggagatgtttcagccctgttgcctttagtctcgaggcaacttagacaacggagtattgatctgagcacagcagggtgtgagctgtttgaagatactggggttgggggtgaagaaactgcagaggactaactgggctgagacccagtggtaatgttttagggcctaaggagtgcctctaaaaatctagatggacaattttgactttgagaaaagagaggtggaaatgaggaaaatgacttttctttattagattccagtagaaagaactttcatctttccctcatttttgttgttttaaaacatctatctggaggcaggacaagtatggtcgttaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgcgcgcgcgcgtgtgtgtgtgtgtgtcagcgtgtgtttcttttaacgtcttcagcctacaacatacagggttcatggtggcaagaagatagcaagatttaaattatggccagtgactagtgcttgaaggggaacaactacctgcatttaatgggaaggcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactgcagtggccagtgccctgtcctccagataccactgagctcactgcccatgattcagagctttcaaggataggctttattctgcaagcaatacaaataataaatctattctgctgagagatcacacatgattttcttcagctcttttttttacatctttttaaatatatgagccacaaagggtttatattgagggaagtgtgtatgtgtatttctgcatgcctgtttgtgtttgtggtgtgtgcatgctcctcatttatttttatatgagatgtgcattttgatgagcaaataaaagcagtaaagacacttgtacacgggagttctgcaagtgggagtaaatggtgtaggagaaatccg

SEQ ID NO: 199 - DNA, 4773 bp, Homo sapiens
  misc_feature 1233..1238: note = GATA1 binding site
  misc_feature 2672..2677: note = GATA1 binding site
  misc_feature 2686..2691: note = GATA1 binding site
  misc_feature 2715..2723: note = FKLF transcription factor binding motif
  misc_feature 2747..2754: note = CP1/Coup TFII binding motif
  misc_feature 2748..2760: note = HPFH deletion site (13 bp del -114 to -102)
  misc_feature 2915..2917: note = Start codon
  source 1..4773: mol_type = other DNA
ttatgtcattaccagagttaaaattctataatggcttctcactccctaccactgaggacaagtttatgtccttaggtttatgcttccctgaaacaataccacctgctattctccactttacatatcaacggcactggttctttatctaactctctggcacagcaggagtttgttttcttctgcttcagagctttgaatttactatttcagcttctaaactttatttggcaatgccttcccatggcagattccttctgtcattttgcctctgttcgaatactttctccttaatttcattcttagttaataatatctgaaattattttgttgtttaacttaattattaattttatgtatgttctacctagattataatcttcagaggaaagttttattctctgacttatttaacttaaatgcccactactttaaaaattatgacatttatttaacagatatttgctgaacaaatgtttgaaaatacatgggaaagaatgcttgaaaacacttgaaattgcttgtgtaaagaaacagttttatcagttaggatttaatcaatgtcagaagcaatgatataggaaaaatcgaggaataagacagttatggataaggagaaatcaacaaactcttaaaagatattgcctcaaaagcataagaggaaataagggtttatacatgacttttagaacactgccttggtttttggataaatggggaagttgtttgaaaacaggagggatcctagatattccttagtctgaggaggagcaattaagattcacttgtttagaggctgggagtggtggctcacgcctgtaatcccagaattttgggaggccaaggcaggcagatcacctgaggtcaagagttcaagaccaacctggccaacatggtgaaatcccatctctacaaaaatacaaaaattagacaggcatgatggcaagtgcctgtaatcccagctacttgggaggctgaggaaggagaattgcttgaacctggaaggcaggagttgcagtgagccgagatcataccactgcactccagcctgggtgacagaacaagactctgtctcaaaaaaaaaaaagagagattcaaaagattcacttgtttaggccttagcgggcttagacaccagtctctgacacattcttaaaggtcaggctctacaaatggaacccaaccagactctcagatatggccaaagatctatacacacccatctcacagatcccctatcttaaagagaccctaatttgggttcacctcagtctctataatctgtaccagcataccaataaaaatctttctcacccatccttagattgagagaagtcacttattattatgtgagtaactggaagatactgataagttgacaaatctttttctttcctttcttattcaacttttattttaacttccaaagaacaagtgcaatatgtgcagctttgttgcgcaggtcaacatgtatctttctggtcttttagccgcctaacactttgagcagatataagccttacacaggattatgaagtctgaaaggattccaccaatattattataattcctatcaacctgataggttaggggaaggtagagctctcctccaataagccagatttccagagtttctgacgtcataatctaccaaggtcatggatcgagttcagagaaaaaacaaaagcaaaaccaaacctaccaaaaaataaaaatcccaaagaaaaaataaagaaaaaaacagcatgaatacttcctgccatgttaagtggccaatatgtcagaaacagcactgagttacagataaagatgtctaaactacagtgacatcccagctgtcacagtgtgtggactattagtcaataaaacagtccctgcctcttaagagttgttttccatgcaaatacatgtcttatgtcttagaataagattccctaagaagtgaacctagcatttatacaagataattaattctaatccatagtatctggtaaagagcattctaccatcatctttaccgagcatagaagagctacaccaaaaccctgggtcatcagccagcacatacacttatccagtgataaatacacatcatcgggtgcctacatacatacctgaatataaaaaaaatacttttgctgagatgaaacaggcgtgatttatttcaaataggtacggataagtagatattgaagtaaggattcagtcttatattatattacataacattaatctattcctgcactgaaactgttgctttataggatttttcactacactaatgagaacttaagagataatggcctaaaaccacagagagtatattcaaagataagtatagcacttcttatttggaaaccaatgcttactaaatgagactaagacgtgtcccatcaaaaatcctggacctatgcctaaaacacatttcacaatccctgaacttttcaaaaattggtacatgctttaactttaaactacaggcctcactggagctacagacaagaaggtgaaaaacggctgacaaaagaagtcctggtatcttctatggtgggagaagaaaactagctaaagggaagaataaattagagaaaaattggaatgactgaatcggaacaaggcaaaggctataaaaaaaattaagcagcagtatcctcttgggggccccttccccacactatctcaatgcaaatatctgtctgaaacggtccctggctaaactccacccatgggttggccagccttgccttgaccaatagccttgacaaggcaaacttgaccaatagtcttagagtatccagtgaggccaggggccggcggctggctagggatgaagaataaaaggaagcacccttcagcagttccacacactcgcttctggaacgtctgaggttatcaataagctcctagtccagacgccatgggtcatttcacagaggaggacaaggctactatcacaagcctgtggggcaaggtgaatgtggaagatgctggaggagaaaccctgggaaggtaggctctggtgaccaggacaagggagggaaggaaggaccctgtgcctggcaaaagtccaggtcgcttctcaggatttgtggcaccttctgactgtcaaactgttcttgtcaatctcacaggctcctggttgtctacccatggacccagaggttctttgacagctttggcaacctgtcctctgcctctgccatcatgggcaaccccaaagtcaaggcacatggcaagaaggtgctgacttccttgggagatgccataaagcacctggatgatctcaagggcacctttgcccagctgagtgaactgcactgtgacaagctgcatgtggatcctgagaacttcaaggtgagtccaggagatgtttcagcactgttgcctttagtctcgaggcaacttagacaactgagtattgatctgagcacagcagggtgtgagctgtttgaagatactggggttgggagtgaagaaactgcagaggactaactgggctgagacccagtggcaatgttttagggcctaaggagtgcctctgaaaatctagatggacaactttgactttgagaaaagagaggtggaaatgaggaaaatgacttttctttattagatttcggtagaaagaactttcacctttcccctatttttgttattcgttttaaaacatctatctggaggcaggacaagtatggtcattaaaaagatgcaggcagaaggcatatattggctcagtcaaagtggggaactttggtggccaaacatacattgctaaggctattcctatatcagctggacacatataaaatgctgctaatgcttcattacaaacttatatcctttaattccagatgggggcaaagtatgtccaggggtgaggaacaattgaaacatttgggctggagtagattttgaaagtcagctctgtgtgtgtgtgtgtgtgtgtgcgcgcgtgtgtttgtgtgtgtgtgagagcgtgtgtttcttttaacgttttcagcctacagcatacagggttcatggtggcaagaagataacaagatttaaattatggccagtgactagtgctgcaagaagaacaactacctgcatttaatgggaaagcaaaatctcaggctttgagggaagttaacataggcttgattctgggtggaagcttggtgtgtagttatctggaggccaggctggagctctcagctcactatgggttcatctttattgtctcctttcatctcaacagctcctgggaaatgtgctggtgaccgttttggcaatccatttcggcaaagaattcacccctgaggtgcaggcttcctggcagaagatggtgactggagtggccagtgccctgtcctccagataccactgagctcactgcccatgatgcagagctttcaaggataggctttattctgcaagcaatcaaataataaatctattctgctaagagatcacacatggttgtcttcagttctttttttatgtctttttaaatatatgagccacaaagggttttatgttgagggatgtgtttatgtgtatttatacatggctatgtgtgtttgtgtcatgtgcacactccacacttttttgtttacgttagatgtgggttttgatgagcaaataaaagaactaggcaataaagaaacttgtacatgggagttctgcaagtgggagtaaaaggtgcaggagaaatctggttggaagaaagacctctataggacaggactcctcagaaacagatgttttggaagagatggggaaaggttcagtgaa

SEQ ID NO: 200 - protein, 1332 aa, synthetic construct
  REGION 1..1332: note = His-Cas12a-nNLS
MKHHHHHHMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKG

SEQ ID NO: 201 - protein, 1333 aa, synthetic construct
  REGION 1..1333: note = His-Cas12a-sNLS-sNLS
MKHHHHHHMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 202 - RNA, 20 nt, synthetic construct
  misc_feature 1..20: note = HBG1-1 gRNA (OLI13620) targeting domain
  source 1..20: mol_type = other RNA
ccttgtcaaggctattggtc

SEQ ID NO: 203 - protein, 16 aa, synthetic construct
  REGION 1..16: note = Nucleoplasmin NLS
KRPAATKKAGQAKKKK

SEQ ID NO: 204 - protein, 7 aa, synthetic construct
  REGION 1..7: note = Simian Virus 40 NLS
PKKKRKV

SEQ ID NO: 205 - protein, 1324 aa, synthetic construct
  REGION 1..1324: note = His-Cas12a-sNLS
  REGION 3..8: note = MISC_FEATURE - His tag
  REGION 1318..1324: note = MISC_FEATURE - SV40 NLS
MKHHHHHHMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKV

SEQ ID NO: 206 - protein, 1326 aa, synthetic construct
  REGION 1..1326: note = His-sNLS-Cas12a
  REGION 3..8: note = MISC_FEATURE - His tag
  REGION 11..17: note = MISC_FEATURE - SV40 NLS
MKHHHHHHGSPKKKRKVGSMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 207 - protein, 1333 aa, synthetic construct
  REGION 1..1333: note = His-sNLS-sNLS-Cas12a
  REGION 3..8: note = MISC_FEATURE - His tag
  REGION 11..17: note = MISC_FEATURE - SV40 NLS
  REGION 20..26: note = MISC_FEATURE - SV40 NLS
MKHHHHHHGSPKKKRKVGSPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 208 - protein, 1327 aa, synthetic construct
  REGION 1..1327: note = sNLS-sNLS-Cas12a
  REGION 5..11: note = MISC_FEATURE - SV40 NLS
  REGION 14..20: note = MISC_FEATURE - SV40 NLS
MKGSPKKKRKVGSPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 209 - protein, 1332 aa, synthetic construct
  REGION 1..1332: note = His-sNLS-Cas12a-sNLS
  REGION 3..8: note = MISC_FEATURE - His tag
  REGION 10..16: note = MISC_FEATURE - SV40 NLS
  REGION 1325..1331: note = MISC_FEATURE - SV40 NLS
MKHHHHHHMPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGPKKKRKVG

SEQ ID NO: 210 - protein, 1352 aa, synthetic construct
  REGION 1..1352: note = His-sNLS-sNLS-Cas12a-sNLS-sNLS
  REGION 3..8: note = MISC_FEATURE - His tag
  REGION 11..17: note = MISC_FEATURE - SV40 NLS
  REGION 20..26: note = MISC_FEATURE - SV40 NLS
  REGION 1337..1343: note = MISC_FEATURE - SV40 NLS
  REGION 1346..1352: note = MISC_FEATURE - SV40 NLS
MGHHHHHHGSPKKKRKVGSPKKKRKVGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 211 - protein, 1332 aa, synthetic construct
  REGION 1..1332: note = His-Cas12a-nNLS Cys-less
  REGION 3..8: note = MISC_FEATURE - His tag
  REGION 1316..1331: note = MISC_FEATURE - Nucleoplasmin NLS
MKHHHHHHMTQFEGFTNLYQVSKTLRFELIPQGKTLKHIQEQGFIEEDKARNDHYKELKPIIDRIYKTYADQSLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENSHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFSKYKTLLRNENVLETAEALFNELNSIDLTHIFISHKKLETISSALSDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKSSTQLKAVTAHFQTHTTPILLSNNFIEPLEITKEIYDLNNPEKEPKKFQTAYAKKTGDQKGYREALSKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNSLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVSFDSRFQNPEWPMDADANGAYHIALKGQLLLNHLKESKDLKLQNGISNQDWLAYIQELRNKRPAATKKAGQAKKKKG

SEQ ID NO: 212 - protein, 1332 aa, synthetic construct
  REGION 1..1332: note = His-Cas12a-nNLS Cys-low
  REGION 3..8: note = MISC_FEATURE - His tag
  REGION 1316..1331: note = MISC_FEATURE - Nucleoplasmin NLS
MKHHHHHHMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFSKYKTLLRNENVLETAEALFNELNSIDLTHIFISHKKLETISSALSDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALSKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNKRPAATKKAGQAKKKKG

SEQ ID NO: 213 - protein, 1307 aa, Acidaminococcus sp.
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRN

SEQ ID NO: 214 - protein, 1228 aa, synthetic construct
MSKLEKFTNCYSLSKTLRFKAIPVGKTQENIDNKRLLVEDEKRAEDYKGVKKLLDRYYLSFINDVLHSIKLKNLNNYISLFRKKTRTEKENKELENLEINLRKEIAKAFKGNEGYKSLFKKDIIETILPEFLDDKDEIALVNSFNGFTTAFTGFFDNRENMFSEEAKSTSIAFRCINENLTRYISNMDIFEKVDAIFDKHEVQEIKEKILNSDYDVEDFFEGEFFNFVLTQEGIDVYNAIIGGFVTESGEKIKGLNEYINLYNQKTKQKLPKFKPLYKQVLSDRESLSFYGEGYTSDEEVLEVFRNTLNKNSEIFSSIKKLEKLFKNFDEYSSAGIFVKNGPAISTISKDIFGEWNVIRDKWNAEYDDIHLKKKAVVTEKYEDDRRKSFKKIGSFSLEQLQEYADADLSVVEKLKEIIIQKVDEIYKVYGSSEKLFDADFVLEKSLKKNDAVVAIMKDLLDSVKSFENYIKAFFGEGKETNRDESFYGDFVLAYDILLKVDHIYDAIRNYVTQKPYSKDKFKLYFQNPQFMGGWDKDKETDYRATILRYGSKYYLAIMDKKYAKCLQKIDKDDVNGNYEKINYKLLPGPNKMLPKVFFSKKWMAYYNPSEDIQKIYKNGTFKKGDMFNLNDCHKLIDFFKDSISRYPKWSNAYDFNFSETEKYKDIAGFYREVEEQGYKVSFESASKKEVDKLVEEGKLYMFQIYNKDFSDKSHGTPNLHTMYFKLLFDENNHGQIRLSGGAELFMRRASLKKEELVVHPANSPIANKNPDNPKKTTTLSYDVYKDKRFSEDQYELHIPIAINKCPKNIFKINTEVRVLLKHDDNPYVIGIDRGERNLLYIVVVDGKGNIVEQYSLNEIINNFNGIRIKTDYHSLLDKKEKERFEARQNWTSIENIKELKAGYISQVVHKICELVEKYDAVIALEDLNSGFKNSRVKVEKQVYQKFEKMLIDKLNYMVDKKSNPCATGGALKGYQITNKFESFKSMSTQNGFIFYIPAWLTSKIDPSTGFVNLLKTKYTSIADSKKFISSFDRIMYVPEEDLFEFALDYKNFSRTDADYIKKWKLYSYGNRIRIFRNPKKNNVFDWEEVCLTSAYKELFNKYGINYQQGDIRALLCEQSDKAFYSSFMALMSLMLQMRNSITGRTDVDFLISPVKNSDGIFYDSRNYEAQENAILPKNADANGAYNIARKVLWAIGQFKKAEDEKLDKVKIAISNKEWLEYAQTSVKH

SEQ ID NO: 215 - protein, 1206 aa, synthetic construct
MYYESLTKQYPVSKTIRNELIPIGKTLDNIRQNNILESDVKRKQNYEHVKGILDEYHKQLINEALDNCTLPSLKIAAEIYLKNQKEVSDREDFNKTQDLLRKEVVEKLKAHENFTKIGKKDILDLLEKLPSISEDDYNALESFRNFYTYFTSYNKVRENLYSDKEKSSTVAYRLINENFPKFLDNVKSYRFVKTAGILADGLGEEEQDSLFIVETFNKTLTQDGIDTYNSQVGKINSSINLYNQKNQKANGFRKIPKMKMLYKQILSDREESFIDEFQSDEVLIDNVESYGSVLIESLKSSKVSAFFDALRESKGKNVYVKNDLAKTAMSNIVFENWRTFDDLLNQEYDLANENKKKDDKYFEKRQKELKKNKSYSLEHLCNLSEDSCNLIENYIHQISDDIENIIINNETFLRIVINEHDRSRKLAKNRKAVKAIKDFLDSIKVLERELKLINSSGQELEKDLIVYSAHEELLVELKQVDSLYNMTRNYLTKKPFSTEKVKLNFNRSTLLNGWDRNKETDNLGVLLLKDGKYYLGIMNTSANKAFVNPPVAKTEKVFKKVDYKLLPVPNQMLPKVFFAKSNIDFYNPSSEIYSNYKKGTHKKGNMFSLEDCHNLIDFFKESISKHEDWSKFGFKFSDTASYNDISEFYREVEKQGYKLTYTDIDETYINDLIERNELYLFQIYNKDFSMYSKGKLNLHTLYFMMLFDQRNIDDVVYKLNGEAEVFYRPASISEDELIIHKAGEEIKNKNPNRARTKETSTFSYDIVKDKRYSKDKFTLHIPITMNFGVDEVKRFNDAVNSAIRIDENVNVIGIDRGERNLLYVVVIDSKGNILEQISLNSIINKEYDIETDYHALLDEREGGRDKARKDWNTVENIRDLKAGYLSQVVNVVAKLVLKYNAIICLEDLNFGFKRGRQKVEKQVYQKFEKMLIDKLNYLVIDKSREQTSPKELGGALNALQLTSKFKSFKELGKQSGVIYYVPAYLTSKIDPTTGFANLFYMKCENVEKSKRFFDGFDFIRFNALENVFEFGFDYRSFTQRACGINSKWTVCTNGERIIKYRNPDKNNMFDEKVVVVTDEMKNLFEQYKIPYEDGRNVKDMIISNEEAEFYRRLYRLLQQTLQMRNSTSDGTRDYIISPVKNKREAYFNSELSDGSVPKDADANGAYNIARKGLWVLEQIRQKSEGEKINLAMTNAEWLEYAQTHLL

SEQ ID NO: 216 - DNA, 4059 bp, Acidaminococcus sp.
  source 1..4059: mol_type = other DNA
atgacccagttcgagggcttcaccaacctgtaccaggtgagcaagaccctgcggttcgagctgatcccccagggcaagaccctgaagcacatccaggagcagggcttcatcgaggaggacaaggcccggaacgaccactacaaggagctgaagcccatcatcgaccggatctacaagacctacgccgaccagtgcctgcagctggtgcagctggactgggagaacctgagcgccgccatcgacagctaccggaaggagaagaccgaggagacccggaacgccctgatcgaggagcaggccacctaccggaacgccatccacgactacttcatcggccggaccgacaacctgaccgacgccatcaacaagcggcacgccgagatctacaagggcctgttcaaggccgagctgttcaacggcaaggtgctgaagcagctgggcaccgtgaccaccaccgagcacgagaacgccctgctgcggagcttcgacaagttcaccacctacttcagcggcttctacgagaaccggaagaacgtgttcagcgccgaggacatcagcaccgccatcccccaccggatcgtgcaggacaacttccccaagttcaaggagaactgccacatcttcacccggctgatcaccgccgtgcccagcctgcgggagcacttcgagaacgtgaagaaggccatcggcatcttcgtgagcaccagcatcgaggaggtgttcagcttccccttctacaaccagctgctgacccagacccagatcgacctgtacaaccagctgctgggcggcatcagccgggaggccggcaccgagaagatcaagggcctgaacgaggtgctgaacctggccatccagaagaacgacgagaccgcccacatcatcgccagcctgccccaccggttcatccccctgttcaagcagatcctgagcgaccggaacaccctgagcttcatcctggaggagttcaagagcgacgaggaggtgatccagagcttctgcaagtacaagaccctgctgcggaacgagaacgtgctggagaccgccgaggccctgttcaacgagctgaacagcatcgacctgacccacatcttcatcagccacaagaagctggagaccatcagcagcgccctgtgcgaccactgggacaccctgcggaacgccctgtacgagcggcggatcagcgagctgaccggcaagatcaccaagagcgccaaggagaaggtgcagcggagcctgaagcacgaggacatcaacctgcaggagatcatcagcgccgccggcaaggagctgagcgaggccttcaagcagaagaccagcgagatcctgagccacgcccacgccgccctggaccagcccctgcccaccaccctgaagaagcaggaggagaaggagatcctgaaaagccagctggacagcctgctgggcctgtaccacctgctggactggttcgccgtggacgagagcaacgaggtggaccccgagttcagcgcccggctgaccggcatcaagctggagatggagcccagcctgagcttctacaacaaggcccggaactacgccaccaagaagccctacagcgtggagaagttcaagctgaacttccagatgcccaccctggccagcggctgggacgtgaacaaggagaagaacaacggcgccatcctgttcgtgaagaacggcctgtactacctgggcatcatgcccaagcagaagggccggtacaaggccctgagcttcgagcccaccgagaagaccagcgagggcttcgacaagatgtactacgactacttccccgacgccgccaagatgatccccaagtgcagcacccagctgaaggccgtgaccgcccacttccagacccacaccacccccatcctgctgagcaacaacttcatcgagcccctggagatcaccaaggagatctacgacctgaacaaccccgagaaggagcccaagaagttccagaccgcctacgccaagaagaccggcgaccagaagggctaccgggaggccctgtgcaagtggatcgacttcacccgggacttcctgagcaagtacaccaagaccaccagcatcgacctgagcagcctgcggcccagcagccagtacaaggacctgggcgagtactacgccgagctgaaccccctgctgtaccacatcagcttccagcggatcgccgagaaggagatcatggacgccgtggagaccggcaagctgtacctgttccagatctacaacaaggacttcgccaagggccaccacggcaagcccaacctgcacaccctgtactggaccggcctgttcagccccgagaacctggccaagaccagcatcaagctgaacggccaggccgagctgttctaccggcccaagagccggatgaagcggatggcccaccggctgggcgagaagatgctgaacaagaagctgaaggaccagaagacccccatccccgacaccctgtaccaggagctgtacgactacgtgaaccaccggctgagccacgacctgagcgacgaggcccgggccctgctgcccaacgtgatcaccaaggaggtgagccacgagatcatcaaggaccggcggttcaccagcgacaagttcttcttccacgtgcccatcaccctgaactaccaggccgccaacagccccagcaagttcaaccagcgggtgaacgcctacctgaaggagcaccccgagacccccatcatcggcatcgaccggggcgagcggaacctgatctacatcaccgtgatcgacagcaccggcaagatcctggagcagcggagcctgaacaccatccagcagttcgactaccagaagaagctggacaaccgggagaaggagcgggtggccgcccggcaggcctggagcgtggtgggcaccatcaaggacctgaagcagggctacctgagccaggtgatccacgagatcgtggacctgatgatccactaccaggccgtggtggtgctggagaacctgaacttcggcttcaagagcaagcggaccggcatcgccgagaaggccgtgtaccagcagttcgagaagatgctgatcgacaagctgaactgcctggtgctgaaggactaccccgccgagaaggtgggcggcgtgctgaacccctaccagctgaccgaccagttcaccagcttcgccaagatgggcacccagagcggcttcctgttctacgtgcccgccccctacaccagcaagatcgaccccctgaccggcttcgtggaccccttcgtgtggaagaccatcaagaaccacgagagccggaagcacttcctggagggcttcgacttcctgcactacgacgtgaagaccggcgacttcatcctgcacttcaagatgaaccggaacctgagcttccagcggggcctgcccggcttcatgcccgcctgggacatcgtgttcgagaagaacgagacccagttcgacgccaagggcacccccttcatcgccggcaagcggatcgtgcccgtgatcgagaaccaccggttcaccggccggtaccgggacctgtaccccgccaacgagctgatcgccctgctggaggagaagggcatcgtgttccgggacggcagcaacatcctgcccaagctgctggagaacgacgacagccacgccatcgacaccatggtggccctgatccggagcgtgctgcagatgcggaacagcaacgccgccaccggcgaggactacatcaacagccccgtgcgggacctgaacggcgtgtgcttcgacagccggttccagaaccccgagtggcccatggacgccgacgccaacggcgcctaccacatcgccctgaagggccagctgctgctgaaccacctgaaggagagcaaggacctgaagctgcagaacggcatcagcaaccaggactggctggcctacatccaggagctgcggaacaagcggcccgccgccaccaagaaggccggccaggccaagaagaagaagggcagctacccctacgacgtgcccgactacgcctacccctacgacgtgcccgactacgcctacccctacgacgtgcccgactacgcctga

SEQ ID NO: 217 - DNA, 3822 bp, synthetic construct
  source 1..3822: mol_type = other DNA
atgagcaagctggagaagttcaccaactgctacagcctgagcaagaccctgcggttcaaggccatccccgtgggcaagacccaggagaacatcgacaacaagcggctgctggtggaggacgagaagcgggccgaggactacaagggcgtgaagaagctgctggaccggtactacctgagcttcatcaacgacgtgctgcacagcatcaagctgaagaacctgaacaactacatcagcctgttccggaagaagacccggaccgagaaggagaacaaggagctggagaacctggagatcaacctgcggaaggagatcgccaaggccttcaagggcaacgagggctacaagagcctgttcaagaaggacatcatcgagaccatcctgcccgagttcctggacgacaaggacgagatcgccctggtgaacagcttcaacggcttcaccaccgccttcaccggcttcttcgacaaccgggagaacatgttcagcgaggaggccaagagcaccagcatcgccttccggtgcatcaacgagaacctgacccggtacatcagcaacatggacatcttcgagaaggtggacgccatcttcgacaagcacgaggtgcaggagatcaaggagaagatcctgaacagcgactacgacgtggaggacttcttcgagggcgagttcttcaacttcgtgctgacccaggagggcatcgacgtgtacaacgccatcatcggcggcttcgtgaccgagagcggcgagaagatcaagggcctgaacgagtacatcaacctgtacaaccagaagaccaagcagaagctgcccaagttcaagcccctgtacaagcaggtgctgagcgaccgggagagcctgagcttctacggcgagggctacaccagcgacgaggaggtgctggaggtgttccggaacaccctgaacaagaacagcgagatcttcagcagcatcaagaagctggagaagctgttcaagaacttcgacgagtacagcagcgccggcatcttcgtgaagaacggccccgccatcagcaccatcagcaaggacatcttcggcgagtggaacgtgatccgggacaagtggaacgccgagtacgacgacatccacctgaagaagaaggccgtggtgaccgagaagtacgaggacgaccggcggaaaagcttcaagaagatcggcagcttcagcctggagcagctgcaggagtacgccgacgccgacctgagcgtggtggagaagctgaaggagatcatcatccagaaggtggacgagatctacaaggtgtacggcagcagcgagaagctgttcgacgccgacttcgtgctggagaaaagcctgaagaagaacgacgccgtggtggccatcatgaaggacctgctggacagcgtgaaaagcttcgagaactacatcaaggccttcttcggcgagggcaaggagaccaaccgggacgagagcttctacggcgacttcgtgctggcctacgacatcctgctgaaggtggaccacatctacgacgccatccggaactacgtgacccagaagccctacagcaaggacaagttcaagctgtacttccagaacccccagttcatgggcggctgggacaaggacaaggagaccgactaccgggccaccatcctgcggtacggcagcaagtactacctggccatcatggacaagaagtacgccaagtgcctgcagaagatcgacaaggacgacgtgaacggcaactacgagaagatcaactacaagctgctgcccggccccaacaagatgctgcccaaggtgttcttcagcaagaagtggatggcctactacaaccccagcgaggacatccagaagatctacaagaacggcaccttcaagaagggcgacatgttcaacctgaacgactgccacaagctgatcgacttcttcaaggacagcatcagccggtaccccaagtggagcaacgcctacgacttcaacttcagcgagaccgagaagtacaaggacatcgccggcttctaccgggaggtggaggagcagggctacaaggtgagcttcgagagcgccagcaagaaggaggtggacaagctggtggaggagggcaagctgtacatgttccagatctacaacaaggacttcagcgacaagagccacggcacccccaacctgcacaccatgtacttcaagctgctgttcgacgagaacaaccacggccagatccggctgagcggcggcgccgagctgttcatgcggcgggccagcctgaagaaggaggagctggtggtgcaccccgccaacagccccatcgccaacaagaaccccgacaaccccaagaagaccaccaccctgagctacgacgtgtacaaggacaagcggttcagcgaggaccagtacgagctgcacatccccatcgccatcaacaagtgccccaagaacatcttcaagatcaacaccgaggtgcgggtgctgctgaagcacgacgacaacccctacgtgatcggcatcgaccggggcgagcggaacctgctgtacatcgtggtggtggacggcaagggcaacatcgtggagcagtacagcctgaacgagatcatcaacaacttcaacggcatccggatcaagaccgactaccacagcctgctggacaagaaggagaaggagcggttcgaggcccggcagaactggaccagcatcgagaacatcaaggagctgaaggccggctacatcagccaggtggtgcacaagatctgcgagctggtggagaagtacgacgccgtgatcgccctggaggacctgaacagcggcttcaagaacagccgggtgaaggtggagaagcaggtgtaccagaagttcgagaagatgctgatcgacaagctgaactacatggtggacaagaaaagcaacccctgcgccaccggcggcgccctgaagggctaccagatcaccaacaagttcgagagcttcaagagcatgagcacccagaacggcttcatcttctacatccccgcctggctgaccagcaagatcgaccccagcaccggcttcgtgaacctgctgaagaccaagtacaccagcatcgccgacagcaagaagttcatcagcagcttcgaccggatcatgtacgtgcccgaggaggacctgttcgagttcgccctggactacaagaacttcagccggaccgacgccgactacatcaagaagtggaagctgtacagctacggcaaccggatccggatcttccggaaccccaagaagaacaacgtgttcgactgggaggaggtgtgcctgaccagcgcctacaaggagctgttcaacaagtacggcatcaactaccagcagggcgacatccgggccctgctgtgcgagcagagcgacaaggccttctacagcagcttcatggccctgatgagcctgatgctgcagatgcggaacagcatcaccggccggaccgacgtggacttcctgatcagccccgtgaagaacagcgacggcatcttctacgacagccggaactacgaggcccaggagaacgccatcctgcccaagaacgccgacgccaacggcgcctacaacatcgcccggaaggtgctgtgggccatcggccagttcaagaaggccgaggacgagaagctggacaaggtgaagatcgccatcagcaacaaggagtggctggagtacgcccagaccagcgtgaagcacaagcggcccgccgccaccaagaaggccggccaggccaagaagaagaagggcagctacccctacgacgtgcccgactacgcctacccctacgacgtgcccgactacgcctacccctacgacgtgcccgactacgcctga

SEQ ID NO: 218 - DNA, 3756 bp, synthetic construct
  source 1..3756: mol_type = other DNA
atgtactacgagagcctgaccaagcagtaccccgtgagcaagaccatccggaacgagctgatccccatcggcaagaccctggacaacatccggcagaacaacatcctggagagcgacgtgaagcggaagcagaactacgagcacgtgaagggcatcctggacgagtaccacaagcagctgatcaacgaggccctggacaactgcaccctgcccagcctgaagatcgccgccgagatctacctgaagaaccagaaggaggtgagcgaccgggaggacttcaacaagacccaggacctgctgcggaaggaggtggtggagaagctgaaggcccacgagaacttcaccaagatcggcaagaaggacatcctggacctgctggagaagctgcccagcatcagcgaggacgactacaacgccctggagagcttccggaacttctacacctacttcaccagctacaacaaggtgcgggagaacctgtacagcgacaaggagaaaagcagcaccgtggcctaccggctgatcaacgagaacttccccaagttcctggacaacgtgaaaagctaccggttcgtgaagaccgccggcatcctggccgacggcctgggcgaggaggagcaggacagcctgttcatcgtggagaccttcaacaagaccctgacccaggacggcatcgacacctacaacagccaggtgggcaagatcaacagcagcatcaacctgtacaaccagaagaaccagaaggccaacggcttccggaagatccccaagatgaagatgctgtacaagcagatcctgagcgaccgggaggagagcttcatcgacgagttccagagcgacgaggtgctgatcgacaacgtggagagctacggcagcgtgctgatcgagagcctgaaaagcagcaaggtgagcgccttcttcgacgccctgcgggagagcaagggcaagaacgtgtacgtgaagaacgacctggccaagaccgccatgagcaacatcgtgttcgagaactggcggaccttcgacgacctgctgaaccaggagtacgacctggccaacgagaacaagaagaaggacgacaagtacttcgagaagcggcagaaggagctgaagaagaacaagagctacagcctggagcacctgtgcaacctgagcgaggacagctgcaacctgatcgagaactacatccaccagatcagcgacgacatcgagaacatcatcatcaacaacgagaccttcctgcggatcgtgatcaacgagcacgaccggagccggaagctggccaagaaccggaaggccgtgaaggccatcaaggacttcctggacagcatcaaggtgctggagcgggagctgaagctgatcaacagcagcggccaggagctggagaaggacctgatcgtgtacagcgcccacgaggagctgctggtggagctgaagcaggtggacagcctgtacaacatgacccggaactacctgaccaagaagcccttcagcaccgagaaggtgaagctgaacttcaaccggagcaccctgctgaacggctgggaccggaacaaggagaccgacaacctgggcgtgctgctgctgaaggacggcaagtactacctgggcatcatgaacaccagcgccaacaaggccttcgtgaacccccccgtggccaagaccgagaaggtgttcaagaaggtggactacaagctgctgcccgtgcccaaccagatgctgcccaaggtgttcttcgccaagagcaacatcgacttctacaaccccagcagcgagatctacagcaactacaagaagggcacccacaagaagggcaacatgttcagcctggaggactgccacaacctgatcgacttcttcaaggagagcatcagcaagcacgaggactggagcaagttcggcttcaagttcagcgacaccgccagctacaacgacatcagcgagttctaccgggaggtggagaagcagggctacaagctgacctacaccgacatcgacgagacctacatcaacgacctgatcgagcggaacgagctgtacctgttccagatctacaacaaggacttcagcatgtacagcaagggcaagctgaacctgcacaccctgtacttcatgatgctgttcgaccagcggaacatcgacgacgtggtgtacaagctgaacggcgaggccgaggtgttctaccggcccgccagcatcagcgaggacgagctgatcatccacaaggccggcgaggagatcaagaacaagaaccccaaccgggcccggaccaaggagaccagcaccttcagctacgacatcgtgaaggacaagcggtacagcaaggacaagttcaccctgcacatccccatcaccatgaacttcggcgtggacgaggtgaagcggttcaacgacgccgtgaacagcgccatccggatcgacgagaacgtgaacgtgatcggcatcgaccggggcgagcggaacctgctgtacgtggtggtgatcgacagcaagggcaacatcctggagcagatcagcctgaacagcatcatcaacaaggagtacgacatcgagaccgactaccacgccctgctggacgagcgggagggcggccgggacaaggcccggaaggactggaacaccgtggagaacatccgggacctgaaggccggctacctgagccaggtggtgaacgtggtggccaagctggtgctgaagtacaacgccatcatctgcctggaggacctgaacttcggcttcaagcggggccggcagaaggtggagaagcaggtgtaccagaagttcgagaagatgctgatcgacaagctgaactacctggtgatcgacaagagccgggagcagaccagccccaaggagctgggcggcgccctgaacgccctgcagctgaccagcaagttcaagagcttcaaggagctgggcaagcagagcggcgtgatctactacgtgcccgcctacctgaccagcaagatcgaccccaccaccggcttcgccaacctgttctacatgaagtgcgagaacgtggagaaaagcaagcggttcttcgacggcttcgacttcatccggttcaacgccctggagaacgtgttcgagttcggcttcgactaccggagcttcacccagcgggcctgcggcatcaacagcaagtggaccgtgtgcaccaacggcgagcggatcatcaagtaccggaaccccgacaagaacaacatgttcgacgagaaggtggtggtggtgaccgacgagatgaagaacctgttcgagcagtacaagatcccctacgaggacggccggaacgtgaaggacatgatcatcagcaacgaggaggccgagttctaccggcggctgtaccggctgctgcagcagaccctgcagatgcggaacagcaccagcgacggcacccgggactacatcatcagccccgtgaagaacaagcgggaggcctacttcaacagcgagctgagcgacggcagcgtgcccaaggacgccgacgccaacggcgcctacaacatcgcccggaagggcctgtgggtgctggagcagatccggcagaaaagcgagggcgagaagatcaacctggccatgaccaacgccgagtggctggagtacgcccagacccacctgctgaagcggcccgccgccaccaagaaggccggccaggccaagaagaagaagggcagctacccctacgacgtgcccgactacgcctacccctacgacgtgcccgactacgcctacccctacgacgtgcccgactacgcctga

SEQ ID NO: 219 - RNA, 40 nt, synthetic construct
  misc_feature 1..40: note = HBG1-1 full-length gRNA
  source 1..40: mol_type = other RNA
taatttctactcttgtagatccttgtcaaggctattggtc

SEQ ID NO: 220 - RNA, 41 nt, synthetic construct
  misc_feature 1..41: note = HBG1-1 modified full-length gRNA
  misc_feature 40: note = Modified to include 2'-O-methyl
  misc_feature 40..41: note = Linked by phosphorodithioate (PS2) bond
  misc_feature 41: note = Modified to include 2'-O-methyl
  source 1..41: mol_type = other RNA
taatttctactcttgtagatccttgtcaaggctattggtca

SEQ ID NO: 221 - protein, 1334 aa, synthetic construct
  REGION 1..1334: note = His-Cas12a-sNLS-sNLS H800A
  REGION 3..8: note = MISC_FEATURE - His tag
  REGION 1319..1325: note = MISC_FEATURE - sNLS
  REGION 1328..1334: note = MISC_FEATURE - sNLS
MGHHHHHHGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 222 - protein, 1333 aa, synthetic construct; identical to SEQ ID NO: 201
  REGION 1..1333: note = Cas12a His-Cas12a-NLS-NLS

SEQ ID NO: 223 - protein, 1332 aa, synthetic construct
  REGION 1..1332: note = Cas12a S542R/K607R (Cas12a RR)
MKHHHHHHMPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNKEKNNGAILFVKNGLYYLGIMPKQKGRYKALSFEPTEKTSEGFDKMYYDYFPDAAKMIPRCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGPKKKRKVG

SEQ ID NO: 224 - protein, 1332 aa, synthetic construct
  REGION 1..1332: note = Cas12a S542R/K548V/N552R (Cas12a RVR)
MKHHHHHHMPKKKRKVMTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGPKKKRKVG

SEQ ID NO: 225 - protein, 1334 aa, synthetic construct
  REGION 1..1334: note = His-Cas12a-2xNLS-S542R/K607R/H800A (Cas12a RR-H800A)
MGHHHHHHGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNKEKNNGAILFVKNGLYYLGIMPKQKGRYKALSFEPTEKTSEGFDKMYYDYFPDAAKMIPRCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 226 - protein, 1334 aa, synthetic construct
  REGION 1..1334: note = His-Cas12a-2xNLS-S542R-K548V-N552R-H800A (Cas12a RVR H800A)
MGHHHHHHGSTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLARGWDVNVEKNRGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGSPKKKRKVGSPKKKRKV

SEQ ID NO: 227 - RNA, 40 nt, synthetic construct; identical to SEQ ID NO: 219
  misc_feature 1..40: note = gRNA
  misc_feature 39: note = Modified to contain 2'-O-methyl
  misc_feature 39..40: note = Linked by phosphorodithioate (PS2) bond
  misc_feature 40: note = Modified to contain 2'-O-methyl
  source 1..40: mol_type = other RNA

SEQ ID NO: 228 - RNA, 45 nt, synthetic construct
  misc_feature 1..45: note = gRNA
  source 1..45: mol_type = other RNA
ctttttaatttctactcttgtagatccttgtcaaggctattggtc

SEQ ID NO: 229 - RNA, 50 nt, synthetic construct
  misc_feature 1..50: note = gRNA
  source 1..50: mol_type = other RNA
aagacctttttaatttctactcttgtagatccttgtcaaggctattggtc

SEQ ID NO: 230 - RNA, 65 nt, synthetic construct
  misc_feature 1..65: note = gRNA
  source 1..65: mol_type = other RNA
atgtgtttttgtcaaaagacctttttaatttctactcttgtagatccttgtcaaggctattggtc

SEQ ID NO: 231 - RNA, 100 nt, synthetic construct
  misc_feature 1..100: note = gRNA
  source 1..100: mol_type = other RNA
aggccagcttgccggttttttagtcgtgctgcttcatgtgtttttgtcaaaagacctttttaatttctactcttgtagatccttgtcaaggctattggtc

SEQ ID NO: 232 - DNA, 45 bp, synthetic construct
  misc_feature 1..45: note = gRNA
  misc_feature 1..5: note = DNA nucleotides
  misc_feature 6..45: note = RNA nucleotides
  source 1..45: mol_type = other DNA
ctttttaatttctactcttgtagatccttgtcaaggctattggtc

SEQ ID NO: 233 - DNA, 50 bp, synthetic construct
  misc_feature 1..50: note = gRNA
  misc_feature 1..10: note = DNA nucleotides
  misc_feature 11..50: note = RNA nucleotides
  source 1..50: mol_type = other DNA
aagacctttttaatttctactcttgtagatccttgtcaaggctattggtc

SEQ ID NO: 234 - DNA, 65 bp, synthetic construct
  misc_feature 1..65: note = gRNA
  misc_feature 1..25: note = DNA nucleotides
  misc_feature 26..65: note = RNA nucleotides
  source 1..65: mol_type = other DNA
atgtgtttttgtcaaaagacctttttaatttctactcttgtagatccttgtcaaggctattggtc

SEQ ID NO: 235 - DNA, 100 bp, synthetic construct
  misc_feature 1..100: note = gRNA
  misc_feature 1..60: note = DNA nucleotides
  misc_feature 61..100: note = RNA nucleotides
  source 1..100: mol_type = other DNA
aggccagcttgccggttttttagtcgtgctgcttcatgtgtttttgtcaaaagacctttttaatttctactcttgtagatccttgtcaaggctattggtc

SEQ ID NO: 236 - DNA, 66 bp, synthetic construct
  misc_feature 1..66: note = gRNA
  misc_feature 1..25: note = DNA nucleotides
  misc_feature 26..66: note = RNA nucleotides
  misc_feature 65: note = Modified to contain 2'-O-methyl
  misc_feature 65..66: note = Linked by phosphorodithioate (PS2) bond
  misc_feature 66: note = Modified to contain 2'-O-methyl
  source 1..66: mol_type = other DNA
atgtgtttttgtcaaaagacctttttaatttctactcttgtagatccttgtcaaggctattggtca

SEQ ID NO: 237 - DNA, 66 bp, synthetic construct; identical to SEQ ID NO: 236
  misc_feature 1..66: note = gRNA
  misc_feature 1..25: note = DNA nucleotides
  misc_feature 26..66: note = RNA nucleotides
  modified_base 65..66: mod_base = OTHER; note = linked by phosphorothioate (PS) bond
  misc_feature 66: note = Modified to contain 2'-O-methyl
  source 1..66: mol_type = other DNA

SEQ ID NO: 238 - DNA, 60 bp, synthetic construct
  misc_feature 1..60: note = gRNA
  misc_feature 1..20: note = DNA nucleotides
  misc_feature 21..60: note = RNA nucleotides
  source 1..60: mol_type = other DNA
tttttgtcaaaagacctttttaatttctactcttgtagatccttgtcaaggctattggtc

SEQ ID NO: 239 - DNA, 70 bp, synthetic construct
  misc_feature 1..70: note = gRNA
  misc_feature 1..30: note = DNA nucleotides
  misc_feature 31..70: note = RNA nucleotides
  source 1..70: mol_type = other DNA
gcttcatgtgtttttgtcaaaagacctttttaatttctactcttgtagatccttgtcaaggctattggtc

SEQ ID NO: 240 - DNA, 90 bp, synthetic construct
  misc_feature 1..90: note = gRNA
  misc_feature 1..50: note = DNA nucleotides
  misc_feature 51..90: note = RNA nucleotides
  source 1..90: mol_type = other DNA
gccggttttttagtcgtgctgcttcatgtgtttttgtcaaaagacctttttaatttctactcttgtagatccttgtcaaggctattggtc

SEQ ID NO: 241 - DNA, 80 bp, synthetic construct
  misc_feature 1..80: note = gRNA
  misc_feature 1..40: note = DNA nucleotides
  misc_feature 41..80: note = RNA nucleotides
  source 1..80: mol_type = other DNA
tagtcgtgctgcttcatgtgtttttgtcaaaagacctttttaatttctactcttgtagatccttgtcaaggctattggtc

SEQ ID NO: 242 - DNA, 60 bp, synthetic construct
  misc_feature 1..60: note = gRNA
  misc_feature 1..20: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 21..60: note = RNA nucleotides
  source 1..60: mol_type = other DNA
ccgaagttttcttcggtttttaatttctactcttgtagatccttgtcaaggctattggtc

SEQ ID NO: 243 - DNA, 65 bp, synthetic construct
  misc_feature 1..65: note = gRNA
  misc_feature 1..25: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 26..65: note = RNA nucleotides
  source 1..65: mol_type = other DNA
tttttccgaagttttcttcggtttttaatttctactcttgtagatccttgtcaaggctattggtc

SEQ ID NO: 244 - DNA, 70 bp, synthetic construct
  misc_feature 1..70: note = gRNA
  misc_feature 1..30: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 31..70: note = RNA nucleotides
  source 1..70: mol_type = other DNA
aacgctttttccgaagttttcttcggtttttaatttctactcttgtagatccttgtcaaggctattggtc

SEQ ID NO: 245 - DNA, 81 bp, synthetic construct
  misc_feature 1..81: note = gRNA
  misc_feature 1..41: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 42..81: note = RNA nucleotides
  source 1..81: mol_type = other DNA
gcgttgttttcaacgctttttccgaagttttcttcggtttttaatttctactcttgtagatccttgtcaaggctattggtc

SEQ ID NO: 246 - DNA, 102 bp, synthetic construct
  misc_feature 1..102: note = gRNA
  misc_feature 1..62: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 63..102: note = RNA nucleotides
  source 1..102: mol_type = other DNA
ggcttcttttgaagcctttttgcgttgttttcaacgctttttccgaagttttcttcggtttttaatttctactcttgtagatccttgtcaaggctattggtc

SEQ ID NO: 247 - RNA, 40 nt, synthetic construct; identical to SEQ ID NO: 219
  misc_feature 1..40: note = gRNA
  misc_feature 1: note = Modified to contain 2'-O-methyl
  source 1..40: mol_type = other RNA

SEQ ID NO: 248 - RNA, 40 nt, synthetic construct; identical to SEQ ID NO: 219
  misc_feature 1..40: note = gRNA
  misc_feature 1: note = Modified to contain 2'-O-methyl
  modified_base 1..2: mod_base = OTHER; note = linked by phosphorothioate (PS) bond
  source 1..40: mol_type = other RNA

SEQ ID NO: 249 - RNA, 40 nt, synthetic construct; identical to SEQ ID NO: 219
  misc_feature 1..40: note = gRNA
  misc_feature 1: note = Modified to contain 2'-O-methyl
  misc_feature 2: note = Modified to contain 2'-O-methyl
  source 1..40: mol_type = other RNA

SEQ ID NO: 250 - RNA, 40 nt, synthetic construct; identical to SEQ ID NO: 219
  misc_feature 1..40: note = gRNA
  misc_feature 1: note = Modified to contain 2'-O-methyl
  modified_base 1..2: mod_base = OTHER; note = linked by phosphorothioate (PS) bond
  misc_feature 2: note = Modified to contain 2'-O-methyl
  source 1..40: mol_type = other RNA

SEQ ID NO: 251 - RNA, 40 nt, synthetic construct; identical to SEQ ID NO: 219
  misc_feature 1..40: note = gRNA
  misc_feature 1: note = Modified to contain 2'-O-methyl
  misc_feature 2: note = Modified to contain 2'-O-methyl
  misc_feature 3: note = Modified to contain 2'-O-methyl
  source 1..40: mol_type = other RNA

SEQ ID NO: 252 - RNA, 40 nt, synthetic construct; identical to SEQ ID NO: 219
  misc_feature 1..40: note = gRNA
  misc_feature 1: note = Modified to contain 2'-O-methyl
  modified_base 1..2: mod_base = OTHER; note = linked by phosphorothioate (PS) bond
  modified_base 2..3: mod_base = OTHER; note = linked by phosphorothioate (PS) bond
  misc_feature 2: note = Modified to contain 2'-O-methyl
  modified_base 3..4: mod_base = OTHER; note = linked by phosphorothioate (PS) bond
  misc_feature 3: note = Modified to contain 2'-O-methyl
  source 1..40: mol_type = other RNA

SEQ ID NO: 253 - DNA, 65 bp, synthetic construct; identical to SEQ ID NO: 234
  misc_feature 1..65: note = gRNA
  misc_feature 1..25: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 26..65: note = RNA nucleotides
  source 1..65: mol_type = other DNA

SEQ ID NO: 254 - DNA, 66 bp, synthetic construct; identical to SEQ ID NO: 236
  misc_feature 1..66: note = gRNA
  misc_feature 1..25: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 26..66: note = RNA nucleotides
  modified_base 65..66: mod_base = OTHER; note = linked by phosphorothioate (PS) bond
  misc_feature 66: note = Modified to contain 2'-O-methyl
  source 1..66: mol_type = other DNA

SEQ ID NO: 255 - RNA, 41 nt, synthetic construct; identical to SEQ ID NO: 220
  misc_feature 1..41: note = gRNA
  modified_base 40..41: mod_base = OTHER; note = linked by phosphorothioate (PS) bond
  misc_feature 41: note = Modified to contain 2'-O-methyl
  source 1..41: mol_type = other RNA

SEQ ID NO: 256 - RNA, 42 nt, synthetic construct
  misc_feature 1..42: note = gRNA
  misc_feature 41: note = Modified to contain 2'-O-methyl
  modified_base 41..42: mod_base = OTHER; note = linked by phosphorothioate (PS) bond
  source 1..42: mol_type = other RNA
taatttctactcttgtagatccttgtcaaggctattggtcat

SEQ ID NO: 257 - RNA, 41 nt, synthetic construct
  misc_feature 1..41: note = gRNA
  source 1..41: mol_type = other RNA
taatttctactcttgtagatccttgtcaaggctattggtct

SEQ ID NO: 258 - RNA, 42 nt, synthetic construct; identical to SEQ ID NO: 256
  misc_feature 1..42: note = gRNA
  source 1..42: mol_type = other RNA

SEQ ID NO: 259 - RNA, 44 nt, synthetic construct
  misc_feature 1..44: note = gRNA
  source 1..44: mol_type = other RNA
taatttctactcttgtagatccttgtcaaggctattggtcaagt

SEQ ID NO: 260 - DNA, 66 bp, synthetic construct
  misc_feature 1..66: note = gRNA
  misc_feature 1..25: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 26..66: note = RNA nucleotides
  source 1..66: mol_type = other DNA
atgtgtttttgtcaaaagacctttttaatttctactcttgtagatccttgtcaaggctattggtct

SEQ ID NO: 261 - DNA, 69 bp, synthetic construct
  misc_feature 1..69: note = gRNA
  misc_feature 1..25: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 26..69: note = RNA nucleotides
  source 1..69: mol_type = other DNA
atgtgtttttgtcaaaagacctttttaatttctactcttgtagatccttgtcaaggctattggtcaagt

SEQ ID NO: 262 - RNA, 41 nt, synthetic construct; identical to SEQ ID NO: 220
  misc_feature 1..41: note = gRNA
  source 1..41: mol_type = other RNA

SEQ ID NO: 263 - RNA, 43 nt, synthetic construct
  misc_feature 1..43: note = gRNA
  source 1..43: mol_type = other RNA
taatttctactcttgtagatccttgtcaaggctattggtcaag

SEQ ID NO: 264 - DNA, 68 bp, synthetic construct
  misc_feature 1..68: note = gRNA
  misc_feature 1..25: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 26..68: note = RNA nucleotides
  source 1..68: mol_type = other DNA
atgtgtttttgtcaaaagacctttttaatttctactcttgtagatccttgtcaaggctattggtcaag

SEQ ID NO: 265 - RNA, 44 nt, synthetic construct
  misc_feature 1..44: note = gRNA
  source 1..44: mol_type = other RNA
taatttctactcttgtagatccttgtcaaggctattggtctttt

SEQ ID NO: 266 - RNA, 44 nt, synthetic construct; identical to SEQ ID NO: 265
  misc_feature 1..44: note = gRNA
  misc_feature 41: note = Modified to contain 2'-O-methyl
  modified_base 41..42: mod_base = OTHER; note = linked by phosphorothioate (PS) bond
  modified_base 42..43: mod_base = OTHER; note = linked by phosphorothioate (PS) bond
  misc_feature 42: note = Modified to contain 2'-O-methyl
  misc_feature 43: note = Modified to contain 2'-O-methyl
  modified_base 43..44: mod_base = OTHER; note = linked by phosphorothioate (PS) bond
  source 1..44: mol_type = other RNA

SEQ ID NO: 267 - DNA, 69 bp, synthetic construct
  misc_feature 1..69: note = gRNA
  misc_feature 1..25: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 26..69: note = RNA nucleotides
  source 1..69: mol_type = other DNA
atgtgtttttgtcaaaagacctttttaatttctactcttgtagatccttgtcaaggctattggtctttt

SEQ ID NO: 268 - DNA, 66 bp, synthetic construct
  misc_feature 1..66: note = gRNA
  misc_feature 1..25: note = DNA nucleotides
  misc_feature 26..66: note = RNA nucleotides
  modified_base 65..66: mod_base = OTHER; note = linked by phosphorothioate (PS) bond
  misc_feature 66: note = Modified to contain 2'-O-methyl
  source 1..66: mol_type = other DNA
aaaaaaaaaaaaaaaaaaaaaaaaataatttctactcttgtagatccttgtcaaggctattggtca

SEQ ID NO: 269 - DNA, 66 bp, synthetic construct
  misc_feature 1..66: note = gRNA
  misc_feature 1..25: note = DNA nucleotides
  misc_feature 26..66: note = RNA nucleotides
  modified_base 65..66: mod_base = OTHER; note = linked by phosphorothioate (PS) bond
  misc_feature 66: note = Modified to contain 2'-O-methyl
  source 1..66: mol_type = other DNA
ttttttttttttttttttttttttttaatttctactcttgtagatccttgtcaaggctattggtca

SEQ ID NO: 270 - DNA, 61 bp, synthetic construct
  misc_feature 1..61: note = gRNA
  misc_feature 1..20: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 21..61: note = RNA nucleotides
  modified_base 60..61: mod_base = OTHER; note = linked by phosphorothioate (PS) bond
  misc_feature 61: note = Modified to contain 2'-O-methyl
  source 1..61: mol_type = other DNA
ccgaagttttcttcggtttttaatttctactcttgtagatccttgtcaaggctattggtca

SEQ ID NO: 271 - RNA, 41 nt, synthetic construct
  misc_feature 1..41: note = gRNA
  modified_base 40..41: mod_base = OTHER; note = linked by phosphorothioate (PS) bond
  misc_feature 41: note = Modified to contain 2'-O-methyl
  source 1..41: mol_type = other RNA
taatttctactcttgtagatagacagatatttgcattgaga

SEQ ID NO: 272 - DNA, 66 bp, synthetic construct
  misc_feature 1..66: note = gRNA
  misc_feature 1..25: note = DNA nucleotides
  misc_feature 26..66: note = RNA nucleotides
  modified_base 65..66: mod_base = OTHER; note = linked by phosphorothioate (PS) bond
  misc_feature 66: note = Modified to contain 2'-O-methyl
  source 1..66: mol_type = other DNA
atgtgtttttgtcaaaagacctttttaatttctactcttgtagatagacagatatttgcattgaga

SEQ ID NO: 273 - DNA, 61 bp, synthetic construct
  misc_feature 1..61: note = gRNA
  misc_feature 1..20: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 21..61: note = RNA nucleotides
  modified_base 60..61: mod_base = OTHER; note = linked by phosphorothioate (PS) bond
  misc_feature 61: note = Modified to contain 2'-O-methyl
  source 1..61: mol_type = other DNA
ccgaagttttcttcggtttttaatttctactcttgtagatagacagatatttgcattgaga

SEQ ID NO: 274 - RNA, 41 nt, synthetic construct
  misc_feature 1..41: note = gRNA
  modified_base 40..41: mod_base = OTHER; note = linked by phosphorothioate (PS) bond
  misc_feature 41: note = Modified to contain 2'-O-methyl
  source 1..41: mol_type = other RNA
taatttctactcttgtagatcattgagatagtgtggggaag

SEQ ID NO: 275 - DNA, 66 bp, synthetic construct
  misc_feature 1..66: note = gRNA
  misc_feature 1..25: note = DNA nucleotides
  misc_feature 26..66: note = RNA nucleotides
  modified_base 65..66: mod_base = OTHER; note = linked by phosphorothioate (PS) bond
  misc_feature 66: note = Modified to contain 2'-O-methyl
  source 1..66: mol_type = other DNA
atgtgtttttgtcaaaagacctttttaatttctactcttgtagatcattgagatagtgtggggaag

SEQ ID NO: 276 - DNA, 61 bp, synthetic construct
  misc_feature 1..61: note = gRNA
  misc_feature 1..20: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 21..61: note = RNA nucleotides
  modified_base 60..61: mod_base = OTHER; note = linked by phosphorothioate (PS) bond
  misc_feature 61: note = Modified to contain 2'-O-methyl
  source 1..61: mol_type = other DNA
ccgaagttttcttcggtttttaatttctactcttgtagatcattgagatagtgtggggaag

SEQ ID NO: 277 - RNA, 41 nt, synthetic construct
  misc_feature 1..41: note = gRNA
  modified_base 40..41: mod_base = OTHER; note = linked by phosphorothioate (PS) bond
  misc_feature 41: note = Modified to contain 2'-O-methyl
  source 1..41: mol_type = other RNA
taatttctactcttgtagatcttctcccatcatagaggata

SEQ ID NO: 278 - DNA, 66 bp, synthetic construct
  misc_feature 1..66: note = gRNA
  misc_feature 1..25: note = DNA nucleotides
  misc_feature 26..66: note = RNA nucleotides
  modified_base 65..66: mod_base = OTHER; note = linked by phosphorothioate (PS) bond
  misc_feature 66: note = Modified to contain 2'-O-methyl
  source 1..66: mol_type = other DNA
atgtgtttttgtcaaaagacctttttaatttctactcttgtagatcttctcccatcatagaggata

SEQ ID NO: 279 - DNA, 61 bp, synthetic construct
  misc_feature 1..61: note = gRNA
  misc_feature 1..20: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 21..61: note = RNA nucleotides
  modified_base 60..61: mod_base = OTHER; note = linked by phosphorothioate (PS) bond
  misc_feature 61: note = Modified to contain 2'-O-methyl
  source 1..61: mol_type = other DNA
ccgaagttttcttcggtttttaatttctactcttgtagatcttctcccatcatagaggata

SEQ ID NO: 280 - protein, 1363 aa, synthetic construct
  REGION 1..1363: note = Cas12a
  REGION 1310..1331: note = MISC_FEATURE - NLS
  REGION 1358..1363: note = MISC_FEATURE - His tag
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 281 - protein, 1363 aa, synthetic construct
  REGION 1..1363: note = Cas12a
  REGION 1310..1331: note = MISC_FEATURE - NLS
  REGION 1358..1363: note = MISC_FEATURE - His tag
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 282 - protein, 1363 aa, synthetic construct
  REGION 1..1363: note = Cas12a
  REGION 1310..1331: note = MISC_FEATURE - NLS
  REGION 1358..1363: note = MISC_FEATURE - His tag
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 283 - protein, 1331 aa, synthetic construct
  REGION 1..1331: note = Cas12a
  REGION 1310..1331: note = MISC_FEATURE - NLS
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 284 - DNA, 67 bp, synthetic construct
  misc_feature 1..67: note = gRNA
  misc_feature 1..25: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 26..67: note = RNA nucleotides
  modified_base 66..67: mod_base = OTHER; note = linked by phosphorothioate (PS) bond
  misc_feature 66: note = Modified to contain 2'-O-methyl
  source 1..67: mol_type = other DNA
atgtgtttttgtcaaaagacctttttaatttctactcttgtagatccttgtcaaggctattggtcat

SEQ ID NO: 285 - DNA, 68 bp, synthetic construct
  misc_feature 1..68: note = gRNA
  misc_feature 1..25: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 26..68: note = RNA nucleotides
  modified_base 66..67: mod_base = OTHER; note = linked by phosphorothioate (PS) bond
  misc_feature 66: note = Modified to contain 2'-O-methyl
  misc_feature 67: note = Modified to contain 2'-O-methyl
  modified_base 67..68: mod_base = OTHER; note = linked by phosphorothioate (PS) bond
  source 1..68: mol_type = other DNA
atgtgtttttgtcaaaagacctttttaatttctactcttgtagatccttgtcaaggctattggtcaat

SEQ ID NO: 286 - DNA, 66 bp, synthetic construct; identical to SEQ ID NO: 260
  misc_feature 1..66: note = gRNA
  misc_feature 1..25: note = DNA nucleotides
  modified_base 1..2: mod_base = OTHER; note = linked by phosphorothioate (PS) bond
  misc_feature 2..3: note = Linked by phosphorothioate (PS) bond
  misc_feature 26..66: note = RNA nucleotides
  misc_feature 65: note = Modified to contain 2'-O-methyl
  misc_feature 65..66: note = Linked by phosphorothioate (PS) bond
  source 1..66: mol_type = other DNA

SEQ ID NO: 287 - DNA, 65 bp, synthetic construct; identical to SEQ ID NO: 234
  misc_feature 1..65: note = gRNA
  misc_feature 1..25: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 26..65: note = RNA nucleotides
  modified_base 64..65: mod_base = OTHER; note = linked by phosphorothioate (PS) bond
  misc_feature 65: note = Modified to contain 2'-O-methyl
  source 1..65: mol_type = other DNA

SEQ ID NO: 288 - DNA, 67 bp, synthetic construct
  misc_feature 1..67: note = gRNA
  misc_feature 1..25: note = DNA nucleotides
  modified_base 1..2: mod_base = OTHER; note = linked by phosphorothioate (PS) bond
  modified_base 2..3: mod_base = OTHER; note = linked by phosphorothioate (PS) bond
  misc_feature 26..67: note = RNA nucleotides
  modified_base 65..66: mod_base = OTHER; note = linked by phosphorothioate (PS) bond
  modified_base 66..67: mod_base = OTHER; note = linked by phosphorothioate (PS) bond
  misc_feature 66: note = Modified to contain 2'-O-methyl
  misc_feature 67: note = Modified to contain 2'-O-methyl
  source 1..67: mol_type = other DNA
atgtgtttttgtcaaaagacctttttaatttctactcttgtagatccttgtcaaggctattggtcaa

SEQ ID NO: 289 - RNA, 66 nt, synthetic construct
  misc_feature 1..66: note = gRNA
  modified_base 1..2: mod_base = OTHER; note = linked by phosphorothioate (PS) bond
  misc_feature 1: note = Modified to contain 2'-O-methyl
  misc_feature 2: note = Modified to contain 2'-O-methyl
  misc_feature 2..3: note = Linked by phosphorothioate (PS) bond
  misc_feature 65..66: note = Linked by phosphorothioate (PS) bond
  misc_feature 66: note = Modified to contain 2'-O-methyl
  source 1..66: mol_type = other RNA
atgtgtttttgtcaaaagacctttttaatttctactcttgtagatccttgtcaaggctattggtca

SEQ ID NO: 290 - RNA, 66 nt, synthetic construct
  misc_feature 1..66: note = gRNA
  modified_base 1..2: mod_base = OTHER; note = linked by phosphorothioate (PS) bond
  misc_feature 1: note = Modified to contain 2'-O-methyl
  misc_feature 2: note = Modified to contain 2'-O-methyl
  modified_base 2..3: mod_base = OTHER; note = linked by phosphorothioate (PS) bond
  modified_base 65..66: mod_base = OTHER; note = linked by phosphorothioate (PS) bond
  misc_feature 66: note = Modified to contain 2'-O-methyl
  source 1..66: mol_type = other RNA
aaaaaaaaaaaaaaaaaaaaaaaaataatttctactcttgtagatccttgtcaaggctattggtca

SEQ ID NO: 291 - RNA, 66 nt, synthetic construct
  misc_feature 1..66: note = gRNA
  modified_base 1..2: mod_base = OTHER; note = linked by phosphorothioate (PS) bond
  misc_feature 1: note = Modified to contain 2'-O-methyl
  misc_feature 2: note = Modified to contain 2'-O-methyl
  modified_base 2..3: mod_base = OTHER; note = linked by phosphorothioate (PS) bond
  modified_base 65..66: mod_base = OTHER; note = linked by phosphorothioate (PS) bond
  misc_feature 66: note = Modified to contain 2'-O-methyl
  source 1..66: mol_type = other RNA
ttttttttttttttttttttttttttaatttctactcttgtagatccttgtcaaggctattggtca

SEQ ID NO: 292 - DNA, 42 bp, synthetic construct
  misc_feature 1..42: note = gRNA
  modified_base 1..2: mod_base = OTHER; note = linked by phosphorothioate (PS) bond
  modified_base 2..3: mod_base = OTHER; note = linked by phosphorothioate (PS) bond
  source 1..42: mol_type = other DNA
taatttctactcttgtagatccttgtcaggctattggtcaag

SEQ ID NO: 293 - protein, 1331 aa, synthetic construct
  REGION 1..1331: note = Cas12a
  REGION 1310..1331: note = MISC_FEATURE - NLS
STQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKV

SEQ ID NO: 294 - protein, 1363 aa, synthetic construct
  REGION 1..1363: note = Cas12a
  REGION 1310..1331: note = MISC_FEATURE - NLS
  REGION 1358..1363: note = MISC_FEATURE - His tag
STQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGRSSDDEATADSQHAAPPKKKRKVGGSGGSGGSGGSGGSGGSGGSGGSLEHHHHHH

SEQ ID NO: 295 - protein, 1361 aa, synthetic construct
  REGION 1..1361: note = Cas12a
  REGION 1342..1348: note = MISC_FEATURE - NLS
  REGION 1356..1361: note = MISC_FEATURE - His tag
MGRDPGKPIPNPLLGLDSTAPKKKRKVGIHGVPAATQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQMPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAHRLGEKMLNKKLKDQKTPIPDTLYQELYDYVNHRLSHDLSDEARALLPNVITKEVSHEIIKDRRFTSDKFFFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNPKKKRKVKLAAALEHHHHHH

SEQ ID NO: 296 - DNA, 4002 bp, synthetic construct
  misc_feature 1..4002: note = His-Cas12a-sNLS-sNLS H800A
  misc_feature 7..24: note = His tag
  misc_feature 3955..3975: note = sNLS
  misc_feature 3982..4002: note = sNLS
  source 1..4002: mol_type = other DNA
atgggccatcatcatcaccatcacggtagcacccagtttgaaggctttaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacgtacgcagatcagtgtctgcagctggttcagctggattgggaaaatctgagcgcagcaattgatagttatcgcaaagagaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacacagattgatctgtataaccagctgttaggtggtattagccgtgaagcaggcaccgaaaaaatcaaaggtctgaatgaagtgctgaatctggccattcagaaaaatgatgaaaccgcacatattattgcaagcctgccgcatcgttttattccgctgtttaagcagattctgagcgatcgtaataccctgagctttattctggaagaattcaaatccgatgaagaggtgattcagagcttctgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgagccatgcacatgcagcactggatcagccgctgccgaccacactgaaaaaacaagaagaaaaagagatcctgaaaagccagctggatagcctgctgggtctgtatcatctgctggactggtttgcagttgatgaaagcaatgaagttgatccggaatttagcgcacgtctgaccggcattaagctggaaatggaaccgagcctgagtttttacaataaagcccgtaattatgccaccaaaaaaccgtatagcgtggaaaaattcaagctgaacttccagatgccgacactggcaagcggttgggatgttaataaagaaaagaacaatggtgccatcctgttcgtgaaaaatggcctgtattatctgggtatcatgccgaaacagaaaggtcgttataaagcactgagctttgaaccgacagaaaaaaccagtgaaggcttcgataaaatgtactatgattactttccggacgcagccaaaatgattccgaaatgcagcacccagctgaaagcagttaccgcacattttcagacccataccacaccgattctgctgagcaataactttattgaaccgctggaaatcaccaaagagatctacgatctgaataacccggaaaaagagccgaaaaagtttcagaccgcctatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaataatggatgcagttgaaaccggtaagctgtacctgttccagatttataacaaagactttgccaaaggccatcatggtaaaccgaatctgcataccctgtattggaccggtctgtttagccctgaaaatctggcaaaaaccagcattaaactgaatggtcaggcggaactgttttatcgtccgaaaagccgtatgaaacgtatggcagcacgtctgggtgaaaaaatgctgaataaaaagctgaaggatcagaaaaccccgattccggatacactgtatcaagagctgtatgattatgtgaatcatcgtctgagtcatgatctgagtgatgaagcacgtgccctgctgccgaatgttattaccaaagaagttagccacgagatcattaaagatcgtcgttttaccagcgacaagttttttttccatgttccgattacgctgaattatcaggcagcaaatagcccgagcaaatttaaccagcgtgtgaatgcatatctgaaagaacatccggaaacaccgattattggtattgatcgtggtgaacgcaacctgatttatatcaccgttattgatagcaccggcaagatcctggaacagcgtagcctgaataccattcagcagtttgattatcagaaaaagctggacaaccgtgaaaaagaacgtgttgcagcccgtcaggcatggtcagttgttggtacaattaaagacctgaaacagggttatctgagccaggttattcatgaaattgtggatctgatgattcattaccaggcagttgttgtgctggaaaacctgaattttggctttaaaagcaaacgtaccggcattgcggaaaaagcagtttatcagcagttcgaaaagatgctgatcgataaactgaactgtctggtgctgaaagattatccggctgaaaaagttggcggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggttttgatttcctgcattacgacgttaagaccggtgattttattctgcactttaagatgaatcggaacctgtcatttcagcgtggtctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgttagagaatgatgatagccatgccattgataccatggttgccctgattcgtagcgttctgcagatgcgtaatagcaatgcagcaaccggtgaagattacattaatagtccggttcgtgatctgaatggtgtttgttttgatagccgttttcagaatccggaatggccgatggatgcggatgcaaatggtgcatatcatattgcactgaaaggacagctgctgctgaaccacctgaaagaaagtaaagatttgaagctgcagaatggcattagcaatcaggattggctggcatatatccaagaactgcgtaacggttctcctaagaaaaagcgcaaagttggcagtccgaagaagaaacgtaaggtt

SEQ ID NO: 297 - DNA, 4089 bp, synthetic construct
  misc_feature 1..4089: note = Cas12a
  misc_feature 3928..3993: note = NLS
  misc_feature 4072..4089: note = His tag
  source 1..4089: mol_type = other DNA
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagttggtggtagcggtggttcaggtggtagtggcggtagtggtggctcagggggttctggtggctctggtggtagcctcgagcaccaccaccaccaccac

SEQ ID NO: 298 - DNA, 4089 bp, synthetic construct
  misc_feature 1..4089: note = Cas12a
  misc_feature 3928..3993: note = NLS
  misc_feature 4072..4089: note = His tag
  source 1..4089: mol_type = other DNA
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagatgccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcttttttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagttggtggtagcggtggttcaggtggtagtggcggtagtggtggctcagggggttctggtggctctggtggtagcctcgagcaccaccaccaccaccac

SEQ ID NO: 299 - DNA, 4089 bp, synthetic construct
  misc_feature 1..4089: note = Cas12a
  misc_feature 3928..3993: note = NLS
  misc_feature 4072..4089: note = His tag
  source 1..4089: mol_type = other DNA
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcagctcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagttggtggtagcggtggttcaggtggtagtggcggtagtggtggctcagggggttctggtggctctggtggtagcctcgagcaccaccaccaccaccac

SEQ ID NO: 300 - DNA, 3993 bp, synthetic construct
  misc_feature 1..3993: note = Cas12a
  misc_feature 3928..3993: note = NLS
  source 1..3993: mol_type = other DNA
atgacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcagctcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagtt

SEQ ID NO: 301 - DNA, 3993 bp, synthetic construct
  misc_feature 1..3993: note = Cas12a
  misc_feature 3928..3993: note = NLS
  source 1..3993: mol_type = other DNA
agcacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagtt

SEQ ID NO: 302 - DNA, 4089 bp, synthetic construct
  misc_feature 1..4089: note = Cas12a
  misc_feature 3928..3993: note = NLS
  misc_feature 4072..4089: note = His tag
  source 1..4089: mol_type = other DNA
agcacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagcgtccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcctgtttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaacggtcgtagcagtgatgatgaagcaaccgcagatagccagcatgcagcaccgcctaaaaagaaacgtaaagttggtggtagcggtggttcaggtggtagtggcggtagtggtggctcagggggttctggtggctctggtggtagcctcgagcaccaccaccaccaccac

SEQ ID NO: 303 - DNA, 4083 bp, synthetic construct
  misc_feature 1..4083: note = Cas12a
  misc_feature 4024..4044: note = NLS
  misc_feature 4066..4083: note = His tag
  source 1..4083: mol_type = other DNA
atgggtcgggatccaggtaaaccgattccgaatccgctgctgggtctggatagcaccgcaccgaaaaaaaaacgtaaagttggtattcatggtgttccggcagcaacccagtttgaaggtttcaccaatctgtatcaggttagcaaaaccctgcgttttgaactgattccgcagggtaaaaccctgaaacatattcaagaacagggcttcatcgaagaggataaagcacgtaacgatcactacaaagaactgaaaccgattatcgaccgcatctataaaacctatgcagatcagtgtctgcagctggttcagctggattgggaaaatctgagcgcagcaattgatagttatcgcaaagaaaaaaccgaagaaacccgtaatgcactgattgaagaacaggcaacctatcgtaatgccatccatgattatttcattggtcgtaccgataatctgaccgatgcaattaacaaacgtcacgccgaaatctataaaggcctgtttaaagccgaactgtttaatggcaaagttctgaaacagctgggcaccgttaccaccaccgaacatgaaaatgcactgctgcgtagctttgataaattcaccacctatttcagcggcttttatgagaatcgcaaaaacgtgtttagcgcagaagatattagcaccgcaattccgcatcgtattgtgcaggataatttcccgaaattcaaagagaactgccacatttttacccgtctgattaccgcagttccgagcctgcgtgaacattttgaaaacgttaaaaaagccatcggcatctttgttagcaccagcattgaagaagtttttagcttcccgttttacaatcagctgctgacccagacccagattgatctgtataaccaactgctgggtggtattagccgtgaagcaggcaccgaaaaaatcaaaggtctgaatgaagtgctgaatctggccattcagaaaaatgatgaaaccgcacatattattgcaagcctgccgcatcgttttattccgctgttcaaacaaattctgagcgatcgtaataccctgagctttattctggaagaattcaaatccgatgaagaggtgattcagagcttttgcaaatacaaaacgctgctgcgcaatgaaaatgttctggaaactgccgaagcactgtttaacgaactgaatagcattgatctgacccacatctttatcagccacaaaaaactggaaaccatttcaagcgcactgtgtgatcattgggataccctgcgtaatgccctgtatgaacgtcgtattagcgaactgaccggtaaaattaccaaaagcgcgaaagaaaaagttcagcgcagtctgaaacatgaggatattaatctgcaagagattattagcgcagccggtaaagaactgtcagaagcatttaaacagaaaaccagcgaaattctgtcacatgcacatgcagcactggatcagccgctgccgaccaccctgaaaaaacaagaagaaaaagaaatcctgaaaagccagctggatagcctgctgggtctgtatcatctgctggactggtttgcagttgatgaaagcaatgaagttgatccggaatttagcgcacgtctgaccggcattaaactggaaatggaaccgagcctgagcttttataacaaagcccgtaattatgccaccaaaaaaccgtatagcgtcgaaaaattcaaactgaactttcagatgccgaccctggcaagcggttgggatgttaataaagaaaaaaacaacggtgccatcctgttcgtgaaaaatggcctgtattatctgggtattatgccgaaacagaaaggtcgttataaagcgctgagctttgaaccgacggaaaaaaccagtgaaggttttgataaaatgtactacgactattttccggatgcagccaaaatgattccgaaatgtagcacccagctgaaagcagttaccgcacattttcagacccataccaccccgattctgctgagcaataactttattgaaccgctggaaatcaccaaagagatctacgatctgaataacccggaaaaagagccgaaaaaattccagaccgcatatgcaaaaaaaaccggtgatcagaaaggttatcgtgaagcgctgtgtaaatggattgatttcacccgtgattttctgagcaaatacaccaaaaccaccagtatcgatctgagcagcctgcgtccgagcagccagtataaagatctgggcgaatattatgcagaactgaatccgctgctgtatcatattagctttcagcgtattgccgagaaagaaatcatggacgcagttgaaaccggtaaactgtacctgttccagatctacaataaagattttgccaaaggccatcatggcaaaccgaatctgcataccctgtattggaccggtctgtttagccctgaaaatctggcaaaaacctcgattaaactgaatggtcaggcggaactgttttatcgtccgaaaagccgtatgaaacgtatggcacatcgtctgggtgaaaaaatgctgaacaaaaaactgaaagaccagaaaaccccgatcccggatacactgtatcaagaactgtatgattatgtgaaccatcgtctgagccatgatctgagtgatgaagcacgtgccctgctgccgaatgttattaccaaagaagttagccacgagatcattaaagatcgtcgttttaccagcgacaaattcttttttcatgtgccgattaccctgaattatcaggcagcaaatagcccgagcaaatttaaccagcgtgttaatgcatatctgaaagaacatccagaaacgccgattattggtattgatcgtggtgaacgtaacctgatttatatcaccgttattgatagcaccggcaaaatcctggaacagcgtagcctgaataccattcagcagtttgattaccagaaaaaactggataatcgcgagaaagaacgtgttgcagcacgtcaggcatggtcagttgttggtacaattaaagacctgaaacagggttatctgagccaggttattcatgaaattgtggatctgatgattcactatcaggccgttgttgtgctggaaaacctgaattttggctttaaaagcaaacgtaccggcattgcagaaaaagcagtttatcagcagttcgagaaaatgctgattgacaaactgaattgcctggtgctgaaagattatccggctgaaaaagttggtggtgttctgaatccgtatcagctgaccgatcagtttaccagctttgcaaaaatgggcacccagagcggatttctgttttatgttccggcaccgtatacgagcaaaattgatccgctgaccggttttgttgatccgtttgtttggaaaaccatcaaaaaccatgaaagccgcaaacattttctggaaggtttcgattttctgcattacgacgttaaaacgggtgatttcatcctgcactttaaaatgaatcgcaatctgagttttcagcgtggcctgcctggttttatgcctgcatgggatattgtgtttgagaaaaacgaaacacagttcgatgcaaaaggcaccccgtttattgcaggtaaacgtattgttccggtgattgaaaatcatcgtttcaccggtcgttatcgcgatctgtatccggcaaatgaactgatcgcactgctggaagagaaaggtattgtttttcgtgatggctcaaacattctgccgaaactgctggaaaatgatgatagccatgcaattgataccatggttgcactgattcgtagcgttctgcagatgcgtaatagcaatgcagcaaccggtgaagattacattaatagtccggttcgtgatctgaatggtgtttgttttgatagccgttttcagaatccggaatggccgatggatgcagatgcaaatggtgcatatcatattgcactgaaaggacagctgctgctgaaccacctgaaagaaagcaaagatctgaaactgcaaaacggcattagcaatcaggattggctggcatatatccaagaactgcgtaaccctaaaaaaaaacgcaaagtgaagcttgcggccgcactcgagcaccaccaccaccaccac

SEQ ID NO: 304 - skipped

SEQ ID NO: 305 - DNA, 10 bp, synthetic construct
  misc_feature 1..10: note = +10 DNA gRNA 5' extension
  source 1..10: mol_type = other DNA
aagacctttt

SEQ ID NO: 306 - DNA, 25 bp, synthetic construct
  misc_feature 1..25: note = +25 DNA gRNA 5' extension
  source 1..25: mol_type = other DNA
atgtgtttttgtcaaaagacctttt

SEQ ID NO: 307 - DNA, 60 bp, synthetic construct
  misc_feature 1..60: note = +60 DNA gRNA 5' extension
  source 1..60: mol_type = other DNA
aggccagcttgccggttttttagtcgtgctgcttcatgtgtttttgtcaaaagacctttt

SEQ ID NO: 308 - DNA, 20 bp, synthetic construct
  misc_feature 1..20: note = +20 DNA gRNA 5' extension
  source 1..20: mol_type = other DNA
tttttgtcaaaagacctttt

SEQ ID NO: 309 - DNA, 30 bp, synthetic construct
  misc_feature 1..30: note = +30 DNA gRNA 5' extension
  source 1..30: mol_type = other DNA
gcttcatgtgtttttgtcaaaagacctttt

SEQ ID NO: 310 - DNA, 50 bp, synthetic construct
  misc_feature 1..50: note = +50 DNA gRNA 5' extension
  source 1..50: mol_type = other DNA
gccggttttttagtcgtgctgcttcatgtgtttttgtcaaaagacctttt

SEQ ID NO: 311 - DNA, 40 bp, synthetic construct
  misc_feature 1..40: note = +40 DNA gRNA 5' extension
  source 1..40: mol_type = other DNA
tagtcgtgctgcttcatgtgtttttgtcaaaagacctttt

SEQ ID NO: 312 - DNA, 20 bp, synthetic construct
  misc_feature 1..20: note = +20 DNA/2xPS gRNA 5' extension
  modified_base 1..2: mod_base = OTHER; note = linked by phosphorothioate (PS) bond
  modified_base 2..3: mod_base = OTHER; note = linked by phosphorothioate (PS) bond
  source 1..20: mol_type = other DNA
ccgaagttttcttcggtttt

SEQ ID NO: 313 - DNA, 25 bp, synthetic construct
  misc_feature 1..25: note = +25 DNA/2xPS gRNA 5' extension
  modified_base 1..2: mod_base = OTHER; note = linked by phosphorothioate (PS) bond
  modified_base 2..3: mod_base = OTHER; note = linked by phosphorothioate (PS) bond
  source 1..25: mol_type = other DNA
tttttccgaagttttcttcggtttt

SEQ ID NO: 314 - DNA, 30 bp, synthetic construct
  misc_feature 1..30: note = +30 DNA/2xPS gRNA 5' extension
  modified_base 1..2: mod_base = OTHER; note = linked by phosphorothioate (PS) bond
  modified_base 2..3: mod_base = OTHER; note = linked by phosphorothioate (PS) bond
  source 1..30: mol_type = other DNA
aacgctttttccgaagttttcttcggtttt

SEQ ID NO: 315 - DNA, 41 bp, synthetic construct
  misc_feature 1..41: note = +41 DNA/2xPS gRNA 5' extension
  modified_base 1..2: mod_base = OTHER; note = linked by phosphorothioate (PS) bond
  modified_base 2..3: mod_base = OTHER; note = linked by phosphorothioate (PS) bond
  source 1..41: mol_type = other DNA
gcgttgttttcaacgctttttccgaagttttcttcggtttt

SEQ ID NO: 316 - DNA, 62 bp, synthetic construct
  misc_feature 1..62: note = +62 DNA/2xPS gRNA 5' extension
  modified_base 1..2: mod_base = OTHER; note = linked by phosphorothioate (PS) bond
  modified_base 2..3: mod_base = OTHER; note = linked by phosphorothioate (PS) bond
  source 1..62: mol_type = other DNA
ggcttcttttgaagcctttttgcgttgttttcaacgctttttccgaagttttcttcggtttt

SEQ ID NO: 317 - DNA, 25 bp, synthetic construct
  misc_feature 1..25: note = +25 DNA/2xPS gRNA 5' extension
  modified_base 1..2: mod_base = OTHER; note = linked by phosphorothioate (PS) bond
  modified_base 2..3: mod_base = OTHER; note = linked by phosphorothioate (PS) bond
  source 1..25: mol_type = other DNA
atgtgtttttgtcaaaagacctttt

SEQ ID NO: 318 - DNA, 25 bp, synthetic construct
  misc_feature 1..25: note = +25 A gRNA 5' extension
  source 1..25: mol_type = other DNA
aaaaaaaaaaaaaaaaaaaaaaaaa

SEQ ID NO: 319 - DNA, 25 bp, synthetic construct
  misc_feature 1..25: note = +25 T gRNA 5' extension
  source 1..25: mol_type = other DNA
ttttttttttttttttttttttttt

SEQ ID NO: 320 - RNA, 21 nt, synthetic construct
  misc_feature 1..21: note = HBG1-1 21mer targeting domain
  source 1..21: mol_type = other RNA
ccttgtcaaggctattggtca

SEQ ID NO: 321 - RNA, 22 nt, synthetic construct
  misc_feature 1..22: note = HBG1-1 22mer targeting domain
  source 1..22: mol_type = other RNA
ccttgtcaaggctattggtcaa

SEQ ID NO: 322 - RNA, 23 nt, synthetic construct
  misc_feature 1..23: note = HBG1-1 23mer targeting domain
  source 1..23: mol_type = other RNA
ccttgtcaaggctattggtcaag

SEQ ID NO: 323 - RNA, 21 nt, synthetic construct
  misc_feature 1..21: note = Cas12a HBG1 promoter-1 21mer targeting domain
  source 1..21: mol_type = other RNA
agacagatatttgcattgaga

SEQ ID NO: 324 - RNA, 21 nt, synthetic construct
  misc_feature 1..21: note = Cas12a HBG1 promoter-2 21mer targeting domain
  source 1..21: mol_type = other RNA
cattgagatagtgtggggaag

SEQ ID NO: 325 - RNA, 21 nt, synthetic construct
  misc_feature 1..21: note = Cas12a HBG1 promoter-6 21mer targeting domain
  source 1..21: mol_type = other RNA
cttctcccatcatagaggata

SEQ ID NO: 326 - protein, 1334 aa, Listeria innocua
  REGION 10..21: note = misc_feature - N-terminal RuvC-like domain
  REGION 762..769: note = misc_feature - RuvC-like domain
  REGION 840..866: note = misc_feature - HNH-like domain
  REGION 985..992: note = misc_feature - RuvC-like domain
MKKPYTIGLDIGTNSVGWAVLTDQYDLVKRKMKIAGDSEKKQIKKNFWGVRLFDEGQTAADRRMARTARRRIERRRNRISYLQGIFAEEMSKTDANFFCRLSDSFYVDNEKRNSRHPFFATIEEEVEYHKNYPTIYHLREELVNSSEKADLRLVYLALAHIIKYRGNFLIEGALDTQNTSVDGIYKQFIQTYNQVFASGIEDGSLKKLEDNKDVAKILVEKVTRKEKLERILKLYPGEKSAGMFAQFISLIVGSKGNFQKPFDLIEKSDIECAKDSYEEDLESLLALIGDEYAELFVAAKNAYSAVVLSSIITVAETETNAKLSASMIERFDTHEEDLGELKAFIKLHLPKHYEEIFSNTEKHGYAGYIDGKTKQADFYKYMKMTLENIEGADYFIAKIEKENFLRKQRTFDNGAIPHQLHLEELEAILHQQAKYYPFLKENYDKIKSLVTFRIPYFVGPLANGQSEFAWLTRKADGEIRPWNIEEKVDFGKSAVDFIEKMTNKDTYLPKENVLPKHSLCYQKYLVYNELTKVRYINDQGKTSYFSGQEKEQIFNDLFKQKRKVKKKDLELFLRNMSHVESPTIEGLEDSFNSSYSTYHDLLKVGIKQEILDNPVNTEMLENIVKILTVFEDKRMIKEQLQQFSDVLDGVVLKKLERRHYTGWGRLSAKLLMGIRDKQSHLTILDYLMNDDGLNRNLMQLINDSNLSFKSIIEKEQVTTADKDIQSIVADLAGSPAIKKGILQSLKIVDELVSVMGYPPQTIVVEMARENQTTGKGKNNSRPRYKSLEKAIKEFGSQILKEHPTDNQELRNNRLYLYYLQNGKDMYTGQDLDIHNLSNYDIDHIVPQSFITDNSIDNLVLTSSAGNREKGDDVPPLEIVRKRKVFWEKLYQGNLMSKRKFDYLTKAERGGLTEADKARFIHRQLVETRQITKNVANILHQRFNYEKDDHGNTMKQVRIVTLKSALVSQFRKQFQLYKVRDVNDYHHAHDAYLNGVVANTLLKVYPQLEPEFVYGDYHQFDWFKANKATAKKQFYTNIMLFFAQKDRIIDENGEILWDKKYLDTVKKVMSYRQMNIVKKTEIQKGEFSKATIKPKGNSSKLIPRKTNWDPMKYGGLDSPNMAYAVVIEYAKGKNKLVFEKKIIRVTIMERKAFEKDEKAFLEEQGYRQPKVLAKLPKYTLYECEEGRRRMLASANEAQKGNQQVLPNHLVTLLHHAANCEVSDGKSLDYIESNREMFAELLAHVSEFAKRYTLAEANLNKINQLFEQNKEGDIKAIAQSFVDLMAFNAMGAPASFKFFETTIERKRYNNLKELLNSTIIYQSITGLYESRKRLDD

SEQ ID NO: 327 - RNA, 24 nt, synthetic construct
  misc_feature 1..24: note = HBG1-1 gRNA (OLI13620) targeting domain plus PAM
  source 1..24: mol_type = other RNA
tttgccttgtcaaggctattggtc

SEQ ID NO: 328 - skipped

SEQ ID NO: 329 - RNA, 10 nt, synthetic construct
  misc_feature 1..10: note = +10 RNA gRNA 5' extension
  source 1..10: mol_type = other RNA
aagacctttt

SEQ ID NO: 330 - RNA, 25 nt, synthetic construct
  misc_feature 1..25: note = +25 RNA gRNA 5' extension
  source 1..25: mol_type = other RNA
atgtgtttttgtcaaaagacctttt

SEQ ID NO: 331 - RNA, 60 nt, synthetic construct
  misc_feature 1..60: note = +60 RNA gRNA 5' extension
  source 1..60: mol_type = other RNA
aggccagcttgccggttttttagtcgtgctgcttcatgtgtttttgtcaaaagacctttt

SEQ ID NO: 332 - RNA, 25 nt, synthetic construct
  misc_feature 1..25: note = +25 RNA/2xPS gRNA 5' extension
  modified_base 1..2: mod_base = OTHER; note = linked by phosphorothioate (PS) bond
  misc_feature 1: note = Modified to contain 2'-O-methyl
  misc_feature 2: note = Modified to contain 2'-O-methyl
  modified_base 2..3: mod_base = OTHER; note = linked by phosphorothioate (PS) bond
  source 1..25: mol_type = other RNA
atgtgtttttgtcaaaagacctttt

SEQ ID NO: 333 - RNA, 25 nt, synthetic construct
  misc_feature 1..25: note = +25 polyA RNA/2xPS gRNA 5' extension
  modified_base 1..2: mod_base = OTHER; note = linked by phosphorothioate (PS) bond
  misc_feature 1: note = Modified to contain 2'-O-methyl
  misc_feature 2: note = Modified to contain 2'-O-methyl
  modified_base 2..3: mod_base = OTHER; note = linked by phosphorothioate (PS) bond
  source 1..25: mol_type = other RNA
aaaaaaaaaaaaaaaaaaaaaaaaa

SEQ ID NO: 334 - RNA, 25 nt, synthetic construct
  misc_feature 1..25: note = +25 polyU RNA/2xPS gRNA 5' extension
  modified_base 1..2: mod_base = OTHER; note = Linked by phosphorothioate (PS) bond
  misc_feature 1: note = Modified to contain 2'-O-methyl
  modified_base 2: mod_base = OTHER; note = Modified to contain 2'-O-methyl
  modified_base 2..3: mod_base = OTHER; note = Linked by phosphorothioate (PS) bond
  source 1..25: mol_type = other RNA
ttttttttttttttttttttttttt

SEQ ID NO: 335 - protein, 1309 aa, synthetic construct
  REGION 1..1309: note = Cas12a
MTQFEGFTNLYQVSKTLRFELIPQGKTLKHIQEQGFIEEDKARNDHYKELKPIIDRIYKTYADQCLQLVQLDWENLSAAIDSYRKEKTEETRNALIEEQATYRNAIHDYFIGRTDNLTDAINKRHAEIYKGLFKAELFNGKVLKQLGTVTTTEHENALLRSFDKFTTYFSGFYENRKNVFSAEDISTAIPHRIVQDNFPKFKENCHIFTRLITAVPSLREHFENVKKAIGIFVSTSIEEVFSFPFYNQLLTQTQIDLYNQLLGGISREAGTEKIKGLNEVLNLAIQKNDETAHIIASLPHRFIPLFKQILSDRNTLSFILEEFKSDEEVIQSFCKYKTLLRNENVLETAEALFNELNSIDLTHIFISHKKLETISSALCDHWDTLRNALYERRISELTGKITKSAKEKVQRSLKHEDINLQEIISAAGKELSEAFKQKTSEILSHAHAALDQPLPTTLKKQEEKEILKSQLDSLLGLYHLLDWFAVDESNEVDPEFSARLTGIKLEMEPSLSFYNKARNYATKKPYSVEKFKLNFQRPTLASGWDVNKEKNNGAILFVKNGLYYLGIMPKQKGRYKALSFEPTEKTSEGFDKMYYDYFPDAAKMIPKCSTQLKAVTAHFQTHTTPILLSNNFIEPLEITKEIYDLNNPEKEPKKFQTAYAKKTGDQKGYREALCKWIDFTRDFLSKYTKTTSIDLSSLRPSSQYKDLGEYYAELNPLLYHISFQRIAEKEIMDAVETGKLYLFQIYNKDFAKGHHGKPNLHTLYWTGLFSPENLAKTSIKLNGQAELFYRPKSRMKRMAARLGEKMLNKKLKDQKTPIPDTLYQELYDYVNHRLSHDLSDEARALLPNVITKEVSHEIIKDRRFTSDKFLFHVPITLNYQAANSPSKFNQRVNAYLKEHPETPIIGIDRGERNLIYITVIDSTGKILEQRSLNTIQQFDYQKKLDNREKERVAARQAWSVVGTIKDLKQGYLSQVIHEIVDLMIHYQAVVVLENLNFGFKSKRTGIAEKAVYQQFEKMLIDKLNCLVLKDYPAEKVGGVLNPYQLTDQFTSFAKMGTQSGFLFYVPAPYTSKIDPLTGFVDPFVWKTIKNHESRKHFLEGFDFLHYDVKTGDFILHFKMNRNLSFQRGLPGFMPAWDIVFEKNETQFDAKGTPFIAGKRIVPVIENHRFTGRYRDLYPANELIALLEEKGIVFRDGSNILPKLLENDDSHAIDTMVALIRSVLQMRNSNAATGEDYINSPVRDLNGVCFDSRFQNPEWPMDADANGAYHIALKGQLLLNHLKESKDLKLQNGISNQDWLAYIQELRNGR

SEQ ID NO: 336 - protein, 3927 aa, synthetic construct
  REGION 1..3927: note = Cas12a
ATGACCCAGTTTGAAGGTTTCACCAATCTGTATCAGGTTAGCAAAACCCTGCGTTTTGAACTGATTCCGCAGGGTAAAACCCTGAAACATATTCAAGAACAGGGCTTCATCGAAGAGGATAAAGCACGTAACGATCACTACAAAGAACTGAAACCGATTATCGACCGCATCTATAAAACCTATGCAGATCAGTGTCTGCAGCTGGTTCAGCTGGATTGGGAAAATCTGAGCGCAGCAATTGATAGTTATCGCAAAGAAAAAACCGAAGAAACCCGTAATGCACTGATTGAAGAACAGGCAACCTATCGTAATGCCATCCATGATTATTTCATTGGTCGTACCGATAATCTGACCGATGCAATTAACAAACGTCACGCCGAAATCTATAAAGGCCTGTTTAAAGCCGAACTGTTTAATGGCAAAGTTCTGAAACAGCTGGGCACCGTTACCACCACCGAACATGAAAATGCACTGCTGCGTAGCTTTGATAAATTCACCACCTATTTCAGCGGCTTTTATGAGAATCGCAAAAACGTGTTTAGCGCAGAAGATATTAGCACCGCAATTCCGCATCGTATTGTGCAGGATAATTTCCCGAAATTCAAAGAGAACTGCCACATTTTTACCCGTCTGATTACCGCAGTTCCGAGCCTGCGTGAACATTTTGAAAACGTTAAAAAAGCCATCGGCATCTTTGTTAGCACCAGCATTGAAGAAGTTTTTAGCTTCCCGTTTTACAATCAGCTGCTGACCCAGACCCAGATTGATCTGTATAACCAACTGCTGGGTGGTATTAGCCGTGAAGCAGGCACCGAAAAAATCAAAGGTCTGAATGAAGTGCTGAATCTGGCCATTCAGAAAAATGATGAAACCGCACATATTATTGCAAGCCTGCCGCATCGTTTTATTCCGCTGTTCAAACAAATTCTGAGCGATCGTAATACCCTGAGCTTTATTCTGGAAGAATTCAAATCCGATGAAGAGGTGATTCAGAGCTTTTGCAAATACAAAACGCTGCTGCGCAATGAAAATGTTCTGGAAACTGCCGAAGCACTGTTTAACGAACTGAATAGCATTGATCTGACCCACATCTTTATCAGCCACAAAAAACTGGAAACCATTTCAAGCGCACTGTGTGATCATTGGGATACCCTGCGTAATGCCCTGTATGAACGTCGTATTAGCGAACTGACCGGTAAAATTACCAAAAGCGCGAAAGAAAAAGTTCAGCGCAGTCTGAAACATGAGGATATTAATCTGCAAGAGATTATTAGCGCAGCCGGTAAAGAACTGTCAGAAGCATTTAAACAGAAAACCAGCGAAATTCTGTCACATGCACATGCAGCACTGGATCAGCCGCTGCCGACCACCCTGAAAAAACAAGAAGAAAAAGAAATCCTGAAAAGCCAGCTGGATAGCCTGCTGGGTCTGTATCATCTGCTGGACTGGTTTGCAGTTGATGAAAGCAATGAAGTTGATCCGGAATTTAGCGCACGTCTGACCGGCATTAAACTGGAAATGGAACCGAGCCTGAGCTTTTATAACAAAGCCCGTAATTATGCCACCAAAAAACCGTATAGCGTCGAAAAATTCAAACTGAACTTTCAGCGTCCGACCCTGGCAAGCGGTTGGGATGTTAATAAAGAAAAAAACAACGGTGCCATCCTGTTCGTGAAAAATGGCCTGTATTATCTGGGTATTATGCCGAAACAGAAAGGTCGTTATAAAGCGCTGAGCTTTGAACCGACGGAAAAAACCAGTGAAGGTTTTGATAAAATGTACTACGACTATTTTCCGGATGCAGCCAAAATGATTCCGAAATGTAGCACCCAGCTGAAAGCAGTTACCGCACATTTTCAGACCCATACCACCCCGATTCTGCTGAGCAATAACTTTATTGAACCGCTGGAAATCACCAAAGAGATCTACGATCTGAATAACCCGGAAAAAGAGCCGAAAAAATTCCAGACCGCATATGCAAAAAAAACCGGTGATCAGAAAGGTTATCGTGAAGCGCTGTGTAAATGGATTGATTTCACCCGTGATTTTCTGAGCAAATACACCAAAACCACCAGTATCGATCTGAGCAGCCTGCGTCCGAGCAGCCAGTATAAAGATCTGGGCGAATATTATGCAGAACTGAATCCGCTGCTGTATCATATTAGCTTTCAGCGTATTGCCGAGAAAGAAATCATGGACGCAGTTGAAACCGGTAAACTGTACCTGTTCCAGATCTACAATAAAGATTTTGCCAAAGGCCATCATGGCAAACCGAATCTGCATACCCTGTATTGGACCGGTCTGTTTAGCCCTGAAAATCTGGCAAAAACCTCGATTAAACTGAATGGTCAGGCGGAACTGTTTTATCGTCCGAAAAGCCGTATGAAACGTATGGCAGCTCGTCTGGGTGAAAAAATGCTGAACAAAAAACTGAAAGACCAGAAAACCCCGATCCCGGATACACTGTATCAAGAACTGTATGATTATGTGAACCATCGTCTGAGCCATGATCTGAGTGATGAAGCACGTGCCCTGCTGCCGAATGTTATTACCAAAGAAGTTAGCCACGAGATCATTAAAGATCGTCGTTTTACCAGCGACAAATTCCTGTTTCATGTGCCGATTACCCTGAATTATCAGGCAGCAAATAGCCCGAGCAAATTTAACCAGCGTGTTAATGCATATCTGAAAGAACATCCAGAAACGCCGATTATTGGTATTGATCGTGGTGAACGTAACCTGATTTATATCACCGTTATTGATAGCACCGGCAAAATCCTGGAACAGCGTAGCCTGAATACCATTCAGCAGTTTGATTACCAGAAAAAACTGGATAATCGCGAGAAAGAACGTGTTGCAGCACGTCAGGCATGGTCAGTTGTTGGTACAATTAAAGACCTGAAACAGGGTTATCTGAGCCAGGTTATTCATGAAATTGTGGATCTGATGATTCACTATCAGGCCGTTGTTGTGCTGGAAAACCTGAATTTTGGCTTTAAAAGCAAACGTACCGGCATTGCAGAAAAAGCAGTTTATCAGCAGTTCGAGAAAATGCTGATTGACAAACTGAATTGCCTGGTGCTGAAAGATTATCCGGCTGAAAAAGTTGGTGGTGTTCTGAATCCGTATCAGCTGACCGATCAGTTTACCAGCTTTGCAAAAATGGGCACCCAGAGCGGATTTCTGTTTTATGTTCCGGCACCGTATACGAGCAAAATTGATCCGCTGACCGGTTTTGTTGATCCGTTTGTTTGGAAAACCATCAAAAACCATGAAAGCCGCAAACATTTTCTGGAAGGTTTCGATTTTCTGCATTACGACGTTAAAACGGGTGATTTCATCCTGCACTTTAAAATGAATCGCAATCTGAGTTTTCAGCGTGGCCTGCCTGGTTTTATGCCTGCATGGGATATTGTGTTTGAGAAAAACGAAACACAGTTCGATGCAAAAGGCACCCCGTTTATTGCAGGTAAACGTATTGTTCCGGTGATTGAAAATCATCGTTTCACCGGTCGTTATCGCGATCTGTATCCGGCAAATGAACTGATCGCACTGCTGGAAGAGAAAGGTATTGTTTTTCGTGATGGCTCAAACATTCTGCCGAAACTGCTGGAAAATGATGATAGCCATGCAATTGATACCATGGTTGCACTGATTCGTAGCGTTCTGCAGATGCGTAATAGCAATGCAGCAACCGGTGAAGATTACATTAATAGTCCGGTTCGTGATCTGAATGGTGTTTGTTTTGATAGCCGTTTTCAGAATCCGGAATGGCCGATGGATGCAGATGCAAATGGTGCATATCATATTGCACTGAAAGGACAGCTGCTGCTGAACCACCTGAAAGAAAGCAAAGATCTGAAACTGCAAAACGGCATTAGCAATCAGGATTGGCTGGCATATATCCAAGAACTGCGTAACGGTCGT

(19) United States
(12) Patent Application Publication
(10) Pub. No.: US 2026/0256948 A1
(43) Pub. Date: Sep. 3, 2026

(54) SYSTEMS AND METHODS FOR THE TREATMENT OF HEMOGLOBINOPATHIES

(71) Applicant: EDITAS MEDICINE, INC., Cambridge, MA (US)

(72) Inventor: KaiHsin CHANG, Cambridge, MA (US)

(73) Assignee: EDITAS MEDICINE, INC., Cambridge, MA (US)

(21) Appl. No.: 19/491,219

(22) PCT Filed: Jun. 7, 2024
(86) PCT No.: PCT/US2024/033161

§ 371 Date: Dec. 8, 2025

Related U.S. Application Data
(60) Provisional application No. 63/507,092, filed on Jun. 8, 2023.
(60) Provisional application No. 63/593,940, filed on Oct. 27, 2023.
(60) Provisional application No. 63/606,586, filed on Dec. 5, 2023.
(60) Provisional application No. 63/646,703, filed on May 13, 2024.

(51) Int. Cl. A61K 48/00; A61K 35/28; A61K 38/00; A61P 7/06; C07K 14/805; C12N 5/0789; C12N 9/22; C12N 15/11

(52) U.S. Cl. CPC A61K 48/005; A61K 35/28; A61P 7/06; C07K 14/805; C12N 5/0647; C12N 9/226; C12N 15/111; A61K 38/00; C12N 2310/20; C12N 2310/315; C12N 2310/321; C12N 2510/00

(57) ABSTRACT

Genome editing systems, guide RNAs, and CRISPR-mediated methods are provided for altering portions of the HBG1 and HBG2 loci in cells and increasing expression of fetal hemoglobin.

### PRIORITY CLAIM

This application claims the benefit of U.S. Provisional Application No. 63/507,092, filed Jun. 8, 2023, U.S. Provisional Application No. 63/593,940, filed Oct. 27, 2023, U.S. Provisional Application No. 63/606,586, filed Dec. 5, 2023, and U.S. Provisional Application No. 63/646,703, filed May 13, 2024, all of which are incorporated herein by reference in their entirety, including drawings.

### SEQUENCE LISTING

This application contains a ST.26 compliant Sequence Listing, which was submitted in XML format via Patent Center, and is hereby incorporated by reference in its entirety. The XML copy, created on Jun. 7, 2024, is named 1189458028W000.xml and is 738,000 bytes in size.

### FIELD

This disclosure relates to genome editing systems and methods for altering a target nucleic acid sequence, or modulating expression of a target nucleic acid sequence, and applications thereof in connection with the alteration of genes encoding hemoglobin subunits and/or treatment of hemoglobinopathies.

### BACKGROUND

Hemoglobin (Hb) carries oxygen in erythrocytes or red blood cells (RBCs) from the lungs to tissues. During prenatal development and until shortly after birth, hemoglobin is present in the form of fetal hemoglobin (HbF), a tetrameric protein composed of two alpha (α)-globin chains and two gamma (γ)-globin chains. HbF is largely replaced by adult hemoglobin (HbA), a tetrameric protein in which the γ-globin chains of HbF are replaced with beta (β)-globin chains, through a process known as globin switching. The average adult makes less than 1% HbF out of total hemoglobin (Thein 2009). The α-hemoglobin gene is located on chromosome 16, while the β-hemoglobin gene (HBB), A gamma (Aγ)-globin chain (HBG1, also known as gamma globin A), and G gamma (Gγ)-globin chain (HBG2, also known as gamma globin G) are located on chromosome 11 within the globin gene cluster (also referred to as the globin locus).

Mutations in HBB can cause hemoglobin disorders (i.e., β-hemoglobinopathies) including sickle cell disease (SCD) and beta-thalassemia (β-Thal). Approximately 93,000 people in the United States are have a β-hemoglobinopathy. Worldwide, 300,000 children are born with hemoglobinopathies every year (Angastiniotis 1998). Because these conditions are associated with HBB mutations, their symptoms typically do not manifest until after globin switching from HbF to HbA.

SCD is the most common inherited hematologic disease in the United States, affecting approximately 80,000 people (Brousseau 2010). SCD is most common in people of African ancestry, for whom the prevalence of SCD is 1 in 500. In Africa, the prevalence of SCD is 15 million (Aliyu 2008). SCD is also more common in people of Indian, Saudi Arabian and Mediterranean descent. In those of Hispanic-American descent, the prevalence of sickle cell disease is 1 in 1,000 (Lewis 2014).

SCD is caused by a single homozygous mutation in the HBB gene, c.17A>T (HbS mutation). The sickle mutation is a point mutation (GAG>GTG) on HBB that results in substitution of valine for glutamic acid at amino acid position 6 in exon 1. The valine at position 6 of the β-hemoglobin chain is hydrophobic and causes a change in conformation of the β-globin protein when it is not bound to oxygen. This change of conformation causes HbS proteins to polymerize in the absence of oxygen, leading to deformation (i.e., sickling) of RBCs. SCD is inherited in an autosomal recessive manner, so that only patients with two HbS alleles have the disease. Heterozygous subjects have sickle cell trait, and may suffer from anemia and/or painful crises if they are severely dehydrated or oxygen deprived.

Sickle shaped RBCs cause multiple symptoms, including anemia, sickle cell crises, vaso-occlusive crises, aplastic crises, and acute chest syndrome. Sickle shaped RBCs are less elastic than wild-type RBCs and therefore cannot pass as easily through capillary beds and cause occlusion and ischemia (i.e., vaso-occlusion). Vaso-occlusive crisis occurs when sickle cells obstruct blood flow in the capillary bed of an organ leading to pain, ischemia, and necrosis. These episodes typically last 5-7 days. The spleen plays a role in clearing dysfunctional RBCs, and is therefore typically enlarged during early childhood and subject to frequent vaso-occlusive crises. By the end of childhood, the spleen in SCD patients is often infarcted, which leads to autosplenectomy. Hemolysis is a constant feature of SCD and causes anemia. Sickle cells survive for 10-20 days in circulation, while healthy RBCs survive for 90-120 days. SCD subjects are transfused as necessary to maintain adequate hemoglobin levels. Frequent transfusions place subjects at risk for infection with HIV, Hepatitis B, and Hepatitis C. Subjects may also suffer from acute chest crises and infarcts of extremities, end organs, and the central nervous system.

Subjects with SCD have decreased life expectancies. The prognosis for patients with SCD is steadily improving with careful, life-long management of crises and anemia. As of 2001, the average life expectancy of subjects with sickle cell disease was the mid-to-late 50's. Current treatments for SCD involve hydration and pain management during crises, and transfusions as needed to correct anemia.

Thalassemias (e.g., β-Thal, δ-Thal, and β/δ-Thal) cause chronic anemia. β-Thal is estimated to affect approximately 1 in 100,000 people worldwide. Its prevalence is higher in certain populations, including those of European descent, where its prevalence is approximately 1 in 10,000. β-Thal major, the more severe form of the disease, is life-threatening unless treated with lifelong blood transfusions and chelation therapy. In the United States, there are approximately 3,000 subjects with β-Thal major. β-Thal intermedia does not require blood transfusions, but it may cause growth delay and significant systemic abnormalities, and it frequently requires lifelong chelation therapy. Although HbA makes up the majority of hemoglobin in adult RBCs, approximately 3% of adult hemoglobin is in the form of HbA2, an HbA variant in which the two γ-globin chains are replaced with two delta (Δ)-globin chains. δ-Thal is associated with mutations in the Δ hemoglobin gene (HBD) that cause a loss of HBD expression. Co-inheritance of the HBD mutation can mask a diagnosis of β-Thal (i.e., β/δ-Thal) by decreasing the level of HbA2 to the normal range (Bouva 2006). β/δ-Thal is usually caused by deletion of the HBB and HBD sequences in both alleles. In homozygous (δo/δo βo/βo) patients, HBG is expressed, leading to production of HbF alone.

Like SCD, β-Thal is caused by mutations in the HBB gene. The most common HBB mutations leading to β-Thal are: c.-136C>G, c.92+1G>A, c.92+6T>C, c.93−21G>A, c.118C>T, c.316−106C>G, c.25_26delAA, c.27_28insG, c.92+5G>C, c.118C>T, c.135delC, c.315+1G>A, c.-78A>G, c.52A>T, c.59A>G, c.92+5G>C, c.124_127delTTCT, c.316−197C>T, c.-78A>G, c.52A>T, c.124_127delTTCT, c.316−197C>T, c.-138C>T, c.-79A>G, c.92+5G>C, c.75T>A, c.316−2A>G, and c.316−2A>C. These and other mutations associated with β-Thal cause mutated or absent β-globin chains, which causes a disruption of the normal Hb α-hemoglobin to β-hemoglobin ratio. Excess α-globin chains precipitate in erythroid precursors in the bone marrow.

In β-Thal major, both alleles of HBB contain nonsense, frameshift, or splicing mutations that leads to complete absence of β-globin production (denoted β⁰/β⁰). β-Thal major results in severe reduction in β-globin chains, leading to significant precipitation of α-globin chains in RBCs and more severe anemia.

β-Thal intermedia results from mutations in the 5′ or 3′ untranslated region of HBB, mutations in the promoter region or polyadenylation signal of HBB, or splicing mutations within the HBB gene. Patient genotypes are denoted βo/β+ or β+/β+. βo represents absent expression of a β-globin chain; β+ represents a dysfunctional but present β-globin chain. Phenotypic expression varies among patients. Since there is some production of β-globin, β-Thal intermedia results in less precipitation of α-globin chains in the erythroid precursors and less severe anemia than β-Thal major. However, there are more significant consequences of erythroid lineage expansion secondary to chronic anemia.

Subjects with β-Thal major present between the ages of 6 months and 2 years, and suffer from failure to thrive, fevers, hepatosplenomegaly, and diarrhea. Adequate treatment includes regular transfusions. Therapy for J-Thal major also includes splenectomy and treatment with hydroxyurea. If patients are regularly transfused, they will develop normally until the beginning of the second decade. At that time, they require chelation therapy (in addition to continued transfusions) to prevent complications of iron overload. Iron overload may manifest as growth delay or delay of sexual maturation. In adulthood, inadequate chelation therapy may lead to cardiomyopathy, cardiac arrhythmias, hepatic fibrosis and/or cirrhosis, diabetes, thyroid and parathyroid abnormalities, thrombosis, and osteoporosis. Frequent transfusions also put subjects at risk for infection with HIV, hepatitis B and hepatitis C.

β-Thal intermedia subjects generally present between the ages of 2-6 years. They do not generally require blood transfusions. However, bone abnormalities occur due to chronic hypertrophy of the erythroid lineage to compensate for chronic anemia. Subjects may have fractures of the long bones due to osteoporosis. Extramedullary erythropoiesis is common and leads to enlargement of the spleen, liver, and lymph nodes. It may also cause spinal cord compression and neurologic problems. Subjects also suffer from lower extremity ulcers and are at increased risk for thrombotic events, including stroke, pulmonary embolism, and deep vein thrombosis. Treatment of β-Thal intermedia includes splenectomy, folic acid supplementation, hydroxyurea therapy, and radiotherapy for extramedullary masses. Chelation therapy is used in subjects who develop iron overload.

Life expectancy is often diminished in β-Thal patients. Subjects with β-Thal major who do not receive transfusion therapy generally die in their second or third decade. Subjects with β-Thal major who receive regular transfusions and adequate chelation therapy can live into their fifth decade and beyond. Cardiac failure secondary to iron toxicity is the leading cause of death in β-Thal major subjects due to iron toxicity.

A variety of new treatments are currently in development for SCD and β-Thal. Delivery of an anti-sickling HBB gene via gene therapy is currently being investigated in clinical trials. However, the long-term efficacy and safety of this approach is unknown. Transplantation with hematopoietic stem cells (HSCs) from an HLA-matched allogencic stem cell donor has been demonstrated to cure SCD and β-Thal, but this procedure involves risks including those associated with ablation therapy, which is required to prepare the subject for transplant, increases risk of life-threatening opportunistic infections, and risk of graft vs. host disease after transplantation. In addition, matched allogeneic donors often cannot be identified. Thus, there is a need for improved methods of managing these and other hemoglobinopathies.

### SUMMARY

Provided herein in certain aspects are methods of inducing expression of fetal hemoglobin (HbF) in a population of cells, e.g., CD34+ hematopoietic stem and progenitor cells (HSPCs) and/or red blood cells, in a subject. In certain embodiments, the subject may be suffering from a β-hemoglobinopathy. In certain embodiments, the β-hemoglobinopathy may be sickle cell disease (SCD) or β-Thal. In certain embodiments, the method may comprise administering to the subject a population of modified cells comprising a plurality of modified CD34+ or hematopoietic stem cells comprising an indel in an HBG promoter, thereby inducing expression of HbF in the population of cells (e.g., HSPCs or RBCs). In certain embodiments, the HbF as a percentage of total hemoglobin (% HbF) in the subject (e.g., in the blood or peripheral blood of the subject) may be about 5%, 10%, 15%, 20%, 25%, 30%, 35%, 40%, 45%, 50%, 55%, 60%, 65%, 70%, 75%, or 80% HbF. In certain embodiments, the % HbF in the subject (e.g., in the blood or peripheral blood of the subject) may be from about 10% to about 30%, from about 20% to about 40%, from about 30% to about 50%, from about 40% to about 60%, from about 50% to about 70%, from about 60% to about 80%, from about 10% to about 20%, from about 20% to about 30%, from about 30% to about 40%, from about 40% to about 50%, from about 50% to about 60%, from about 60% to about 70%, from about 70% to about 80% HbF, or a range defined by any of the two preceding values. In certain embodiments, a concentration of total hemoglobin in the subject (e.g., in the blood or peripheral blood of the subject) may be about 10 g/dL, 11 g/dL, 12 g/dL, 13 g/dL, 14 g/dL, 15 g/dL, 16 g/dL, 17 g/dL, 18 g/dL, 19 g/dL, or 20 g/dL. In certain embodiments, a concentration of total hemoglobin in the subject (e.g., in the blood or peripheral blood of the subject) may be from about 10.0 to about 20.0 g/dL, from about 13.6 to about 18.0 g/dL (for males), from about 12.0 to about 16.0 g/dL (for females), or a range defined by any of the two preceding values. In certain embodiments, a percentage of F-cells among circulating RBCs in the subject may be about 5%, 10%, 15%, 20%, 25%, 30%, 35%, 40%, 45%, 50%, 55%, 60%, 65%, 70%, 75%, 80%, 85%, 90%, 95%, 100% F-cells. In certain embodiments, a percentage of F-cells among circulating RBCs in the subject may be from about 50% to about 100%, from about 60% to about 100%, from about 70% to about 100%, from about 80% to about 100%, from about 50% to about 60%, from about 60% to about 70%, from about 70% to about 80%, from about 80% to about 90%, from about 90% to about 100%, or a range defined by any of the two preceding values. In certain embodiments, a Hemoglobin F concentration in the subject may be about 1 g/dL, 2 g/dL, 3 g/dL, 4 g/dL, 5 g/dL, 6 g/dL, 7 g/dL, 8 g/dL, 9 g/dL, 10 g/dL, 11 g/dL, 12 g/dL, 13 g/dL, 14 g/dL, 15 g/dL, 16 g/dL, 17 g/dL, 18 g/dL, 19 g/dL, or 20 g/dL. In certain embodiments, a Hemoglobin F concentration in the subject may be from about 1 g/dL to about 20 g/dL, or a range defined by any of the two preceding values. In certain embodiments, a mean corpuscular HbF (pg/RBC) in the subject may be about 1.0 pg/RBC, 2.0 pg/RBC, 3.0 pg/RBC, 4.0 pg/RBC, 5.0 pg/RBC, 6.0 pg/RBC, 7.0 pg/RBC, 8.0 pg/RBC, 9.0 pg/RBC, 10.0 pg/RBC, 11.0 pg/RBC, 12.0 pg/RBC, 13.0 pg/RBC, 14.0 pg/RBC 15.0 pg/RBC, 16.0 pg/RBC, 17.0 pg/RBC, 18.0 pg/RBC, 19.0 pg/RBC, 20.0 pg/RBC, 25.0 pg/RBC, or 30.0 pg/RBC. In certain embodiments, a mean corpuscular HbF (pg/RBC) in the subject may be from about 1.0 pg/RBC to about 5.00 pg/RBC, from about 5.0 pg/RBC to about 10.00 pg/RBC, from about 10.0 pg/RBC to about 15.00 pg/RBC, from about 15.0 pg/RBC to about 20.00 pg/RBC, from about 20.0 pg/RBC to about 25.00 pg/RBC, from about 25.0 pg/RBC to about 30.00 pg/RBC, or a range defined by any of the two preceding values. In certain embodiments, a mean corpuscular HbF (pg/RBC) in the subject may be >10.0 pg/RBC. In certain embodiments, a mean proportion of HbF as a percentage of total hemoglobin (Hb) in the subject is about 5%, 10%, 15%, 20%, 25%, 30%, 35%, 40%, 45%, 50%, 55%, 60%, 65%, 70%, 75%, 80%, 85%, 90%, 95%. In certain embodiments, a mean proportion of HbS as a percentage of total hemoglobin (Hb) in the subject is about 5%, 10%, 15%, 20%, 25%, 30%, 35%, 40%, 45%, 50%, 55%, 60%, 65%, 70%, 75%, 80%, 85%, 90%, 95%. In certain embodiments, a mean proportion of HbA as a percentage of total hemoglobin (Hb) in the subject is about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%. In certain embodiments, a mean proportion of HbA2 as a percentage of total hemoglobin (Hb) in the subject is about 1%, 2%, 3%, 4%, 5%. In certain embodiments, a lactate dehydrogenase (U/L) in the subject is about 110 U/L, 120 U/L, 130 U/L, 140 U/L, 150 U/L, 160 U/L, 170 U/L, 180 U/L, 190 U/L, 200 U/L, 210 U/L, 220 U/L, or 230 U/L. In certain embodiments, a lactate dehydrogenase (U/L) in the subject is from about 110 U/L to about 230 U/L, or a range defined by any of the two preceding values. In certain embodiments, an indirect bilirubin (mol/L) in the subject is about 0.0 μmol/L, 1 μmol/L, 2 μmol/L, 3 μmol/L, 4 μmol/L, 5 μmol/L, 6 μmol/L, 7 μmol/L, 8 μmol/L, 9 μmol/L, 10 μmol/L, 11 μmol/L, 12 μmol/L, 13 μmol/L, 14 μmol/L, 15 μmol/L, 16 μmol/L, or 17 μmol/L. In certain embodiments, an indirect bilirubin (μmol/L) in the subject is from about 0.0 μmol/L to about 16.6 μmol/L, or a range defined by the two preceding values. In certain embodiments, a haptoglobin (g/L) in the subject is about 0.3 g/L, 0.4 g/L, 0.5 g/L, 0.6 g/L, 0.7 g/L, 0.8 g/L, 0.9 g/L, 1.0 g/L, 1.1 g/L, 1.2 g/L, 1.3 g/L, 1.4 g/L, 1.5 g/L, 1.6 g/L, 1.7 g/L, 1.8 g/L, 1.9 g/L, or 2.0 g/L. In certain embodiments, a haptoglobin (g/L) in the subject is from about 0.3 g/L to about 2.0 g/L or a range defined by the two preceding values. In certain embodiments, a reticulocyte count (%) in the subject is about 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, or 2.3%. In certain embodiments, a reticulocyte count (%) in the subject is from about 0.3% to about 2.3% or a range defined by the two preceding values. In certain embodiments, normalization of total hemoglobin in the subject occurs by at least 1 month, 1.5 months, 2 months, 3 months, 4 months, 5 months after administering the population of modified cells to the subject. In certain embodiments, the subject may undergo myeloablative conditioning with busulfan prior to administering the population of modified cells. In certain embodiments, administering the population of modified cells may comprise a single infusion of the modified population of cells. In certain embodiments, the population of modified cells may be about ≥1×10⁶ cells/kg, ≥2×10⁶ cells/kg, ≥3×10⁶ cells/kg, ≥4×10⁶ cells/kg, ≥5×10⁶ cells/kg, ≥6×10⁶ cells/kg, ≥7×10⁶ cells/kg, ≥8×10⁶ cells/kg, ≥9×10⁶ cells/kg, ≥10×10⁶ cells/kg, ≥11×10⁶ cells/kg, ≥12×10⁶ cells/kg, ≥13×10⁶ cells/kg, ≥14×10⁶ cells/kg, ≥15×10⁶ cells/kg, ≥16×10⁶ cells/kg, ≥17×10⁶ cells/kg, ≥18×10⁶ cells/kg, ≥19×10⁶ cells/kg, ≥20×10⁶ cells/kg, ≥21×10⁶ cells/kg, ≥22×10⁶ cells/kg, ≥23×10⁶ cells/kg, ≥24×10⁶ cells/kg, ≥25×10⁶ cells/kg, 1×10⁶ cells/kg to 25×10⁶ cells/kg, 1×10⁶ cells/kg to 5×10⁶ cells/kg, 3×10⁶ cells/kg to 20×10⁶ cells/kg, 3×10⁶ cells/kg to 15×10⁶ cells/kg, 3×10⁶ cells/kg to 10×10⁶ cells/kg, 3×10⁶ cells/kg to 5×10⁶ cells/kg, 5×10⁶ cells/kg to 10×10⁶ cells/kg, 10×10⁶ cells/kg to 15×10⁶ cells/kg, 15×10⁶ cells/kg to 20×10⁶ cells/kg, 20×10⁶ cells/kg to 25×10⁶ cells/kg. In certain embodiments, the % HbF may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the concentration of total hemoglobin may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the Hemoglobin F concentration may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the percentage of F-cells may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean corpuscular HbF (pg/RBC) may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean proportion of HbF as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean proportion of HbS as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean proportion of HbA as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean proportion of HbA2 as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the lactate dehydrogenase (U/L) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the indirect bilirubin (μmol/L) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the haptoglobin (g/L) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the reticulocyte count (%) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience no vaso-occlusive events (VOEs) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience no severe VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 2, 3, 4, 5, 6, 7, 8, 9, 10, 11, 12, 13, 14, 15, 16, 17, 18, 19, or 20 VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 2, 3, 4, 5, 6, 7, 8, 9, or 10 severe VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 1, 2, 3, 4, 5, 10, 15, 20, 25, 30, 35, 40, 45, 50, 55, 60, 65 treatment emergent adverse events (TEAEs) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 1, 2, 3, 4, 5, 6, 7, 8, 9, or 10 serious TEAEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may be red blood cell (RBC) transfusion free 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, an RNP complex comprising a guide RNA (gRNA) and a Cas12a (also known as Cpf1) protein may be delivered to a population of unmodified cells comprising a plurality of unmodified CD34+ or hematopoietic stem cells from the subject to generate the population of modified cells. In certain embodiments, the gRNA may comprise a targeting domain comprising SEQ ID NO:320. In certain embodiments, the gRNA may comprise a targeting domain comprising SEQ ID NO:202. In certain embodiments, the gRNA may comprise a 5′ end and a 3′ end, a DNA extension at the 5′ end. In certain embodiments, the gRNA may comprise a 2′-O-methyl, phosphorothioate modification, or both at the 3′ end. In certain embodiments, the DNA extension may comprise a sequence selected from the group consisting of SEQ ID NOs:304-319. In certain embodiments, the gRNA may comprise a DNA extension sequence set forth in SEQ ID NO:306. In certain embodiments, the gRNA may comprise SEQ ID NO:237. In certain embodiments, the Cas12a protein may comprise a sequence selected from the group consisting of SEQ ID NO:200, 201, 205-215, 221, 222-226, 280-283, 293-295, and 335. In certain embodiments, the Cas12a protein may comprise SEQ ID NO:283. In certain embodiments, the Cas12a protein may comprise SEQ ID NO:335. In certain embodiments, the Cas12a protein may be encoded by a sequence comprising a sequence selected from the group consisting of SEQ ID NOs:216-218, 296-303, and 336. In certain embodiments, the Cas12a protein may be encoded by a sequence comprising SEQ ID NO:300. In certain embodiments, the Cas12a protein may be encoded by a sequence comprising SEQ ID NO:336. In certain embodiments, the indel in the HBG gene promoter may be in a CCAAT box target region. In certain embodiments, the subject has a single homozygous mutation in the HBB gene, c.17A>T (HbS mutation). In certain embodiments, the subject may be suffering from severe sickle cell disease. In certain embodiments, the subject may be suffering from severe sickle cell disease. In certain embodiments, the subject may be suffering from transfusion-dependent beta-thalassemia (TDT).

Provided herein in certain aspects are methods of treating a β-hemoglobinopathy. In certain embodiments, the β-hemoglobinopathy may be sickle cell disease (SCD). In certain embodiments, the β-hemoglobinopathy may be β-Thal. In certain embodiments, the β-Thal may be transfusion-dependent beta-thalassemia (TDT). In certain embodiments, the method may comprise administering to the subject a population of modified cells comprising a plurality of modified CD34+ or hematopoietic stem cells comprising an indel in an HBG promoter, thereby treating the β-hemoglobinopathy. In certain embodiments, administration of the modified cells may induce expression of fetal hemoglobin (HbF) in a population of cells, e.g., CD34+ hematopoietic stem and progenitor cells (HSPCs) and/or red blood cells. In certain embodiments, an RNP complex comprising a guide RNA (gRNA) and a Cas12a (also known as Cpf1) protein may be delivered to a population of unmodified cells comprising a plurality of unmodified CD34+ or hematopoietic stem cells from the subject to generate the population of modified cells. In certain embodiments, the gRNA may comprise a targeting domain comprising SEQ ID NO:320. In certain embodiments, the gRNA may comprise a targeting domain comprising SEQ ID NO:202. In certain embodiments, the gRNA may comprise a 5′ end and a 3′ end, a DNA extension at the 5′ end. In certain embodiments, the gRNA may comprise a 2′-O-methyl, phosphorothioate modification, or both at the 3′ end. In certain embodiments, the DNA extension may comprise a sequence selected from the group consisting of SEQ ID NOs:304-319. In certain embodiments, the gRNA may comprise a DNA extension sequence set forth in SEQ ID NO:306. In certain embodiments, the gRNA may comprise SEQ ID NO:237. In certain embodiments, the Cas12a protein may comprise a sequence selected from the group consisting of SEQ ID NO:200, 201, 205-215, 221, 222-226, 280-283, 293-295, and 335. In certain embodiments, the Cas12a protein may comprise SEQ ID NO:283. In certain embodiments, the Cas12a protein may comprise SEQ ID NO:335. In certain embodiments, the Cas12a protein may be encoded by a sequence comprising a sequence selected from the group consisting of SEQ ID NOs:216-218, 296-303, and 336. In certain embodiments, the Cas12a protein may be encoded by a sequence comprising SEQ ID NO:300. In certain embodiments, the Cas12a protein may be encoded by a sequence comprising SEQ ID NO:336. In certain embodiments, the indel in the HBG gene promoter may be in a CCAAT box target region. In certain embodiments, the subject has a single homozygous mutation in the HBB gene, c.17A>T (HbS mutation). In certain embodiments, the HbF as a percentage of total hemoglobin (% HbF) in the subject (e.g., in the blood or peripheral blood of the subject) may be about 5%, 10%, 15%, 20%, 25%, 30%, 35%, 40%, 45%, 50%, 55%, 60%, 65%, 70%, 75%, or 80% HbF. In certain embodiments, the % HbF in the subject (e.g., in the blood or peripheral blood of the subject) may be from about 10% to about 30%, from about 20% to about 40%, from about 30% to about 50%, from about 40% to about 60%, from about 50% to about 70%, from about 60% to about 80%, from about 10% to about 20%, from about 20% to about 30%, from about 30% to about 40%, from about 40% to about 50%, from about 50% to about 60%, from about 60% to about 70%, from about 70% to about 80% HbF, or a range defined by any of the two preceding values. In certain embodiments, a concentration of total hemoglobin in the subject (e.g., in the blood or peripheral blood of the subject) may be about 10 g/dL, 11 g/dL, 12 g/dL, 13 g/dL, 14 g/dL, 15 g/dL, 16 g/dL, 17 g/dL, 18 g/dL, 19 g/dL, or 20 g/dL. In certain embodiments, a concentration of total hemoglobin in the subject (e.g., in the blood or peripheral blood of the subject) may be from about 10.0 to about 20.0 g/dL, from about 13.6 to about 18.0 g/dL (for males), from about 12.0 to about 16.0 g/dL (for females), or a range defined by any of the two preceding values. In certain embodiments, a percentage of F-cells among circulating RBCs in the subject may be about 5%, 10%, 15%, 20%, 25%, 30%, 35%, 40%, 45%, 50%, 55%, 60%, 65%, 70%, 75%, 80%, 85%, 90%, 95%, 100% F-cells. In certain embodiments, a percentage of F-cells among circulating RBCs in the subject may be from about 50% to about 100%, from about 60% to about 100%, from about 70% to about 100%, from about 80% to about 100%, from about 50% to about 60%, from about 60% to about 70%, from about 70% to about 80%, from about 80% to about 90%, from about 90% to about 100%, or a range defined by any of the two preceding values. In certain embodiments, a Hemoglobin F concentration in the subject may be about 1 g/dL, 2 g/dL, 3 g/dL, 4 g/dL, 5 g/dL, 6 g/dL, 7 g/dL, 8 g/dL, 9 g/dL, 10 g/dL, 11 g/dL, 12 g/dL, 13 g/dL, 14 g/dL, 15 g/dL, 16 g/dL, 17 g/dL, 18 g/dL, 19 g/dL, or 20 g/dL. In certain embodiments, a Hemoglobin F concentration in the subject may be from about 1 g/dL to about 20 g/dL, or a range defined by any of the two preceding values. In certain embodiments, a mean corpuscular HbF (pg/RBC) in the subject may be about 1.0 pg/RBC, 2.0 pg/RBC, 3.0 pg/RBC, 4.0 pg/RBC, 5.0 pg/RBC, 6.0 pg/RBC, 7.0 pg/RBC, 8.0 pg/RBC, 9.0 pg/RBC, 10.0 pg/RBC, 11.0 pg/RBC, 12.0 pg/RBC, 13.0 pg/RBC, 14.0 pg/RBC 15.0 pg/RBC, 16.0 pg/RBC, 17.0 pg/RBC, 18.0 pg/RBC, 19.0 pg/RBC, 20.0 pg/RBC, 25.0 pg/RBC, or 30.0 pg/RBC. In certain embodiments, a mean corpuscular HbF (pg/RBC) in the subject may be from about 1.0 pg/RBC to about 5.00 pg/RBC, from about 5.0 pg/RBC to about 10.00 pg/RBC, from about 10.0 pg/RBC to about 15.00 pg/RBC, from about 15.0 pg/RBC to about 20.00 pg/RBC, from about 20.0 pg/RBC to about 25.00 pg/RBC, from about 25.0 pg/RBC to about 30.00 pg/RBC, or a range defined by any of the two preceding values. In certain embodiments, a mean corpuscular HbF (pg/RBC) in the subject may be ≥10.0 pg/RBC. In certain embodiments, a mean proportion of HbF as a percentage of total hemoglobin (Hb) in the subject is about 5%, 10%, 15%, 20%, 25%, 30%, 35%, 40%, 45%, 50%, 55%, 60%, 65%, 70%, 75%, 80%, 85%, 90%, 95%. In certain embodiments, a mean proportion of HbS as a percentage of total hemoglobin (Hb) in the subject is about 5%, 10%, 15%, 20%, 25%, 30%, 35%, 40%, 45%, 50%, 55%, 60%, 65%, 70%, 75%, 80%, 85%, 90%, 95%. In certain embodiments, a mean proportion of HbA as a percentage of total hemoglobin (Hb) in the subject is about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%. In certain embodiments, a mean proportion of HbA2 as a percentage of total hemoglobin (Hb) in the subject is about 1%, 2%, 3%, 4%, 5%. In certain embodiments, a lactate dehydrogenase (U/L) in the subject is about 110 U/L, 120 U/L, 130 U/L, 140 U/L, 150 U/L, 160 U/L, 170 U/L, 180 U/L, 190 U/L, 200 U/L, 210 U/L, 220 U/L, or 230 U/L. In certain embodiments, a lactate dehydrogenase (U/L) in the subject is from about 110 U/L to about 230 U/L, or a range defined by any of the two preceding values. In certain embodiments, an indirect bilirubin (μmol/L) in the subject is about 0.0 μmol/L, 1 μmol/L, 2 μmol/L, 3 μmol/L, 4 μmol/L, 5 μmol/L, 6 μmol/L, 7 μmol/L, 8 μmol/L, 9 μmol/L, 10 μmol/L, 11 μmol/L, 12 μmol/L, 13 μmol/L, 14 p mol/L, 15 μmol/L, 16 μmol/L, or 17 μmol/L. In certain embodiments, an indirect bilirubin (μmol/L) in the subject is from about 0.0 μmol/L to about 16.6 μmol/L, or a range defined by the two preceding values. In certain embodiments, a haptoglobin (g/L) in the subject is about 0.3 g/L, 0.4 g/L, 0.5 g/L, 0.6 g/L, 0.7 g/L, 0.8 g/L, 0.9 g/L, 1.0 g/L, 1.1 g/L, 1.2 g/L, 1.3 g/L, 1.4 g/L, 1.5 g/L, 1.6 g/L, 1.7 g/L, 1.8 g/L, 1.9 g/L, or 2.0 g/L. In certain embodiments, a haptoglobin (g/L) in the subject is from about 0.3 g/L to about 2.0 g/L or a range defined by the two preceding values. In certain embodiments, a reticulocyte count (%) in the subject is about 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, or 2.3%. In certain embodiments, a reticulocyte count (%) in the subject is from about 0.3% to about 2.3% or a range defined by the two preceding values. In certain embodiments, normalization of total hemoglobin in the subject occurs by at least 1 month, 1.5 months, 2 months, 3 months, 4 months, 5 months after administering the population of modified cells to the subject. In certain embodiments, the subject may undergo myeloablative conditioning with busulfan prior to administering the population of modified cells. In certain embodiments, administering the population of modified cells may comprise a single infusion of the modified population of cells. In certain embodiments, the population of modified cells may be about ≥1×10⁶ cells/kg, ≥2×10⁶ cells/kg, ≥3×10⁶ cells/kg, ≥4×10⁶ cells/kg, ≥5×10⁶ cells/kg, ≥6×10⁶ cells/kg, ≥7×10⁶ cells/kg, ≥8×10⁶ cells/kg, ≥9×10⁶ cells/kg, ≥10×10⁶ cells/kg, ≥11×10⁶ cells/kg, ≥12×10⁶ cells/kg, ≥13×10⁶ cells/kg, ≥14×10⁶ cells/kg, ≥15×10⁶ cells/kg, ≥16×10⁶ cells/kg, ≥17×10⁶ cells/kg, ≥18×10⁶ cells/kg, ≥19×10⁶ cells/kg, ≥20×10⁶ cells/kg, ≥21×10⁶ cells/kg, ≥22×10⁶ cells/kg, ≥23×10⁶ cells/kg, ≥24×10⁶ cells/kg, ≥25×10⁶ cells/kg, 1×10⁶ cells/kg to 25×10⁶ cells/kg, 1×10⁶ cells/kg to 5×10⁶ cells/kg, 3×10⁶ cells/kg to 20×10⁶ cells/kg, 3×10⁶ cells/kg to 15×10⁶ cells/kg, 3×10⁶ cells/kg to 10×10⁶ cells/kg, 3×10⁶ cells/kg to 5×10⁶ cells/kg, 5×10⁶ cells/kg to 10×10⁶ cells/kg, 10×10⁶ cells/kg to 15×10⁶ cells/kg, 15×10⁶ cells/kg to 20×10⁶ cells/kg, 20×10⁶ cells/kg to 25×10⁶ cells/kg. In certain embodiments, the % HbF may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the concentration of total hemoglobin may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the Hemoglobin F concentration may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the percentage of F-cells may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean corpuscular HbF (pg/RBC) may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean proportion of HbF as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean proportion of HbS as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean proportion of HbA as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean proportion of HbA2 as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the lactate dehydrogenase (U/L) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the indirect bilirubin (μmol/L) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the haptoglobin (g/L) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the reticulocyte count (%) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience no vaso-occlusive events (VOEs) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience no severe VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 2, 3, 4, 5, 6, 7, 8, 9, 10, 11, 12, 13, 14, 15, 16, 17, 18, 19, or 20 VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 2, 3, 4, 5, 6, 7, 8, 9, or 10 severe VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 1, 2, 3, 4, 5, 10, 15, 20, 25, 30, 35, 40, 45, 50, 55, 60, 65 treatment emergent adverse events (TEAEs) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 1, 2, 3, 4, 5, 6, 7, 8, 9, or 10 serious TEAEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may be red blood cell (RBC) transfusion free 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

Provided herein in certain aspects are methods of producing a modified population of peripheral blood nucleated cells in a subject, the method comprising: administering to the subject a population of modified cells comprising a plurality of modified CD34+ or hematopoietic stem cells comprising an indel in an HBG promoter, thereby producing the modified population of peripheral blood nucleated cells, wherein the modified population of peripheral blood nucleated cells as a percentage of the total population of peripheral blood nucleated cells is at least 50%, 55%, 60%, 65%, 70%, 75%, 80%, 85%, 90% or 95%. In certain embodiments, the subject may undergo myeloablative conditioning with busulfan prior to administering the population of modified cells.

Provided herein in certain aspects are methods of generating a population of F-cells in a subject, the method comprising: administering to the subject a population of modified cells comprising a plurality of modified CD34+ or hematopoietic stem cells comprising an indel in an HBG promoter, wherein the percentage of F-cells among circulating red blood cells six months after said administering is at least 90%. In certain embodiments, mean corpuscular HbF per F-cell in the population of F-cells is at least 18 picograms (pg). In certain embodiments, the subject may exhibit no vaso-occlusive events (VOEs) within six months after said administering. In certain embodiments, the subject may exhibit no VOEs within one year after said administering.

This listing is intended to be exemplary and illustrative rather than comprehensive and limiting. Additional aspects and embodiments may be set out in, or apparent from, the remainder of this disclosure and the claims.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings are intended to provide illustrative, and schematic rather than comprehensive, examples of certain aspects and embodiments of the present disclosure. The drawings are not intended to be limiting or binding to any particular theory or model, and are not necessarily to scale. Without limiting the foregoing, nucleic acids and polypeptides may be depicted as linear sequences, or as schematic two- or three-dimensional structures; these depictions are intended to be illustrative rather than limiting or binding to any particular model or theory regarding their structure.

FIG. 1 depicts, in schematic form, HBG1 and HBG2 gene(s) in the context of the β-globin gene cluster on human chromosome 11. Each gene in the β-globin gene cluster is transcriptionally regulated by a proximal promoter. While not wishing to be bound by any particular theory, it is generally thought that A_{γ} and/or G_{γ} expression is activated by engagement between the proximal promoter with the distal strong erythroid-specific enhancer, the locus control region (LCR). Long-range transactivation by the LCR is thought to be mediated by alteration of chromatin configuration/confirmation. The LCR is marked by 4 crythroid specific Dnasc I hypersensitive sites (HS1-4) and 2 distal enhancer elements (5′ HS and 3′ HS1). β-like gene globin gene expression is regulated in a developmental stage-specific manner, and expression of globin genes changes coincide with changes in the main site of blood production.

FIGS. 2A-2B depict HBG1 and HBG2 genes, coding sequences (CDS) and small deletions and point mutations in and upstream of the HBG1 and HBG2 proximal promoters that have been identified in subjects and associated with elevation of fetal hemoglobin (HbF). Core elements within the proximal promoters (CAAT box, 13 nt sequence) that have been deleted in some subjects with hereditary persistence of fetal hemoglobin (HPFH). The ‘target sequence’ region of each locus, which has been screened for gRNA binding target sites, is also identified.

FIG. 3 depicts editing in the HBG distal CCAAT box region by RNP27 (Table 18). RNP27 comprises a gRNA comprising the sequence set forth in SEQ ID NO:237 complexed with a Cas12a (Cpf1) protein comprising the sequence set forth in SEQ ID NO:283. RNP27 targets HBG1 and HBG2 promoters on chromosome 11, which are 4.9 kb apart. The distal CCAAT box is highlighted with a box. The protospacer adjacent motif (PAM) is bolded in black. The target sequence of the RNP27 gRNA is underlined. The RNP27 cleavage site and the resulting 5′ overhangs are indicated with dotted arrows. The point mutations associated with HPFH are bolded. The 13 bp HPFH deletion is indicated with a dotted line. HPFH=hereditary persistence of fetal hemoglobin; RNP=ribonucleoprotein; bp=base pair.

FIGS. 4A-E depict laboratory parameters for Subjects 1 or 2. FIG. 4A depicts the total hemoglobin and hemoglobin fractionation data for Subject 1 at baseline and at 1, 1.5, 2, 3, 4, and 5 months after autologous RNP27 edited CD34+ cell infusion. The lower portion of the bar (“HbF,” light grey) represents the mean proportion of HbF as a percentage of total hemoglobin (i.e., 5.0% (baseline), 18.7% (1M), 28.1% (1.5 M), 33.5% (2M), 36.7% (3M), 42.6% (4M), 45.4% (5M)); the middle portion of the bar (“HbS,” dark grey) represents the mean proportion of HbS as a percentage of total Hb (i.e., 89.1% (baseline), 18.2% (1M), 26.3% (1.5 M), 32.2% (2M), 37.5% (3M), 49.9% (4M), 51.8% (5M)); and the top portion of the bar (“Other,” medium grey) represents other hemoglobin (i.e., 5.9% (baseline), 63.1% (1M), 45.6% (1.5 M), 34.3% (2M), 25.8% (3M), 7.5% (4M), 2.8% (5M)). Bars show mean Hb (g/dL). Labels indicate mean proportion of HbS and HbF as a percentage of total Hb. Mean total Hb concentrations are shown directly above bars. Hb, hemoglobin; HbF, fetal hemoglobin; HbS, sickle hemoglobin; RBC, red blood cell. The X axis shows months after autologous RNP27 edited CD34+ cell infusion. The dotted lines show the 13.6-18.0 g/dL normal range for men (Central laboratory reference range). FIG. 4B depicts the total hemoglobin levels over time for Subject 1 at baseline and at 1, 1.5, 2, 3, 4, 5, 6, and 8 months after autologous RNP27 edited CD34+ cell infusion. The first portion of the bar (“HbF”) represents the mean proportion of HbF as a percentage of total hemoglobin (i.e., 5.0% (baseline), 18.7% (1M), 28.1% (1.5 M), 33.5% (2M), 36.7% (3M), 42.6% (4M), 45.4% (5M), 44.3% (6M), 44.1% (8M)); the second portion of the bar (“HbS”) represents the mean proportion of HbS as a percentage of total Hb (i.e., 89.1% (baseline), 18.2% (1M), 26.3% (1.5 M), 32.2% (2M), 37.5% (3M), 49.9% (4M), 51.8% (5M), 52.1% (6M), 54.4% (8M)); the third portion of the bar (“HbA”) represents HbA (i.e., 6.3% (baseline), 61.7% (1M), 44.7% (1.5 M), 33.5% (2M), 23.4% (3M), 4.2% (4M), 1.9% (5M), 2.0% (6M), 1.9% (8M)); the fourth portion of the bar represents HbA2 (“HbA2”) (i.e., 3.0% (baseline), 1.7% (1M), 1.6% (1.5 M), 1.4% (2M), 1.7% (3M), 1.7% (4M), 1.6% (5M), 1.7% (6M), 1.9% (8M)); the fifth portion of the bar represents other hemoglobin (“Other Hb”) (i.e., 0% (baseline), 0% (1M), 0% (1.5 M), 0% (2M), 0.7% (3M), 1.6% (4M), 0% (5M), 0% (6M), 0% (8M)). Bars show mean Hb (g/dL). Labels indicate mean proportion of HbS and HbF as a percentage of total Hb. Mean total Hb concentrations are shown directly above bars. Hb, hemoglobin; HbF, fetal hemoglobin; HbS, sickle hemoglobin. The X axis shows months after autologous RNP27 edited CD34+ cell infusion. The region identified between dotted lines indicates the 13.6-18.0 g/dL normal range for men (Central laboratory reference range). FIG. 4C depicts the percentage of F-cells for Subject 1 at baseline and at 1, 1.5, 2, 3, 4, 5, 6, and 8 months after autologous RNP27 edited CD34+ cell infusion. The X axis shows months after autologous RNP27 edited CD34+ cell infusion. Higher pancellularity indicates more red blood cells express HbF for potential clinical benefit. FIG. 4D depicts the percentage of mean corpuscular HbF (pg) concentration for Subject 1 at baseline and at 1, 1.5, 2, 3, 4, and 5 months after autologous RNP27 edited CD34+ cell infusion. Mean Corpuscular HbF: (MCH (pg)*HbF (%))/100%=HbF(pg) per RBC. 10 pg/RBC is the threshold for protection from sickling and is shown by a dotted line (Steinberg 2014). The X axis shows months after autologous RNP27 edited CD34+ cell infusion. FIG. 4E depicts the mean corpuscular HbF (pg) per F-cell for Subject 1 at baseline and at 1, 1.5, 2, 3, 4, 5, 6, and 8 months after autologous RNP27 edited CD34+ cell infusion. Mean corpuscular HbF (pg) is shown directly above bars. Mean Corpuscular HbF: (MCH (pg)*HbF (%))/100%=HbF(pg) per RBC. 10 pg/RBC is the threshold for protection from sickling and is shown by a dotted line (Steinberg 2014). The X axis shows months after autologous RNP27 edited CD34+ cell infusion. FIG. 4F depicts hemoglobin fractionation (%) data for Subject 1 at various study days before and after autologous RNP27 edited CD34+ cell infusion. The lower portion of the bar (“HbF”) represents the mean proportion of HbF as a percentage of total hemoglobin (i.e., 3.5% (Study Day (“SD”) −147), 5.0% (SD −106), 4.6% (SD −85), 1.4% (SD −14), 1.3% (SD 1), 18.7% (SD 31), 28.1% (SD 45), 33.5% (SD 59), 36.7% (SD 80), 42.6% (SD 122), 45.5% (SD 150), 44.3 (SD 191); the middle portion of the bar (“Other”) represents other hemoglobin as a percentage of total hemoglobin (i.e., 24% (SD −147), 5.9% (SD −106), 9.6% (SD −85), 54.9% (SD −14), 73.2% (SD 1), 63.1% (SD 31), 45.6% (SD 45), 34.3% (SD 59), 25.8% (SD 80), 7.5% (SD 122), 2.8% (SD 150), 3.6% (SD 191)); and the top portion of the bar (“HbS”) represents the mean proportion of HbS as a percentage of total Hb (i.e., 72.5% (SD −147), 89.1% (SD −106), 85.8% (SD −85), 43.7% (SD −14), 25.5% (SD 1), 18.2% (SD 31), 26.3% (SD 45), 32.2% (SD 59), 37.5% (SD 80), 49.9% (SD 122), 51.8% (SD 150), 52.1% (SD 191)). Labels indicate mean proportion of HbS and HbF as a percentage of total Hb. The percentage of F-Cells, total hemoglobin (g/dL), and MCH-F/F-cell (pg) are shown directly above the bar graph. Hb, hemoglobin; HbF, fetal hemoglobin; HbS, sickle hemoglobin; MCH, mean corpuscular HbF; RBC, red blood cell. The X axis shows Study Days (“SD”) −147, −106, −85, −14, 1, 31, 45, 59, 80, 122, 150, and 191. Study Day 1 represents the day on which autologous RNP27 edited CD34+ cell infusion was administered. The normal range of total hemoglobin (g/dL) in men is 13.6-18.0 g/dL (Central laboratory reference range). The formula used to calculate MCH-F/F-cell is: (HbF/RBC)*100 divided by % F-cell. 10 pg/RBC is the threshold for protection from sickling (Steinberg 2014). FIG. 4G depicts the total hemoglobin levels over time for Subject 2 at baseline and at 1, 1.5, 2, 3, 4, and 5 months after autologous RNP27 edited CD34+ cell infusion. The first (lower) portion of the bar (“HbF”) represents the mean proportion of HbF as a percentage of total hemoglobin (i.e., 10.8% (baseline), 8.2% (1M), 17.0% (1.5 M), 25.8% (2M), 38.6% (3M), 46.2% (4M), 51.2% (5M)); the second portion of the bar (“HbS”) represents the mean proportion of HbS as a percentage of total Hb (i.e., 77.7% (baseline), 5.4% (1M), 13.6% (1.5 M), 22.4% (2M), 33.2% (3M), 42.1% (4M), 46.7% (5M)); the third portion of the bar (“HbA”) represents HbA (i.e., 10.2% (baseline), 84.8% (1M), 67.7% (1.5 M), 50.5% (2M), 28.8% (3M), 10.6% (4M), 1.9% (5M)); the fourth portion of the bar represents HbA2 (“HbA2”) (i.e., 1.8% (baseline), 2.3% (1M), 2.0% (1.5 M), 1.8% (2M), 1.7% (3M), 1.5% (4M), 1.2% (5M)); the fifth (top) portion of the bar represents other hemoglobin (“Other Hb”) (i.e., 0% (baseline), 0% (1M), 0% (1.5 M), 0% (2M), 0% (3M), 0% (4M), 0% (5M)). Bars show mean Hb (g/dL). Labels indicate mean proportion of HbS and HbF as a percentage of total Hb. Mean total Hb concentrations are shown directly above bars. Hb, hemoglobin; HbF, fetal hemoglobin; HbS, sickle hemoglobin. The X axis shows months after autologous RNP27 edited CD34+ cell infusion. The region identified between dotted lines indicates the 12.0-16.0 g/dL normal range for women (Central laboratory reference range). FIG. 4H depicts the percentage of F-cells for Subject 2 at baseline and at 1, 1.5, 2, 3, and 4 months after autologous RNP27 edited CD34+ cell infusion. The X axis shows months after autologous RNP27 edited CD34+ cell infusion. Higher pancellularity indicates more red blood cells express HbF for potential clinical benefit. FIG. 4I depicts the mean corpuscular HbF (pg) per F-cell for Subject 2 at baseline and at 1, 1.5, 2, 3, and 4 months after autologous RNP27 edited CD34+ cell infusion. Mean corpuscular HbF (pg) is shown directly above bars. Mean Corpuscular HbF: (MCH (pg)*HbF (%))/100%=HbF(pg) per RBC. 10 pg/RBC is the threshold for protection from sickling and is shown by a dotted line (Steinberg 2014). The X axis shows months after autologous RNP27 edited CD34+ cell infusion. FIG. 4J depicts hemoglobin fractionation (%) data for Subject 2 at various study days before and after autologous RNP27 edited CD34+ cell infusion. The lower portion of the bar (“HbF”) represents the mean proportion of HbF as a percentage of total hemoglobin (i.e., 10.5% (Study Day (“SD”) −450), 10.8% (SD −434), 8.4% (SD −9), 2.7% (SD −1), 8.2% (SD 34), 17.0% (SD 44), 25.8% (SD 62), 38.6% (SD 93); the middle portion of the bar (“Other”) represents other hemoglobin as a percentage of total hemoglobin (i.e., 15.7% (Study Day (“SD”) −450), 11.5% (SD −434), 53.4% (SD −9), 87.6% (SD −1), 86.4% (SD 34), 69.4% (SD 44), 51.8% (SD 62), 28.2% (SD 93); and the top portion of the bar (“HbS”) represents other hemoglobin as a percentage of total hemoglobin (i.e., 73.8% (Study Day (“SD”) −450), 77.7% (SD −434), 38.2% (SD −9), 9.7% (SD −1), 5.4% (SD 34), 13.6% (SD 44), 22.4% (SD 62), 33.2% (SD 93). Labels indicate mean proportion of HbS and HbF as a percentage of total Hb. The percentage of F-Cells, total hemoglobin (g/dL), and MCH-F/F-cell (pg) are shown directly above the bar graph. Hb, hemoglobin; HbF, fetal hemoglobin; HbS, sickle hemoglobin; MCH, mean corpuscular HbF; RBC, red blood cell. The X axis shows Study Days (“SD”) −450, −434, −9, 1, 34, 44, 62, and 93. Study Day 1 represents the day on which autologous RNP27 edited CD34+ cell infusion was administered. The normal range of total hemoglobin (g/dL) in women is 12.0-16.0 g/dL. The formula used to calculate MCH-F/F-cell is: (HbF/RBC)*100 divided by % F-cell. 10 pg/RBC is the threshold for protection from sickling (Steinberg 2014).

FIGS. 5A-5F depict the mean hemoglobin levels over time for subjects at baseline and months after autologous RNP27 edited CD34+ cell infusion. For FIGS. 5A-5D: the mean hemoglobin levels over time is depicted for subjects at baseline and months after autologous RNP27 edited CD34+ cell infusion. Bars show mean Hb (g/dL). Labels indicate mean proportion of HbF as a percentage of total Hb. Mean total Hb concentrations are shown directly above bars. Hb, hemoglobin; HbF, fetal hemoglobin; HbS, sickle hemoglobin. The X axis shows months after autologous RNP27 edited CD34+ cell infusion, represented as a black vertical dotted line. The region identified with grey horizontal band indicates the 13.6-18.0 g/dL normal range for men subjects (Central laboratory reference range) (Subjects 1 and 4) or 12.0-16.0 g/dL normal range for female subjects (Subjects 2 and 3). The dark grey arrow shows the day of last red blood cell (RBC) transfusion. For FIG. 5E: the mean hemoglobin levels over time is depicted for subjects at baseline and months after autologous RNP27 edited CD34+ cell infusion. Bars show mean total Hb (g/dL). Labels indicate mean proportion of HbF as a percentage of total Hb. Mean total Hb concentrations are shown directly above bars. For FIG. 5E, the region identified with grey horizontal band indicates the 13.6-18.0 g/dL normal range for men subjects (Central laboratory reference range) or 12.0-16.0 g/dL normal range for female subjects. FIG. 5A depicts data for Subject 1. The first (lowest) portion of the bar (“HbF,” medium grey) represents the mean proportion of HbF as a percentage of total hemoglobin (5.0% (baseline), 18.7% (1M), 28.1% (1.5M), 33.5% (2M), 36.7% (3M), 42.6% (4M), 45.4% (5M), 44.3% (6M), 44.1% (8M), 43.4% (10M)); the second portion of the bar (“HbS,” black) represents the mean proportion of HbS as a percentage of total Hb; the third portion of the bar (“HbA/Transfused blood,” light grey) represents HbA; the fourth portion of the bar represents HbA2 (“HbA2,” medium grey); the fifth portion of the bar (only found at 4M) represents other hemoglobin (“Other Hb,” dark grey). Bars show mean Hb (g/dL). FIG. 5B depicts data for Subject 2. The first (lowest) portion of the bar (“HbF,” medium grey) represents the mean proportion of HbF as a percentage of total hemoglobin (10.8% (baseline), 8.2% (1M), 17.0% (1.5M), 25.8% (2M), 38.6% (3M), 46.2% (4M), 51.2% (5M), 51.3% (6M)); the second portion of the bar (“HbS,” black) represents the mean proportion of HbS as a percentage of total Hb; the third portion of the bar (“HbA/Transfused blood,” light grey) represents HbA; the fourth (top) portion of the bar represents HbA2 (“HbA2,” medium grey). Bars show mean Hb (g/dL). FIG. 5C depicts data for Subject 3. The first (lowest) portion of the bar (“HbF,” medium grey) represents the proportion of HbF as a percentage of total hemoglobin (2.9% (baseline), 12.1% (1M), 23.7% (1.5M), 31.5% (2M), 42.6% (3M)); the second portion of the bar (“HbS,” black) represents the mean proportion of HbS as a percentage of total Hb; the third portion of the bar (“HbA/Transfused blood,” light grey) represents HbA; the fourth (top) portion of the bar represents HbA2 (“HbA2,” medium grey). Bars show mean Hb (g/dL). FIG. 5D depicts data for Subject 4. The first (lowest) portion of the bar (“HbF,” medium grey) represents the mean proportion of HbF as a percentage of total hemoglobin (6.1% (baseline), 6.7% (1M), 17.0% (1.5M), 26.6% (2M)); the second portion of the bar (“HbS,” black) represents the mean proportion of HbS as a percentage of total Hb; the third portion of the bar (“HbA/Transfused blood,” light grey) represents HbA; the fourth (top) portion of the bar represents HbA2 (“HbA2,” medium grey). FIG. 5E depicts data for Subjects 1-10. Number of male subjects=5; number of female subjects=5. Bars show mean Hb (g/dL). Labels inside/next to the bars indicate mean proportion of HbF as a percentage of total Hb (white bar). Mean total Hb concentrations are shown directly above bars. FIG. 5F depicts data for Subjects 1-18. Number of male subjects=9; number of female subjects=9. Bars show mean Hb (g/dL). Labels inside/next to the bars indicate mean proportion of HbF as a percentage of total Hb (white bar). Mean total Hb concentrations are shown directly above bars. * indicates Central laboratory reference range: 13.6-18.0 g/dL for males and 12.0-16.0 g/dL for females. ^{†}The last RBC transfusion in subjects occurred at a mean (SD) of 14.6 (5.6) days after RNP27-edited CD34+ cell infusion as indicated by the black arrow (n=18). ^{‡}At Month 1, one subject did not have central labs performed at because of venous access issues. ^{§}At Month 6, n=10 for HbF percentage; total Hb and absolute HbF were not provided by the central lab at Month 6 for one subject because the sample was clotted.

FIGS. 6A-6D depicts the percentage of F-cells and mean corpuscular HbF for subjects at baseline and at various months after autologous RNP27 edited CD34+ cell infusion. FIG. 6A depicts the percentage of F-cells for Subjects 1-4 at baseline and at various months after autologous RNP27 edited CD34+ cell infusion. Subject 1, medium grey dashed line; Subject 2, dark grey dashed line; Subject 3, solid grey line; and Subject 4, solid black line. The X axis shows months after autologous RNP27 edited CD34+ cell infusion, represented as a black vertical dotted line. An increasing percentage of F-cells indicates that more RBCs are protected from sickling for potential clinical benefit. *Data for Subject 2 at 3 months post-RNP27 edited CD34+ cell infusion were not available due to sample quality (hemolyzed sample). FIG. 6B depicts the mean corpuscular HbF for Subjects 1-4 at baseline and at various months after autologous RNP27 edited CD34+ cell infusion. Subject 1, medium grey long dashed line; Subject 2, black solid line; Subject 3, solid grey line; and Subject 4, light grey short dashed line. The X axis shows months after autologous RNP27 edited CD34+ cell infusion, represented as an grey vertical dotted line. Subjects reach sickling-protective levels of HbF in F-cells by 1 month. 10 pg/F-cell is the threshold for protection from sickling (Steinberg 2014). *Data for Subject 2 at 3 months post-RNP27 edited CD34+ cell infusion were not available due to sample quality (hemolyzed sample). FIG. 6C depicts the mean percentage of F-cells for Subjects 1-18 at baseline (BL) and up to 21 months after autologous RNP27 edited CD34+ cell infusion. The X axis shows months after autologous RNP27 edited CD34+ cell infusion, represented as a black vertical dotted line. An increasing percentage of F-cells indicates that more RBCs are protected from sickling for potential clinical benefit. As shown in FIG. 6C, the percentage of F-cells increased early. SEM=Standard error of the mean. FIG. 6D depicts the mean corpuscular HbF for Subjects 1-18 at baseline (BL) and at various months after autologous RNP27 edited CD34+ cell infusion. MCH-F/F-cell was sustained above the anti-sickling threshold. 10 pg/F-cell is the threshold for protection from sickling (Steinberg 2014). The X axis shows months after autologous RNP27 edited CD34+ cell infusion, represented as a black vertical dotted line. SEM=Standard error of the mean.

FIG. 7 depicts the percentage of HBG1 and HBG2 promoter editing for Subjects 1-4 at various months after autologous RNP27 edited CD34+ cell infusion. Subject 1, medium grey long dashed line; Subject 2, black solid line; Subject 3, lower dot; and Subject 4, upper dot. The X axis shows months after autologous RNP27 edited CD34+ cell infusion. Persistent, high levels of editing in peripheral blood nucleated cells indicate robust editing of HSPCs, predicting durable clinical benefit.

FIGS. 8A-8C depicts the percentage of HBG1 and HBG2 promoter editing. FIG. 8A depicts the percentage of HBG1 and HBG2 promoter editing in the drug product and bone marrow (BM) of Subject 1. The bone marrow was sampled at 4 months post-RNP27 edited CD34+ cell infusion in the phase 1/2 clinical study. FIG. 8B depicts the percentage of HBG1 and HBG2 promoter editing in the peripheral blood nucleated cells of Subject 1 (dark grey medium dash line), Subject 2 (black solid line), Subject 3 (medium grey solid line), Subject 4 (light grey medium dash line), Subject 5 (dark grey long dashed line), Subject 7 (light grey dotted line), Subject 8 (dark grey dotted line). The X axis shows months after autologous RNP27 edited CD34+ cell infusion. FIG. 8C depicts the percentage of HBG1 and HBG2 promoter editing in the BM of Subject 1 (dark grey medium dashed line), Subject 2 (dark grey solid line), Subject 3 (grey dot), Subject 4 (dot at ~90%). The X axis shows months after autologous RNP27 edited CD34+ cell infusion.

FIGS. 9A-9C depicts the safety profile for subjects treated with RNP27 edited CD34+ cell infusion. FIG. 9A depicts the safety profile for Subjects 1-4 treated with RNP27 edited CD34+ cell infusion. The safety profile is consistent with that of HSCT and myeloablative conditioning with bulsulfan. The majority of TEAEs (E=26) occurred within the first 30 days after RNP27 edited CD34+ cell infusion. No TEAEs were reported as related to RNP27 edited CD34+ cell infusion. No TEAEs occurred after RNP27 edited CD34+ cell infusion. Grade 3 TEAEs related to busulfan: n=2, Preferred Term “mucosal inflammation”. Grade 3 TEAEs unrelated to busulfan or RNP27 edited CD34+ cells: n=1, Preferred Term “Blood bilirubin unconjugated increased”. E, number of events; HSCT, hematopoietic stem cell transplantation; TEAE, treatment emergent adverse event; TESAE, treatment emergent serious adverse event. FIG. 9B depicts the safety profile for Subjects 1-10 treated with RNP27 edited CD34+ cell infusion. The safety profile is consistent with that of HSCT and myeloablative conditioning with bulsulfan. The only TEAE related to RNP27 edited CD34+ cell infusion occurred in one subject and was Grade 2 polycythaemia. The subject presented asymptomatically and has remained clinically stable. TEAE, treatment emergent adverse event. FIG. 9C depicts the safety profile for Subjects 1-18 treated with RNP27 edited CD34+ cell infusion. The safety profile is consistent with that of HSCT and myeloablative conditioning with bulsulfan. One subject experienced a non-serious TEAE of Grade 1 Alanine aminotransferase increased (1.2×ULN), which was reported to be causally related to RNP27 edited CD34+ cell infusion and busulfan. The TEAE has resolved, and alanine aminotransferase level normalized. ^{†}As of the data cut, serious TEAEs in the phase 1/2/3 trial included gastroenteritis, gastroenteritis viral, pneumonia, sepsis, chills, and hyperglycemia. TEAE, treatment emergent adverse event. ULN, upper limit of normal. FIG. 9D depicts the vaso-occlusive events (VOE) profile for Subjects 1-10 treated with RNP27 edited CD34+ cell infusion. At the time of the prior data cutoff, all 10 subjects who had been infused with RNP27 edited CD34+ cells and reached the Month 1 visit were completely VOE-free. This is compared with a mean of 4 severe VOEs/year and a mean of 1.5 non-severe VOEs/year in the 2 years before enrollment. Left panel ends at informed consent date: *=Day of informed consent. Right panel starts at infusion date: \hat{ }=Day of RNP27 edited CD34+ cells infusion. Filled in diamond=severe VOE; open diamond=non-severe VOE. FIG. 9E depicts the vaso-occlusive events (VOE) profile for Subjects 1-18 treated with RNP27 edited CD34+ cell infusion. At the time of the most recent data cutoff, all 18 subjects who had been infused with RNP27 edited CD34+ cells were completely VOE-free for up to 22.8 months. Left panel ends at informed consent date: * indicates the Day of informed consent. Right panel starts at infusion date: \hat{ }indicates Day of RNP27 edited CD34+ cells infusion. Filled in diamond indicates severe VOE; open diamond indicates non-severe VOE. **A severe VOE requiring medical attention (despite hydroxyurea or other supportive care measures in the pre-treatment period) is defined as: an acute episode of pain with no cause other than a vaso-occlusion, resulting in either a ≥24-h hospital or Emergency Room (ER) observation unit or ≥2 visits to a day unit or ER over 72 h with both visits requiring administration of pain medications; acute priapism lasting >2 h and requiring a visit to a medical facility (with or without hospitalization); acute chest syndrome (ACS), which is defined as chest-wall pain in association with findings of a new pulmonary infiltrate on chest X-ray films associated with fever and/or respiratory symptom; or hepatic or splenic sequestration, which is defined as a sudden increase in organ size associated with pain in the area of the organ, decrease in the hemoglobin concentration of ≥2 g/dL within a 24-h period, and, for liver sequestration, abnormal change in liver function tests, including conjugated bilirubin, not due to biliary tract disease. ***Non-Severe VOE is defined as an acute episode of pain with no medically determined cause other than a vaso-occlusion.

FIGS. 10A-10D depict the total hemoglobin levels over time for subjects in the transfusion-dependent beta-thalassemia (TDT) phase 1/2 study in Example 2 at baseline and months after autologous RNP27 edited CD34+ cell infusion. FIG. 10A depicts the total hemoglobin levels over time for Subject 1 of the transfusion-dependent beta-thalassemia (TDT) phase 1/2 study in Example 2 at baseline and months after autologous RNP27 edited CD34+ cell infusion. The first (bottom) portion of the bar (“HbF”) represents the mean proportion of HbF as a percentage of total hemoglobin (i.e., baseline (2.2%), 1M (13.9%), 1.5M (34.9%); the second portion of the bar (dark grey) (“HbA/Transfused blood”) represents HbA (i.e., baseline (93.5%), 1M (81.2%), 1.5M (65.0%); the third portion of the bar represents HbA2 (“HbA2”) (i.e., baseline (2.7%), 1M (2.3%), 1.5M (0.0%); the fourth (top) portion of the bar represents other hemoglobin (“Other Hb”) (i.e., baseline (1.6%), 1M (2.6%), 1.5M (1.8%). Bars show mean Hb (g/dL). Labels inside/to the right of the bars indicate mean proportion of HbF as a percentage of total Hb. Mean total Hb concentrations are shown directly above bars. Hb, hemoglobin; HbF, fetal hemoglobin. The X axis shows months after autologous RNP27 edited CD34+ cell infusion, represented as a horizontal dotted line. The region identified between horizontal dotted lines indicates the 13.6-18.0 g/dL normal range for men (Central laboratory reference range). The arrow shows the day of last red blood cell (RBC) transfusion. FIG. 10B depicts the total hemoglobin levels over time for Subjects (n=6) of the transfusion-dependent beta-thalassemia (TDT) phase 1/2 study in Example 2 at baseline and months after autologous RNP27 edited CD34+ cell infusion. The white portion of the bar (“HbF”) represents the mean total of HbF; the dark grey portion of the bar (“Total Hb”) represents mean total Hb. Bars show Hb (g/dL). Total Hb concentrations are shown directly above bars. Hb, hemoglobin; HbF, fetal hemoglobin. The X axis shows months after autologous RNP27 edited CD34+ cell infusion, represented as a vertical dotted line. The horizontal dotted line indicates the transfusion threshold of 9 g/dL. FIG. 10C depicts the total hemoglobin levels over time for Subjects (n=7) of the transfusion-dependent beta-thalassemia (TDT) phase 1/2 study in Example 2 at baseline and months after autologous RNP27 edited CD34+ cell infusion. The white portion of the bar (“HbF”) represents the mean total of HbF; the dark grey portion of the bar (“Total Hb”) represents the remainder of the mean total Hb. Bars show Hb (g/dL). Total Hb concentrations are shown directly above bars. Hb, hemoglobin; HbF, fetal hemoglobin. The X axis shows months after autologous RNP27 edited CD34+ cell infusion, represented as a vertical dotted line. The horizontal dotted line indicates the transfusion independence threshold of total Hb: 9 g/dL. The mean day the last RBC infusion was administered is shown as an arrow. The last RBC transfusion in subjects was administered a mean (SD) of 29.4 (19.1) days after RNP27 edited CD34+ cell infusion (n=7). * indicates that at Baseline, n=6 for HbF. FIG. 10D shows TDT subjects were transfusion-free for up to 12.8 months after RNP27 edited CD34+ cell infusion. Black bars represent time from RNP27 edited CD34+ cell infusion to last RBC transfusion support. White bars represent time without RBC transfusions starting from last transfusion. Labels inside bars indicate the number of months of post RNP-edited CD34+ cell infusion follow-up. * indicates the number of transfusion units annualized over 2 years.

FIGS. 11A-11B depicts the safety profile for subjects treated with RNP27 edited CD34+ cell infusion. FIG. 11A depicts that the safety profile for Subjects 1-6 when ranging from 0 to 8 months after RNP27 edited CD34+ cell infusion. As seen in FIG. 11A, the safety profile for treatment with RNP27 edited CD34+ cell infusion is consistent with that of hematopoietic stem cell transplantation (HSCT) and myeloablative conditioning with bulsulfan. No treatment-emergent adverse events (TEAEs) were reported as related to RNP27 edited CD34+ cell infusion. No TEAEs occurred after RNP27 edited CD34+ cell infusion. FIG. 11B depicts that the safety profile for Subjects 1-7 when ranging from one to twelve months after RNP27 edited CD34+ cell infusion. As seen in FIG. 11B, the safety profile is consistent with that of HSCT and myeloablative conditioning with bulsulfan. No serious TEAEs (sTEAEs) were reported as related to RNP27 edited CD34+ cell infusion. * indicates that one subject experienced 2 non-serious TEAEs (Grade 2 CD4 lymphocytes decreased, and Grade 3 lymphocyte count decreased), which were reported to be causally related to RNP27 edited CD34+ cell infusion and busulfan. Both TEAEs are ongoing as of the data cutoff date. ** indicates that serious TEAEs were COVID-19, device-related infection, and pneumonitis.

FIG. 12 depicts the percentage of HBG1 and HBG2 promoter editing from representative RNP27 edited CD34+ cell production lots from subjects from the SCD phase 1/2/3 study in Example 1 or the TDT phase 1/2 study in Example 2. Clinical Production Lots 1-9 indicate subjects from the SCD phase 1/2/3 study in Example 1 and Clinical Production Lots 10-12 indicate subjects from the TDT phase 1/2 study in Example 2.

FIG. 13 depicts the percentage of HBG1 and HBG2 promoter editing from RNP27 edited CD34+ cell drug product lots from non-clinical CD34+ lots (black open circle), nonclinical long-term mouse bone marrow (BM) (grey open circles), subjects from the SCD phase 1/2/3 study in Example 1 (closed circles, second from right) or subjects from the TDT phase 1/2 study in Example 2 (closed circles, far right).

FIG. 14 depicts the sequences of Cas12a proteins set forth in Table 17. Nuclear localization sequences are shown as bolded letters, six-histidine sequences are shown as underlined letters. Additional permutations of the identity and N-terminal/C-terminal positions of NLS sequences, e.g., appending two or more nNLS sequences or combinations of nNLS and sNLS sequences (or other NLS sequences) to either the N-terminal/C-terminal positions, as well as sequences with and without purification sequences, e.g., six-histidine sequences, are within the scope of the instantly disclosed subject matter.

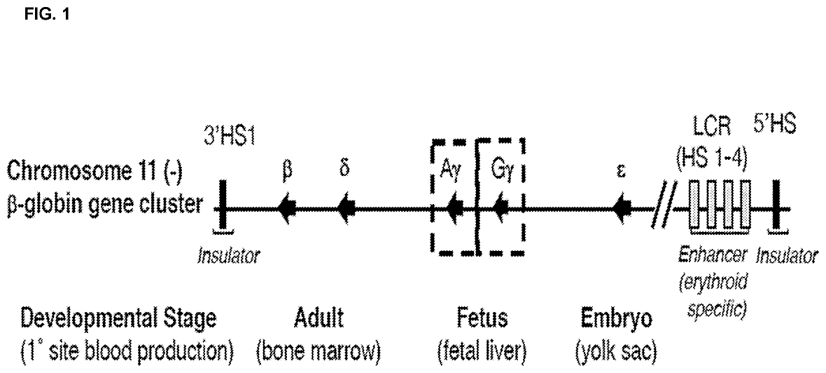

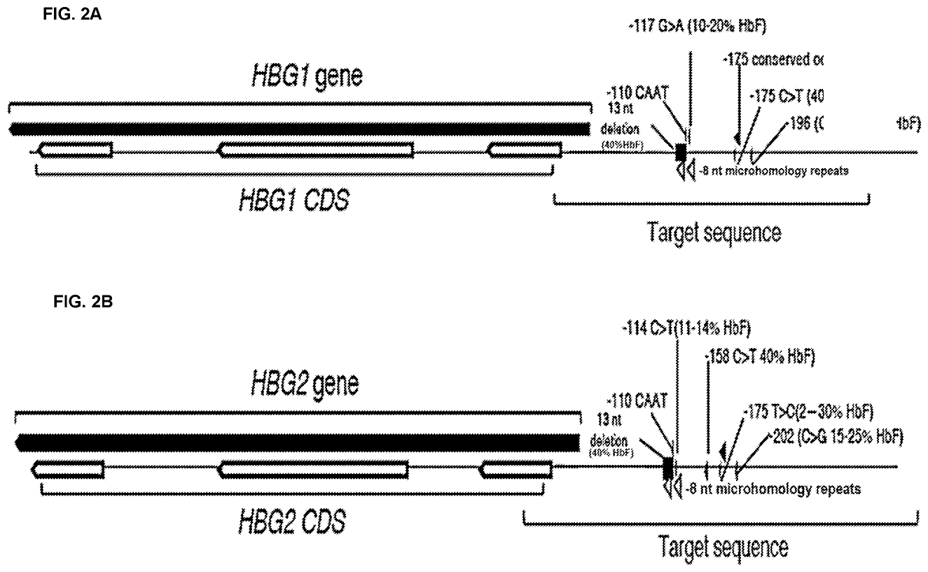

### DETAILED DESCRIPTION

### Definitions and Abbreviations

Unless otherwise specified, each of the following terms has the meaning associated with it in this section.

The indefinite articles “a” and “an” refer to at least one of the associated noun, and are used interchangeably with the terms “at least one” and “one or more.” For example, “a module” means at least one module, or one or more modules.

The conjunctions “or” and “and/or” are used interchangeably as non-exclusive disjunctions.

“Domain” is used to describe a segment of a protein or nucleic acid. Unless otherwise indicated, a domain is not required to have any specific functional property.

An “indel” is an insertion and/or deletion in a nucleic acid sequence. An indel may be the product of the repair of a DNA double strand break, such as a double strand break formed by a genome editing system of the present disclosure. An indel is most commonly formed when a break is repaired by an “error prone” repair pathway such as the NHEJ pathway described below.

“Gene conversion” refers to the alteration of a DNA sequence by incorporation of an endogenous homologous sequence (e.g. a homologous sequence within a gene array). “Gene correction” refers to the alteration of a DNA sequence by incorporation of an exogenous homologous sequence, such as an exogenous single- or double stranded donor template DNA. Gene conversion and gene correction are products of the repair of DNA double-strand breaks by HDR pathways such as those described below.

Indels, gene conversion, gene correction, and other genome editing outcomes are typically assessed by sequencing (most commonly by “next-gen” or “sequencing-by-synthesis” methods, though Sanger sequencing may still be used) and are quantified by the relative frequency of numerical changes (e.g., ±1, ±2 or more bases) at a site of interest among all sequencing reads. DNA samples for sequencing may be prepared by a variety of methods known in the art, and may involve the amplification of sites of interest by polymerase chain reaction (PCR), the capture of DNA ends generated by double strand breaks, as in the GUIDEseq process described in Tsai 2016 (incorporated by reference herein) or by other means well known in the art. Genome editing outcomes may also be assessed by in situ hybridization methods such as the FiberComb™ system commercialized by Genomic Vision (Bagneux, France), and by any other suitable methods known in the art.

“Alt-HDR,” “alternative homology-directed repair,” or “alternative HDR” are used interchangeably to refer to the process of repairing DNA damage using a homologous nucleic acid (e.g., an endogenous homologous sequence, e.g., a sister chromatid, or an exogenous nucleic acid, e.g., a template nucleic acid). Alt-HDR is distinct from canonical HDR in that the process utilizes different pathways from canonical HDR, and can be inhibited by the canonical HDR mediators, RAD51 and BRCA2. Alt-HDR is also distinguished by the involvement of a single-stranded or nicked homologous nucleic acid template, whereas canonical HDR generally involves a double-stranded homologous template.

“Canonical HDR,” “canonical homology-directed repair” or “cHDR” refer to the process of repairing DNA damage using a homologous nucleic acid (e.g., an endogenous homologous sequence, e.g., a sister chromatid, or an exogenous nucleic acid, e.g., a template nucleic acid). Canonical HDR typically acts when there has been significant resection at the double strand break, forming at least one single stranded portion of DNA. In a normal cell, cHDR typically involves a series of steps such as recognition of the break, stabilization of the break, resection, stabilization of single stranded DNA, formation of a DNA crossover intermediate, resolution of the crossover intermediate, and ligation. The process requires RAD51 and BRCA2, and the homologous nucleic acid is typically double-stranded.

Unless indicated otherwise, the term “HDR” as used herein encompasses both canonical HDR and alt-HDR.

“Non-homologous end joining” or “NHEJ” refers to ligation mediated repair and/or non-template mediated repair including canonical NHEJ (cNHEJ) and alternative NHEJ (altNHEJ), which in turn includes microhomology-mediated end joining (MMEJ), single-strand annealing (SSA), and synthesis-dependent microhomology-mediated end joining (SD-MMEJ).

“Replacement” or “replaced,” when used with reference to a modification of a molecule (e.g. a nucleic acid or protein), does not require a process limitation but merely indicates that the replacement entity is present.

“Subject” means a human, mouse, or non-human primate. A human subject can be any age (e.g., an infant, child, young adult, or adult), and may suffer from a disease, or may be in need of alteration of a gene.

“Treat,” “treating,” and “treatment” mean the treatment of a disease in a subject (e.g., a human subject), including one or more of inhibiting the disease, i.e., arresting or preventing its development or progression; relieving the disease, i.e., causing regression of the disease state; relieving one or more symptoms of the disease; and curing the disease.

“Prevent,” “preventing,” and “prevention” refer to the prevention of a disease in a subject, including (a) avoiding or precluding the disease; (b) affecting the predisposition toward the disease; or (c) preventing or delaying the onset of at least one symptom of the disease.

A “kit” refers to any collection of two or more components that together constitute a functional unit that can be employed for a specific purpose. By way of illustration (and not limitation), one kit according to this disclosure can include a guide RNA complexed or able to complex with an RNA-guided nuclease, and accompanied by (e.g., suspended in, or suspendable in) a pharmaceutically acceptable carrier. In certain embodiments, the kit may include a booster element. The kit can be used to introduce the complex into, for example, a cell or a subject, for the purpose of causing a desired genomic alteration in such cell or subject. The components of a kit can be packaged together, or they may be separately packaged. Kits according to this disclosure also optionally include directions for use (DFU) that describe the use of the kit e.g., according to a method of this disclosure. The DFU can be physically packaged with the kit, or it can be made available to a user of the kit, for instance by electronic means.

The terms “polynucleotide”, “nucleotide sequence”, “nucleic acid”, “nucleic acid molecule”, “nucleic acid sequence”, and “oligonucleotide” refer to a series of nucleotide bases (also called “nucleotides”) in DNA and RNA, and mean any chain of two or more nucleotides. The polynucleotides, nucleotide sequences, nucleic acids etc. can be chimeric mixtures or derivatives or modified versions thereof, single-stranded or double-stranded. They can be modified at the base moiety, sugar moiety, or phosphate backbone, for example, to improve stability of the molecule, its hybridization parameters, etc. A nucleotide sequence typically carries genetic information, including, but not limited to, the information used by cellular machinery to make proteins and enzymes. These terms include double- or single-stranded genomic DNA, RNA, any synthetic and genetically manipulated polynucleotide, and both sense and antisense polynucleotides. These terms also include nucleic acids containing modified bases.

Conventional IUPAC notation is used in nucleotide sequences presented herein, as shown in Table 1, below (see also Cornish-Bowden A, Nucleic Acids Res. 1985 May 10; 13(9):3021-30, incorporated by reference herein). It should be noted, however, that “T” denotes “Thymine or Uracil” in those instances where a sequence may be encoded by either DNA or RNA, for example, in gRNA, for example, in a gRNA targeting domain.

TABLE 1
IUPAC nucleic acid notation

| Character | Base |
|---|---|
| A | Adenine |
| T | Thymine or Uracil |
| G | Guanine |
| C | Cytosine |
| U | Uracil |
| K | G or T/U |
| M | A or C |
| R | A or G |
| Y | C or T/U |
| S | C or G |
| W | A or T/U |
| B | C, G or T/U |
| V | A, C or G |
| H | A, C or T/U |
| D | A, G or T/U |
| N | A, C, G or T/U |

The terms “protein,” “peptide” and “polypeptide” are used interchangeably to refer to a sequential chain of amino acids linked together via peptide bonds. The terms include individual proteins, groups or complexes of proteins that associate together, as well as fragments or portions, variants, derivatives and analogs of such proteins. Peptide sequences are presented herein using conventional notation, beginning with the amino or N-terminus on the left, and proceeding to the carboxyl or C-terminus on the right. Standard one-letter or three-letter abbreviations can be used.

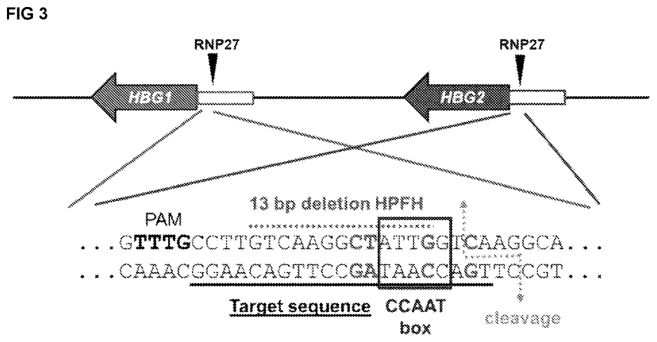

The notation “CCAAT box target region” and the like refer to a sequence that is 5′ of the transcription start site (TSS) of the HBG1 and/or HBG2 gene. The distal CCAAT box of HBG1 and HBG2 is shown in the schematic in FIG. 3. CCAAT boxes are highly conserved motifs within the promoter region of a-like and f-like globin genes. The regions within or near the CCAAT box play important roles in globin gene regulation. For example, the γ-globin distal CCAAT box is associated with hereditary persistence of fetal hemoglobin. A number of transcription factors have been reported to bind to the duplicated CCAAT box region of the γ-globin promoter, e.g., NF-Y, COUP-TFII (NF-E3), CDP, GATA1/NF-E1 and DRED (Martyn 2017). While not wishing to be bound by theory, it is believed that the binding sites of the transcriptional activator NF-Y overlaps with transcriptional repressors at the γ-globin promoter. HPFH mutations present within the distal γ-globin promoter region, e.g., within or near the CCAAT box, may alter the competitive binding of those factors and thus contribute to the increased γ-globin expression and elevated levels of HbF. Genomic locations provided herein for HBG1 and HBG2 are based on the coordinates provided in NCBI Reference Sequence NC_000011, “Homo sapiens chromosome 11, GRCh38.p12 Primary Assembly,” (Version NC_000011.10). The distal CCAAT box of HBG1 and HBG2 is positioned at HBG1 and HBG2 c.-111 to −115 (Genomic location is Hg38 Chr11:5,249,968 to Chr11:5,249,972 and Hg38 Chr11:5,254,892 to Chr11:5,254,896, respectively). The HBG1c.-111 to -115 region is exemplified in SEQ ID NO:198 (HBG1) at positions 2823-2827, and the HBG2 c.-111 to -115 region is exemplified in SEQ ID NO:199 (HBG2) at positions 2747-2751. In certain embodiments, the “CCAAT box target region” denotes the region that is at or near the distal CCAAT box and includes the nucleotides of the distal CCAAT box and 25 nucleotides upstream (5′) and 25 nucleotides downstream (3′) of the distal CCAAT box (i.e., HBG1/2 c.-86 to -140) (Genomic location is Hg38 Chr11:5249943 to Hg38 Chr11:5249997 and Hg38 Chr11:5254867 to Hg38 Chr11:5254921, respectively). The HBG1c.-86 to -140 region is exemplified in SEQ ID NO:198 (HBG1) at positions 2798-2852, and the HBG2 c.-86 to -140 region is exemplified in SEQ ID NO:199 (HBG2) at positions 2723-2776. In other embodiments, the “CCAAT box target region” denotes the region that is at or near the distal CCAAT box and includes the nucleotides of the distal CCAAT box and 5 nucleotides upstream (5′) and 5 nucleotides downstream (3′) of the distal CCAAT box (i.e., HBG1/2 c.-106 to -120 (Genomic location is Hg38 Chr11:5249963 to Hg38 Chr11:5249977 (HGB1 and Hg38 Chr11:5254887 to Hg38 Chr11:5254901, respectively)). The HBG1c.-106 to -120 region is exemplified in SEQ ID NO:198 (HBG1) at positions 2818-2832, and the HBG2 c.-106 to -120 region is exemplified in SEQ ID NO:199 (HBG2) at positions 2742-2756. The term “CCAAT box target site alteration” and the like refer to alterations (e.g., deletions, insertions, mutations) of one or more nucleotides of the CCAAT box target region. Examples of exemplary CCAAT box target region alterations include, without limitation, the 1 nt deletion, 4 nt deletion, 11 nt deletion, 13 nt deletion, and 18 nt deletion, and -117 G>A alteration. As used herein, the terms “CCAAT box” and “CAAT box” can be used interchangeably.

The notations “c.-114 to -102 region,” “c.-102 to -114 region,” “-102: -114,” “13 nt target region” and the like refer to a sequence that is 5′ of the transcription start site (TSS) of the HBG1 and/or HBG2 gene at the genomic location Hg38 Chr11:5,249,959 to Hg38 Chr11:5,249,971 and Hg38 Chr11:5,254,883 to Hg38 Chr11:5,254,895, respectively. The HBG1c.-102 to -114 region is exemplified in SEQ ID NO:198 (HBG1) at positions 2824-2836 and the HBG2 c.-102 to -114 region is exemplified in SEQ ID NO:199 (HBG2) at positions 2748-2760. The term “13 nt deletion” and the like refer to deletions of the 13 nt target region.

The notations “c.-121 to -104 region,” “c.-104 to -121 region,” “-104: -121,” “18 nt target region,” and the like refer to a sequence that is 5′ of the transcription start site (TSS) of the HBG1 and/or HBG2 gene at the genomic location Hg38 Chr11:5,249,961 to Hg38 Chr11:5,249,978 and Hg38 Chr11:5,254,885 to Hg38 Chr11: 5,254,902, respectively. The HBG1c.-104 to -121 region is exemplified in SEQ ID NO:198 (HBG1) at positions 2817-2834, and the HBG2 c.-104 to -121 region is exemplified in SEQ ID NO:199 (HBG2) at positions 2741-2758. The term “18 nt deletion” and the like refer to deletions of the 18 nt target region.

The notations “c.-105 to -115 region,” “c.-115 to -105 region,” “-105: -115,” “11 nt target region,” and the like refer to a sequence that is 5′ of the transcription start site (TSS) of the HBG1 and/or HBG2 gene at the genomic location Hg38 Chr11:5,249,962 to Hg38 Chr11:5,249,972 and Hg38 Chr11:5,254,886 to Hg38 Chr11:5,254,896, respectively. The HBG1c.-105 to -115 region is exemplified in SEQ ID NO:198 (HBG1) at positions 2823-2833, and the HBG2 c.-105 to -115 region is exemplified in SEQ ID NO:199 (HBG2) at positions 2747-2757. The term “11 nt deletion” and the like refer to deletions of the 11 nt target region.

The notations “c.-115 to -112 region,” “c.-112 to -115 region,” “-112: -115,” “4 nt target region,” and the like refer to a sequence that is 5′ of the transcription start site (TSS) of the HBG1 and/or HBG2 gene at the genomic location Hg38 Chr11:5,249,969 to Hg38 Chr11:5,249,972 and Hg38 Chr11:5,254,893 to Hg38 Chr11:5,254,896, respectively. The HBG1c.-112 to -115 region is exemplified in SEQ ID NO:198 at positions 2823-2826, and the HBG2 c.-112 to -115 region is exemplified in SEQ ID NO:199 (HBG2) at positions 2747-2750. The term “4 nt deletion” and the like refer to deletions of the 4 nt target region.

The notations “c.-116 region,” “HBG-116,” “1 nt target region,” and the like refer to a sequence that is 5′ of the transcription start site (TSS) of the HBG1 and/or HBG2 gene at the genomic location Hg38 Chr11:5,249,973 and Hg38 Chr11:5,254,897, respectively. The HBG1c.-116 region is exemplified in SEQ ID NO:198 at position 2822, and the HBG2 c.-116 region is exemplified in SEQ ID NO:199 (HBG2) at position 2746. The term “1 nt deletion” and the like refer to deletions of the 1 nt target region.

The notations “c.-117 G>A region,” “HBG-117 G>A,” “−117 G>A target region” and the like refer to a sequence that is 5′ of the transcription start site (TSS) of the HBG1 and/or HBG2 gene at the genomic location Hg38 Chr11:5,249,974 to Hg38 Chr11:5,249,974 and Hg38 Chr11:5,254,898 to Hg38 Chr11:5,254,898, respectively. The HBG1c.-117 G>A region is exemplified by a substitution from guanine (G) to adenine (A) in SEQ ID NO: 198 at position 2821, and the HBG2 c.-117 G>A region is exemplified by a substitution from G to A in SEQ ID NO:199 (HBG2) at position 2745. The term “−117 G>A alteration” and the like refer to a substitution from G to A at the −117G>A target region.

The term “proximal HBG1/2 promoter target sequence” denotes the region within 50, 100, 200, 300, 400, or 500 bp of a proximal HBG1/2 promoter sequence including the 13 nt target region. Alterations by genome editing systems according to this disclosure facilitate (e.g., cause, promote or tend to increase the likelihood of) upregulation of HbF production in erythroid progeny.

Where ranges are provided herein, endpoints are included. Furthermore, it is to be understood that unless otherwise indicated or otherwise evident from the context and/or the understanding of one of ordinary skill in the art, values that are expressed as ranges can assume any specific value within the stated ranges in different embodiments of the invention, to the tenth of the unit of the lower limit of the range, unless the context clearly dictates otherwise. It is also to be understood that unless otherwise indicated or otherwise evident from the context and/or the understanding of one of ordinary skill in the art, values expressed as ranges can assume any subrange within the given range, wherein the endpoints of the subrange are expressed to the same degree of accuracy as the tenth of the unit of the lower limit of the range.

### Overview

The various embodiments of this disclosure generally relate to genome editing systems configured to introduce alterations (e.g., a deletion or insertion, or other mutation) into chromosomal DNA that enhance transcription of the HBG1 and/or HBG2 genes, which encode the Aγ and Gγ subunits of hemoglobin, respectively. In certain embodiments, increased expression of one or more γ-globin genes (e.g., HBG1, HBG2) using the methods provided herein results in preferential formation of HbF over HbA and/or increased HbF levels as a percentage of total hemoglobin. In certain embodiments, the disclosure generally relates to the use of RNP complexes comprising a gRNA complexed to a Cas12a protein. In certain embodiments, the gRNA may be unmodified or modified. In certain embodiments, the gRNA may comprise a sequence set forth in Table 20. In certain embodiments, a Cas12a protein may comprise a sequence set forth in SEQ ID NOs:200, 201, 205-215, 221-226, 280-283, 293-295, 335 (Cas12a polypeptide sequences). In certain embodiments a Cas12a protein may be encoded by a sequence comprising SEQ ID NOs:216-218, 296-303, 336 (Cas12a polynucleotide sequences). In certain embodiments, the RNP complex may comprise an RNP complex set forth in Table 18. For example, the RNP complex may include a gRNA comprising the sequence set forth in SEQ ID NO:237 and a Cas12a protein comprising a sequence set forth in SEQ ID NO:283 (RNP27, Table 18).

It has previously been shown that patients with the condition Hereditary Persistence of Fetal Hemoglobin (HPFH) contain mutations in an γ-globin regulatory element that results in fetal γ-globin expression throughout life, rather than being repressed around the time of birth (Martyn 2017). This results in elevated fetal hemoglobin (HbF) expression. HPFH mutations may be deletional or non-deletional (e.g., point mutations). Subjects with HPFH exhibit lifelong expression of HbF, i.e., they do not undergo or undergo only partial globin switching, with no symptoms of anemia.

HbF expression can be induced through point mutations in an γ-globin regulatory element that is associated with a naturally occurring HPFH variant, including, for example, HBG1c.-114 C>T; c.-117 G>A; c.-158 C>T; c.-167 C>T; c.-170 G>A; c.-175 T>G; c.-175 T>C; c.-195 C>G; c.-196 C>T; c.-197 C>T; c.-198 T>C; c.-201 C>T; c.-202 C>T; c.-211 C>T, c.-251 T>C; or c.-499 T>A; or HBG2 c.-109 G>T; c.-110 A>C; c.-114 C>A; c.-114 C>T; c.-114 C>G; c.-157 C>T; c.-158 C>T; c.-167 C>T; c.-167 C>A; c.-175 T>C; c.-197 C>T; c.-200+C; c.-202 C>G; c.-211 C>T; c.-228 T>C; c.-255 C>G; c.-309 A>G; c.-369 C>G; or c.-567 T>G.

Naturally occurring mutations at the distal CCAAT box motif found within the promoter of the HBG1 and/or HBG2 genes (i.e., HBG1/2 c.-111 to -115) have also been shown to result in continued γ-globin expression and the HPFH condition. It is thought that alteration (mutation or deletion) of the CCAAT box may disrupt the binding of one or more transcriptional repressors, resulting in continued expression of the γ-globin gene and elevated HbF expression (Martyn 2017). For example, a naturally occurring 13 base pair del c.-114 to -102 (“13 nt deletion”) has been shown to be associated with elevated levels of HbF (Martyn 2017). The distal CCAAT box likely overlaps with the binding motifs within and surrounding the CCAAT box of negative regulatory transcription factors that are expressed in adulthood and repress HBG (Martyn 2017).

The genome editing systems of this disclosure can include an RNA-guided nuclease such as Cas12a protein and one or more gRNAs having a targeting domain that is complementary to a sequence in or near the target region, and optionally one or more of a DNA donor template that encodes a specific mutation (such as a deletion or insertion) in or near the target region, and/or an agent that enhances the efficiency with which such mutations are generated including, without limitation, a random oligonucleotide, a small molecule agonist or antagonist of a gene product involved in DNA repair or a DNA damage response, or a peptide agent.

The treatment of hemoglobinopathies by gene therapy and/or genome editing is complicated by the fact that the cells that are phenotypically affected by the disease, erythrocytes or RBCs, are enucleated, and do not contain genetic material encoding either the aberrant hemoglobin protein (Hb) subunits nor the Aγ or Gγ subunits targeted in the exemplary genome editing approaches described above. This complication is addressed, in certain embodiments of this disclosure, by the alteration of cells that are competent to differentiate into, or otherwise give rise to, erythrocytes. Cells within the erythroid lineage that are altered according to various embodiments of this disclosure include, without limitation, hematopoietic stem and progenitor cells (HSCs), erythroblasts (including basophilic, polychromatic and/or orthochromatic erythroblasts), proerythroblasts, polychromatic erythrocytes or reticulocytes, embryonic stem (ES) cells, and/or induced pluripotent stem (iPSC) cells. These cells may be altered in situ (e.g. within a tissue of a subject) or ex vivo. Implementations of genome editing systems for in situ and ex vivo alteration of cells is described under the heading “Implementation of genome editing systems: delivery, formulations, and routes of administration” below.

In certain embodiments, alterations that result in induction of Aγ and/or Gγ expression are obtained through the use of a genome editing system comprising an RNA-guided nuclease and at least one gRNA having a targeting domain complementary to a sequence within the CCAAT box target region of HBG1 and/or HBG2 or proximate thereto (e.g., within 10, 20, 30, 40, or 50, 100, 200, 300, 400 or 500 bases of the CCAAT box target region). As is discussed in greater detail below, the RNA-guided nuclease and gRNA form a complex that is capable of associating with and altering the CCAAT box target region or a region proximate thereto. Examples of suitable gRNAs and gRNA targeting domains directed to the CCAAT box target region of HBG1 and/or HBG2 or proximate thereto for use in the embodiments disclosed herein include those set forth herein.

In certain embodiments, alterations that result in induction of Aγ and/or Gγ expression are obtained through the use of a genome editing system comprising an RNA-guided nuclease and at least one gRNA having a targeting domain complementary to a sequence within the 13 nt target region of HBG1 and/or HBG2 or proximate thereto (e.g., within 10, 20, 30, 40, or 50, 100, 200, 300, 400 or 500 bases of the 13 nt target region). As is discussed in greater detail below, the RNA-guided nuclease and gRNA form a complex that is capable of associating with and altering the 13 nt target region or a region proximate thereto. Examples of suitable gRNAs and gRNA targeting domains directed to the 13 nt target region of HBG1 and/or HBG2 or proximate thereto for use in the embodiments disclosed herein include those set forth herein.

The genome editing system can be implemented in a variety of ways, as is discussed below in detail. As an example, a genome editing system of this disclosure can be implemented as a ribonucleoprotein complex or a plurality of complexes in which multiple gRNAs are used. This ribonucleoprotein complex can be introduced into a target cell using art-known methods, including electroporation, as described in commonly-assigned International Patent Publication No. WO 2016/182959 by Jennifer Gori (“Gori”), published Nov. 17, 2016, which is incorporated by reference in its entirety herein.

The ribonucleoprotein complexes within these compositions are introduced into target cells by art-known methods, including without limitation electroporation (e.g. using the Nuclcofection™ technology commercialized by Lonza, Basel, Switzerland or similar technologies commercialized by, for example, Maxcyte Inc. Gaithersburg, Maryland) and lipofection (e.g. using Lipofectamine™ reagent commercialized by Thermo Fisher Scientific, Waltham Massachusetts). Alternatively, or additionally, ribonucleoprotein complexes are formed within the target cells themselves following introduction of nucleic acids encoding the RNA-guided nuclease and/or gRNA. These and other delivery modalities are described in general terms below and in Gori.

Cells that have been altered ex vivo according to this disclosure can be manipulated (e.g. expanded, passaged, frozen, differentiated, de-differentiated, transduced with a transgene, etc.) prior to their delivery to a subject. The cells are, variously, delivered to a subject from which they are obtained (in an “autologous” transplant), or to a recipient who is immunologically distinct from a donor of the cells (in an “allogeneic” transplant).

In some cases, an autologous transplant includes the steps of obtaining, from the subject, a plurality of cells, either circulating in peripheral blood, or within the marrow or other tissue (e.g. spleen, skin, etc.), and optionally manipulating those cells to enrich for cells in the erythroid lineage (e.g. by induction to generate iPSCs, purification of cells expressing certain cell surface markers such as CD34, CD90, CD49f and/or not expressing surface markers characteristic of non-erythroid lineages such as CD10, CD14, CD38, etc.). The cells are, optionally or additionally, expanded, transduced with a transgene, exposed to a cytokine or other peptide or small molecule agent, and/or frozen/thawed prior to transduction with a genome editing system targeting the CCAAT box target region, the 13 nt target region, and/or proximal HBG1/2 promoter target sequence. The genome editing system can be implemented or delivered to the cells in any suitable format, including as a ribonucleoprotein complex, as separated protein and nucleic acid components, and/or as nucleic acids encoding the components of the genome editing system.

In certain embodiments, CD34+ hematopoietic stem and progenitor cells (HSPCs) that have been edited using the genome editing methods disclosed herein may be used for the treatment of a β-hemoglobinopathy in a subject in need thereof. In certain embodiments, the f-hemoglobinopathy may be severe sickle cell disease (SCD) or thalassemia, such as β-thalassemia, δ-thalassemia, or β/δ-thalassemia. In certain embodiments, an exemplary protocol for treatment of a β-hemoglobinopathy may include harvesting CD34+ HSPCs from a subject in need thereof, ex vivo editing of the autologous CD34+ HSPCs using the genome editing methods disclosed herein, followed by reinfusion of the edited autologous CD34+ HSPCs into the subject. In certain embodiments, treatment with edited autologous CD34+ HSPCs may result in increased HbF induction.

Prior to harvesting CD34+ HSPCs, in certain embodiments, a subject may discontinue treatment with hydroxyurea, if applicable, and receive blood transfusions to maintain sufficient hemoglobin (Hb) levels. In certain embodiments, a subject may be administered intravenous plerixafor (e.g., 0.24 mg/kg) to mobilize CD34+ HSPCs from bone marrow into peripheral blood. In certain embodiments, a subject may undergo one or more leukapheresis cycles. In certain embodiments, a subject may undergo approximately one month between leukapheresis cycles, with one cycle defined as two plerixafor-mobilized leukapheresis collections performed on consecutive days. In certain embodiments, a subject may undergo approximately one month between leukapheresis cycles, with one cycle defined as two, three, or more plerixafor-mobilized leukapheresis collections performed on consecutive dates or with one day, two days, three days, four days, or five days between leukapheresis collections. In certain embodiments, the number of leukapheresis cycles performed for a subject may be the number required to achieve a dose of edited autologous CD34+ HSPCs (e.g., ≥2×10⁶ cells/kg, ≥3×10⁶ cells/kg, ≥4×10⁶ cells/kg, ≥5×10⁶ cells/kg, ≥6×10⁶ cells/kg, ≥7×10⁶ cells/kg, ≥8×10⁶ cells/kg, ≥9×10⁶ cells/kg, ≥10×10⁶ cells/kg, ≥11×10⁶ cells/kg, ≥12×10⁶ cells/kg, ≥13×10⁶ cells/kg, ≥14×10⁶ cells/kg, ≥15×10⁶ cells/kg, ≥16×10⁶ cells/kg, ≥17×10⁶ cells/kg, ≥18×10⁶ cells/kg, ≥19×10⁶ cells/kg, ≥20×10⁶ cells/kg, 2×10⁶ cells/kg to 3×10⁶ cells/kg, 3×10⁶ cells/kg to 20×10⁶ cells/kg, 3×10⁶ cells/kg to 10×10⁶ cells/kg, 3×10⁶ cells/kg to 4×10⁶ cells/kg, 4×10⁶ cells/kg to 5×10⁶ cells/kg) to be reinfused back into the subject, along with, e.g., one, two or more doses of unedited autologous CD34+ HSPCs/kg for backup storage (e.g., ≥1.5×10⁶ cells/kg, ≥2.0×10⁶ cells/kg, ≥3.0×10⁶ cells/kg, ≥4.0×10⁶ cells/kg). In certain embodiments, the CD34+ HSPCs harvested from the subject may be edited using any of the genome editing methods discussed herein. In certain embodiments, any one or more of the gRNAs and one or more of the RNA-guided nucleases disclosed herein may be used in the genome editing methods.

In certain embodiments, the treatment may include an autologous stem cell transplant. In certain embodiments, a subject may undergo myeloablative conditioning with busulfan conditioning (e.g., dose-adjusted based on first-dose pharmacokinetic analysis, with a test dose of 1 mg/kg). In certain embodiments, conditioning may occur for four consecutive days. In certain embodiments, following a busulfan washout period, edited autologous CD34+ HSPCs may be administered to the subject. In certain embodiments, the busulfan washout period may be, for example, 1 day, 2 days, 3 days, 4 days, 5 days, 6 days, 7 days, 8 days, 9 days, 10 days, 11 days, 12 days, 13 days, 14 days or longer. In certain embodiments, following a busulfan washout period, edited autologous CD34+ HSPCs (e.g., about ≥1×10⁶ cells/kg, ≥2×10⁶ cells/kg, ≥3×10⁶ cells/kg, ≥4×10⁶ cells/kg, ≥5×10⁶ cells/kg, ≥6×10⁶ cells/kg, ≥7×10⁶ cells/kg, ≥8×10⁶ cells/kg, ≥9×10⁶ cells/kg, ≥10×10⁶ cells/kg, ≥11×10⁶ cells/kg, ≥12×10⁶ cells/kg, ≥13×10⁶ cells/kg, ≥14×10⁶ cells/kg, ≥15×10⁶ cells/kg, ≥16×10⁶ cells/kg, ≥17×10⁶ cells/kg, ≥18×10⁶ cells/kg, ≥19×10⁶ cells/kg, ≥20×10⁶ cells/kg, ≥21×10⁶ cells/kg, ≥22×10⁶ cells/kg, ≥23×10⁶ cells/kg, ≥24×10⁶ cells/kg, ≥25×10⁶ cells/kg, 1×10⁶ cells/kg to 25×10⁶ cells/kg, 1×10⁶ cells/kg to 5×10⁶ cells/kg, 3×10⁶ cells/kg to 20×10⁶ cells/kg, 3×10⁶ cells/kg to 15×10⁶ cells/kg, 3×10⁶ cells/kg to 10×10⁶ cells/kg, 3×10⁶ cells/kg to 5×10⁶ cells/kg, 5×10⁶ cells/kg to 10×10⁶ cells/kg, 10×10⁶ cells/kg to 15×10⁶ cells/kg, 15×10⁶ cells/kg to 20×10⁶ cells/kg, 20×10⁶ cells/kg to 25×10⁶ cells/kg) may be reinfused into the subject (e.g., into peripheral blood). In certain embodiments, the edited autologous CD34+ HSPCs may be manufactured and cryopreserved for a particular subject. In certain embodiments, a subject may attain neutrophil engraftment following a sequential myeloablative conditioning regimen and infusion of edited autologous CD34+ cells. Neutrophil engraftment may be defined as three consecutive measurements of ANC≥0.5×10⁹/L on different days.

However it is implemented, a genome editing system may include, or may be co-delivered with, one or more factors that improve the viability of the cells during and after editing, including without limitation an aryl hydrocarbon receptor antagonist such as StemRegenin-1 (SRI), UM171, LGC0006, alpha-napthoflavone, and CH-223191, and/or an innate immune response antagonist such as cyclosporin A, dexamethasone, reservatrol, a MyD88 inhibitory peptide, an RNAi agent targeting Myd88, a B18R recombinant protein, a glucocorticoid, OxPAPC, a TLR antagonist, rapamycin, BX795, and a RLR shRNA. These and other factors that improve the viability of the cells during and after editing are described in Gori, under the heading “I. Optimization of Stem Cells” from page 36 through page 61, which is incorporated by reference herein.

The cells, following delivery of the genome editing system, are optionally manipulated e.g. to enrich for HSCs and/or cells in the erythroid lineage and/or for edited cells, to expand them, freeze/thaw, or otherwise prepare the cells for return to the subject. The edited cells are then returned to the subject, for instance in the circulatory system by means of intravenous delivery or delivery or into a solid tissue such as bone marrow.

Functionally, alteration of the CCAAT box target region, the 13 nt target region, and/or proximal HBG1/2 promoter target sequence using the compositions, methods and genome editing systems of this disclosure results in significant induction, among hemoglobin-expressing cells, of Aγ and/or Gγ subunits (referred to interchangeably as HbF expression), e.g. at least 5%, 10%, 15%, 20%, 25%, 30%, 35%, 40%, 45%, 50% or greater induction of Aγ and/or Gγ subunit expression relative to unmodified controls. This induction of protein expression is generally the result of alteration of the CCAAT box target region, 13 nt target region, and/or proximal HBG1/2 promoter target sequence (expressed, e.g. in terms of the percentage of total genomes comprising indel mutations within the plurality of cells) in some or all of the plurality of cells that are treated, e.g. at least 5%, 10%, 15%, 20%, 25%, 30%, 35%, 40%, 45%, 50% of the plurality of cells comprise at least one allele comprising a sequence alteration, including, without limitation, an indel, insertion, or deletion in or near the CCAAT box target region, 13 nt target region, and/or proximal HBG1/2 promoter target sequence.

The functional effects of alterations caused or facilitated by the genome editing systems and methods of the present disclosure can be assessed in any number of suitable ways. For example, the effects of alterations on expression of fetal hemoglobin can be assessed at the protein or mRNA level. Expression of HBG1 and HBG2 mRNA can be assessed by digital droplet PCR (ddPCR), which is performed on cDNA samples obtained by reverse transcription of mRNA harvested from treated or untreated samples. Primers for HBG1, HBG2, HBB, and/or HBA may be used individually or multiplexed using methods known in the art. For example, ddPCR analysis of samples may be conducted using the QX200™ ddPCR system commercialized by Bio Rad (Hercules, CA), and associated protocols published by BioRad. Fetal hemoglobin protein may be assessed by high pressure liquid chromatography (HPLC), for example, according to the methods discussed on pp. 143-44 in Chang 2017 (incorporated by reference herein), or fast protein liquid chromatography (FPLC), using ion-exchange and/or reverse phase columns to resolve HbF, HbB and HbA and/or Aγ and Gγ globin chains as is known in the art.

It should be noted that the rate at which the CCAAT box target region is altered in the target cells can be modified by the use of optional genome editing system components such as oligonucleotide donor templates. Donor template design is described in general terms below under the heading “Donor template design.”

The embodiments described herein may be used in all classes of vertebrate including, but not limited to, primates, mice, rats, rabbits, pigs, dogs, and cats.

This overview has focused on a handful of exemplary embodiments that illustrate the principles of genome editing systems and CRISPR-mediated methods of altering cells. For clarity, however, this disclosure encompasses modifications and variations that have not been expressly addressed above, but will be evident to those of skill in the art. With that in mind, the following disclosure is intended to illustrate the operating principles of genome editing systems more generally. What follows should not be understood as limiting, but rather illustrative of certain principles of genome editing systems and CRISPR-mediated methods utilizing these systems, which, in combination with the instant disclosure, will inform those of skill in the art about additional implementations and modifications that are within its scope.

### Guide RNAs

This overview has focused on a handful of exemplary embodiments that illustrate the principles of genome editing systems and CRISPR-mediated methods of altering cells. For clarity, however, this disclosure encompasses modifications and variations that have not been expressly addressed above, but will be evident to those of skill in the art. With that in mind, the following disclosure is intended to illustrate the operating principles of genome editing systems more generally. What follows should not be understood as limiting, but rather illustrative of certain principles of genome editing systems and CRISPR-mediated methods utilizing these systems, which, in combination with the instant disclosure, will inform those of skill in the art about additional implementations and modifications that are within its scope.

### Genome Editing Systems

The term “genome editing system” refers to any system having RNA-guided DNA editing activity. Genome editing systems of the present disclosure include at least two components adapted from naturally occurring CRISPR systems: a guide RNA (gRNA) and an RNA-guided nuclease. These two components form a complex that is capable of associating with a specific nucleic acid sequence and editing the DNA in or around that nucleic acid sequence, for instance by making one or more of a single-strand break (an SSB or nick), a double-strand break (a DSB) and/or a point mutation.

Genome editing systems can be implemented (e.g. administered or delivered to a cell or a subject) in a variety of ways, and different implementations may be suitable for distinct applications. For instance, a genome editing system is implemented, in certain embodiments, as a protein/RNA complex (a ribonucleoprotein, or RNP), which can be included in a pharmaceutical composition that optionally includes a pharmaceutically acceptable carrier and/or an encapsulating agent, such as, without limitation, a lipid or polymer micro- or nano-particle, micelle, or liposome. In certain embodiments, a genome editing system is implemented as one or more nucleic acids encoding the RNA-guided nuclease and guide RNA components described above (optionally with one or more additional components); in certain embodiments, the genome editing system is implemented as one or more vectors comprising such nucleic acids, for instance a viral vector such as an adeno-associated virus (see section below under the heading “Implementation of genome editing systems: delivery, formulations, and routes of administration”); and in certain embodiments, the genome editing system is implemented as a combination of any of the foregoing. Additional or modified implementations that operate according to the principles set forth herein will be apparent to the skilled artisan and are within the scope of this disclosure.

It should be noted that the genome editing systems of the present disclosure can be targeted to a single specific nucleotide sequence, or may be targeted to—and capable of editing in parallel—two or more specific nucleotide sequences through the use of two or more guide RNAs. The use of multiple gRNAs is referred to as “multiplexing” throughout this disclosure, and can be employed to target multiple, unrelated target sequences of interest, or to form multiple SSBs or DSBs within a single target domain and, in some cases, to generate specific edits within such target domain. For example, International Patent Publication No. WO 2015/138510 by Maeder et al. (“Maeder”), which is incorporated by reference herein, describes a genome editing system for correcting a point mutation (C.299I+1655A to G) in the human CEP290 gene that results in the creation of a cryptic splice site, which in turn reduces or eliminates the function of the gene. The genome editing system of Maeder utilizes two guide RNAs targeted to sequences on either side of (i.e. flanking) the point mutation, and forms DSBs that flank the mutation. This, in turn, promotes deletion of the intervening sequence, including the mutation, thereby eliminating the cryptic splice site and restoring normal gene function.

As another example, WO 2016/073990 by Cotta-Ramusino et al. (“Cotta-Ramusino”), which is incorporated by reference herein, describes a genome editing system that utilizes two gRNAs in combination with a Cas9 nickase (a Cas9 that makes a single strand nick such as S. pyogenes D10A), an arrangement termed a “dual-nickase system.” The dual-nickase system of Cotta-Ramusino is configured to make two nicks on opposite strands of a sequence of interest that are offset by one or more nucleotides, which nicks combine to create a double strand break having an overhang (5′ in the case of Cotta-Ramusino, though 3′ overhangs are also possible). The overhang, in turn, can facilitate homology directed repair events in some circumstances. And, as another example, WO 2015/070083 by Palestrant et al. (incorporated by reference herein) describes a gRNA targeted to a nucleotide sequence encoding Cas9 (referred to as a “governing RNA”), which can be included in a genome editing system comprising one or more additional gRNAs to permit transient expression of a Cas9 that might otherwise be constitutively expressed, for example in some virally transduced cells. These multiplexing applications are intended to be exemplary, rather than limiting, and the skilled artisan will appreciate that other applications of multiplexing are generally compatible with the genome editing systems described here.

Genome editing systems can, in some instances, form double strand breaks that are repaired by cellular DNA double-strand break mechanisms such as NHEJ or HDR. These mechanisms are described throughout the literature (see, e.g., Davis & Maizels 2014 (describing Alt-HDR); Frit 2014 (describing Alt-NHEJ); Iyama & Wilson 2013 (describing canonical HDR and NHEJ pathways generally)).

Where genome editing systems operate by forming DSBs, such systems optionally include one or more components that promote or facilitate a particular mode of double-strand break repair or a particular repair outcome. For instance, Cotta-Ramusino also describes genome editing systems in which a single stranded oligonucleotide “donor template” is added; the donor template is incorporated into a target region of cellular DNA that is cleaved by the genome editing system, and can result in a change in the target sequence.

Guide RNA (gRNA) Molecules

The terms “guide molecule,” “guide RNA” and “gRNA” refer to any nucleic acid that promotes the specific association (or “targeting”) of an RNA-guided nuclease such as a Cas12a protein to a target sequence such as a genomic or episomal sequence in a cell. gRNAs can be unimolecular (comprising a single RNA molecule, and referred to alternatively as chimeric), or modular (comprising more than one, and typically two, separate RNA molecules, such as a crRNA and a tracrRNA, which are usually associated with one another, for instance by duplexing). gRNAs and their component parts are described throughout the literature (see, e.g., Briner 2014, which is incorporated by reference; Cotta-Ramusino). Examples of modular and unimolecular gRNAs that may be used according to the embodiments herein include, without limitation, the sequences set forth in SEQ ID NOs:21-23 and 30-43. Examples of gRNA proximal and tail domains that may be used according to the embodiments herein include, without limitation, the sequences set forth in SEQ ID NOs:24-29. The guide molecule can be an RNA molecule. The guide molecule can also comprise one or more nucleotides other than RNA nucleotides, for example, the guide molecule can be a DNA/RNA hybrid molecule, and/or the guide molecule can comprise one or more modified nucleotides (including, but not limited to, one or more modified DNA or RNA nucleotides).

In bacteria and archea, type II CRISPR systems generally comprise an RNA-guided nuclease protein such as Cas9, a CRISPR RNA (crRNA) that includes a 5′ region that is complementary to a foreign sequence, and a trans-activating crRNA (tracrRNA) that includes a 5′ region that is complementary to, and forms a duplex with, a 3′ region of the crRNA. While not intending to be bound by any theory, it is thought that this duplex facilitates the formation of—and is necessary for the activity of—the Cas9/gRNA complex. As type II CRISPR systems were adapted for use in gene editing, it was discovered that the crRNA and tracrRNA could be joined into a single unimolecular or chimeric guide RNA, in one non-limiting example, by means of a four nucleotide (e.g., GAAA) “tetraloop” or “linker” sequence bridging complementary regions of the crRNA (at its 3′ end) and the tracrRNA (at its 5′ end). (Mali 2013; Jiang 2013; Jinek 2012; all incorporated by reference herein).

Guide RNAs, whether unimolecular or modular, include a “targeting domain” that is fully or partially complementary to a target domain within a target sequence, such as a DNA sequence in the genome of a cell where editing is desired. Targeting domains are referred to by various names in the literature, including without limitation “guide sequences” (Hsu 2013, incorporated by reference herein), “complementarity regions” (Cotta-Ramusino), “spacers” (Briner 2014) and generically as “crRNAs” (Jiang). Irrespective of the names they are given, targeting domains are typically 10-30 nucleotides in length, and in certain embodiments are 16-24 nucleotides in length (for instance, 16, 17, 18, 19, 20, 21, 22, 23 or 24 nucleotides in length), and are at or near the 5′ terminus of in the case of a Cas9 gRNA, and at or near the 3′ terminus in the case of a Cas12a gRNA.

In addition to the targeting domains, gRNAs typically (but not necessarily, as discussed below) include a plurality of domains that may influence the formation or activity of gRNA/Cas complexes. For instance, as mentioned above, the duplexed structure formed by first and secondary complementarity domains of a gRNA (also referred to as a repeat:anti-repeat duplex) interacts with the recognition (REC) lobe of Cas9 and can mediate the formation of Cas9/gRNA complexes (Nishimasu 2014; Nishimasu 2015; both incorporated by reference herein). It should be noted that the first and/or second complementarity domains may contain one or more poly-A tracts, which can be recognized by RNA polymerases as a termination signal. The sequence of the first and second complementarity domains are, therefore, optionally modified to eliminate these tracts and promote the complete in vitro transcription of gRNAs, for instance through the use of A-G swaps as described in Briner 2014, or A-U swaps. These and other similar modifications to the first and second complementarity domains are within the scope of the present disclosure.

Along with the first and second complementarity domains, Cas9 gRNAs typically include two or more additional duplexed regions that are involved in nuclease activity in vivo but not necessarily in vitro. (Nishimasu 2015). A first stem-loop one near the 3′ portion of the second complementarity domain is referred to variously as the “proximal domain,” (Cotta-Ramusino) “stem loop 1” (Nishimasu 2014 and 2015) and the “nexus” (Briner 2014). One or more additional stem loop structures are generally present near the 3′ end of the gRNA, with the number varying by species: S. pyogenes gRNAs typically include two 3′ stem loops (for a total of four stem loop structures including the repeat:anti-repeat duplex), while S. aureus and other species have only one (for a total of three stem loop structures). A description of conserved stem loop structures (and gRNA structures more generally) organized by species is provided in Briner 2014.

While the foregoing description has focused on gRNAs for use with Cas9, it should be appreciated that other RNA-guided nucleases exist which utilize gRNAs that differ in some ways from those described to this point. For instance, Cas12a (also known as Cpf1, “CRISPR from Prevotella and franciscella 1”) is a RNA-guided nuclease that does not require a tracrRNA to function. (Zetsche 2015, incorporated by reference herein). A gRNA for use in a Cas12a genome editing system generally includes a targeting domain and a complementarity domain (alternately referred to as a “handle”). It should also be noted that, in gRNAs for use with Cas12a, the targeting domain is usually present at or near the 3′ end, rather than the 5′ end as described above in connection with Cas9 gRNAs (the handle is at or near the 5′ end of a Cas12a gRNA).

Those of skill in the art will appreciate, however, that although structural differences may exist between gRNAs from different prokaryotic species, or between Cas12a and Cas9 gRNAs, the principles by which gRNAs operate are generally consistent. Because of this consistency of operation, gRNAs can be defined, in broad terms, by their targeting domain sequences, and skilled artisans will appreciate that a given targeting domain sequence can be incorporated in any suitable gRNA, including a unimolecular or chimeric gRNA, or a gRNA that includes one or more chemical modifications and/or sequential modifications (substitutions, additional nucleotides, truncations, etc.). Thus, for economy of presentation in this disclosure, gRNAs may be described solely in terms of their targeting domain sequences.

More generally, skilled artisans will appreciate that some aspects of the present disclosure relate to systems, methods and compositions that can be implemented using multiple RNA-guided nucleases. For this reason, unless otherwise specified, the term gRNA should be understood to encompass any suitable gRNA that can be used with any RNA-guided nuclease, and not only those gRNAs that are compatible with a particular RNA-guided nuclease, e.g., a particular species of Cas9 or Cas12a. By way of illustration, the term gRNA can, in certain embodiments, include a gRNA for use with any RNA-guided nuclease occurring in a Class 2 CRISPR system, such as a type II or type V CRISPR system, or an RNA-guided nuclease derived or adapted therefrom.

gRNA Design

Methods for selection and validation of target sequences as well as off-target analyses have been described previously (see, e.g., Mali 2013; Hsu 2013; Fu 2014; Heigwer 2014; Bae 2014; Xiao 2014). Each of these references is incorporated by reference herein. As a non-limiting example, gRNA design may involve the use of a software tool to optimize the choice of potential target sequences corresponding to a user's target sequence, e.g., to minimize total off-target activity across the genome. While off-target activity is not limited to cleavage, the cleavage efficiency at each off-target sequence can be predicted, e.g., using an experimentally-derived weighting scheme. These and other guide selection methods are described in detail in Maeder and Cotta-Ramusino.

Targeting domain sequences of gRNAs that were designed to target disruption of the CCAAT box target region include, but are not limited to, SEQ ID NO:202 or SEQ ID NO:320. In certain embodiments, gRNAs comprising the sequence set forth in SEQ ID NO:202 may be complexed with a Cas12a protein to generate alterations at the CCAAT box target region. In certain embodiments, gRNAs comprising the sequence set forth in SEQ ID NO:320 may be complexed with a Cas12a protein to generate alterations at the CCAAT box target region. In certain embodiments, gRNAs comprising any of the Cas12a gRNAs set forth in Table 20 may be complexed with a Cas12a protein forming an RNP (“gRNA-Cas12a-RNP”) to generate alterations at the CCAAT box target region. In certain embodiments, the Cas12a protein may be His-AsCas12a-nNLS (SEQ ID NO: 200) or His-AsCas12a-sNLS-sNLS (SEQ ID NO:201). In certain embodiments, the Cas12a protein of the gRNA-Cas12a-RNP may comprise a sequence set forth in SEQ ID NOs:200, 201, 205-215, 221, 222-226, 335 (Cas12a polypeptide sequences). In certain embodiments, the Cas12a protein may be encoded by a sequence set forth in SEQ ID NOs:216-218, 336 (Cas12a polynucleotide sequences).

In certain embodiments, a targeting domain of a gRNA molecule has at least 50%, 60%, 70%, 75%, 80%, 85%, 90%, or 95% sequence identity to a nucleotide sequence set forth in SEQ ID NO: 202. In certain embodiments, a targeting domain of a gRNA molecule has 1, 2, 3, 4, 5, 6, 7, 8, 9, or 10 mutations relative to a nucleotide sequence set forth in SEQ ID NO: 202. In certain embodiments, a targeting domain of a gRNA molecule has less than 2, 3, 4, 5, 6, 7, 8, 9, or 10 mutations relative to a nucleotide sequence set forth in SEQ ID NO: 202.

In certain embodiments, a targeting domain of a gRNA molecule has at least 50%, 60%, 70%, 75%, 80%, 85%, 90%, or 95% sequence identity to a nucleotide sequence set forth in SEQ ID NO: 320. In certain embodiments, a targeting domain of a gRNA molecule has 1, 2, 3, 4, 5, 6, 7, 8, 9, or 10 mutations relative to a nucleotide sequence set forth in SEQ ID NO: 320. In certain embodiments, a targeting domain of a gRNA molecule has less than 2, 3, 4, 5, 6, 7, 8, 9, or 10 mutations relative to a nucleotide sequence set forth in SEQ ID NO: 320.

gRNA Modifications

The activity, stability, or other characteristics of gRNAs can be altered through the incorporation of certain modifications. As one example, transiently expressed or delivered nucleic acids can be prone to degradation by, e.g., cellular nucleases. Accordingly, the gRNAs described herein can contain one or more modified nucleosides or nucleotides which introduce stability toward nucleases. While not wishing to be bound by theory it is also believed that certain modified gRNAs described herein can exhibit a reduced innate immune response when introduced into cells. Those of skill in the art will be aware of certain cellular responses commonly observed in cells, e.g., mammalian cells, in response to exogenous nucleic acids, particularly those of viral or bacterial origin. Such responses, which can include induction of cytokine expression and release and cell death, may be reduced or eliminated altogether by the modifications presented herein.

Certain exemplary modifications discussed in this section can be included at any position within a gRNA sequence including, without limitation at or near the 5′ end (e.g., within 1-10, 1-5, or 1-2 nucleotides of the 5′ end) and/or at or near the 3′ end (e.g., within 1-10, 1-5, or 1-2 nucleotides of the 3′ end). In some cases, modifications are positioned within functional motifs, such as the repeat-anti-repeat duplex of a Cas9 gRNA, a stem loop structure of a Cas9 or Cas12a gRNA, and/or a targeting domain of a gRNA.

As one example, the 5′ end of a gRNA can include a eukaryotic mRNA cap structure or cap analog (e.g., a G(5′)ppp(5′)G cap analog, a m7G(5′)ppp(5′)G cap analog, or a 3′-O-Me-m7G(5′)ppp(5′)G anti reverse cap analog (ARCA)), as shown below:

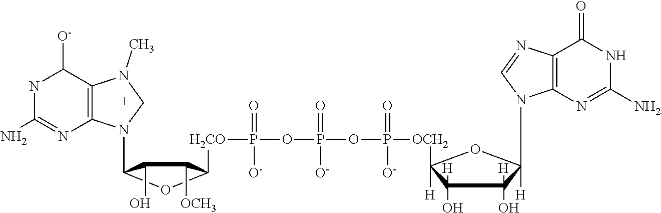

The cap or cap analog can be included during either chemical synthesis or in vitro transcription of the gRNA.

Along similar lines, the 5′ end of the gRNA can lack a 5 triphosphate group. For instance, in vitro transcribed gRNAs can be phosphatase-treated (e.g., using calf intestinal alkaline phosphatase) to remove a 5′ triphosphate group.

Another modification involves the addition, at the 3′ end of a gRNA, of a plurality (e.g., 1-10, 10-20, or 25-200) of adenine (A) residues referred to as a polyA tract. The polyA tract can be added to a gRNA during chemical synthesis, following in vitro transcription using a polyadenosine polymerase (e.g., E. coli Poly(A)Polymerase), or in vivo by means of a polyadenylation sequence, as described in Maeder.

It should be noted that the modifications described herein can be combined in any suitable manner, e.g. a gRNA, whether transcribed in vivo from a DNA vector, or in vitro transcribed gRNA, can include either or both of a 5′ cap structure or cap analog and a 3′ polyA tract.

Guide RNAs can be modified at a 3′ terminal U ribose. For example, the two terminal hydroxyl groups of the U ribose can be oxidized to aldehyde groups and a concomitant opening of the ribose ring to afford a modified nucleoside as shown below:

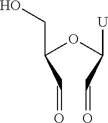

wherein “U” can be an unmodified or modified uridine.

The 3′ terminal U ribose can be modified with a 2′3′ cyclic phosphate as shown below:

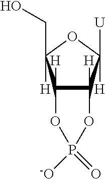

wherein “U” can be an unmodified or modified uridine.

Guide RNAs can contain 3′ nucleotides which can be stabilized against degradation, e.g., by incorporating one or more of the modified nucleotides described herein. In certain embodiments, uridines can be replaced with modified uridines, e.g., 5-(2-amino)propyl uridine, and 5-bromo uridine, or with any of the modified uridines described herein; adenosines and guanosines can be replaced with modified adenosines and guanosines, e.g., with modifications at the 8-position, e.g., 8-bromo guanosine, or with any of the modified adenosines or guanosines described herein.

In certain embodiments, sugar-modified ribonucleotides can be incorporated into the gRNA, e.g., wherein the 2′ OH-group is replaced by a group selected from H, —OR, —R (wherein R can be, e.g., alkyl, cycloalkyl, aryl, aralkyl, heteroaryl or sugar), halo, —SH, —SR (wherein R can be, e.g., alkyl, cycloalkyl, aryl, aralkyl, heteroaryl or sugar), amino (wherein amino can be, e.g., NH₂; alkylamino, dialkylamino, heterocyclyl, arylamino, diarylamino, heteroarylamino, diheteroarylamino, or amino acid); or cyano (—CN). In certain embodiments, the phosphate backbone can be modified as described herein, e.g., with a phosphorothioate (PhTx) group. In certain embodiments, one or more of the nucleotides of the gRNA can each independently be a modified or unmodified nucleotide including, but not limited to 2′-sugar modified, such as, 2′-O-methyl, 2′-O-methoxyethyl, or 2′-Fluoro modified including, e.g., 2′-F or 2′-O-methyl, adenosine (A), 2′-F or 2′-O-methyl, cytidine (C), 2′-F or 2′-O-methyl, uridine (U), 2′-F or 2′-O-methyl, thymidine (T), 2′-F or 2′-O-methyl, guanosine (G), 2′-O-methoxyethyl-5-methyluridine (Teo), 2′-O-methoxyethyladenosine (Aeo), 2′-O-methoxyethyl-5-methylcytidine (m5Ceo), and any combinations thereof.

Guide RNAs can also include “locked” nucleic acids (LNA) in which the 2′ OH-group can be connected, e.g., by a C1-6 alkylene or C1-6 heteroalkylene bridge, to the 4′ carbon of the same ribose sugar. Any suitable moiety can be used to provide such bridges, include without limitation methylene, propylene, ether, or amino bridges; O-amino (wherein amino can be, e.g., NH₂; alkylamino, dialkylamino, heterocyclyl, arylamino, diarylamino, heteroarylamino, or diheteroarylamino, ethylenediamine, or polyamino) and aminoalkoxy or O(CH₂)ₙ-amino (wherein amino can be, e.g., NH₂; alkylamino, dialkylamino, heterocyclyl, arylamino, diarylamino, heteroarylamino, or diheteroarylamino, ethylenediamine, or polyamino).

In certain embodiments, a gRNA can include a modified nucleotide which is multicyclic (e.g., tricyclo; and “unlocked” forms, such as glycol nucleic acid (GNA) (e.g., R-GNA or S-GNA, where ribose is replaced by glycol units attached to phosphodiester bonds), or threose nucleic acid (TNA, where ribose is replaced with (t-L-threofuranosyl-(3′→2′)).

Generally, gRNAs include the sugar group ribose, which is a 5-membered ring having an oxygen. Exemplary modified gRNAs can include, without limitation, replacement of the oxygen in ribose (e.g., with sulfur (S), selenium (Se), or alkylene, such as, e.g., methylene or ethylene); addition of a double bond (e.g., to replace ribose with cyclopentenyl or cyclohexenyl); ring contraction of ribose (e.g., to form a 4-membered ring of cyclobutane or oxetane); ring expansion of ribose (e.g., to form a 6- or 7-membered ring having an additional carbon or heteroatom, such as for example, anhydrohexitol, altritol, mannitol, cyclohexanyl, cyclohexenyl, and morpholino that also has a phosphoramidate backbone). Although the majority of sugar analog alterations are localized to the 2′ position, other sites are amenable to modification, including the 4′ position. In certain embodiments, a gRNA comprises a 4′-S, 4′-Se or a 4′-C-aminomethyl-2′-O-Me modification.

In certain embodiments, deaza nucleotides, e.g., 7-deaza-adenosine, can be incorporated into the gRNA. In certain embodiments, O- and N-alkylated nucleotides, e.g., N6-methyl adenosine, can be incorporated into the gRNA. In certain embodiments, one or more or all of the nucleotides in a gRNA are deoxynucleotides.

In certain embodiments, gRNAs as used herein may be modified or unmodified gRNAs. In certain embodiments, a gRNA may include one or more modifications. In certain embodiments, the one or more modifications may include a phosphorothioate linkage modification, a phosphorodithioate (PS2) linkage modification, a 2′-O-methyl modification, or combinations thereof. In certain embodiments, the one or more modifications may be at the 5′ end of the gRNA, at the 3′ end of the gRNA, or combinations thereof.

In certain embodiments, a gRNA modification may comprise one or more phosphorodithioate (PS2) linkage modifications.

In some embodiments, a gRNA used herein includes one or more or a stretch of deoxyribonucleic acid (DNA) bases, also referred to herein as a “DNA extension.” In some embodiments, a gRNA used herein includes a DNA extension at the 5′ end of the gRNA, the 3′ end of the gRNA, or a combination thereof. In certain embodiments, the DNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 DNA bases long. For example, in certain embodiments, the DNA extension may be 1, 2, 3, 4, 5, 10, 15, 20, 25, 30, or 35 DNA bases long. In certain embodiments, the DNA extension may be about 25 DNA bases long. In certain embodiments, the DNA extension may include one or more DNA bases selected from adenine (A), guanine (G), cytosine (C), or thymine (T). In certain embodiments, the DNA extension includes the same DNA bases. For example, the DNA extension may include a stretch of adenine (A) bases. In certain embodiments, the DNA extension may include a stretch of thymine (T) bases. In certain embodiments, the DNA extension includes a combination of different DNA bases. In certain embodiments, a DNA extension may comprise or consist of a sequence set forth in Table 19. For example, a DNA extension may comprise or consist of a sequence set forth in SEQ ID NOs:304-319. In certain embodiments, the DNA extension may comprise or consist of SEQ ID NO:306. In certain embodiments, a gRNA used herein includes a DNA extension as well as one or more phosphorothioate linkage modifications, one or more phosphorodithioate (PS2) linkage modifications, one or more 2′-O-methyl modifications, or combinations thereof. In certain embodiments, the one or more modifications may be at the 5′ end of the gRNA, at the 3′ end of the gRNA, or combinations thereof. In certain embodiments, a gRNA including a DNA extension may comprise a sequence set forth in Table 19 that includes a DNA extension. In a particular embodiment, a gRNA including a DNA extension may comprise the sequence set forth in SEQ ID NO:237. In certain embodiments, a gRNA including a DNA extension may comprise a sequence selected from the group consisting of SEQ ID NOs:232-246, 253, 254, 260, 261, 264, 267-270, 272-273, 275-276, 278-279, 284-288, and 292. Without wishing to be bound by theory, it is contemplated that any DNA extension may be used herein, so long as it does not hybridize to the target nucleic acid being targeted by the gRNA. In some embodiments, a gRNA comprising the DNA extension additionally exhibits an increase in editing efficiency, e.g., via changes to gRNA stability, uptake, and/or activity, at the target nucleic acid site relative to a gRNA which does not comprise such a DNA extension.

In some embodiments, a gRNA used herein includes one or more or a stretch of ribonucleic acid (RNA) bases, also referred to herein as an “RNA extension.” In some embodiments, a gRNA used herein includes an RNA extension at the 5′ end of the gRNA, the 3′ end of the gRNA, or a combination thereof. In certain embodiments, the RNA extension may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 RNA bases long. For example, in certain embodiments, the RNA extension may be 1, 2, 3, 4, 5, 10, 15, 20, or 25 RNA bases long. In certain embodiments, the RNA extension may include one or more RNA bases selected from adenine (rA), guanine (rG), cytosine (rC), or uracil (rU), in which the “r” represents RNA, 2′-hydroxy. In certain embodiments, the RNA extension includes the same RNA bases. For example, the RNA extension may include a stretch of adenine (rA) bases. In certain embodiments, the RNA extension includes a combination of different RNA bases. In certain embodiments, an RNA extension may comprise or consist of a sequence set forth in Table 19. For example, an RNA extension may comprise a sequence set forth in 328-334, In certain embodiments, a gRNA used herein includes an RNA extension as well as one or more phosphorothioate linkage modifications, one or more phosphorodithioate (PS2) linkage modifications, one or more 2′-O-methyl modifications, or combinations thereof. In certain embodiments, the one or more modifications may be at the 5′ end of the gRNA, at the 3′ end of the gRNA, or combinations thereof. In certain embodiments, a gRNA including a RNA extension may comprise a sequence set forth in Table 19 that includes an RNA extension. gRNAs including an RNA extension at the 5′ end of the gRNA may comprise a sequence selected from the group consisting of SEQ ID NOs:228-231, 289-291. gRNAs including an RNA extension at the 3′ end of the gRNA may comprise a sequence selected from the group consisting of SEQ ID NOs:256-261, 265, 267, 284-286.

It is contemplated that gRNAs used herein may also include an RNA extension and a DNA extension. In certain embodiments, the RNA extension and DNA extension may both be at the 5′ end of the gRNA, the 3′ end of the gRNA, or a combination thereof. In certain embodiments, the RNA extension is at the 5′ end of the gRNA and the DNA extension is at the 3′ end of the gRNA. In certain embodiments, the RNA extension is at the 3′ end of the gRNA and the DNA extension is at the 5′ end of the gRNA.

In some embodiments, a gRNA which includes both a phosphorothioate modification at the 3′ end as well as a DNA extension at the 5′ end is complexed with a RNA-guided nuclease. e.g., Cas12a, to form an RNP, which is then employed to edit a hematopoietic stem cell (HSC) or a CD34+ cell ex vivo (i.e., outside the body of a subject from whom such a cell is derived), at the HBG locus.

An example of a gRNA as used herein comprises the sequence set forth in SEQ ID NO:237.

### RNA-Guided Nucleases

RNA-guided nucleases according to the present disclosure include, but are not limited to, naturally-occurring Class 2 CRISPR nucleases such as Cas12a, and Cas9, as well as other nucleases derived or obtained therefrom.

In functional terms, RNA-guided nucleases are defined as those nucleases that: (a) interact with (e.g. complex with) a gRNA; and (b) together with the gRNA, associate with, and optionally cleave or modify, a target region of a DNA that includes (i) a sequence complementary to the targeting domain of the gRNA and, optionally, (ii) an additional sequence referred to as a “protospacer adjacent motif,” or “PAM,” which is described in greater detail below. As the following examples will illustrate, RNA-guided nucleases can be defined, in broad terms, by their PAM specificity and cleavage activity, even though variations may exist between individual RNA-guided nucleases that share the same PAM specificity or cleavage activity. Skilled artisans will appreciate that some aspects of the present disclosure relate to systems, methods and compositions that can be implemented using any suitable RNA-guided nuclease having a certain PAM specificity and/or cleavage activity. For this reason, unless otherwise specified, the term RNA-guided nuclease should be understood as a generic term, and not limited to any particular type (e.g. Cas9 vs. Cas12a), species (e.g. S. pyogenes vs. S. aureus) or variation (e.g. full-length vs. truncated or split; naturally-occurring PAM specificity vs. engineered PAM specificity, etc.) of RNA-guided nuclease. For example, in certain embodiments, the RNA-guided nuclease may be Cas-Φ (Pausch 2020).

Various RNA-guided nucleases may require different sequential relationships between PAMs and protospacers. In general, Cas9s recognize PAM sequences that are 3′ of the protospacer. Cas12a, on the other hand, generally recognizes PAM sequences that are 5′ of the protospacer.

In addition to recognizing specific sequential orientations of PAMs and protospacers, RNA-guided nucleases can also recognize specific PAM sequences. S. aureus Cas9, for instance, recognizes a PAM sequence of NNGRRT or NNGRRV, wherein the N residues are immediately 3′ of the region recognized by the gRNA targeting domain. S. pyogenes Cas9 recognizes NGG PAM sequences. And F. novicida Cas12a recognizes a TTN PAM sequence. PAM sequences have been identified for a variety of RNA-guided nucleases, and a strategy for identifying novel PAM sequences has been described by Shmakov 2015. It should also be noted that engineered RNA-guided nucleases can have PAM specificities that differ from the PAM specificities of reference molecules (for instance, in the case of an engineered RNA-guided nuclease, the reference molecule may be the naturally occurring variant from which the RNA-guided nuclease is derived, or the naturally occurring variant having the greatest amino acid sequence homology to the engineered RNA-guided nuclease). Examples of PAMs that may be used according to the embodiments herein include, without limitation, the sequences set forth in SEQ ID NOs:191-197.

In addition to their PAM specificity, RNA-guided nucleases can be characterized by their DNA cleavage activity: naturally-occurring RNA-guided nucleases typically form DSBs in target nucleic acids, but engineered variants have been produced that generate only SSBs (discussed above and in Ran & Hsu 2013, incorporated by reference herein), or that do not cut at all.

### Cas9

Crystal structures have been determined for S. pyogenes Cas9 (Jinek 2014), and for S. aureus Cas9 in complex with a unimolecular guide RNA and a target DNA (Nishimasu 2014; Anders 2014; and Nishimasu 2015).

A naturally occurring Cas9 protein comprises two lobes: a recognition (REC) lobe and a nuclease (NUC) lobe; each of which comprise particular structural and/or functional domains. The REC lobe comprises an arginine-rich bridge helix (BH) domain, and at least one REC domain (e.g. a REC1 domain and, optionally, a REC2 domain). The REC lobe does not share structural similarity with other known proteins, indicating that it is a unique functional domain. While not wishing to be bound by any theory, mutational analyses suggest specific functional roles for the BH and REC domains: the BH domain appears to play a role in gRNA:DNA recognition, while the REC domain is thought to interact with the repeat:anti-repeat duplex of the gRNA and to mediate the formation of the Cas9/gRNA complex.

The NUC lobe comprises a RuvC domain, an HNH domain, and a PAM-interacting (PI) domain. The RuvC domain shares structural similarity to retroviral integrase superfamily members and cleaves the non-complementary (i.e. bottom) strand of the target nucleic acid. It may be formed from two or more split RuvC motifs (such as RuvC I, RuvCII, and RuvCIII in S. pyogenes and S. aureus). The HNH domain, meanwhile, is structurally similar to HNN endonuclease motifs, and cleaves the complementary (i.e. top) strand of the target nucleic acid. The PI domain, as its name suggests, contributes to PAM specificity. Examples of Cas9 RuvC-like and Cas9 HNH-like domain polypeptide sequences are set forth in SEQ ID NOs:7-15, 44-115 (RuvC-like domains) and SEQ ID NOs:16-20, 116-190 (HNH-like domains).

While certain functions of Cas9 are linked to (but not necessarily fully determined by) the specific domains set forth above, these and other functions may be mediated or influenced by other Cas9 domains, or by multiple domains on either lobe. For instance, in S. pyogenes Cas9, as described in Nishimasu 2014, the repeat:antirepeat duplex of the gRNA falls into a groove between the REC and NUC lobes, and nucleotides in the duplex interact with amino acids in the BH, PI, and REC domains. Some nucleotides in the first stem loop structure also interact with amino acids in multiple domains (PI, BH and REC1), as do some nucleotides in the second and third stem loops (RuvC and PI domains). Examples of Cas9 polypeptide sequences are set forth in SEQ ID NOs:1-6, 326.

### Cas12a (Cpf1)

The crystal structure of Acidaminococcus sp. Cas12a in complex with crRNA and a double-stranded (ds) DNA target including a TTTN PAM sequence has been solved by Yamano 2016 (incorporated by reference herein). Cas12a, like Cas9, has two lobes: a REC (recognition) lobe, and a NUC (nuclease) lobe. The REC lobe includes REC1 and REC2 domains, which lack similarity to any known protein structures. The NUC lobe, meanwhile, includes three RuvC domains (RuvC-I, -II and -III) and a BH domain. However, in contrast to Cas9, the Cas12a REC lobe lacks an HNH domain, and includes other domains that also lack similarity to known protein structures: a structurally unique PI domain, three Wedge (WED) domains (WED-I, -II and -III), and a nuclease (Nuc) domain.

While Cas9 and Cas12a share similarities in structure and function, it should be appreciated that certain Cas12a activities are mediated by structural domains that are not analogous to any Cas9 domains. For instance, cleavage of the complementary strand of the target DNA appears to be mediated by the Nuc domain, which differs sequentially and spatially from the HNH domain of Cas9. Additionally, the non-targeting portion of Cas12a gRNA (the handle) adopts a pseudoknot structure, rather than a stem loop structure formed by the repeat:antirepeat duplex in Cas9 gRNAs.

In certain embodiments, a Cas12a protein may include one or more modifications. In certain embodiments the modifications may be, without limitation, one or more mutations in a Cas12a nucleotide sequence or Cas12a amino acid sequence, one or more additional sequences such as a His tag or a nuclear localization signal (NLS), or a combination thereof.

In certain embodiments, the Cas12a protein may be derived from a Cas12a protein selected from the group consisting of Acidaminococcus sp. strain BV3L6 Cas12a protein (AsCas12a), Lachnospiraceae bacterium ND2006 Cas12a protein (LbCas12a), and Lachnospiraceae bacterium MA2020 (Lb2Cas12a). In certain embodiments, the Cas12a protein may comprise a sequence selected from the group consisting of SEQ ID NOs:213-215, having the codon-optimized nucleic acid sequences of SEQ ID NOs:216-218, respectively.

In certain embodiments, the Cas12a protein may comprise a nuclear localization signal (NLS). For example, but not by way of limitation, NLS sequences useful in connection with the methods and compositions disclosed herein will comprise an amino acid sequence capable of facilitating protein import into the cell nucleus. NLS sequences useful in connection with the methods and compositions disclosed herein are known in the art. Examples of such NLS sequences include the nucleoplasmin NLS having the amino acid sequence: KRPAATKKAGQAKKKK (SEQ ID NO:203) and the simian virus 40 “SV40” NLS having the amino acid sequence PKKKRKV (SEQ ID NO:204).

In certain embodiments, the NLS sequence of the Cas12a protein is positioned at or near the C-terminus of the Cas12a protein sequence. For example, but not by way of limitation, the Cas12a protein can be selected from the following: His-AsCas12a-nNLS (SEQ ID NO:200); His-AsCas12a-sNLS (SEQ ID NO:205) and His-AsCas12a-sNLS-sNLS (SEQ ID NO:201), where “His” refers to a six-histidine purification sequence, “AsCas12a” refers to the Acidaminococcus sp. Cas12a protein sequence, “nNLS” refers to the nucleoplasmin NLS, and “sNLS” refers to the SV40 NLS. Additional permutations of the identity and C-terminal positions of NLS sequences, e.g., appending two or more nNLS sequences or combinations of nNLS and sNLS sequences (or other NLS sequences), as well as sequences with and without purification sequences, e.g., six-histidine sequences, are within the scope of the instantly disclosed subject matter.

In certain embodiments, the NLS sequence of the Cas12a protein may be positioned at or near the N-terminus of the Cas12a protein sequence. For example, but not by way of limitation, the Cas12a protein may be selected from the following: His-sNLS-AsCas12a (SEQ ID NO:206), His-sNLS-sNLS-AsCas12a (SEQ ID NO:207), and sNLS-sNLS-AsCas12a (SEQ ID NO:208). Additional permutations of the identity and N-terminal positions of NLS sequences, e.g., appending two or more nNLS sequences or combinations of nNLS and sNLS sequences (or other NLS sequences), as well as sequences with and without purification sequences, e.g., six-histidine sequences, are within the scope of the instantly disclosed subject matter.

In certain embodiments, the Cas12a protein may comprise NLS sequences positioned at or near both the N-terminus and C-terminus of the Cas12a protein sequence. For example, but not by way of limitation, the Cas12a protein may be selected from the following: His-sNLS-AsCas12a-sNLS (SEQ ID NO:209) and His-sNLS-sNLS-AsCas12a-sNLS-sNLS (SEQ ID NO:210). Additional permutations of the identity and N-terminal/C-terminal positions of NLS sequences, e.g., appending two or more nNLS sequences or combinations of nNLS and sNLS sequences (or other NLS sequences) to either the N-terminal/C-terminal positions, as well as sequences with and without purification sequences, e.g., six-histidine sequences, are within the scope of the instantly disclosed subject matter.

In certain embodiments, the Cas12a protein may comprise an alteration (e.g., a deletion or substitution) at one or more cysteine residues of the Cas12a protein sequence. For example, but not by way of limitation, Cas12a protein may comprise an alteration at a position selected from the group consisting of: C65, C205, C334, C379, C608, C674, C1025, and C1248. In certain embodiments, the Cas12a protein may comprise a substitution of one or more cysteine residues for a serine or alanine. In certain embodiments, the Cas12a protein may comprise an alteration selected from the group consisting of: C65S, C205S, C334S, C379S, C608S, C674S, C1025S, and C1248S. In certain embodiments, the Cas12a protein may comprise an alteration selected from the group consisting of: C65A, C205A, C334A, C379A, C608A, C674A, C1025A, and C1248A. In certain embodiments, the Cas12a protein may comprise alterations at positions C334 and C674 or C334, C379, and C674. In certain embodiments, the Cas12a protein may comprise the following alterations: C334S and C674S, or C334S, C379S, and C674S. In certain embodiments, the Cas12a protein may comprise the following alterations: C334A and C674A, or C334A, C379A, and C674A. In certain embodiments, the Cas12a protein may comprise both one or more cysteine residue alteration as well as the introduction of one or more NLS sequences, e.g., His-AsCas12a-nNLS Cys-less (SEQ ID NO:211) or His-AsCas12a-nNLS Cys-low (SEQ ID NO:212). In various embodiments, the Cas12a protein comprising a deletion or substitution in one or more cysteine residues exhibits reduced aggregation.

In certain embodiments, other Cas12a proteins known in the art may be used with the methods and systems described herein. For example, in certain embodiments, the Cas12a may be Cas12a containing the mutation S542R/K548V/N552R (“Cas12a RVR”). Cas12a RVR has been shown to cleave target sites with a TATV PAM. In certain embodiments, the Cas12a may be Cas12a containing the mutation S542R/K607R (“Cas12a RR”). Cas12a RR has been shown to cleave target sites with a TYCV/CCCC PAM.

In some embodiments, a Cas12a is used herein, wherein the Cas12a comprises mutations at one or more residues of AsCas12a (Acidaminococcus sp. BV3L6) selected from the group consisting of 11, 12, 13, 14, 15, 16, 17, 34, 36, 39, 40, 43, 46, 47, 50, 54, 57, 58, 111, 126, 127, 128, 129, 130, 131, 132, 133, 134, 135, 136, 157, 158, 159, 160, 161, 162, 163, 164, 165, 166, 167, 168, 169, 170, 171, 172, 173, 174, 175, 176, 177, 178, 532, 533, 534, 535, 536, 537, 538, 539, 540, 541, 542, 543, 544, 545, 546, 547, 548, 549, 550, 551, 552, 553, 554, 555, 556, 565, 566, 567, 568, 569, 570, 571, 572, 573, 574, 575, 592, 593, 594, 595, 596, 597, 598, 599, 600, 601, 602, 603, 604, 605, 606, 607, 608, 609, 610, 611, 612, 613, 614, 615, 616, 617, 618, 619, 620, 626, 627, 628, 629, 630, 631, 632, 633, 634, 635, 636, 637, 638, 642, 643, 644, 645, 646, 647, 648, 649, 651, 652, 653, 654, 655, 656, 676, 679, 680, 682, 683, 684, 685, 686, 687, 688, 689, 690, 691, 692, 693, 707, 711, 714, 715, 716, 717, 718, 719, 720, 721, 722, 739, 765, 768, 769, 773, 777, 778, 779, 780, 781, 782, 783, 784, 785, 786, 870, 871, 872, 873, 874, 875, 876, 877, 878, 879, 880, 881, 882, 883, 884, or 1048 or the corresponding position of an AsCas12a orthologue, homologue, or variant. In certain embodiments, a Cas12a comprises a mutation corresponding to a M537R substitution, an H800A substitution, and/or an F870L substitution (numbering according to AsCas12a wild-type sequence).

In certain embodiments, a Cas12a as used herein may include any of the Cas12a proteins described in International Publication Number WO 2017/184768 A1 by Zhang et al. (“'768 Publication”), which is incorporated by reference herein.

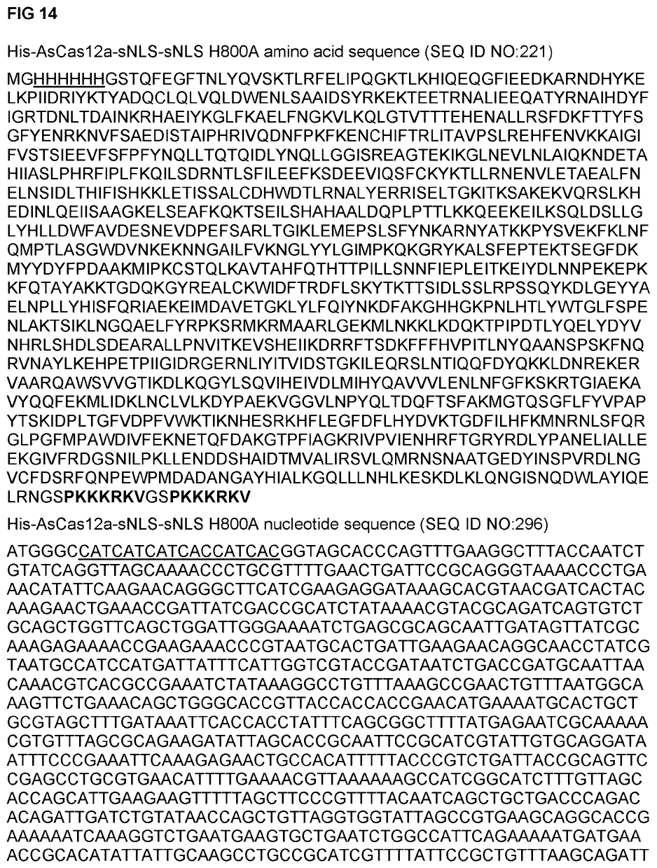

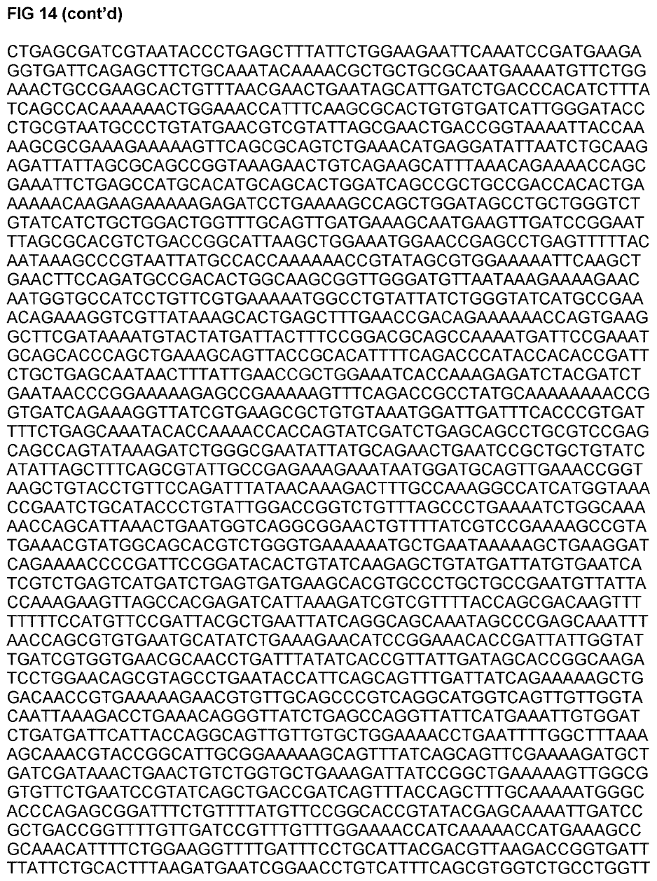

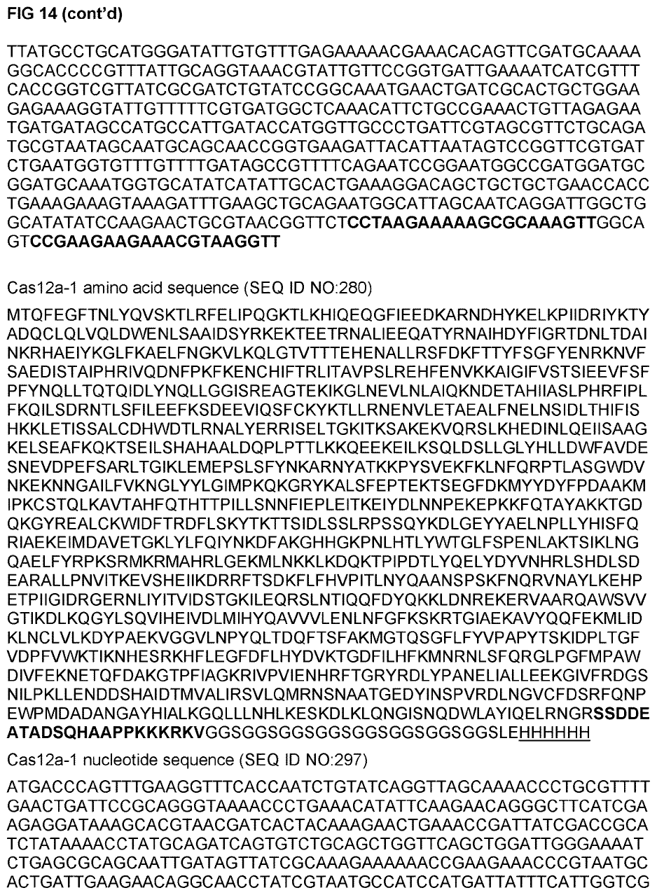

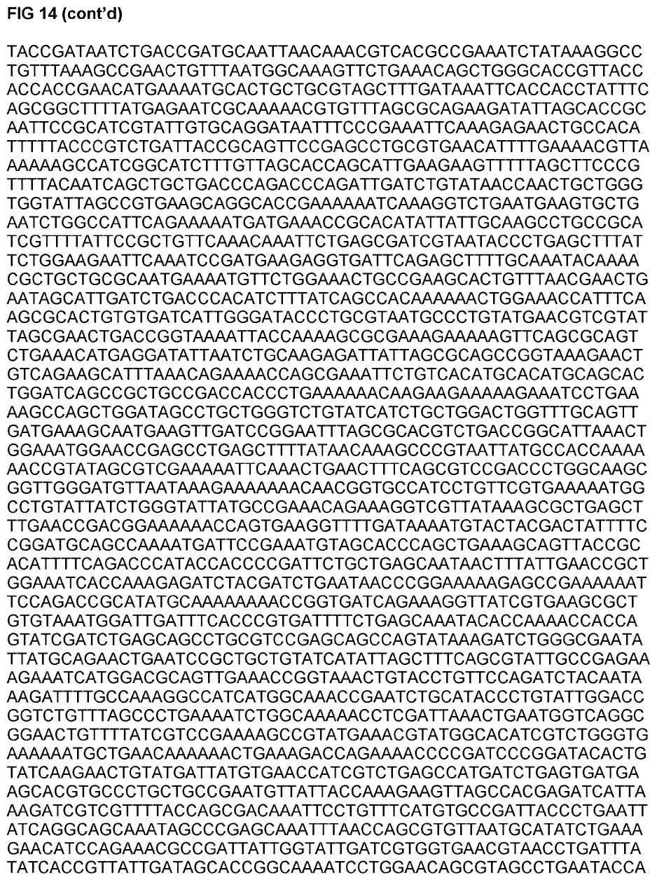

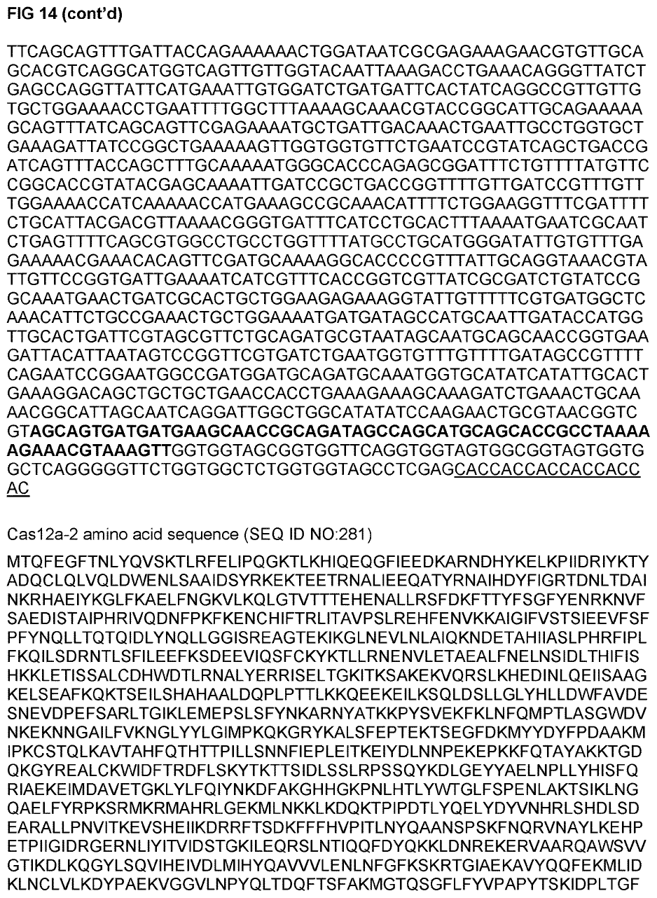

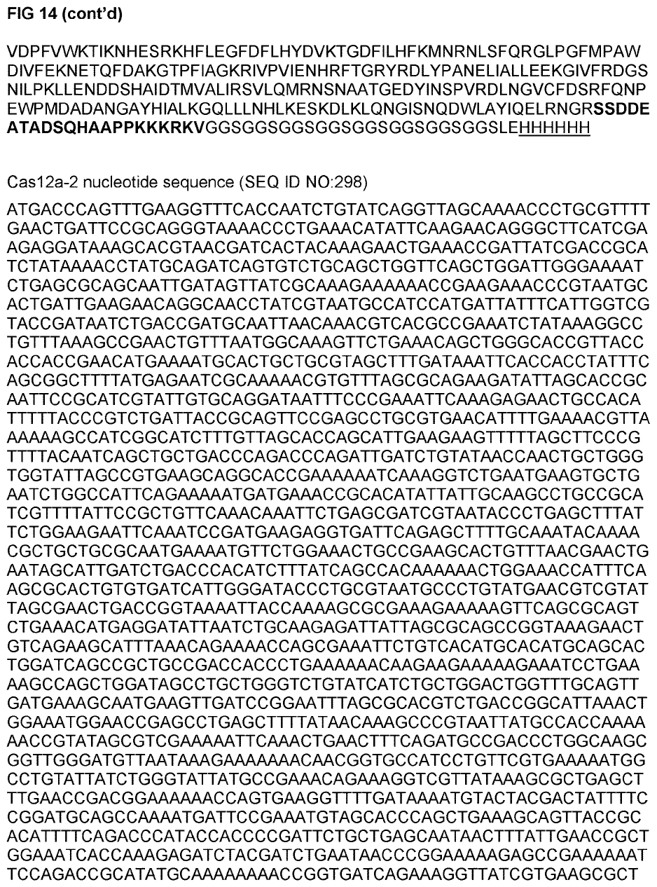

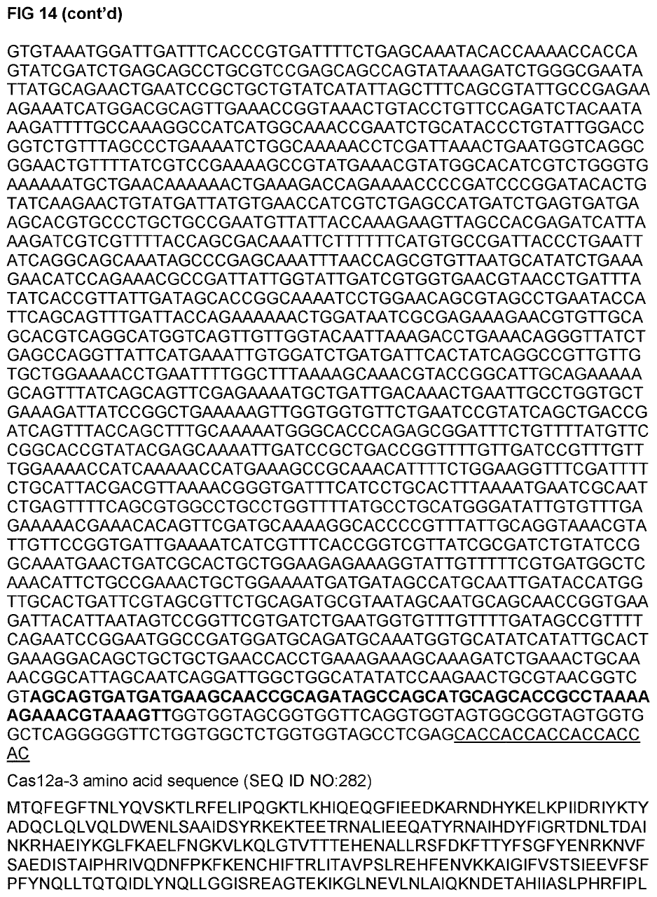

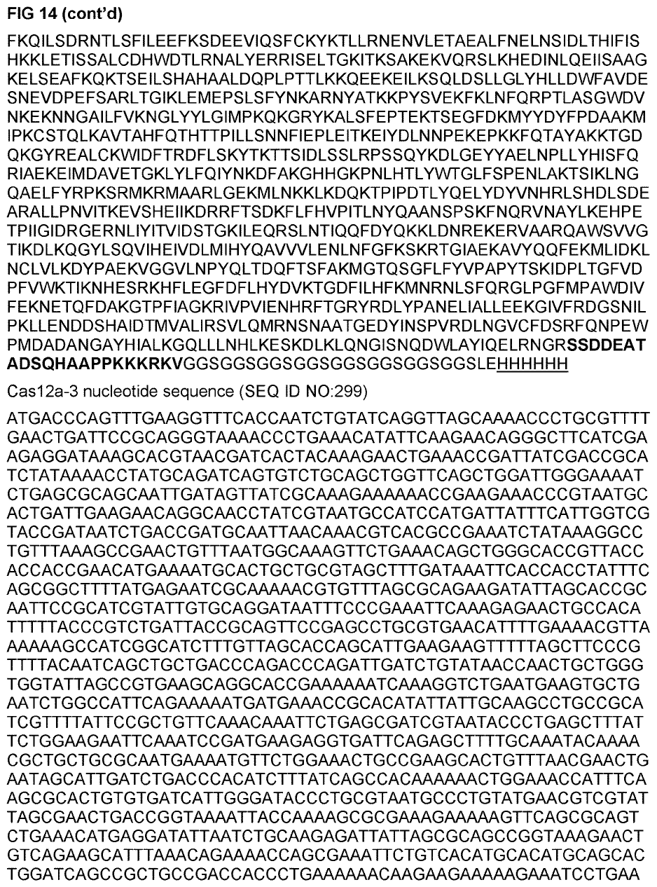

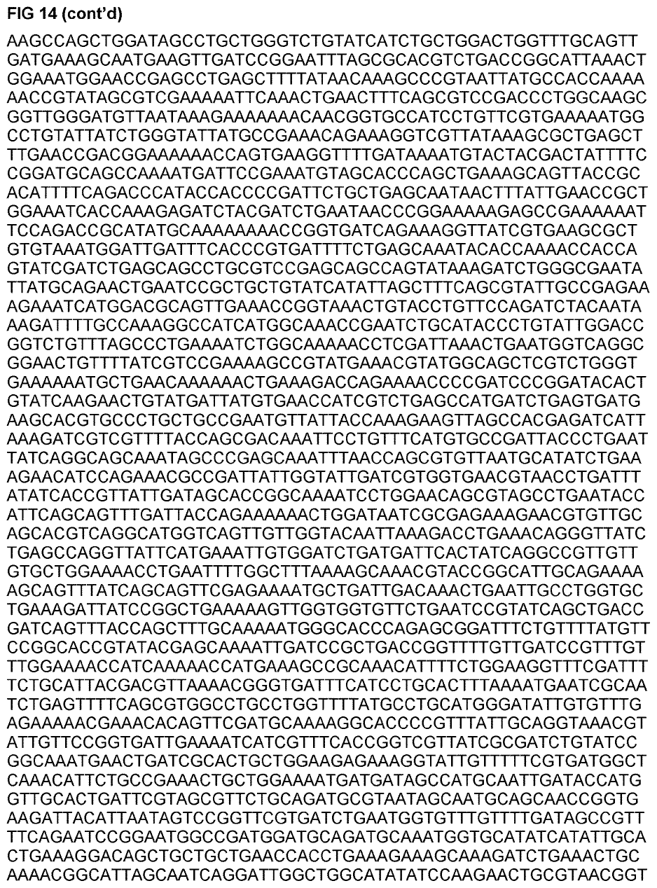

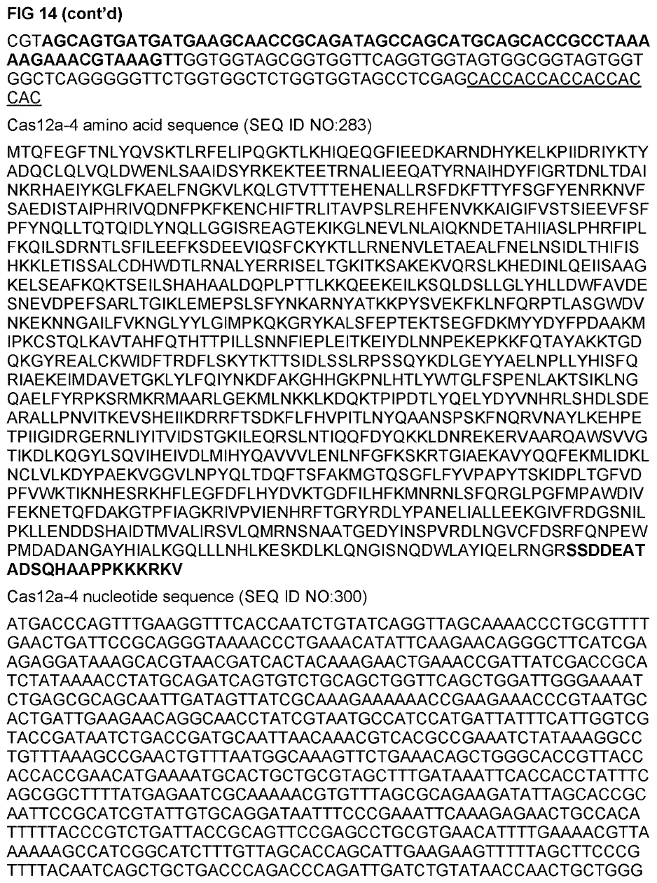

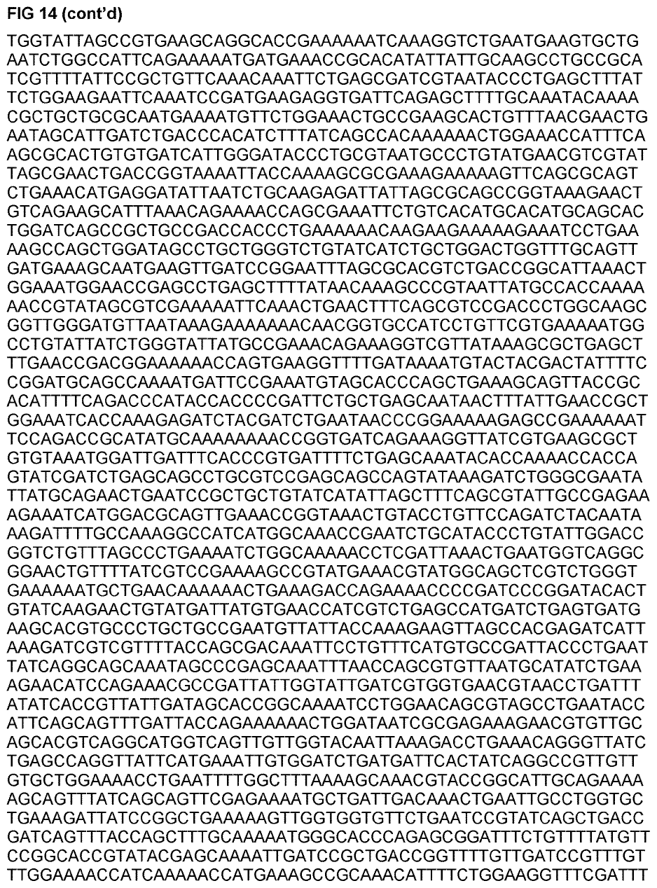

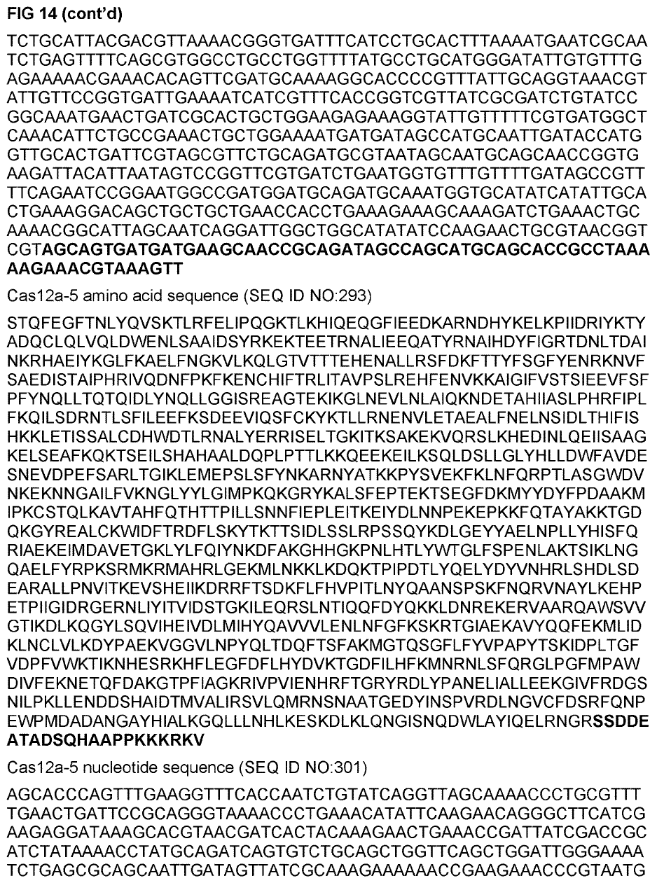

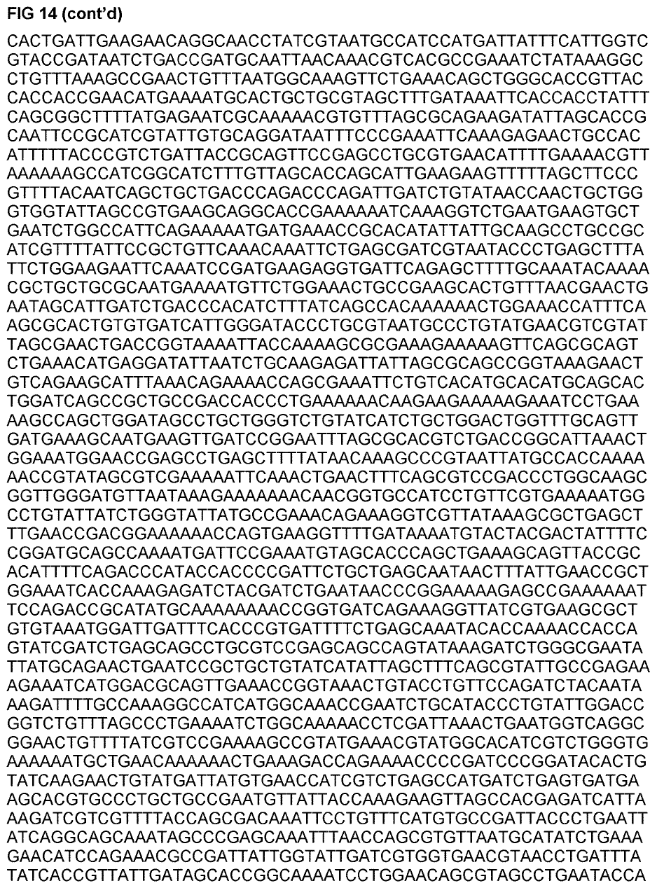

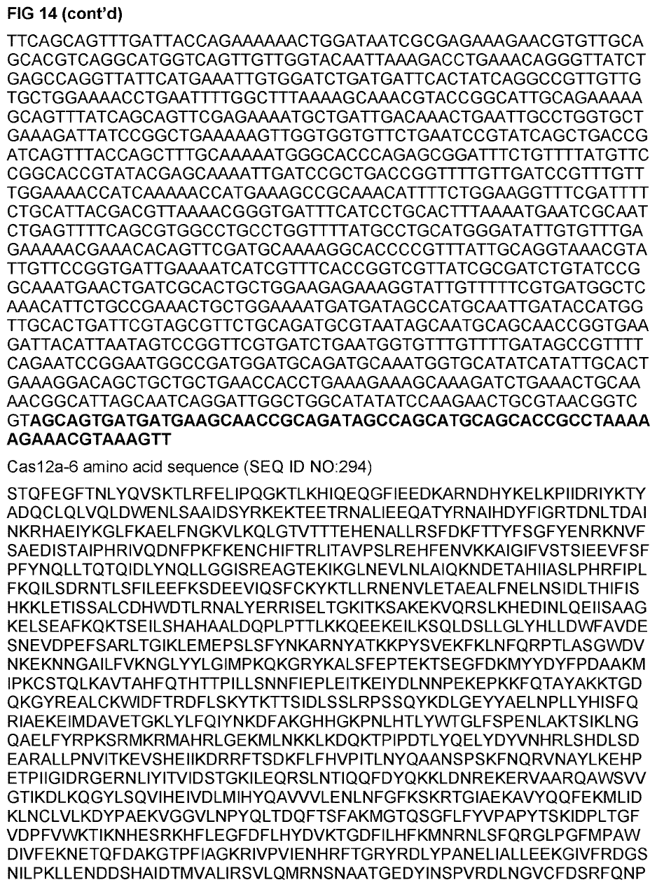

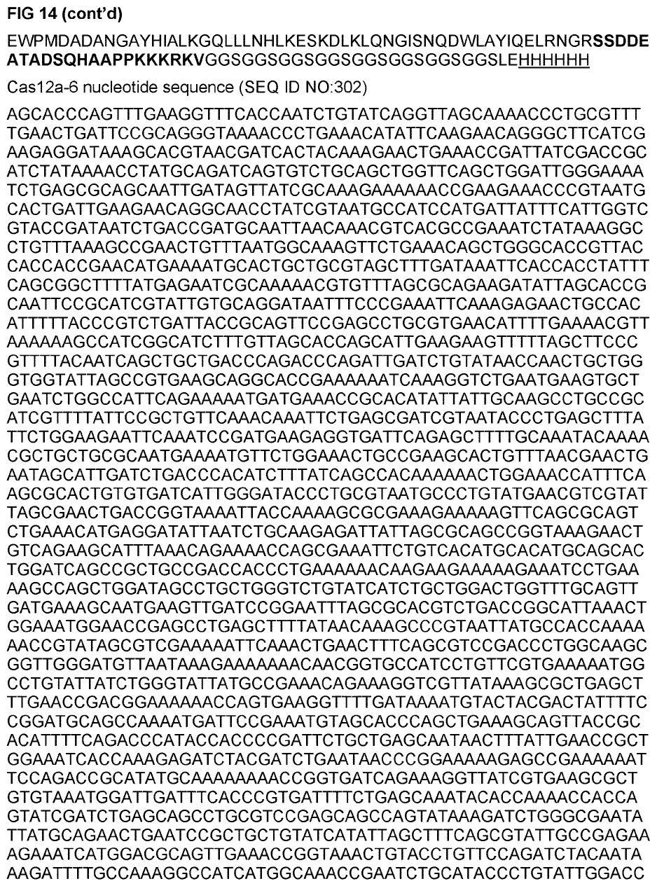

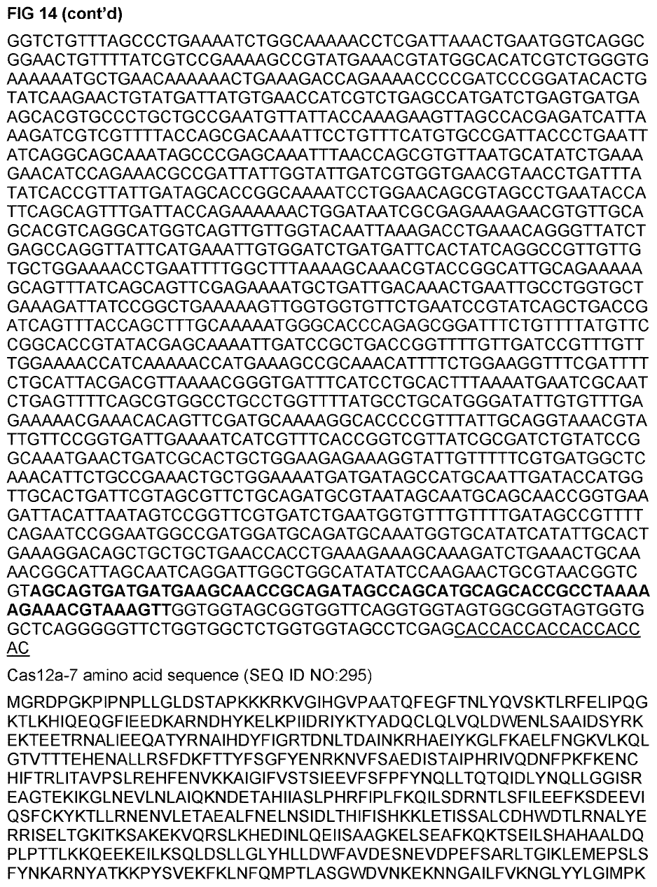

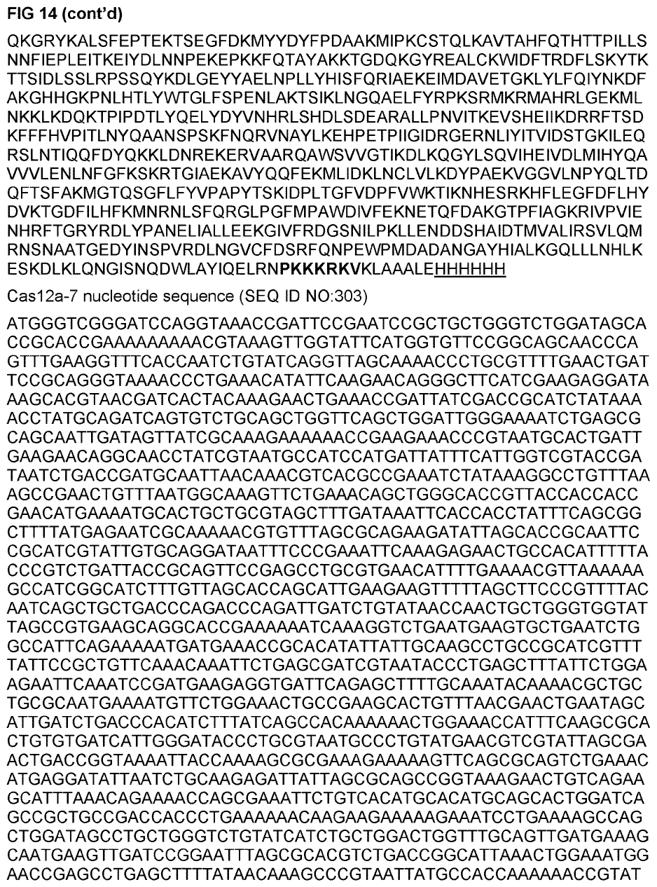

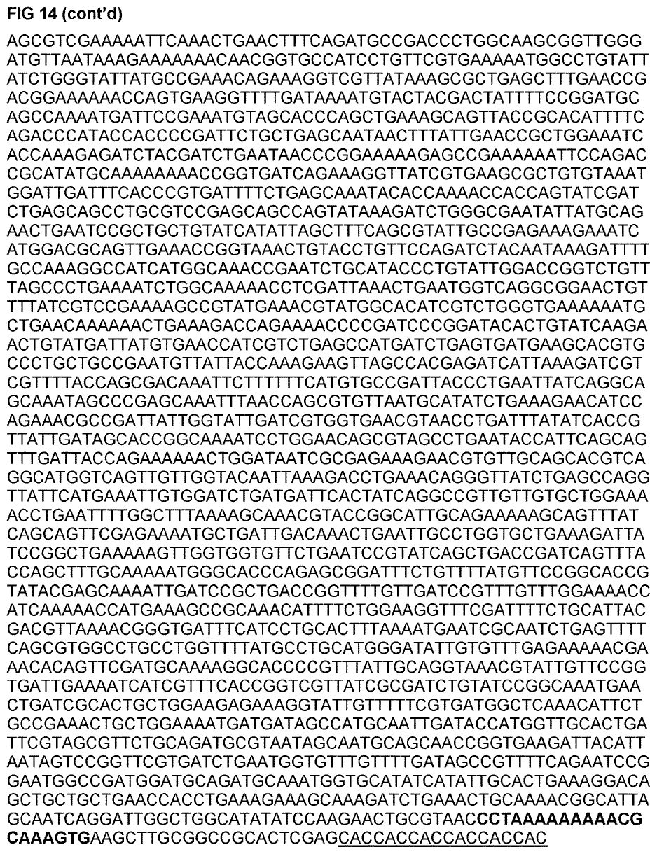

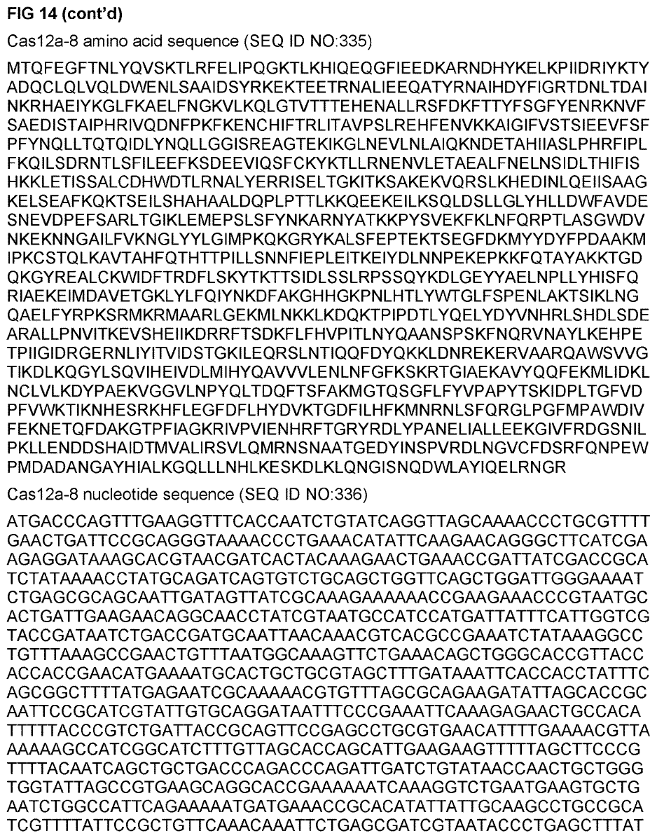

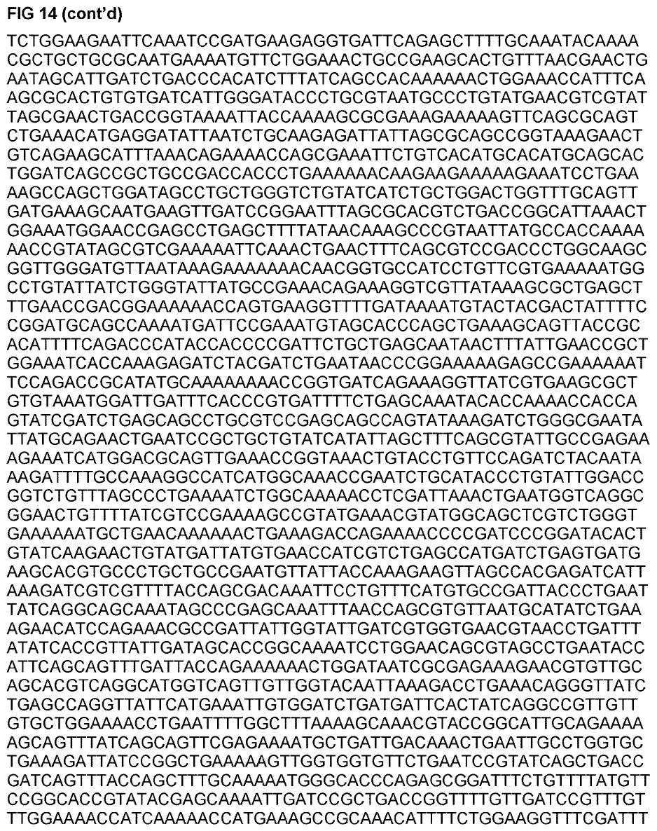

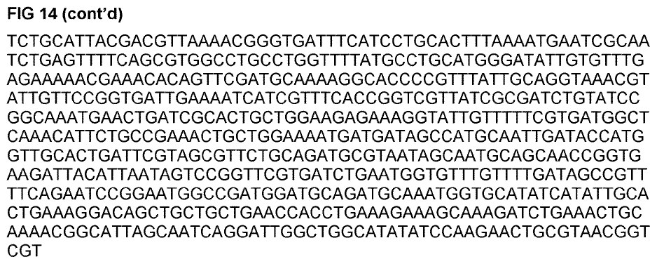

In certain embodiments, a Cas12a protein (also referred to herein as a Cas12a molecule) used herein may comprise any of the sequences set forth in SEQ ID NOs:200, 201, 205-215, 221, 222-226, 280-283, 293-295, 335 (Cas12a polypeptide sequences). In certain embodiments, a Cas12a protein may be encoded by a sequence comprising any of SEQ ID NOs:216-218, 296-303, 336 (Cas12a polynucleotide sequences). Table 17 sets forth exemplary Cas12a amino acid and nucleotide sequences. These sequences are set forth in FIG. 14, which details the positions of six-histidine sequences (underlined letters) and NLS sequences (bolded letters). Additional permutations of the identity and N-terminal/C-terminal positions of NLS sequences, e.g., appending two or more nNLS sequences or combinations of nNLS and sNLS sequences (or other NLS sequences) to either the N-terminal/C-terminal positions, as well as sequences with and without purification sequences, e.g., six-histidine sequences, are within the scope of the instantly disclosed subject matter.

In certain embodiments, any of the Cas12a proteins disclosed herein may be complexed with one or more gRNA comprising the targeting domain set forth in SEQ ID NOs: 202 and/or 327 to alter a CCAAT box target region. In certain embodiments, any of the Cas12a proteins disclosed herein may be complexed with one or more gRNA comprising a sequence set forth in Table 20. In certain embodiments, the Cas12a protein may be His-AsCas12a-nNLS (comprising SEQ ID NO:200) or His-AsCas12a-sNLS-sNLS (comprising SEQ ID NO:201). In certain embodiments, a Cas12a protein used herein may comprise any of the sequences set forth in SEQ ID NOs:200, 201, 205-215, 221, 222-226, 280-283, 293-295, 335 (Cas12a polypeptide sequences). In certain embodiments, a Cas12a protein used herein may be encoded by any of the sequences set forth in SEQ ID NOs:216-218, 296-303, 336 (Cas12a polynucleotide sequences). In certain embodiments, the Cas12a protein may comprise the sequence set forth in SEQ ID NO:283.

In certain embodiments, the Cas12a protein may include a Cas12a described in Kleinstiver 2019. For example, without limitation, in certain embodiments, the Cas12a protein may be enAsCas12a. In certain embodiments, the Cas12a protein may cleave target sites with a TTTV PAM. In certain embodiments, the Cas12a protein may cleave target sites with a NWYN PAM.

In certain embodiments, an RNA-guided nuclease has at least 80%, at least 85%, at least 86%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sequence identity relative to a wild-type RNA-guided nuclease and/or an RNA-guided nuclease disclosed herein (e.g., an RNA-guided nuclease comprising an amino acid sequence selected from the group consisting of SEQ ID NOs:200, 201, 205-215, 221, 222-226, 280-283, 293-295, 335). In some embodiments, an RNA-guided nuclease has 1, 2, 3, 4, 5, 6, 7, 8, 9, 10, 11, 12, 13, 14, 15, 16, 17, 18, 19, or 20 mutations relative to a wild-type RNA-guided nuclease and/or an RNA-guided nuclease disclosed herein (e.g., an RNA-guided nuclease comprising an amino acid sequence selected from the group consisting of SEQ ID NOs:200, 201, 205-215, 221, 222-226, 280-283, 293-295, 335). In some embodiments, an RNA-guided nuclease has less than 2, 3, 4, 5, 6, 7, 8, 9, 10, 11, 12, 13, 14, 15, 16, 17, 18, 19, or 20 mutations relative to a wild-type RNA-guided nuclease and/or an RNA-guided nuclease disclosed herein (e.g., an RNA-guided nuclease comprising an amino acid sequence selected from the group consisting of SEQ ID NOs:200, 201, 205-215, 221, 222-226, 280-283, 293-295, 335).

### Modifications of RNA-Guided Nucleases

The RNA-guided nucleases described above have activities and properties that can be useful in a variety of applications, but the skilled artisan will appreciate that RNA-guided nucleases can also be modified in certain instances, to alter cleavage activity, PAM specificity, or other structural or functional features.

Turning first to modifications that alter cleavage activity, mutations that reduce or eliminate the activity of domains within the NUC lobe have been described above. Exemplary mutations that may be made in the RuvC domains, in the Cas9 HNH domain, or in the Cas12a Nuc domain are described in Ran & Hsu 2013 and Yamano 2016, as well as in Cotta-Ramusino. In general, mutations that reduce or eliminate activity in one of the two nuclease domains result in RNA-guided nucleases with nickase activity, but it should be noted that the type of nickase activity varies depending on which domain is inactivated. As one example, inactivation of a RuvC domain of a Cas9 will result in a nickase that cleaves the complementary or top strand as shown below (where C denotes the site of cleavage).

On the other hand, inactivation of a Cas9 HNH domain results in a nickase that cleaves the bottom or non-complementary strand.

Modifications of PAM specificity relative to naturally occurring Cas9 reference molecules has been described by Kleinstiver et al. for both S. pyogenes (Kleinstiver 2015a) and S. aureus (Kleinstiver 2015b). Kleinstiver et al. have also described modifications that improve the targeting fidelity of Cas9 (Kleinstiver 2016). Kleinstiver et al. have also described modifications of Cas12a that provide increased activity and improved targeting ranges (Kleinstiver 2019). Each of these references is incorporated by reference herein.

RNA-guided nucleases have been split into two or more parts, as described by Zetsche 2015 and Fine 2015 (both incorporated by reference herein).

RNA-guided nucleases can be, in certain embodiments, size-optimized or truncated, for instance via one or more deletions that reduce the size of the nuclease while still retaining gRNA association, target and PAM recognition, and cleavage activities. In certain embodiments, RNA guided nucleases are bound, covalently or non-covalently, to another polypeptide, nucleotide, or other structure, optionally by means of a linker. Exemplary bound nucleases and linkers are described by Guilinger 2014, incorporated by reference herein for all purposes.

RNA-guided nucleases also optionally include a tag, such as, but not limited to, a nuclear localization signal to facilitate movement of RNA-guided nuclease protein into the nucleus. In certain embodiments, the RNA-guided nuclease can incorporate C- and/or N-terminal nuclear localization signals. Nuclear localization sequences are known in the art and are described in Maeder and elsewhere.

The foregoing list of modifications is intended to be exemplary in nature, and the skilled artisan will appreciate, in view of the instant disclosure, that other modifications may be possible or desirable in certain applications. For brevity, therefore, exemplary systems, methods and compositions of the present disclosure are presented with reference to particular RNA-guided nucleases, but it should be understood that the RNA-guided nucleases used may be modified in ways that do not alter their operating principles. Such modifications are within the scope of the present disclosure.

### Nucleic Acids Encoding RNA-Guided Nucleases

Nucleic acids encoding RNA-guided nucleases, e.g., Cas9, Cas12a or functional fragments thereof, are provided herein. Exemplary nucleic acids encoding RNA-guided nucleases have been described previously (see, e.g., Cong 2013; Wang 2013; Mali 2013; Jinek 2012).

In some cases, a nucleic acid encoding an RNA-guided nuclease can be a synthetic nucleic acid sequence. For example, the synthetic nucleic acid molecule can be chemically modified. In certain embodiments, an mRNA encoding an RNA-guided nuclease will have one or more (e.g., all) of the following properties: it can be capped; polyadenylated; and substituted with 5-methylcytidine and/or pseudouridine.

Synthetic nucleic acid sequences can also be codon optimized, e.g., at least one non-common codon or less-common codon has been replaced by a common codon. For example, the synthetic nucleic acid can direct the synthesis of an optimized messenger mRNA, e.g., optimized for expression in a mammalian expression system, e.g., described herein. Examples of codon optimized Cas9 coding sequences are presented in Cotta-Ramusino.

In addition, or alternatively, a nucleic acid encoding an RNA-guided nuclease may comprise a nuclear localization sequence (NLS). Nuclear localization sequences are known in the art.

### Methods

Provided herein in certain aspects are methods of treating a β-hemoglobinopathy. In certain embodiments, the β-hemoglobinopathy may be sickle cell disease (SCD) or β-Thal. In certain embodiments, the β-Thal may be transfusion-dependent beta-thalassemia (TDT). Provided herein in certain aspects are methods of inducing expression of fetal hemoglobin (HbF) in a population of cells, e.g., CD34+ hematopoietic stem and progenitor cells (HSPCs) and/or red blood cells, in a subject. In certain embodiments, the subject may be suffering from a β-hemoglobinopathy. In certain embodiments, the β-hemoglobinopathy may be sickle cell disease (SCD) or β-Thal (e.g., TDT). In certain embodiments, the method (e.g., method of treating a β-hemoglobinopathy, method of inducing expression of HbF in a population of cells) may comprise administering to the subject a population of modified cells comprising a plurality of modified CD34+ or hematopoietic stem cells comprising an indel in an HBG promoter, thereby inducing expression of HbF in the population of cells (e.g., HSPCs or RBCs). In certain embodiments, the HbF as a percentage of total hemoglobin (% HbF) in the subject (e.g., in the blood or peripheral blood of the subject) may be about 5%, 10%, 15%, 20%, 25%, 30%, 35%, 40%, 45%, 50%, 55%, 60%, 65%, 70%, 75%, or 80% HbF. In certain embodiments, the % HbF in the subject (e.g., in the blood or peripheral blood of the subject) may be from about 10% to about 30%, from about 20% to about 40%, from about 30% to about 50%, from about 40% to about 60%, from about 50% to about 70%, from about 60% to about 80%, from about 10% to about 20%, from about 20% to about 30%, from about 30% to about 40%, from about 40% to about 50%, from about 50% to about 60%, from about 60% to about 70%, from about 70% to about 80% HbF, or a range defined by any of the two preceding values. In certain embodiments, a concentration of total hemoglobin in the subject (e.g., in the blood or peripheral blood of the subject) may be about 10 g/dL, 11 g/dL, 12 g/dL, 13 g/dL, 14 g/dL, 15 g/dL, 16 g/dL, 17 g/dL, 18 g/dL, 19 g/dL, or 20 g/dL. In certain embodiments, a concentration of total hemoglobin in the subject (e.g., in the blood or peripheral blood of the subject) may be from about 10.0 to about 20.0 g/dL, from about 13.6 to about 18.0 g/dL (for males), from about 12.0 to about 16.0 g/dL (for females), or a range defined by any of the two preceding values. In certain embodiments, a percentage of F-cells among circulating RBCs in the subject may be about 5%, 10%, 15%, 20%, 25%, 30%, 35%, 40%, 45%, 50%, 55%, 60%, 65%, 70%, 75%, 80%, 85%, 90%, 95%, 100% F-cells. In certain embodiments, a percentage of F-cells among circulating RBCs in the subject may be from about 50% to about 100%, from about 60% to about 100%, from about 70% to about 100%, from about 80% to about 100%, from about 50% to about 60%, from about 60% to about 70%, from about 70% to about 80%, from about 80% to about 90%, from about 90% to about 100%, or a range defined by any of the two preceding values. In certain embodiments, a Hemoglobin F concentration in the subject may be about 1 g/dL, 2 g/dL, 3 g/dL, 4 g/dL, 5 g/dL, 6 g/dL, 7 g/dL, 8 g/dL, 9 g/dL, 10 g/dL, 11 g/dL, 12 g/dL, 13 g/dL, 14 g/dL, 15 g/dL, 16 g/dL, 17 g/dL, 18 g/dL, 19 g/dL, or 20 g/dL. In certain embodiments, a Hemoglobin F concentration in the subject may be from about 1 g/dL to about 20 g/dL, or a range defined by any of the two preceding values. In certain embodiments, a mean corpuscular HbF (pg/RBC) in the subject may be about 1.0 pg/RBC, 2.0 pg/RBC, 3.0 pg/RBC, 4.0 pg/RBC, 5.0 pg/RBC, 6.0 pg/RBC, 7.0 pg/RBC, 8.0 pg/RBC, 9.0 pg/RBC, 10.0 pg/RBC, 11.0 pg/RBC, 12.0 pg/RBC, 13.0 pg/RBC, 14.0 pg/RBC 15.0 pg/RBC, 16.0 pg/RBC, 17.0 pg/RBC, 18.0 pg/RBC, 19.0 pg/RBC, 20.0 pg/RBC, 25.0 pg/RBC, or 30.0 pg/RBC. In certain embodiments, a mean corpuscular HbF (pg/RBC) in the subject may be from about 1.0 pg/RBC to about 5.00 pg/RBC, from about 5.0 pg/RBC to about 10.00 pg/RBC, from about 10.0 pg/RBC to about 15.00 pg/RBC, from about 15.0 pg/RBC to about 20.00 pg/RBC, from about 20.0 pg/RBC to about 25.00 pg/RBC, from about 25.0 pg/RBC to about 30.00 pg/RBC, or a range defined by any of the two preceding values. In certain embodiments, a mean corpuscular HbF (pg/RBC) in the subject may be ≥10.0 pg/RBC. In certain embodiments, a mean proportion of HbF as a percentage of total hemoglobin (Hb) in the subject is about 5%, 10%, 15%, 20%, 25%, 30%, 35%, 40%, 45%, 50%, 55%, 60%, 65%, 70%, 75%, 80%, 85%, 90%, 95%. In certain embodiments, a mean proportion of HbS as a percentage of total hemoglobin (Hb) in the subject is about 5%, 10%, 15%, 20%, 25%, 30%, 35%, 40%, 45%, 50%, 55%, 60%, 65%, 70%, 75%, 80%, 85%, 90%, 95%. In certain embodiments, a mean proportion of HbA as a percentage of total hemoglobin (Hb) in the subject is about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%. In certain embodiments, a mean proportion of HbA2 as a percentage of total hemoglobin (Hb) in the subject is about 1%, 2%, 3%, 4%, 5%. In certain embodiments, a lactate dehydrogenase (U/L) in the subject is about 110 U/L, 120 U/L, 130 U/L, 140 U/L, 150 U/L, 160 U/L, 170 U/L, 180 U/L, 190 U/L, 200 U/L, 210 U/L, 220 U/L, or 230 U/L. In certain embodiments, a lactate dehydrogenase (U/L) in the subject is from about 110 U/L to about 230 U/L, or a range defined by any of the two preceding values. In certain embodiments, an indirect bilirubin (μmol/L) in the subject is about 0.0 μmol/L, 1 μmol/L, 2 μmol/L, 3 μmol/L, 4 μmol/L, 5 μmol/L, 6 μmol/L, 7 μmol/L, 8 μmol/L, 9 μmol/L, 10 μmol/L, 11 μmol/L, 12 μmol/L, 13 μmol/L, 14 μmol/L, 15 μmol/L, 16 μmol/L, or 17 μmol/L. In certain embodiments, an indirect bilirubin (μmol/L) in the subject is from about 0.0 μmol/L to about 16.6 μmol/L, or a range defined by the two preceding values. In certain embodiments, a haptoglobin (g/L) in the subject is about 0.3 g/L, 0.4 g/L, 0.5 g/L, 0.6 g/L, 0.7 g/L, 0.8 g/L, 0.9 g/L, 1.0 g/L, 1.1 g/L, 1.2 g/L, 1.3 g/L, 1.4 g/L, 1.5 g/L, 1.6 g/L, 1.7 g/L, 1.8 g/L, 1.9 g/L, or 2.0 g/L. In certain embodiments, a haptoglobin (g/L) in the subject is from about 0.3 g/L to about 2.0 g/L or a range defined by the two preceding values. In certain embodiments, a reticulocyte count (%) in the subject is about 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, or 2.3%. In certain embodiments, a reticulocyte count (%) in the subject is from about 0.3% to about 2.3% or a range defined by the two preceding values. In certain embodiments, normalization of total hemoglobin in the subject occurs by at least 1 month, 1.5 months, 2 months, 3 months, 4 months, 5 months after administering the population of modified cells to the subject. In certain embodiments, the subject may undergo myeloablative conditioning with busulfan prior to administering the population of modified cells. In certain embodiments, administering the population of modified cells may comprise a single infusion of the modified population of cells. In certain embodiments, the population of modified cells may be about ≥1×10⁶ cells/kg, ≥2×10⁶ cells/kg, ≥3×10⁶ cells/kg, ≥4×10⁶ cells/kg, ≥5×10⁶ cells/kg, ≥6×10⁶ cells/kg, ≥7×10⁶ cells/kg, ≥8×10⁶ cells/kg, ≥9×10⁶ cells/kg, ≥10×10⁶ cells/kg, ≥11×10⁶ cells/kg, ≥12×10⁶ cells/kg, ≥13×10⁶ cells/kg, ≥14×10⁶ cells/kg, ≥15×10⁶ cells/kg, ≥16×10⁶ cells/kg, ≥17×10⁶ cells/kg, ≥18×10⁶ cells/kg, ≥19×10⁶ cells/kg, ≥20×10⁶ cells/kg, ≥21×10⁶ cells/kg, ≥22×10⁶ cells/kg, ≥23×10⁶ cells/kg, ≥24×10⁶ cells/kg, ≥25×10⁶ cells/kg, 1×10⁶ cells/kg to 25×10⁶ cells/kg, 1×10⁶ cells/kg to 5×10⁶ cells/kg, 3×10⁶ cells/kg to 20×10⁶ cells/kg, 3×10⁶ cells/kg to 15×10⁶ cells/kg, 3×10⁶ cells/kg to 10×10⁶ cells/kg, 3×10⁶ cells/kg to 5×10⁶ cells/kg, 5×10⁶ cells/kg to 10×10⁶ cells/kg, 10×10⁶ cells/kg to 15×10⁶ cells/kg, 15×10⁶ cells/kg to 20×10⁶ cells/kg, 20×10⁶ cells/kg to 25×10⁶ cells/kg. In certain embodiments, the % HbF may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the concentration of total hemoglobin may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the Hemoglobin F concentration may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the percentage of F-cells may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean corpuscular HbF (pg/RBC) may be determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean proportion of HbF as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean proportion of HbS as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean proportion of HbA as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the mean proportion of HbA2 as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the lactate dehydrogenase (U/L) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the indirect bilirubin (μmol/L) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the haptoglobin (g/L) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the reticulocyte count (%) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience no vaso-occlusive events (VOEs) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience no severe VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience no non-severe VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 2, 3, 4, 5, 6, 7, 8, 9, 10, 11, 12, 13, 14, 15, 16, 17, 18, 19, or 20 VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 2, 3, 4, 5, 6, 7, 8, 9, or 10 severe VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 1, 2, 3, 4, 5, 10, 15, 20, 25, 30, 35, 40, 45, 50, 55, 60, 65 treatment emergent adverse events (TEAEs) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 1, 2, 3, 4, 5, 6, 7, 8, 9, or 10 serious TEAEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may be red blood cell (RBC) transfusion free 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

In certain embodiments, an RNP complex comprising a guide RNA (gRNA) and a Cas12a protein may be delivered to a population of unmodified cells comprising a plurality of unmodified CD34+ or hematopoietic stem cells from the subject to generate the population of modified cells described herein. In certain embodiments, the gRNA may comprise a sequence set forth in Table 20. In certain embodiments, the gRNA may comprise a targeting domain comprising SEQ ID NO:320. In certain embodiments, the gRNA may comprise a targeting domain sequence set forth in SEQ ID NO:202. In certain embodiments, the gRNA may comprise a 5′ end and a 3′ end, a DNA extension at the 5′ end. In certain embodiments, the gRNA may comprise a 2′-O-methyl, phosphorothioate modification, or both at the 3′ end. In certain embodiments, the gRNA may comprise a DNA extension sequence set forth in Table 19. In certain embodiments, the DNA extension may comprise a sequence selected from the group consisting of SEQ ID NOs:304-319. In certain embodiments, the gRNA may comprise a DNA extension sequence set forth in SEQ ID NO:306. In certain embodiments, the gRNA may comprise SEQ ID NO:237. In certain embodiments, the Cas12a protein may comprise a sequence selected from the group consisting of SEQ ID NO:200, 201, 205-215, 221, 222-226, 280-283, 293-295, and 335. In certain embodiments, the Cas12a protein may comprise SEQ ID NO:283. In certain embodiments, the Cas12a protein may comprise SEQ ID NO:335. In certain embodiments, the Cas12a protein may be encoded by a sequence comprising a sequence selected from the group consisting of SEQ ID NOs:216-218, 296-303, and 336. In certain embodiments, the Cas12a protein may be encoded by a sequence comprising SEQ ID NO:300. In certain embodiments, the Cas12a protein may be encoded by a sequence comprising SEQ ID NO:336. In certain embodiments, the indel in the HBG gene promoter may be in a CCAAT box target region. In certain embodiments, the subject has a single homozygous mutation in the HBB gene, c.17A>T (HbS mutation). In certain embodiments, the subject may be suffering from severe sickle cell disease. In certain embodiments, the subject may be suffering from transfusion-dependent beta-thalassemia (TDT).

Provided herein in certain aspects are methods of producing a modified population of peripheral blood nucleated cells in a subject, the method comprising: administering to the subject a population of modified cells comprising a plurality of modified CD34+ or hematopoietic stem cells comprising an indel in an HBG promoter, thereby producing the modified population of peripheral blood nucleated cells, wherein the modified population of peripheral blood nucleated cells as a percentage of the total population of peripheral blood nucleated cells is at least 50%, 55%, 60%, 65%, 70%, 75%, 80%, 85%, 90% or 95%. In certain embodiments, the subject may undergo myeloablative conditioning with busulfan prior to administering the population of modified cells.

Provided herein in certain aspects are methods of generating a population of F-cells in a subject, the method comprising: administering to the subject a population of modified cells comprising a plurality of modified CD34+ or hematopoietic stem cells comprising an indel in an HBG promoter, wherein the percentage of F-cells among circulating red blood cells six months after said administering is at least 90%. In certain embodiments, mean corpuscular HbF per F-cell in the population of F-cells is at least 18 pg. In certain embodiments, the subject may exhibit no vaso-occlusive events (VOEs) within 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after said administering. In certain embodiments, the subject may exhibit no VOEs within 6 months after said administering. In certain embodiments, the subject may exhibit no VOEs within 1 year after said administering. In certain embodiments, the subject may exhibit no VOEs within 6, 12, 18, or 24 months after said administering.

In certain embodiments, a VOE is defined as acute episode of pain with no medically determined cause other than a vaso-occlusion. In certain embodiments, the subject may experience no VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, severe VOEs include an acute episode of pain with no cause other than a vaso-occlusion, resulting in either: a ≥24-hour hospital or Emergency Room (ER) observation unit visit; ≥2 visits to a day unit or ER over 72 hours, with both visits requiring oral or parenteral opioids or parenteral non-steroidal anti-inflammatory drugs (NSAIDs); or four visits to any medical facility over any time period (with or without medication requirement) for episodes of pain due to priapism. In certain embodiments, severe VOEs requiring medical attention (despite hydroxyurea or other supportive care measures in the pre-treatment period) may include an acute episode of pain with no cause other than a vaso-occlusion, resulting in either a ≥24-h hospital or Emergency Room (ER) observation unit or ≥2 visits to a day unit or ER over 72 h with both visits requiring administration of pain medications; acute priapism lasting >2 h and requiring a visit to a medical facility (with or without hospitalization); acute chest syndrome (ACS), which is defined as chest-wall pain in association with findings of a new pulmonary infiltrate on chest X-ray films associated with fever and/or respiratory symptom; or hepatic or splenic sequestration, which is defined as a sudden increase in organ size associated with pain in the area of the organ, decrease in the hemoglobin concentration of 2 g/dL within a 24-h period, and, for liver sequestration, abnormal change in liver function tests, including conjugated bilirubin, not due to biliary tract disease. In certain embodiments, the subject may experience no severe VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 2, 3, 4, 5, 6, 7, 8, 9, 10, 11, 12, 13, 14, 15, 16, 17, 18, 19, or 20 VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 2, 3, 4, 5, 6, 7, 8, 9, or 10 severe VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, a non-severe VOE may include an acute episode of pain with no medically determined cause other than a vaso-occlusion. In certain embodiments, the subject may experience no non-severe VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 1, 2, 3, 4, 5, 10, 15, 20, 25, 30, 35, 40, 45, 50, 55, 60, 65 treatment emergent adverse events (TEAEs) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject. In certain embodiments, the subject may experience fewer than 1, 2, 3, 4, 5, 6, 7, 8, 9, or 10 serious treatment emergent adverse events (TEAEs) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

In certain embodiments, the subject may be red blood cell (RBC) transfusion free 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

Candidate RNA-guided nucleases, gRNAs, and complexes thereof, can be evaluated by standard methods known in the art. See, e.g. Cotta-Ramusino. The stability of RNP complexes may be evaluated by differential scanning fluorimetry, as described below.

### Differential Scanning Fluorometry (DSF)

The thermostability of ribonucleoprotein (RNP) complexes comprising gRNAs and RNA-guided nucleases can be measured via DSF. The DSF technique measures the thermostability of a protein, which can increase under favorable conditions such as the addition of a binding RNA molecule, e.g., a gRNA.

A DSF assay can be performed according to any suitable protocol, and can be employed in any suitable setting, including without limitation (a) testing different conditions (e.g. different stoichiometric ratios of gRNA: RNA-guided nuclease protein, different buffer solutions, etc.) to identify optimal conditions for RNP formation; and (b) testing modifications (e.g. chemical modifications, alterations of sequence, etc.) of an RNA-guided nuclease and/or a gRNA to identify those modifications that improve RNP formation or stability. One readout of a DSF assay is a shift in melting temperature of the RNP complex; a relatively high shift suggests that the RNP complex is more stable (and may thus have greater activity or more favorable kinetics of formation, kinetics of degradation, or another functional characteristic) relative to a reference RNP complex characterized by a lower shift. When the DSF assay is deployed as a screening tool, a threshold melting temperature shift may be specified, so that the output is one or more RNPs having a melting temperature shift at or above the threshold. For instance, the threshold can be 5-10° C. (e.g. 5°, 6°, 7°, 8°, 9°, 10°) or more, and the output may be one or more RNPs characterized by a melting temperature shift greater than or equal to the threshold.

Two non-limiting examples of DSF assay conditions are set forth below:

To determine the best solution to form RNP complexes, a fixed concentration (e.g. 2 μM) of Cas9 in water+10×SYPRO Orange® (Life Technologies cat #S-6650) is dispensed into a 384 well plate. An equimolar amount of gRNA diluted in solutions with varied pH and salt is then added. After incubating at room temperature for 10′ and brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

The second assay consists of mixing various concentrations of gRNA with fixed concentration (e.g. 2 μM) Cas9 in optimal buffer from assay 1 above and incubating (e.g. at RT for 10′) in a 384 well plate. An equal volume of optimal buffer+10×SYPRO Orange® (Life Technologies cat #S-6650) is added and the plate sealed with Microseal® B adhesive (MSB-1001). Following brief centrifugation to remove any bubbles, a Bio-Rad CFX384™ Real-Time System C1000 Touch™ Thermal Cycler with the Bio-Rad CFX Manager software is used to run a gradient from 20° C. to 90° C. with a 1° C. increase in temperature every 10 seconds.

### Genome Editing Strategies

The genome editing systems described above are used, in various embodiments of the present disclosure, to generate edits in (i.e. to alter) targeted regions of DNA within or obtained from a cell. Various strategies are described herein to generate particular edits, and these strategies are generally described in terms of the desired repair outcome, the number and positioning of individual edits (e.g. SSBs or DSBs), and the target sites of such edits.

Genome editing strategies that involve the formation of SSBs or DSBs are characterized by repair outcomes including: (a) deletion of all or part of a targeted region; (b) insertion into or replacement of all or part of a targeted region; or (c) interruption of all or part of a targeted region. This grouping is not intended to be limiting, or to be binding to any particular theory or model, and is offered solely for economy of presentation. Skilled artisans will appreciate that the listed outcomes are not mutually exclusive and that some repairs may result in other outcomes. The description of a particular editing strategy or method should not be understood to require a particular repair outcome unless otherwise specified.

Replacement of a targeted region generally involves the replacement of all or part of the existing sequence within the targeted region with a homologous sequence, for instance through gene correction or gene conversion, two repair outcomes that are mediated by HDR pathways. HDR is promoted by the use of a donor template, which can be single-stranded or double stranded, as described in greater detail below. Single or double stranded templates can be exogenous, in which case they will promote gene correction, or they can be endogenous (e.g. a homologous sequence within the cellular genome), to promote gene conversion. Exogenous templates can have asymmetric overhangs (i.e. the portion of the template that is complementary to the site of the DSB may be offset in a 3′ or 5′ direction, rather than being centered within the donor template), for instance as described by Richardson 2016 (incorporated by reference herein). In instances where the template is single stranded, it can correspond to either the complementary (top) or non-complementary (bottom) strand of the targeted region.

Gene conversion and gene correction are facilitated, in some cases, by the formation of one or more nicks in or around the targeted region, as described in Ran & Hsu 2013 and Cotta-Ramusino. In some cases, a dual-nickase strategy is used to form two offset SSBs that, in turn, form a single DSB having an overhang (e.g. a 5′ overhang).

Interruption and/or deletion of all or part of a targeted sequence can be achieved by a variety of repair outcomes. As one example, a sequence can be deleted by simultaneously generating two or more DSBs that flank a targeted region, which is then excised when the DSBs are repaired, as is described in Maeder for the LCA10 mutation. As another example, a sequence can be interrupted by a deletion generated by formation of a double strand break with single-stranded overhangs, followed by exonucleolytic processing of the overhangs prior to repair.

One specific subset of target sequence interruptions is mediated by the formation of an indel within the targeted sequence, where the repair outcome is typically mediated by NHEJ pathways (including Alt-NHEJ). NHEJ is referred to as an “error prone” repair pathway because of its association with indel mutations. In some cases, however, a DSB is repaired by NHEJ without alteration of the sequence around it (a so-called “perfect” or “scarless” repair); this generally requires the two ends of the DSB to be perfectly ligated. Indels, meanwhile, are thought to arise from enzymatic processing of free DNA ends before they are ligated that adds and/or removes nucleotides from either or both strands of either or both free ends.

Because the enzymatic processing of free DSB ends may be stochastic in nature, indel mutations tend to be variable, occurring along a distribution, and can be influenced by a variety of factors, including the specific target site, the cell type used, the genome editing strategy used, etc. Even so, it is possible to draw limited generalizations about indel formation: deletions formed by repair of a single DSB are most commonly in the 1-50 bp range, but can reach greater than 100-200 bp. Insertions formed by repair of a single DSB tend to be shorter and often include short duplications of the sequence immediately surrounding the break site. However, it is possible to obtain large insertions, and in these cases, the inserted sequence has often been traced to other regions of the genome or to plasmid DNA present in the cells.

Indel mutations—and genome editing systems configured to produce indels—are useful for interrupting target sequences, for example, when the generation of a specific final sequence is not required and/or where a frameshift mutation would be tolerated. They can also be useful in settings where particular sequences are preferred, insofar as the certain sequences desired tend to occur preferentially from the repair of an SSB or DSB at a given site. Indel mutations are also a useful tool for evaluating or screening the activity of particular genome editing systems and their components. In these and other settings, indels can be characterized by (a) their relative and absolute frequencies in the genomes of cells contacted with genome editing systems and (b) the distribution of numerical differences relative to the unedited sequence, e.g. ±1, ±2, ±3, etc. As one example, in a lead-finding setting, multiple gRNAs can be screened to identify those gRNAs that most efficiently drive cutting at a target site based on an indel readout under controlled conditions. Guides that produce indels at or above a threshold frequency, or that produce a particular distribution of indels, can be selected for further study and development. Indel frequency and distribution can also be useful as a readout for evaluating different genome editing system implementations or formulations and delivery methods, for instance by keeping the gRNA constant and varying certain other reaction conditions or delivery methods.

### Multiplex Strategies

Genome editing systems according to this disclosure may also be employed for multiplex gene editing to generate two or more DSBs, either in the same locus or in different loci. Any of the RNA-guided nucleases and gRNAs disclosed herein may be used in genome editing systems for multiplex gene editing. Strategies for editing that involve the formation of multiple DSBs, or SSBs, are described in, for instance, Cotta-Ramusino. In certain embodiments, multiple gRNAs and an RNA-guided nuclease may be used in genome editing systems to introduce alterations (e.g., deletions, insertions) into the CCAAT box target region of HBG1 and/or HBG2. In certain embodiments, the RNA-guided nuclease may be a Cas12a protein.

### Donor Template Design

Donor template design is described in detail in the literature, for instance in Cotta-Ramusino. DNA oligomer donor templates (oligodeoxynucleotides or ODNs), which can be single stranded (ssODNs) or double-stranded (dsODNs), can be used to facilitate HDR-based repair of DSBs or to boost overall editing rate, and are particularly useful for introducing alterations into a target DNA sequence, inserting a new sequence into the target sequence, or replacing the target sequence altogether.

Whether single-stranded or double stranded, donor templates generally include regions that are homologous to regions of DNA within or near (e.g. flanking or adjoining) a target sequence to be cleaved. These homologous regions are referred to here as “homology arms,” and are illustrated schematically below:
[5′ homology arm]-[replacement sequence]--[3′ homology arm].

The homology arms can have any suitable length (including 0 nucleotides if only one homology arm is used), and 3′ and 5′ homology arms can have the same length, or can differ in length. The selection of appropriate homology arm lengths can be influenced by a variety of factors, such as the desire to avoid homologies or microhomologies with certain sequences such as Alu repeats or other very common elements. For example, a 5′ homology arm can be shortened to avoid a sequence repeat element. In other embodiments, a 3′ homology arm can be shortened to avoid a sequence repeat element. In some embodiments, both the 5′ and the 3′ homology arms can be shortened to avoid including certain sequence repeat elements. In addition, some homology arm designs can improve the efficiency of editing or increase the frequency of a desired repair outcome. For example, Richardson 2016, which is incorporated by reference herein, found that the relative asymmetry of 3′ and 5′ homology arms of single stranded donor templates influenced repair rates and/or outcomes.

Replacement sequences in donor templates have been described elsewhere, including in Cotta-Ramusino. A replacement sequence can be any suitable length (including zero nucleotides, where the desired repair outcome is a deletion), and typically includes one, two, three or more sequence modifications relative to the naturally-occurring sequence within a cell in which editing is desired. One common sequence modification involves the alteration of the naturally-occurring sequence to repair a mutation that is related to a disease or condition of which treatment is desired. Another common sequence modification involves the alteration of one or more sequences that are complementary to, or then, the PAM sequence of the RNA-guided nuclease or the targeting domain of the gRNA(s) being used to generate an SSB or DSB, to reduce or eliminate repeated cleavage of the target site after the replacement sequence has been incorporated into the target site.

Where a linear ssODN is used, it can be configured to (i) anneal to the nicked strand of the target nucleic acid, (ii) anneal to the intact strand of the target nucleic acid, (iii) anneal to the plus strand of the target nucleic acid, and/or (iv) anneal to the minus strand of the target nucleic acid. An ssODN may have any suitable length, e.g., about, at least, or no more than 80-200 nucleotides (e.g., 80, 90, 100, 110, 120, 130, 140, 150, 160, 170, 180, 190, or 200 nucleotides).

It should be noted that a template nucleic acid can also be a nucleic acid vector, such as a viral genome or circular double stranded DNA, e.g., a plasmid. Nucleic acid vectors comprising donor templates can include other coding or non-coding elements. For example, a template nucleic acid can be delivered as part of a viral genome (e.g., in an AAV or lentiviral genome) that includes certain genomic backbone elements (e.g., inverted terminal repeats, in the case of an AAV genome) and optionally includes additional sequences coding for a gRNA and/or an RNA-guided nuclease. In certain embodiments, the donor template can be adjacent to, or flanked by, target sites recognized by one or more gRNAs, to facilitate the formation of free DSBs on one or both ends of the donor template that can participate in repair of corresponding SSBs or DSBs formed in cellular DNA using the same gRNAs. Exemplary nucleic acid vectors suitable for use as donor templates are described in Cotta-Ramusino, which is incorporated by reference.

Whatever format is used, a template nucleic acid can be designed to avoid undesirable sequences. In certain embodiments, one or both homology arms can be shortened to avoid overlap with certain sequence repeat elements, e.g., Alu repeats, LINE elements, etc.

In certain embodiments, silent, non-pathogenic SNPs may be included in the ssODN donor template to allow for identification of a gene editing event.

In certain embodiments, a donor template may be a non-specific template that is non-homologous to regions of DNA within or near a target sequence to be cleaved.

A donor template or template nucleic acid, as that term is used herein, refers to a nucleic acid sequence which can be used in conjunction with an RNA nuclease molecule and one or more gRNA molecules to alter (e.g., delete, disrupt, or modify) a target DNA sequence. In certain embodiments, the template nucleic acid results in an alteration (e.g., deletion) at the CCAAT box target region of HBG1 and/or HBG2. In certain embodiments, the alteration is a non-naturally occurring alteration. In certain embodiments, the template nucleic acid is an ssODN. In certain embodiments, the ssODN is a positive strand or a negative strand.

In certain embodiments, the 5′ homology arm comprises a 5′ phosphorothioate (PhTx) modification. In certain embodiments, the 3′ homology arm comprises a 3′ PhTx modification. In certain embodiments, the template nucleic acid comprises a 5′ and 3′ PhTx modification.

In certain embodiments, the ssODNs for introducing alterations (e.g., deletions) at the CCAAT box target region may be used in conjunction with an RNA nuclease and one or more gRNAs that target the CCAAT target region.

### Target Cells

Genome editing systems according to this disclosure can be used to manipulate or alter a cell, e.g., to edit or alter a target nucleic acid. The manipulating can occur, in various embodiments, in vivo or ex vivo.

A variety of cell types can be manipulated or altered according to the embodiments of this disclosure, and in some cases, such as in vivo applications, a plurality of cell types are altered or manipulated, for example by delivering genome editing systems according to this disclosure to a plurality of cell types. In other cases, however, it may be desirable to limit manipulation or alteration to a particular cell type or types. For instance, it can be desirable in some instances to edit a cell with limited differentiation potential or a terminally differentiated cell, such as a photoreceptor cell in the case of Maeder, in which modification of a genotype is expected to result in a change in cell phenotype. In other cases, however, it may be desirable to edit a less differentiated, multipotent or pluripotent, stem or progenitor cell. By way of example, the cell may be an embryonic stem cell, induced pluripotent stem cell (iPSC), hematopoietic stem/progenitor cell (HSPC), or other stem or progenitor cell type that differentiates into a cell type of relevance to a given application or indication.

As a corollary, the cell being altered or manipulated is, variously, a dividing cell or a non-dividing cell, depending on the cell type(s) being targeted and/or the desired editing outcome.

When cells are manipulated or altered ex vivo, the cells can be used (e.g. administered to a subject) immediately, or they can be maintained or stored for later use. Those of skill in the art will appreciate that cells can be maintained in culture or stored (e.g. frozen in liquid nitrogen) using any suitable method known in the art.

### Implementation of Genome Editing Systems: Delivery, Formulations, and Routes of Administration

As discussed above, the genome editing systems of this disclosure can be implemented in any suitable manner, meaning that the components of such systems, including without limitation the RNA-guided nuclease, gRNA, and optional donor template nucleic acid, can be delivered, formulated, or administered in any suitable form or combination of forms that results in the transduction, expression or introduction of a genome editing system and/or causes a desired repair outcome in a cell, tissue or subject. Tables 2 and 3 set forth several, non-limiting examples of genome editing system implementations. Those of skill in the art will appreciate, however, that these listings are not comprehensive, and that other implementations are possible. With reference to Table 2 in particular, the table lists several exemplary implementations of a genome editing system comprising a single gRNA and an optional donor template. However, genome editing systems according to this disclosure can incorporate multiple gRNAs, multiple RNA-guided nucleases, and other components such as proteins, and a variety of implementations will be evident to the skilled artisan based on the principles illustrated in the table. In the table, [N/A] indicates that the genome editing system does not include the indicated component.

TABLE 2
Genome Editing System Components

| RNA- | | Donor | |
|---|---|---|---|
| guided | | | |
| Nuclease | gRNA | Template | Comments |
| Protein | RNA | [N/A] | An RNA-guided nuclease protein |
| | | | complexed with a gRNA molecule (an |
| | | | RNP complex) |
| Protein | RNA | DNA | An RNP complex as described above |
| | | | plus a single-stranded or double |
| | | | stranded donor template. |
| Protein | DNA | [N/A] | An RNA-guided nuclease protein plus |
| | | | gRNA transcribed from DNA. |
| Protein | DNA | DNA | An RNA-guided nuclease protein plus |
| | | | gRNA-encoding DNA and a separate |
| | | | DNA donor template. |
| Protein | DNA | | An RNA-guided nuclease protein and |
| | | | a single DNA encoding both a gRNA |
| | | | and a donor template. |
| DNA | | | A DNA or DNA vector encoding an |
| | | | RNA-guided nuclease, a gRNA and a |
| | | | donor template. |
| DNA | DNA | [N/A] | Two separate DNAs, or two separate |
| | | | DNA vectors, encoding the RNA- |
| | | | guided nuclease and the gRNA, |
| | | | respectively. |
| DNA | DNA | DNA | Three separate DNAs, or three |
| | | | separate DNA vectors, encoding the |
| | | | RNA-guided nuclease, the gRNA and |
| | | | the donor template, respectively. |
| DNA | | [N/A] | A DNA or DNA vector encoding an |
| | | | RNA-guided nuclease and a gRNA |
| DNA | | DNA | A first DNA or DNA vector encoding |
| | | | an RNA-guided nuclease and a gRNA, |
| | | | and a second DNA or DNA vector |
| | | | encoding a donor template. |
| DNA | DNA | | A first DNA or DNA vector encoding |
| | | | an RNA-guided nuclease and second |
| | | | DNA or DNA vector encoding a |
| | | | gRNA and a donor template. |
| DNA | | | A first DNA or DNA vector encoding |
| | | | an RNA-guided nuclease and a donor |
| | DNA | | template, and a second DNA or DNA |
| | | | vector encoding a gRNA |
| DNA | | | A DNA or DNA vector encoding an |
| | RNA | | RNA-guided nuclease and a donor |
| | | | template, and a gRNA |

| | | |
|---|---|---|
| RNA | [N/A] | An RNA or RNA vector encoding an |
| | | RNA-guided nuclease and comprising |
| | | a gRNA |
| RNA | DNA | An RNA or RNA vector encoding an |
| | | RNA-guided nuclease and comprising |
| | | a gRNA, and a DNA or DNA vector |
| | | encoding a donor template. |

Table 3 summarizes various delivery methods for the components of genome editing systems, as described herein. Again, the listing is intended to be exemplary rather than limiting.

TABLE 3

| | | Delivery | | | |
|---|---|---|---|---|---|
| | | into Non- | | | Type of |
| | | Dividing | Duration of | Genome | Molecule |
| Delivery Vector/Mode | | Cells | Expression | Integration | Delivered |
| Physical (e.g., electroporation, | | YES | Transient | NO | Nucleic Acids |
| particle gun, Calcium | | | | | and Proteins |
| Phosphate transfection, cell | | | | | |
| compression or squeezing) | | | | | |
| Viral | Retrovirus | NO | Stable | YES | RNA |
| | Lentivirus | YES | Stable | YES/NO with | RNA |
| | | | | modifications | |
| | Adenovirus | YES | Transient | NO | DNA |
| | Adeno- | YES | Stable | NO | DNA |
| | Associated Virus | | | | |
| | (AAV) | | | | |
| | Vaccinia Virus | YES | Very | NO | DNA |
| | | | Transient | | |
| | Herpes Simplex | YES | Stable | NO | DNA |
| | Virus | | | | |
| Non-Viral | Cationic | YES | Transient | Depends on | Nucleic Acids |
| | Liposomes | | | what is | and Proteins |
| | | | | delivered | |
| | Polymeric | YES | Transient | Depends on | Nucleic Acids |
| | Nanoparticles | | | what is | and Proteins |
| | | | | delivered | |
| Biological | Attenuated | YES | Transient | NO | Nucleic Acids |
| Non-Viral | Bacteria | | | | |
| Delivery | Engineered | YES | Transient | NO | Nucleic Acids |
| Vehicles | Bacteriophages | | | | |
| | Mammalian | YES | Transient | NO | Nucleic Acids |
| | Virus-like | | | | |
| | Particles | | | | |
| | Biological | YES | Transient | NO | Nucleic Acids |
| | liposomes: | | | | |
| | Erythrocyte | | | | |
| | Ghosts and | | | | |
| | Exosomes | | | | |

### Nucleic Acid-Based Delivery of Genome Editing Systems

Nucleic acids encoding the various elements of a genome editing system according to the present disclosure can be administered to subjects or delivered into cells by art-known methods or as described herein. For example, RNA-guided nuclease-encoding and/or gRNA-encoding DNA, as well as donor template nucleic acids can be delivered by, e.g., vectors (e.g., viral or non-viral vectors), non-vector based methods (e.g., using naked DNA or DNA complexes), or a combination thereof.

Nucleic acids encoding genome editing systems or components thereof can be delivered directly to cells as naked DNA or RNA, for instance by means of transfection or electroporation, or can be conjugated to molecules (e.g., N-acetylgalactosamine) promoting uptake by the target cells (e.g., erythrocytes, HSCs). Nucleic acid vectors, such as the vectors summarized in Table 3, can also be used.

Nucleic acid vectors can comprise one or more sequences encoding genome editing system components, such as an RNA-guided nuclease, a gRNA and/or a donor template. A vector can also comprise a sequence encoding a signal peptide (e.g., for nuclear localization, nucleolar localization, or mitochondrial localization), associated with (e.g., inserted into or fused to) a sequence coding for a protein. As one example, a nucleic acid vectors can include a Cas9 coding sequence that includes one or more nuclear localization sequences (e.g., a nuclear localization sequence from SV40).

The nucleic acid vector can also include any suitable number of regulatory/control elements, e.g., promoters, enhancers, introns, polyadenylation signals, Kozak consensus sequences, or internal ribosome entry sites (IRES). These elements are well known in the art, and are described in Cotta-Ramusino.

Nucleic acid vectors according to this disclosure include recombinant viral vectors. Exemplary viral vectors are set forth in Table 3, and additional suitable viral vectors and their use and production are described in Cotta-Ramusino. Other viral vectors known in the art can also be used. In addition, viral particles can be used to deliver genome editing system components in nucleic acid and/or peptide form. For example, “empty” viral particles can be assembled to contain any suitable cargo. Viral vectors and viral particles can also be engineered to incorporate targeting ligands to alter target tissue specificity.

In addition to viral vectors, non-viral vectors can be used to deliver nucleic acids encoding genome editing systems according to the present disclosure. One important category of non-viral nucleic acid vectors are nanoparticles, which can be organic or inorganic. Nanoparticles are well known in the art, and are summarized in Cotta-Ramusino. Any suitable nanoparticle design can be used to deliver genome editing system components or nucleic acids encoding such components. For instance, organic (e.g. lipid and/or polymer) nanoparticles can be suitable for use as delivery vehicles in certain embodiments of this disclosure. Exemplary lipids for use in nanoparticle formulations, and/or gene transfer are shown in Table 4 and Table 5 lists exemplary polymers for use in gene transfer and/or nanoparticle formulations.

TABLE 4
Lipids Used for Gene Transfer

| Lipid | Abbreviation | Feature |
|---|---|---|
| 1,2-Dioleoyl-sn-glycero-3-phosphatidylcholine | DOPC | Helper |
| 1,2-Dioleoyl-sn-glycero-3-phosphatidylethanolamine | DOPE | Helper |
| Cholesterol | | Helper |
| N-[1-(2,3-Dioleyloxy)propyl]N,N,N-trimethylammonium chloride | DOTMA | Cationic |
| 1,2-Dioleoyloxy-3-trimethylammonium-propane | DOTAP | Cationic |
| Dioctadecylamidoglycylspermine | DOGS | Cationic |
| N-(3-Aminopropyl)-N,N-dimethyl-2,3-bis(dodecyloxy)-1- | GAP-DLRIE | Cationic |
| propanaminium bromide | | |
| Cetyltrimethylammonium bromide | CTAB | Cationic |
| 6-Lauroxyhexyl ornithinate | LHON | Cationic |
| 1-(2,3-Dioleoyloxypropyl)-2,4,6-trimethylpyridinium | 20c | Cationic |
| 2,3-Dioleyloxy-N-[2(sperminecarboxamido-ethyl]-N,N-dimethyl-1- | DOSPA | Cationic |
| propanaminium trifluoroacetate | | |
| 1,2-Dioleyl-3-trimethylammonium-propane | DOPA | Cationic |
| N-(2-Hydroxyethyl)-N,N-dimethyl-2,3-bis(tetradecyloxy)-1- | MDRIE | Cationic |
| propanaminium bromide | | |
| Dimyristooxypropyl dimethyl hydroxyethyl ammonium bromide | DMRI | Cationic |
| 3β-[N-(N′, N′-Dimethylaminoethane)-carbamoyl]cholesterol | DC-Chol | Cationic |
| Bis-guanidium-tren-cholesterol | BGTC | Cationic |
| 1,3-Diodeoxy-2-(6-carboxy-spermyl)-propylamide | DOSPER | Cationic |
| Dimethyloctadecylammonium bromide | DDAB | Cationic |
| Dioctadecylamidoglicylspermidin | DSL | Cationic |
| rac-[(2,3-Dioctadecyloxypropyl)(2-hydroxyethyl)]-dimethylammonium | CLIP-1 | Cationic |
| chloride | | |
| rac-[2(2,3-Dihexadecyloxypropyl- | CLIP-6 | Cationic |
| oxymethyloxy)ethyl]trimethylammonium bromide | | |
| Ethyldimyristoylphosphatidylcholine | EDMPC | Cationic |
| 1,2-Distearyloxy-N,N-dimethyl-3-aminopropane | DSDMA | Cationic |
| 1,2-Dimyristoyl-trimethylammonium propane | DMTAP | Cationic |
| O,O′-Dimyristyl-N-lysyl aspartate | DMKE | Cationic |
| 1,2-Distearoyl-sn-glycero-3-ethylphosphocholine | DSEPC | Cationic |
| N-Palmitoyl D-erythro-sphingosyl carbamoyl-spermine | CCS | Cationic |
| N-t-Butyl-N0-tetradecyl-3-tetradecylaminopropionamidine | diC14-amidine | Cationic |
| Octadecenolyoxy[ethyl-2-heptadecenyl-3 hydroxyethyl] imidazolinium | DOTIM | Cationic |
| chloride | | |
| N1-Cholesteryloxycarbonyl-3,7-diazanonane-1,9-diamine | CDAN | Cationic |
| 2-(3-[Bis(3-amino-propyl)-amino]propylamino)-N- | RPR209120 | Cationic |
| ditetradecylcarbamoylme-ethyl-acetamide | | |
| 1,2-dilinoleyloxy-3-dimethylaminopropane | DLinDMA | Cationic |
| 2,2-dilinoleyl-4-dimethylaminoethyl-[1,3]-dioxolane | DLin-KC2-DMA | Cationic |
| dilinoleyl-methyl-4-dimethylaminobutyrate | DLin-MC3-DMA | Cationic |

TABLE 5
Polymers Used for Gene Transfer

| Polymer | Abbreviation |
|---|---|
| Poly(ethylene)glycol | PEG |
| Polyethylenimine | PEI |
| Dithiobis(succinimidylpropionate) | DSP |
| Dimethyl-3,3′-dithiobispropionimidate | DTBP |
| Poly(ethylene imine) biscarbamate | PEIC |
| Poly(L-lysine) | PLL |
| Histidine modified PLL | |
| Poly(N-vinylpyrrolidone) | PVP |
| Poly(propylenimine) | PPI |
| Poly(amidoamine) | PAMAM |
| Poly(amido ethylenimine) | SS-PAEI |
| Triethylenetetramine | TETA |
| Poly(β-aminoester) | |
| Poly(4-hydroxy-L-proline ester) | PHP |
| Poly(allylamine) | |
| Poly(α-[4-aminobutyl]-L-glycolic acid) | PAGA |
| Poly(D,L-lactic-co-glycolic acid) | PLGA |
| Poly(N-ethyl-4-vinylpyridinium bromide) | |
| Poly(phosphazene)s | PPZ |
| Poly(phosphoester)s | PPE |
| Poly(phosphoramidate)s | PPA |
| Poly(N-2-hydroxypropylmethacrylamide) | PHPMA |
| Poly (2-(dimethylamino)ethyl methacrylate) | pDMAEMA |
| Poly(2-aminoethyl propylene phosphate) | PPE-EA |
| Chitosan | |
| Galactosylated chitosan | |
| N-Dodacylated chitosan | |
| Histone | |
| Collagen | |
| Dextran-spermine | D-SPM |

Non-viral vectors optionally include targeting modifications to improve uptake and/or selectively target certain cell types. These targeting modifications can include e.g., cell specific antigens, monoclonal antibodies, single chain antibodies, aptamers, polymers, sugars (e.g., N-acetylgalactosamine (GalNAc)), and cell penetrating peptides. Such vectors also optionally use fusogenic and endosome-destabilizing peptides/polymers, undergo acid-triggered conformational changes (e.g., to accelerate endosomal escape of the cargo), and/or incorporate a stimuli-cleavable polymer, e.g., for release in a cellular compartment. For example, disulfide-based cationic polymers that are cleaved in the reducing cellular environment can be used.

In certain embodiments, one or more nucleic acid molecules (e.g., DNA molecules) other than the components of a genome editing system, e.g., the RNA-guided nuclease component and/or the gRNA component described herein, are delivered. In certain embodiments, the nucleic acid molecule is delivered at the same time as one or more of the components of the genome editing system. In certain embodiments, the nucleic acid molecule is delivered before or after (e.g., less than about 30 minutes, 1 hour, 2 hours, 3 hours, 6 hours, 9 hours, 12 hours, 1 day, 2 days, 3 days, 1 week, 2 weeks, or 4 weeks) one or more of the components of the genome editing system are delivered. In certain embodiments, the nucleic acid molecule is delivered by a different means than one or more of the components of the genome editing system, e.g., the RNA-guided nuclease component and/or the gRNA component, are delivered. The nucleic acid molecule can be delivered by any of the delivery methods described herein. For example, the nucleic acid molecule can be delivered by a viral vector, e.g., an integration-deficient lentivirus, and the RNA-guided nuclease molecule component and/or the gRNA component can be delivered by electroporation, e.g., such that the toxicity caused by nucleic acids (e.g., DNAs) can be reduced. In certain embodiments, the nucleic acid molecule encodes a therapeutic protein, e.g., a protein described herein. In certain embodiments, the nucleic acid molecule encodes an RNA molecule, e.g., an RNA molecule described herein.

Delivery of RNPs and/or RNA Encoding Genome Editing System Components

RNPs (complexes of gRNAs and RNA-guided nucleases) and/or RNAs encoding RNA-guided nucleases and/or gRNAs, can be delivered into cells or administered to subjects by art-known methods, some of which are described in Cotta-Ramusino. In vitro, RNA-guided nuclease-encoding and/or gRNA-encoding RNA can be delivered, e.g., by microinjection, electroporation, transient cell compression or squeezing (see, e.g., Lee 2012). Lipid-mediated transfection, peptide-mediated delivery, GalNAc- or other conjugate-mediated delivery, and combinations thereof, can also be used for delivery in vitro and in vivo. A protective, interactive, non-condensing (PINC) system may be used for delivery.

In vitro delivery via electroporation comprises mixing the cells with the RNA encoding RNA-guided nucleases and/or gRNAs, with or without donor template nucleic acid molecules, in a cartridge, chamber or cuvette and applying one or more electrical impulses of defined duration and amplitude. Systems and protocols for electroporation are known in the art, and any suitable electroporation tool and/or protocol can be used in connection with the various embodiments of this disclosure.

### Route of Administration

Genome editing systems, or cells altered or manipulated using such systems, can be administered to subjects by any suitable mode or route, whether local or systemic. Systemic modes of administration include oral and parenteral routes. Parenteral routes include, by way of example, intravenous, intramarrow, intrarterial, intramuscular, intradermal, subcutaneous, intranasal, and intraperitoneal routes. Components administered systemically can be modified or formulated to target, e.g., HSCs, hematopoietic stem/progenitor cells, or erythroid progenitors or precursor cells.

Local modes of administration include, by way of example, intramarrow injection into the trabecular bone or intrafemoral injection into the marrow space, and infusion into the portal vein. In certain embodiments, significantly smaller amounts of the components (compared with systemic approaches) can exert an effect when administered locally (for example, directly into the bone marrow) compared to when administered systemically (for example, intravenously). Local modes of administration can reduce or eliminate the incidence of potentially toxic side effects that may occur when therapeutically effective amounts of a component are administered systemically.

Administration can be provided as a periodic bolus (for example, intravenously) or as continuous infusion from an internal reservoir or from an external reservoir (for example, from an intravenous bag or implantable pump). Components can be administered locally, for example, by continuous release from a sustained release drug delivery device.

In addition, components can be formulated to permit release over a prolonged period of time. A release system can include a matrix of a biodegradable material or a material which releases the incorporated components by diffusion. The components can be homogeneously or heterogeneously distributed within the release system. A variety of release systems can be useful, however, the choice of the appropriate system will depend upon rate of release required by a particular application. Both non-degradable and degradable release systems can be used. Suitable release systems include polymers and polymeric matrices, non-polymeric matrices, or inorganic and organic excipients and diluents such as, but not limited to, calcium carbonate and sugar (for example, trehalose). Release systems may be natural or synthetic. However, synthetic release systems are preferred because generally they are more reliable, more reproducible and produce more defined release profiles. The release system material can be selected so that components having different molecular weights are released by diffusion through or degradation of the material.

Representative synthetic, biodegradable polymers include, for example: polyamides such as poly(amino acids) and poly(peptides); polyesters such as poly(lactic acid), poly(glycolic acid), poly(lactic-co-glycolic acid), and poly(caprolactone); poly(anhydrides); polyorthoesters; polycarbonates; and chemical derivatives thereof (substitutions, additions of chemical groups, for example, alkyl, alkylene, hydroxylations, oxidations, and other modifications routinely made by those skilled in the art), copolymers and mixtures thereof. Representative synthetic, non-degradable polymers include, for example: polyethers such as poly(ethylene oxide), poly(ethylene glycol), and poly(tetramethylene oxide); vinyl polymers-polyacrylates and polymethacrylates such as methyl, ethyl, other alkyl, hydroxyethyl methacrylate, acrylic and methacrylic acids, and others such as poly(vinyl alcohol), poly(vinyl pyrolidone), and poly(vinyl acetate); poly(urethanes); cellulose and its derivatives such as alkyl, hydroxyalkyl, ethers, esters, nitrocellulose, and various cellulose acetates; polysiloxanes; and any chemical derivatives thereof (substitutions, additions of chemical groups, for example, alkyl, alkylene, hydroxylations, oxidations, and other modifications routinely made by those skilled in the art), copolymers and mixtures thereof.

Poly(lactide-co-glycolide) microsphere can also be used. Typically the microspheres are composed of a polymer of lactic acid and glycolic acid, which are structured to form hollow spheres. The spheres can be approximately 15-30 microns in diameter and can be loaded with components described herein. In some embodiments, genome editing systems, system components and/or nucleic acids encoding system components, are delivered with a block copolymer such as a poloxamer or a poloxamine.

### Multi-Modal or Differential Delivery of Components

Skilled artisans will appreciate, in view of the instant disclosure, that different components of genome editing systems disclosed herein can be delivered together or separately and simultaneously or non-simultaneously. Separate and/or asynchronous delivery of genome editing system components can be particularly desirable to provide temporal or spatial control over the function of genome editing systems and to limit certain effects caused by their activity.

Different or differential modes as used herein refer to modes of delivery that confer different pharmacodynamic or pharmacokinetic properties on the subject component molecule, e.g., a RNA-guided nuclease molecule, gRNA, template nucleic acid, or payload. For example, the modes of delivery can result in different tissue distribution, different half-life, or different temporal distribution, e.g., in a selected compartment, tissue, or organ.

Some modes of delivery, e.g., delivery by a nucleic acid vector that persists in a cell, or in progeny of a cell, e.g., by autonomous replication or insertion into cellular nucleic acid, result in more persistent expression of and presence of a component. Examples include viral, e.g., AAV or lentivirus, delivery.

By way of example, the components of a genome editing system, e.g., a RNA-guided nuclease and a gRNA, can be delivered by modes that differ in terms of resulting half-life or persistent of the delivered component the body, or in a particular compartment, tissue or organ. In certain embodiments, a gRNA can be delivered by such modes. The RNA-guided nuclease molecule component can be delivered by a mode which results in less persistence or less exposure to the body or a particular compartment or tissue or organ.

More generally, in certain embodiments, a first mode of delivery is used to deliver a first component and a second mode of delivery is used to deliver a second component. The first mode of delivery confers a first pharmacodynamic or pharmacokinetic property. The first pharmacodynamic property can be, e.g., distribution, persistence, or exposure, of the component, or of a nucleic acid that encodes the component, in the body, a compartment, tissue or organ. The second mode of delivery confers a second pharmacodynamic or pharmacokinetic property. The second pharmacodynamic property can be, e.g., distribution, persistence, or exposure, of the component, or of a nucleic acid that encodes the component, in the body, a compartment, tissue or organ.

In certain embodiments, the first pharmacodynamic or pharmacokinetic property, e.g., distribution, persistence or exposure, is more limited than the second pharmacodynamic or pharmacokinetic property.

In certain embodiments, the first mode of delivery is selected to optimize, e.g., minimize, a pharmacodynamic or pharmacokinetic property, e.g., distribution, persistence or exposure.

In certain embodiments, the second mode of delivery is selected to optimize, e.g., maximize, a pharmacodynamic or pharmacokinetic property, e.g., distribution, persistence or exposure.

In certain embodiments, the first mode of delivery comprises the use of a relatively persistent element, e.g., a nucleic acid, e.g., a plasmid or viral vector, e.g., an AAV or lentivirus. As such vectors are relatively persistent product transcribed from them would be relatively persistent.

In certain embodiments, the second mode of delivery comprises a relatively transient element, e.g., an RNA or protein.

In certain embodiments, the first component comprises gRNA, and the delivery mode is relatively persistent, e.g., the gRNA is transcribed from a plasmid or viral vector, e.g., an AAV or lentivirus. Transcription of these genes would be of little physiological consequence because the genes do not encode for a protein product, and the gRNAs are incapable of acting in isolation. The second component, a RNA-guided nuclease molecule, is delivered in a transient manner, for example as mRNA or as protein, ensuring that the full RNA-guided nuclease molecule/gRNA complex is only present and active for a short period of time.

Furthermore, the components can be delivered in different molecular form or with different delivery vectors that complement one another to enhance safety and tissue specificity.

Use of differential delivery modes can enhance performance, safety, and/or efficacy, e.g., the likelihood of an eventual off-target modification can be reduced. Delivery of immunogenic components, e.g., Cas9 molecules, by less persistent modes can reduce immunogenicity, as peptides from the bacterially-derived Cas enzyme are displayed on the surface of the cell by MHC molecules. A two-part delivery system can alleviate these drawbacks.

Differential delivery modes can be used to deliver components to different, but overlapping target regions. The formation active complex is minimized outside the overlap of the target regions. Thus, in certain embodiments, a first component, e.g., a gRNA is delivered by a first delivery mode that results in a first spatial, e.g., tissue, distribution. A second component, e.g., a RNA-guided nuclease molecule is delivered by a second delivery mode that results in a second spatial, e.g., tissue, distribution. In certain embodiments, the first mode comprises a first element selected from a liposome, nanoparticle, e.g., polymeric nanoparticle, and a nucleic acid, e.g., viral vector. The second mode comprises a second element selected from the group. In certain embodiments, the first mode of delivery comprises a first targeting element, e.g., a cell specific receptor or an antibody, and the second mode of delivery does not include that element. In certain embodiments, the second mode of delivery comprises a second targeting element, e.g., a second cell specific receptor or second antibody.

When the RNA-guided nuclease molecule is delivered in a virus delivery vector, a liposome, or polymeric nanoparticle, there is the potential for delivery to and therapeutic activity in multiple tissues, when it may be desirable to only target a single tissue. A two-part delivery system can resolve this challenge and enhance tissue specificity. If the gRNA and the RNA-guided nuclease molecule are packaged in separated delivery vehicles with distinct but overlapping tissue tropism, the fully functional complex is only be formed in the tissue that is targeted by both vectors.

### EXAMPLES

The principles and embodiments described above are further illustrated by the non-limiting examples that follow:

### Example 1: Autologous CD34+ Cells Edited by RNP27 at the HBG1/HBG2 Promoter Show Early, Promising Efficacy and Safety in a Phase 1/2/3 Study in Subjects with Severe Sickle Cell Disease

Sickle cell disease (SCD) is a genetic disorder caused by mutations in the HBB gene that cause sickling of red blood cells (RBCs), which leads to anemia, hemolysis, and vaso-occlusive events (VOEs) (Kato 2018; Williams 2018). Lifelong complications, multi-organ damage, and comorbidities impact SCD patient's quality of life, ultimately leading to a shortened lifespan. Over 300,000 babies with SCD are born globally, over 100,000 people in the U.S. have SCD, and over 6,000,000 people globally have SCD (Wastnedge 2018; Sickle Cell Disease available on the world wide web at nhlbi.nih.gov/health/sickle-cell-disease (accessed June 2023)). 50% of patients with SCD die before 45 years of age (Jiao 2023). Although advances in supportive care and disease modifying therapies have improved outcomes for patients with SCD, curative therapies have been limited to allogenic HCT.

Sustained increases in levels of hemoglobin and fetal hemoglobin have positive clinical benefits for patients living with SCD. Sustained increases in hemoglobin to the normal physiological range are associated with a reduction in the risk for end-organ damage and a reduction in the risk for negative clinical outcomes in patients with SCD. Sustained increases in fetal hemoglobin levels correlate with reduction or elimination of SCD symptoms, including vaso-occlusive events (VOEs).

To determine the safety, tolerability, and efficacy of treatment with autologous RNP27 edited CD34+ cells, a Phase 1/2/3, multicenter, open-label, single arm study is performed with subjects with SCD. RNP27 (Table 18) comprises a gRNA comprising the sequence set forth in SEQ ID NO:237 complexed with a Cas12a protein comprising the sequence set forth in SEQ ID NO:283 (exemplary nucleotide sequence is SEQ ID NO:300). As shown in FIG. 3, RNP27 cleaves the genomic DNA in the distal CCAAT box region of γ-globin gene promoters (HBG1 and HBG2) on chromosome 11.

Gamma-globin pairs with α-globin to form fetal hemoglobin (HbF, α2γ2) the predominant hemoglobin expressed at birth. γ-globin is typically silenced and replaced by adult D-globin in the first year of life. Mutations (point mutations and the 13 bp hereditary persistence of fetal hemoglobin [HPFH] deletion) in the distal CCAAT box region of the HBG1 and HBG2 promoters are associated with elevated γ-globin and fetal hemoglobin (HbF) expression throughout adulthood due to disruption of the cis-regulatory elements involved in γ-globin repression. The insertions and/or deletions (indels) generated by RNP27 in this region act by an analogous mechanism to HPFH mutations in de-repressing γ-globin expression in adults.

As described herein, autologous RNP27 edited CD34+ cells, administered as a one-time, autologous CD34+ cell therapy has the potential to lead to sustained, elevated levels of HbF in SCD subjects, ameliorating the disease phenotypes.

In the Phase 1/2/3, international, multicenter, open-label, single arm study, 40 subjects are 18-50 years old with a diagnosis of severe SCD defined as a history of ≥2 severe VOEs per year in the 2 year-period prior to provision of informed consent.

The key endpoints in the study are (1) proportion of subjects achieving complete resolution of severe vaso-occlusive events (VOEs) requiring medical attention (time frame: up to 2 years post infusion), and (2) safety and tolerability of treatment with RNP27 edited CD34+ cells. A VOE is defined as an acute episode of pain with no medically determined cause other than a vaso-occlusion. A severe VOE requiring medical attention (despite hydroxyurea or other supportive care measures in the pre-treatment period) is defined as: an acute episode of pain with no cause other than a vaso-occlusion, resulting in either a ≥24-h hospital or Emergency Room (ER) observation unit or ≥2 visits to a day unit or ER over 72 h with both visits requiring administration of pain medications; acute priapism lasting >2 h and requiring a visit to a medical facility (with or without hospitalization); acute chest syndrome (ACS), which is defined as chest-wall pain in association with findings of a new pulmonary infiltrate on chest X-ray films associated with fever and/or respiratory symptom; or hepatic or splenic sequestration, which is defined as a sudden increase in organ size associated with pain in the area of the organ, decrease in the hemoglobin concentration of ≥2 g/dL within a 24-h period, and, for liver sequestration, abnormal change in liver function tests, including conjugated bilirubin, not due to biliary tract disease.

The secondary outcome measures of the study include:
1. Proportion of subjects with mean HbF>20% (HbF/Hb) compared with pre-conditioning Baseline (time frame: up to 2 years post RNP27 edited CD34+ cell infusion);
2. Proportion of subjects with mean Hb≥10 g/dL starting ≥60 days after last packed red blood cell (pRBC) transfusion compared with preconditioning Baseline (time frame: up to 2 years post RNP27 edited CD34+ cell infusion);
3. Annualized number of units of pRBC transfused for SCD-related indications (time frame: up to 2 years post of the HBG1 and HBG2 promoters infusion);
4. Change from baseline in annualized rate of hospitalization for severe VOE (time frame: up to 2 years post RNP27 edited CD34+ cell infusion);
5. Change from baseline in annualized rate of severe VOE by at least 75% (time frame: up to 2 years post RNP27 edited CD34+ cell infusion);
6. Change from baseline in annualized rate of severe VOE by at least 90% (time frame: up to 2 years post RNP27 edited CD34+ cell infusion);
7. Complete resolution of severe VOE (time frame: up to 2 years post RNP27 edited CD34+ cell infusion).

Key inclusion criteria for subjects in the study include:
1. 12-50 years of age;
2. Diagnosis of severe sickle cell disease as defined by:
   Documented severe SCD genotype (β^{S}/β^{S}, β^{S}/β⁰, β^{S}/β⁺, β^{S}β^{C}, β^{S}β^{E}, β^{S}β^{D}, β^{S}β^{OArab})
   History of at least two severe vaso-occlusive crisis events per year requiring medical attention despite hydroxyurea or other supportive care measures in the two year-period prior to provision of informed consent;
3. Karnofsky Performance Status ≥80.

Exclusion criteria include:
1. Available 10/10 HLA-matched related donor;
2. Prior hematopoietic stem cell transplantation (HSCT) or contraindications to autologous HSCT;
3. Any contraindications to the use of plerixafor during the mobilization of hematopoietic stem cells (HSCs) and any contraindications to the use of busulfan and any other medicinal products required during the myeloablative conditioning, including hypersensitivity to the active substances or to any of the excipients;
4. Unable to receive red blood cell (RBC) transfusion for any reason;
5. Unable or unwilling to comply with standard of care changes in background medical treatment in preparation of, during, or following HSCT, including and not limited to discontinuation of hydroxyurea, voxelotor, crizanlizumab, or L-glutamine;
6. Any history of severe cerebral vasculopathy;
7. Inadequate end organ function;
8. Advanced liver disease;
9. Any prior or current malignancy or immunodeficiency disorder;
10. Immediate family member with a known or suspected Familial Cancer Syndrome; and
11. Clinically significant and active bacterial, viral, fungal, or parasitic infection.

Briefly, the overall study includes:
1. screening (obtaining consent and screening of subjects),
2. mobilization and apheresis (HSPC mobilization and apheresis),
3. drug product manufacturing (CD34+ cells edited at HBG1 and HBG2 promoters with RNP27),
4. myeloablation and RNP27 edited CD34+ cell infusion (busulfan myeloablation and RNP27 edited CD34+ cells drug product infusion), and
5. engraftment and 24 month follow-up (24 month follow-up for primary endpoint).

In the Phase 1/2/3 study, autologous CD34+ hematopoietic stem and progenitor cells are collected by apheresis after plerixafor mobilization and then edited at the HBG1/HBG2 promoter with RNP27. Briefly, leukopaks are obtained from SCD subjects mobilized with plerixafor from participating clinical sites. For SCD subjects, the use of G-CSF is contraindicated. To minimize potential complications, mobilization is done with plerixafor alone, which may reduce the yield of CD34+ cells. Subjects undergo as few leukapheresis cycles as needed to ensure a minimum post-thaw dose. CD34+ cells are enriched using the CliniMACS Plus system, aliquoted, cryopreserved in Cryostor CS10, and stored in liquid nitrogen vapor phase. CD34+ cells are thawed, cultured for 2 days in complete media consisting of X-VIVO 10, supplemented with 1×Glutamax, 100 ng/mL stem cell factor (SCF), 100 ng/mL thrombopoietin (TPO), and 100 ng/mL FMS-like tyrosine kinase 3 ligand (Flt3L), mixed with RNP (at a gRNA/protein molar ratio of 2.4) to a final concentration of 6 μM, and electroporated with a Maxcyte GT electroporation device per manufacturer's instruction. The cellular materials are cultured overnight following electroporation, aliquoted, and cryopreserved in Cryostor CS10, and stored in liquid nitrogen vapor phase until ready for infusion.

Following myeloablative conditioning with busulfan, subjects receive a single infusion of autologous RNP27 edited CD34+ cells (≥3×10⁶ CD34+ cells/kg): For example, Subject 1 received a dose of 10.0×10⁶ RNP27 edited CD34+ cells/kg, Subject 2 received a dose of 4.0×10⁶ RNP27 edited CD34+ cells/kg, Subject 3 received a dose of 4.1×10⁶ RNP27 edited CD34+ cells/kg, and Subject 4 received a dose of 3.7×10⁶ RNP27 edited CD34+ cells/kg. Subjects are monitored for engraftment, hematopoietic recovery, total hemoglobin, HbF production, percent (%) F-cells, markers of hemolysis, transfusion requirement, VOEs, and adverse events (AEs). Subjects will be monitored for a 24-month follow-up period.

Results for 18 participants of the phase 1/2/3 study are described herein.

Demographics of Subjects 1-18 are set forth in Table 6.

TABLE 6
Demographics of Subjects 1-18

| Demographics | | Total subjects (N = 18) |
|---|---|---|
| Genotype, n (%) | β^{S}/β^{S} | 17 (94.4) |
| | β^{S}/β⁰ | 1 (5.6) |

| | |
|---|---|
| Sex, n (%) | 9 Female (50.0) |
| Age, years, mean (SD) | 26.3 (4.9) |
| Severe VOEs, pre-study annual rate*, mean | 5.2 (2.9) |
| (SD) | |

SD = standard deviation
*the pre-study period is defined as the 2 year period prior to informed consent.

Demographics of Subjects 1-11 are set forth in Table 7.

TABLE 7
Demographics of Subjects 1-11

| Demographics | Total subjects (N = 11) |
|---|---|
| Genotype, n (%) | 11 (100) |
| β^{S}/β^{S} | |
| Sex, n (%) | 6 Female (54.5) |
| Age, years, mean (SD) | 27.6 (4.2) |

SD = standard deviation

Subjects 1-11 were homozygous for the HbS mutation and have a high pre-enrollment annual rate of VOEs. For example, Subjects 1 and 2 experienced 4 and 3 vaso-occlusive events, respectively, annually prior to enrollment in the trial. Subject 1 had >10 outpatient infusions, emergency department (ED) visits, or hospital admissions for pain crises in the first year prior to treatment and >30 outpatient infusions, ED visits, or hospital admissions for pain crisis in the second year prior to treatment. Subject 2 had ~10 outpatient infusions, ED visits, or hospital admissions for pain crises in the first year prior to treatment and >2 ED visits or hospital admissions for pain crises in the second year prior to treatment. At ten months post infusion in Subject 1 and six months post infusion in Subject 2, the modified cells had decreased sickle cell crises to zero.

Demographics of Subjects 1-4 are set forth in Table 8.

TABLE 8
Demographics of Subjects 1-4

| | Subject | Subject | Subject | Subject | Total subjects |
|---|---|---|---|---|---|
| Demographics | 1 | 2 | 3 | 4 | (N = 4) |
| Genotype | β^{S}/β^{S} | β^{S}/β^{S} | β^{S}/β^{S} | β^{S}/β^{S} | 4 |
| Gender | Male | Female | Female | Male | 2 Male/ |
| | | | | | 2 Female |
| Age, years, median | 25 | 31 | | | 27.5 (24-31) |
| (min, max) | | | | | |
| VOEs Pre-Study | 4 | 3 | | | 4.8 (3-6) |
| (average/year) | | | | | |
| LIC, mg/g of liver, | | | | | 4.2 (2.5-14.9) |
| median (min, max) | | | | | |

VOE = vaso-occlusive event
LIC = liver iron concentration

The infusion and engraftment results for Subjects 1-18 are shown in Table 9. All 18 subjects showed successful neutrophil and platelet engraftment after infusion with autologous RNP27 edited CD34+ cells. For 18 subjects, neutrophil and platelet engraftment were achieved within a median of 23.0 and 24.0 days of infusion, respectively (Table 9). All 18 subjects achieved neutrophil and platelet engraftment within ranges reported for other investigational gene therapies for SCD as well as allogenic HSCT (e.g., Walter 1996 (neutrophil engraftment [median (range)]: 18 days (15-32); platelet engraftment: definition >20,000, 26 days (11-40)); Bernaudin 2007 (neutrophil engraftment: 21.7 (7.7) days)).

TABLE 9
Infusion and Engraftment Results for Subjects 1-18

| Treatment | N = 18 |
|---|---|
| Total RNP27 edited CD34+ cells dose administered, ×10⁶ CD34+ cells/kg, | 4.6 (2.9, 10.0) |
| median (min, max) | |
| Follow-up duration, months, median (min, max) | 7.0 (2.4, 22.8) |
| Time to neutrophil engraftment*, days, median (min, max) | 23.0 (15.0, 29.0) |
| Time to platelet engraftment **, days, median (min, max) | 24.0 (18.0, 51.0) |

SD = Standard Deviation
*Three consecutive measurements with absolute neutrophil count (ANC) ≥0.5 × 10⁹/L.
** Three consecutive measurements with platelet count ≥50 × 10⁹/L starting at least 7 days after the platelet transfusion, and 10 days after thrombopoietin (TPO). No TPO was used for subjects after RNP27 edited CD34+ cell infusion.

The infusion and engraftment results for Subjects 1-11 are shown in Table 10. As shown in Table 10, all subjects who reached the time of the most recent data cutoff (n=10) all showed successful neutrophil and platelet engraftment after infusion with autologous RNP27 edited CD34+ cells. For 10 subjects, neutrophil and platelet engraftment were achieved within a mean of 23.7 and 24.3 days of infusion, respectively (Table 10). All 10 subjects achieved neutrophil and platelet engraftment within ranges reported for other investigational gene therapies for SCD as well as allogenic HSCT (e.g., Walter 1996 (neutrophil engraftment [median (range)]: 18 days (15-32); platelet engraftment: definition >20,000, 26 days (11-40)); Bernaudin 2007 (neutrophil engraftment: 21.7 (7.7) days)).

TABLE 10
Infusion and Engraftment Results for Subjects 1-11

| Treatment | N = 11 |
|---|---|
| Total RNP27 edited CD34+ cells dose administered (10⁶/kg) (SD) | 5.2 (2.5) |
| Time to neutrophil engraftment, days mean (SD) ^{&,} * | 23.7 (2.8) |
| Time to platelet engraftment, days, mean (SD) ^{&,} ** | 24.3 (5.6) |

SD = Standard Deviation
& For one subject who did not reach the one month visit, neither neutrophil engraftment nor platelet engraftment was reached by the time of data cutoff; engraftment values are therefore based on n = 10.
* Three consecutive measurements with absolute neutrophil count (ANC) ≥0.5 × 10⁹/L.
** Three consecutive measurements with platelet count ≥50 × 10⁹/L starting at least 7 days after the platelet transfusion, and 10 days after thrombopoietin (TPO).
SD = standard deviation

As shown in Table 11, Subjects 1-4 all showed successful neutrophil and platelet engraftment after infusion with autologous RNP27 edited CD34+ cells. For Subject 1, neutrophil and platelet engraftment were achieved within 23 and 19 days of infusion, respectively (Table 11). For Subject 2, neutrophil and platelet engraftment were achieved within 29 and 37 days of infusion, respectively (Table 11). For Subject 3, neutrophil and platelet engraftment were achieved within 23 and 23 days of infusion, respectively (Table 11). For Subject 4, neutrophil and platelet engraftment were achieved within 24 and 28 days of infusion, respectively (Table 11). All subjects achieved neutrophil and platelet engraftment within ranges reported for other investigational gene therapies for SCD as well as allogenic HSCT (e.g., Walter 1996 (neutrophil engraftment [median (range)]: 18 days (15-32); platelet engraftment: definition >20,000, 26 days (11-40)); Bernaudin 2007 (neutrophil engraftment: 21.7 (7.7) days)). Neutrophil and platelet engraftment were achieved after a mean (range) of 25 (23-29) and 27 (19-37) days in Subjects 1-4.

TABLE 11
Engraftment Results for Subjects 1-4

| Treatment | Subject 1 | Subject 2 | Subject 3 | Subject 4 |
|---|---|---|---|---|
| Total RNP27 edited | 10.0 | 4.0 | 4.1 | 3.7 |
| CD34+ cells (10⁶/kg) | | | | |
| Neutrophil | 23 | 29 | 23 | 24 |
| engraftment (day)* | | | | |
| Platelet engraftment | 19 | 37 | 23 | 28 |
| (day) ** | | | | |
| Follow-up Duration | 10 | 6 | 3 | 2 |
| (Months) | | | | |
| VOEs Post | None | None | None | None |
| Autologous RNP27 | | | | |
| Edited CD34+ cell | | | | |
| Infusion | | | | |

*3 consecutive measurements with absolute neutrophil count (ANC) ≥ 0.5 × 10⁹/L on different days
**3 consecutive measurements with platelet count ≥ 50 × 10⁹/L starting at least 7 days after the last platelet transfusion, and 10 days after thrombopoietin (TPO).

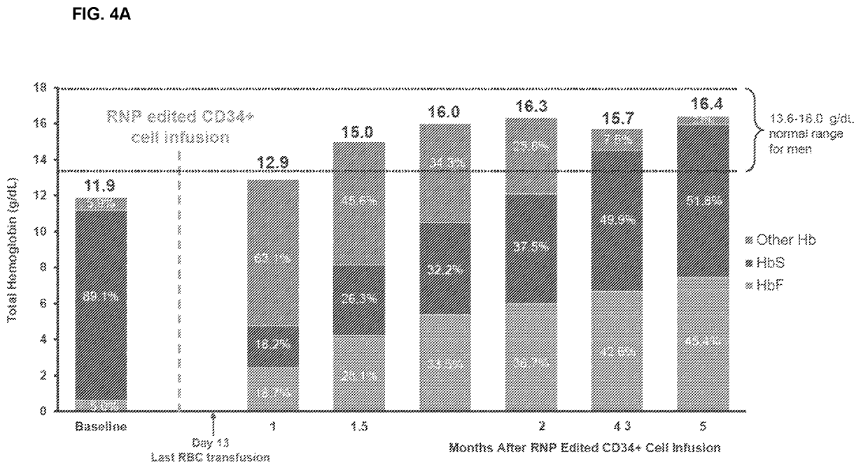

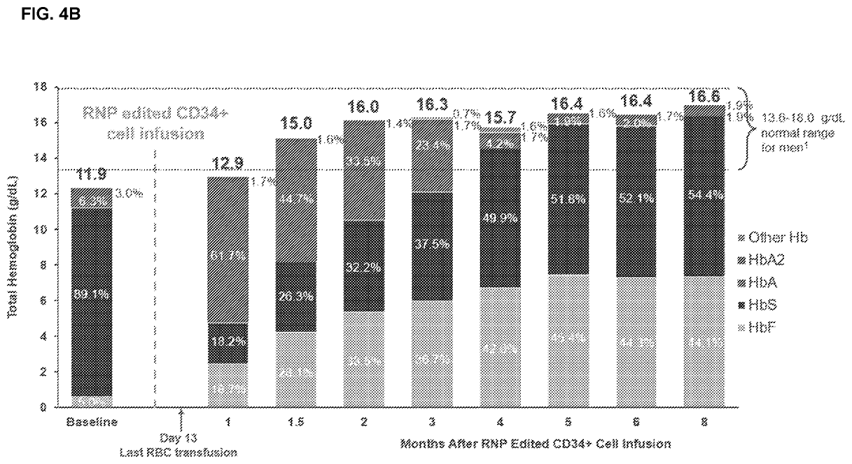

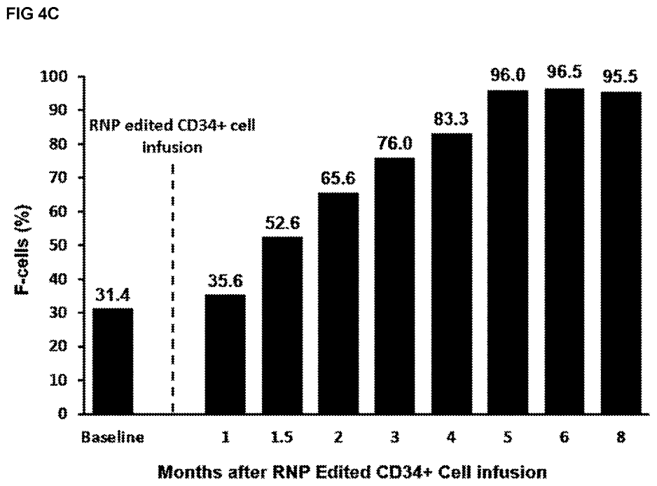

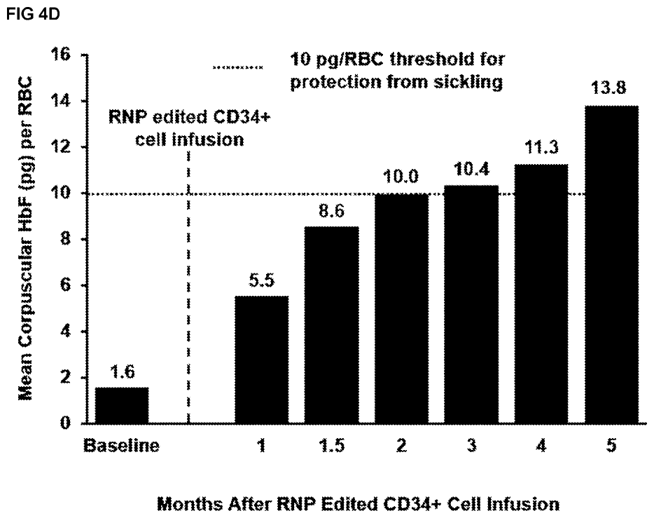

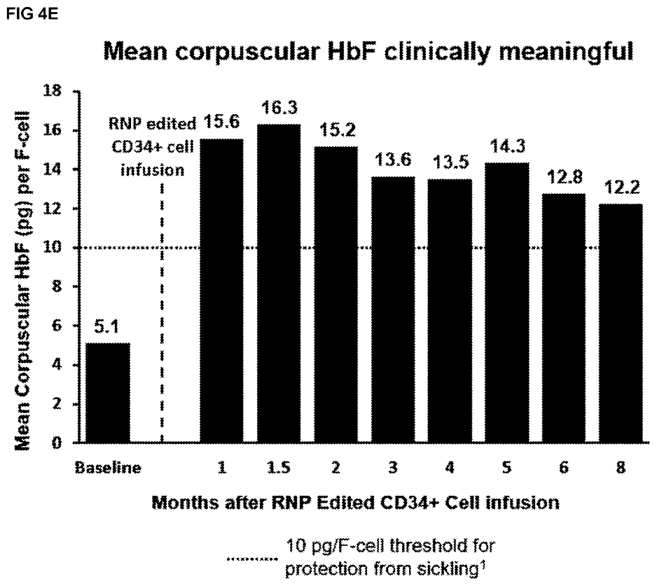

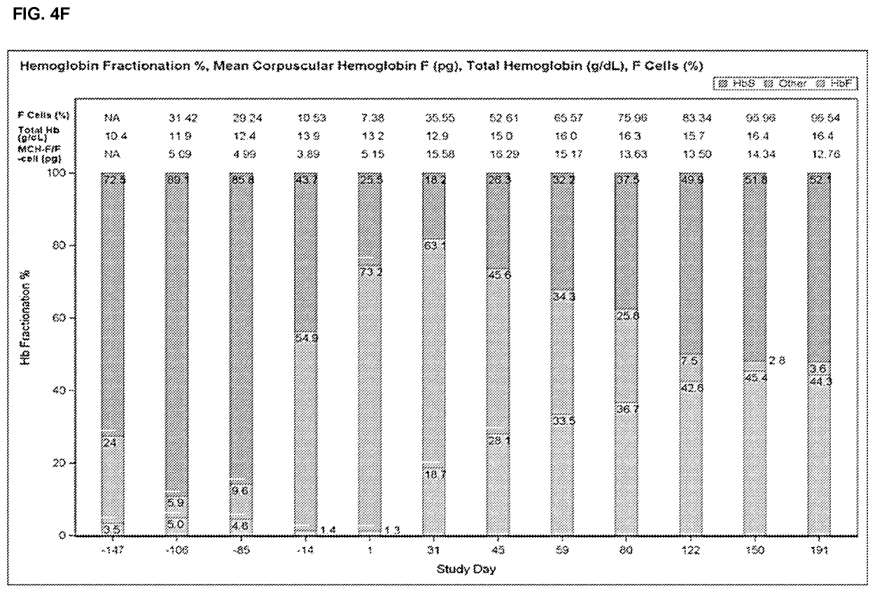

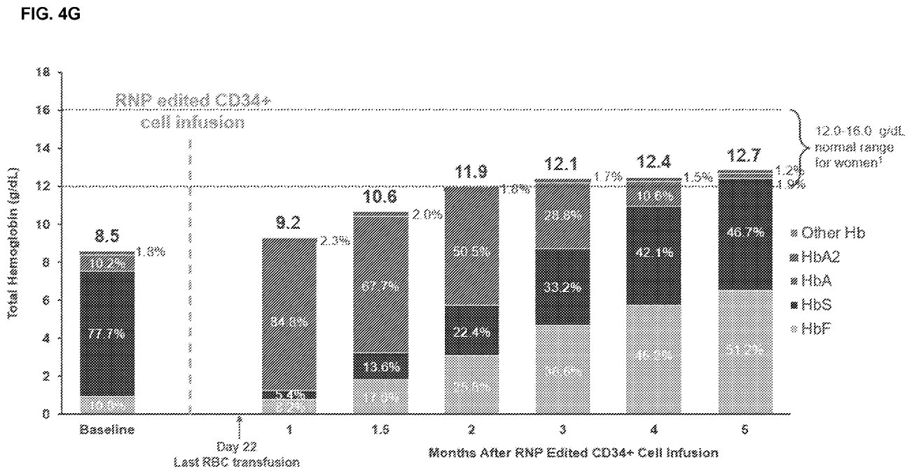

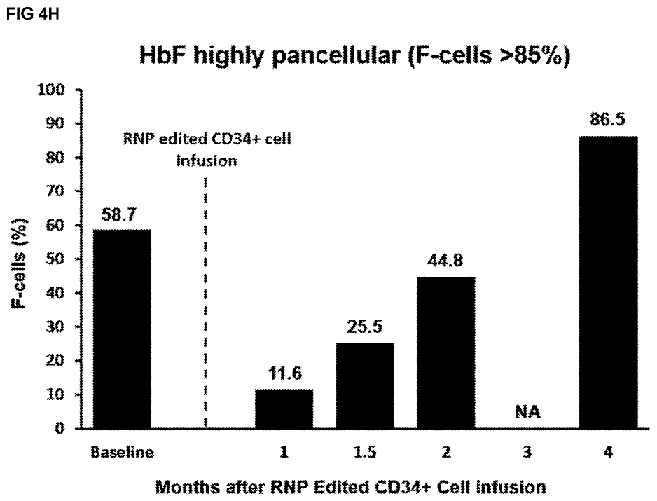

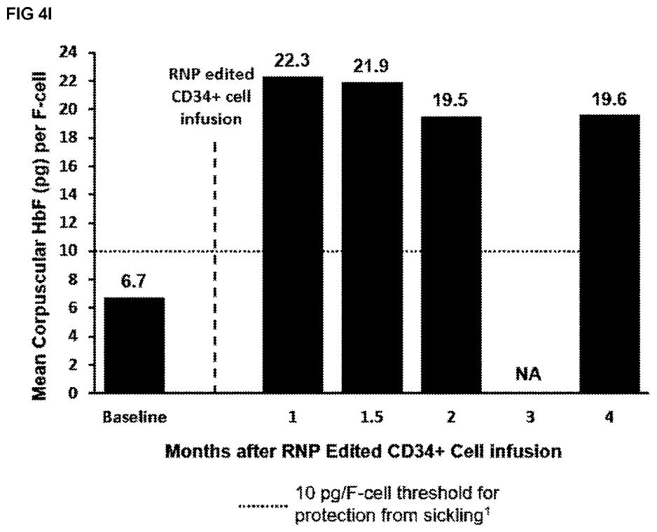

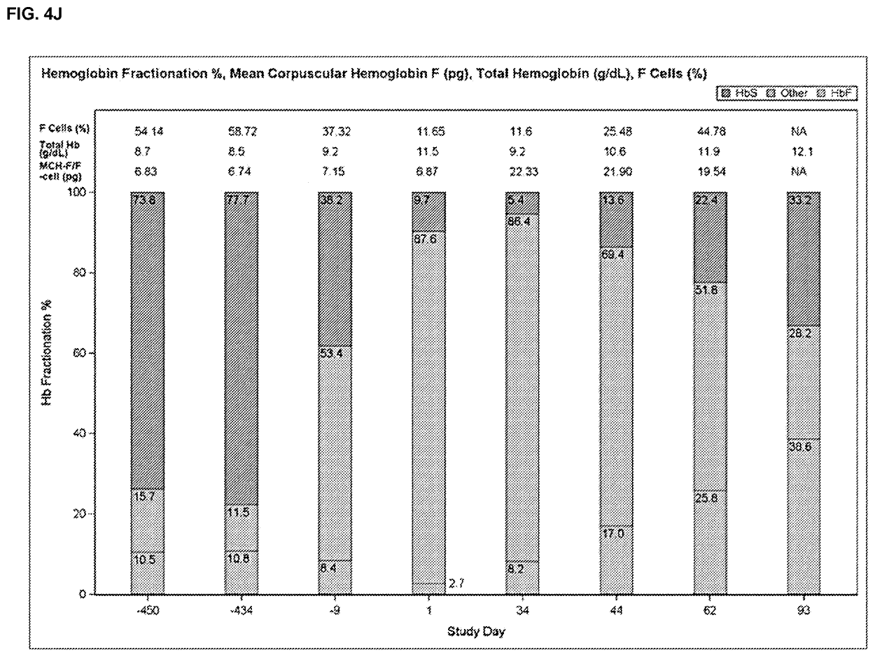

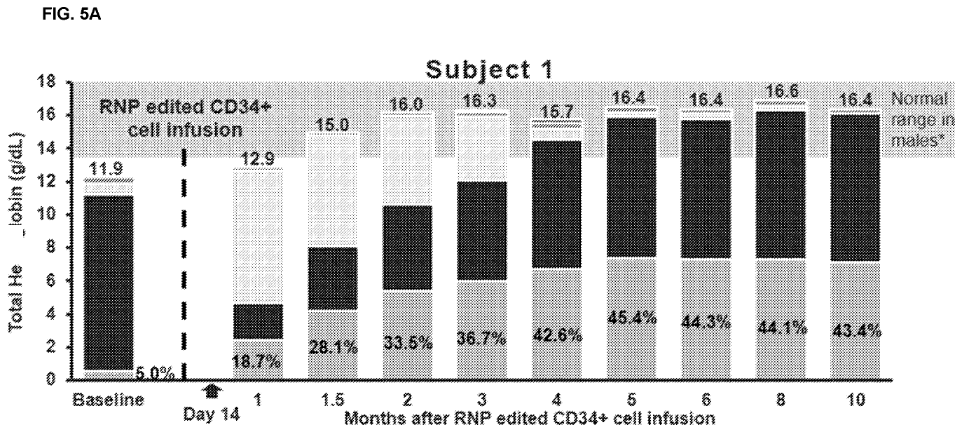

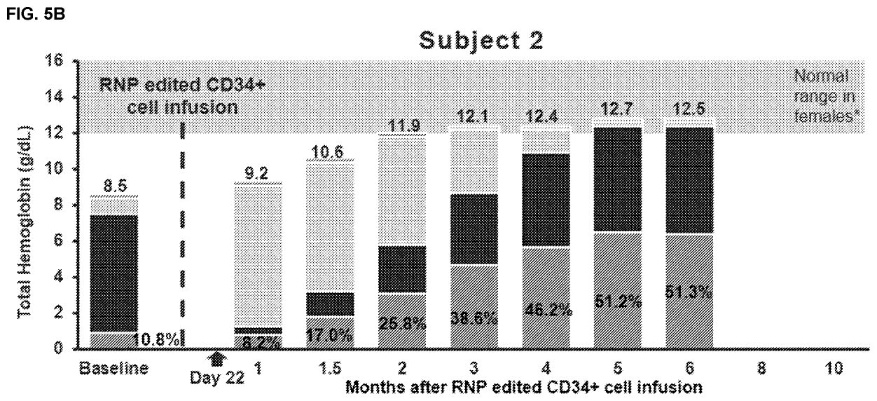

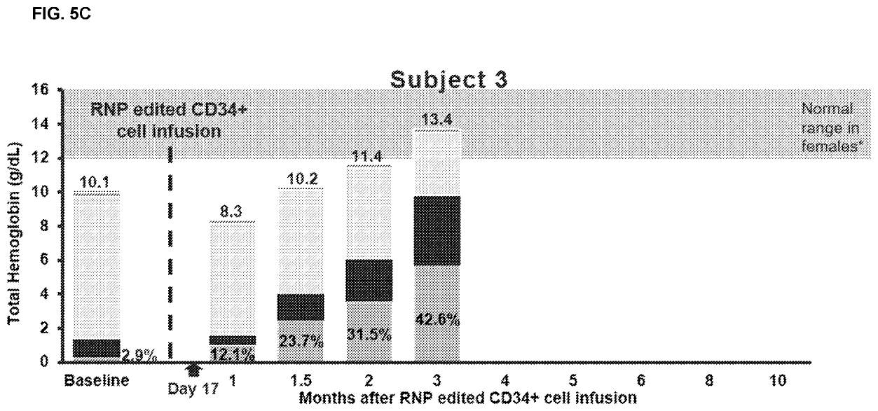

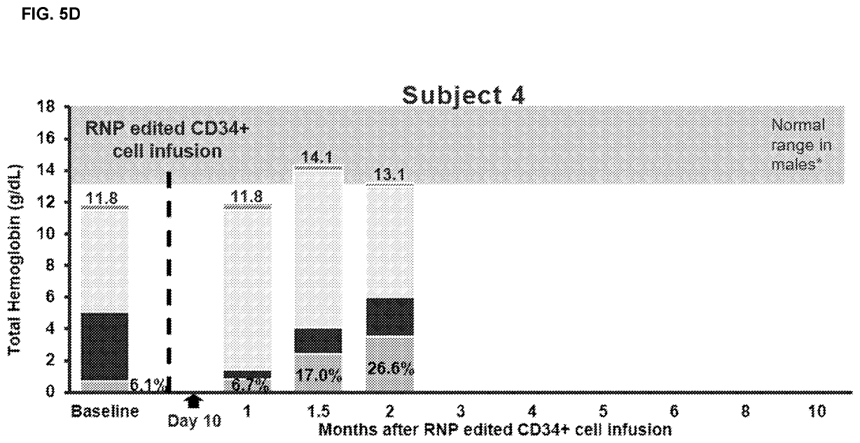

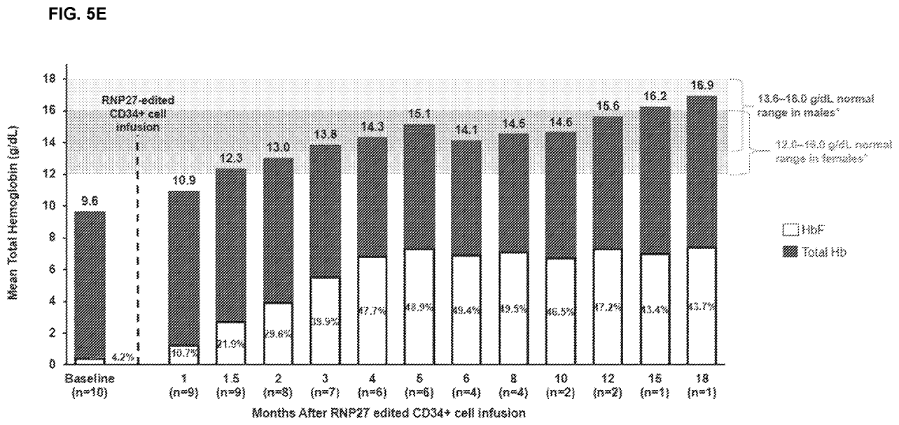

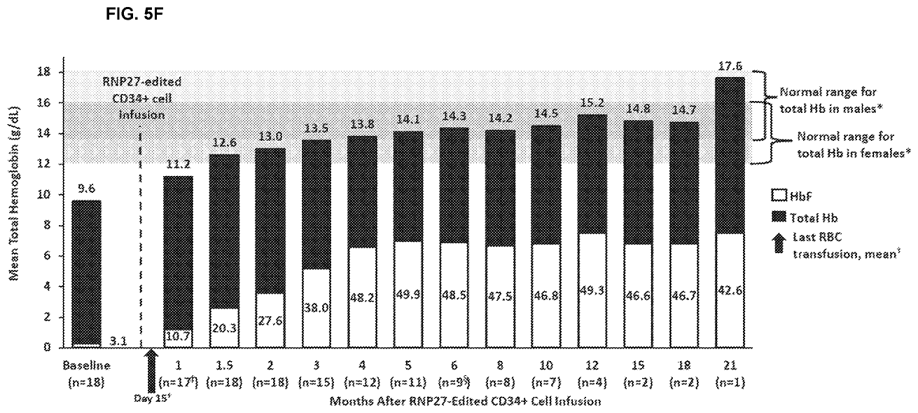

By the 3-month visit post-infusion with autologous RNP27 edited CD34+ cells, total hemoglobin for Subject 1 (male) had increased by 4.4 g/dL from 11.9 g/dL at baseline to a 16.3 g/dL (FIG. 4A), which is in a normal range (total hemoglobin is low in SCD patients with the range being around 6-11 g/dL for HbSS (genotype β^{S}/β^{S}) and normal range for males being around 13.6-18.0 g/dL and females around 12-16 g/dL). At the 5 month and 6 month follow-up visit total hemoglobin for Subject 1 was 16.4 g/dL, at the 8 month follow-up visit total hemoglobin for Subject 1 was 16.6 g/dL (FIGS. 4A and 4B), and at the 10 month follow-up visit total hemoglobin for Subject 1 was 16.4 g/dL (FIG. 5A).

For Subject 1, at the 3 months follow-up visit, HbF level and % F-cells were 36.7% and 76.0%, respectively, vs 5.0% and 31.4% at baseline (FIG. 4A, FIG. 4C). For Subject 1, at the 5 months follow-up visit, HbF level and % F-cells were 45.5% and 96.0%, respectively, vs 5.0% and 31.4% at baseline (FIG. 4A, FIG. 4C). At the 6 months follow-up visit, HbF level and % F-cells were 44.3% and 96.5% respectively and at the 8 months follow-up visit, HbF level and % F-cells were 44.1% and 95.5% respectively (FIG. 4A, FIG. 4C). To compare, the % HbF target is 30 for clinical benefit though lower levels can benefit the patient (Steinberg 2014). A high number of cells (F-cells=75.96%) had detectable levels of HbF at 3 months and by 5 months was highly pancellular (F-cells >95%). At 1 to 8 months post-infusion with autologous RNP27 edited CD34+ cells, the mean corpuscular HbF increase was clinically meaningful for SCD subjects as the mean corpuscular HbF reached above 10 pg/RBC (FIGS. 4D, 4E, 4F) (10 pg/RBC is the threshold for protection from sickling (Steinberg 2014)). FIG. 4F shows hemoglobin fractionation (%) data (HbF, HbS and other hemoglobin as a percentage of total hemoglobin), the percentage of F-Cells, total hemoglobin (g/dL), and MCH-F/F-cell (pg) for Subject 1 at various study days before and after autologous RNP27 edited CD34+ cell infusion. Results from Subject 1 show HbF expressed in >95% of RBCs with concentration above anti-sickling threshold (10 pg/RBC). The data for Subject 1 also indicate a robust increase of HbF, well above the sickling threshold, rapidly normalizing total hemoglobin, well into normal range.

For Subject 2, at 3 months post-infusion (Study Day 93), total hemoglobin was within normal range for the subject's age, and fetal hemoglobin was 38.6% compared to baseline 2.7% (FIG. 4G, 4J). FIG. 4J shows hemoglobin fractionation (%) data (HbF, HbS and other hemoglobin as a percentage of total hemoglobin), the percentage of F-Cells, total hemoglobin (g/dL), and MCH-F/F-cell (pg) for Subject 2 at various study days before and after autologous RNP27 edited CD34+ cell infusion. Fetal hemoglobin was expressed in >85% F-cells of RBCs with concentration above anti-sickling threshold (10 pg/RBC) (FIG. 4H). At 1 to 4 months post-infusion with autologous RNP27 edited CD34+ cells, the mean corpuscular HbF increase was clinically meaningful for SCD subjects as the mean corpuscular HbF reached above 10 pg/RBC (FIGS. 4I, 4J) (10 pg/RBC is the threshold for protection from sickling (Steinberg 2014)). Data for up to 6 months post-infusion with autologous RNP27 edited CD34+ cells for Subject 2 is shown in FIG. 5B. The data for Subject 2 indicate a robust increase of HbF, well above the sickling threshold, rapidly normalizing total hemoglobin, well into normal range.

Data for up to 3 and 2 months post-infusion with autologous RNP27 edited CD34+ cells for Subject 3 and Subject 4, respectively, are shown in FIGS. 5C and 5D, respectively.

Data for up to 18 months post-infusion with autologous RNP27 edited CD34+ cells for 10 subjects (5 male subjects, 5 female subjects) are shown in FIG. 5E. As shown in FIG. 5E, subjects with SCD show clinically meaningful improvements in HbF levels with total Hb returning to the normal range. Following infusion with autologous RNP27 edited CD34+ cells, mean Hb levels rapidly increased from baseline. On an individual subject level, total Hb returned to the normal range as early as 4 months after infusion with autologous RNP27 edited CD34+ cells (FIG. 5E). By Month 4, the mean (SD) HbF concentration increased to 14.3 g/dL and the mean percentage of HbF was >47% (n=6) and was sustained above 40% (FIG. 5E). All subjects achieved normal Hb levels by 5 months. Subjects had a mean MCH-F/F-cell above the anti-sickling threshold from Month 1 (n=8). The percentage of F-cells increased in treated subjects. An increasing percentage of F-cells indicates that more RBCs are protected from sickling for clinical benefit. Subjects showed pancellular expression of HbF in RBCs.

Data for up to 21 months post-infusion with autologous RNP27 edited CD34+ cells for 18 subjects (9 male subjects, 9 female subjects) are shown in FIG. 5F. Subjects had rapid and sustained clinically meaningful improvements in HbF (>40%), with early and durable normalization of Hb. Markers of hemolysis displayed a trend of improvement or have normalized in subjects administered RNP27-edited CD34+ cells.

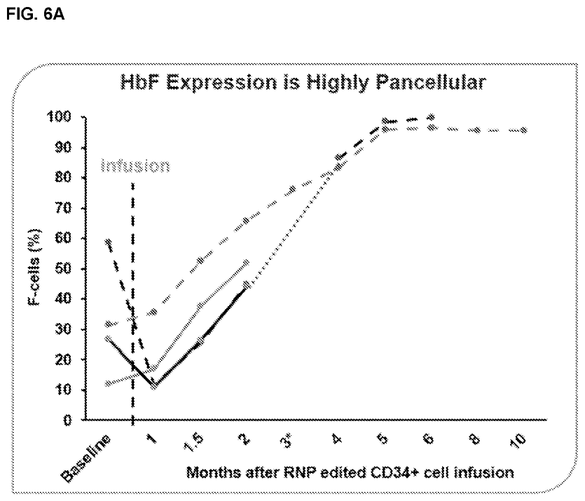

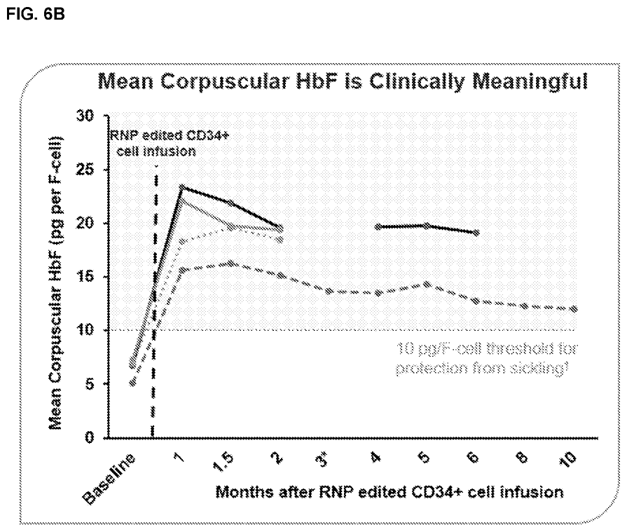

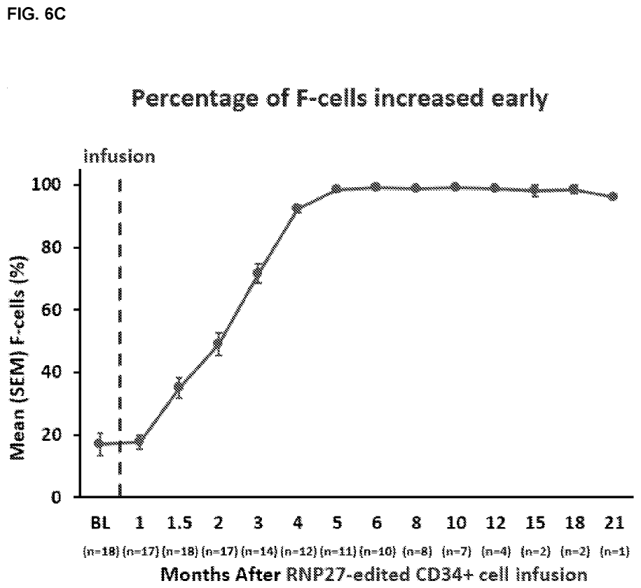

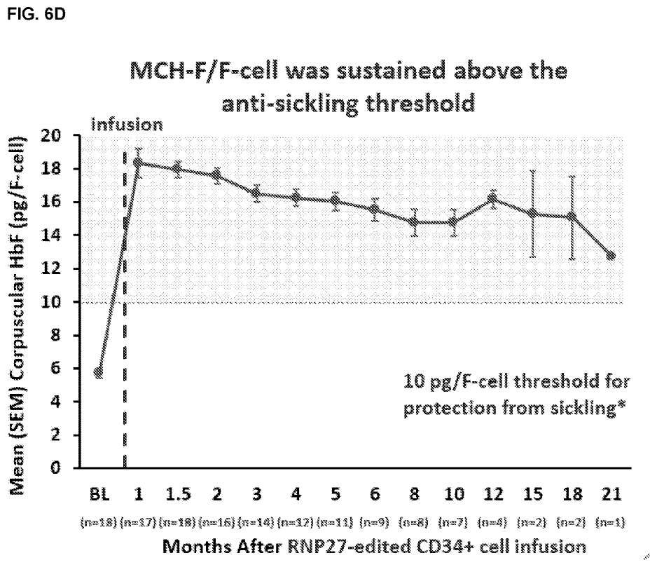

Data for up to 10 months indicate that subjects show pancellular expression of HbF in RBCs (see FIG. 6A for Subjects 1-4). Mean corpuscular HbF is depicted in FIG. 6B (for Subjects 1-4), and is above the 10 pg/RBC threshold for protection from sickling.

Data for up to 21 months indicate that subjects show pancellular distribution of HbF in RBCs (see FIG. 6C for Subjects 1-18). Mean corpuscular HbF is depicted in FIG. 6D (for Subjects 1-18), and is above the 10 pg/RBC threshold for protection from sickling. Subjects also showed sustained high levels of editing in the HBG1 and HBG2 promoter regions: at Month 6, mean (SD) editing levels were 75.7% (8.6%) in peripheral blood (n=8) and 87.6% (3.2%) in bone marrow (n=10), with high editing levels maintained at last follow-up.

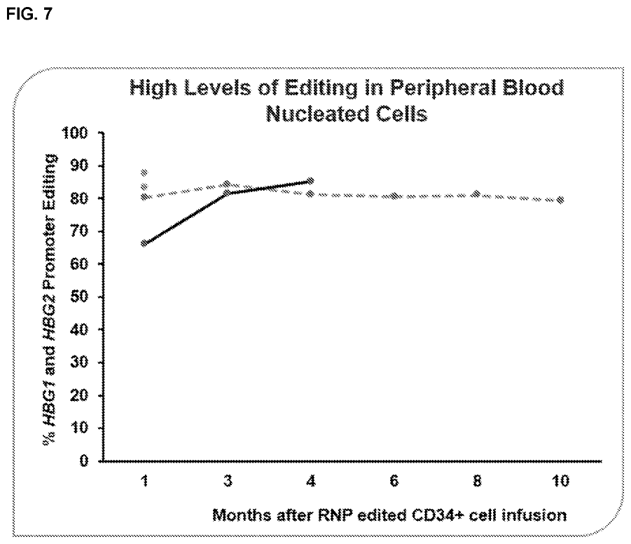

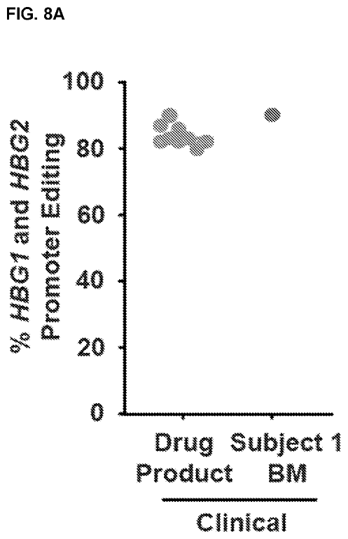

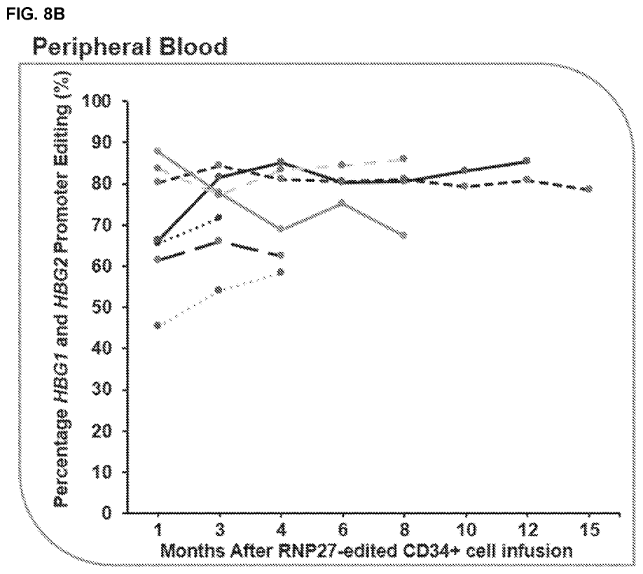

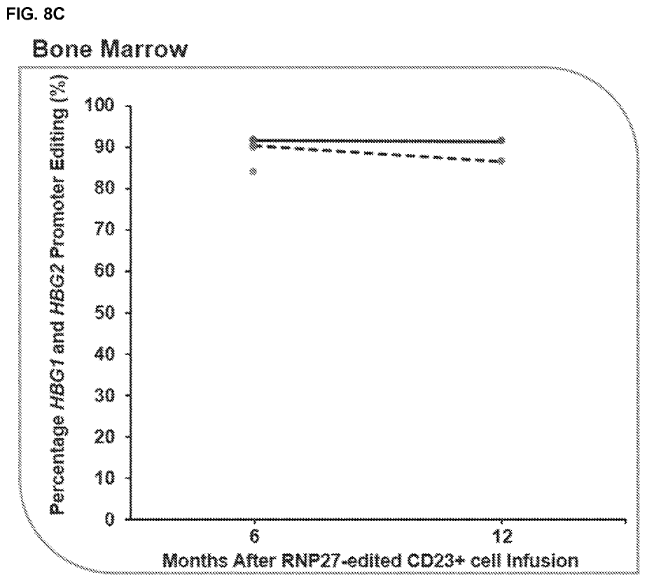

Results indicated that editing CD34+ cells using RNP27 resulted in ≥80% editing and no detectable off-target editing. High levels of HBG1 and HBG2 promoter editing in peripheral blood nucleated cells post RNP27 edited CD34+ cell infusion were observed, predicting durable clinical benefit (FIG. 7). Editing levels in peripheral blood nucleated cells were >80% in both Subjects 1 and 2. Editing levels in Subject 1 bone marrow were >80% (FIG. 8A). After infusion with RNP27 edited CD34+ cells, the level of editing in the HBG1 and HBG2 promoter regions was shown to remain high over time in both subject peripheral blood nucleated cells (FIG. 8B) and subject bone marrow-derived CD34+ cells (FIG. 8C).

Markers of hemolysis observed in Subjects 1 and 2 are set forth in Table 12. In Subject 1, lactate dehydrogenase, percent reticulocyte, and haptoglobin had normalized by three months post-treatment. Improvement was also observed in indirect bilirubin as seen in Table 12. At two months post-treatment follow-up for Subject 2, lactate dehydrogenase, reticulocyte count, and indirect bilirubin had declined to normal range. Although haptoglobin levels remained unchanged from baseline, the overall trend in hemolysis markers suggests a decline in hemolysis, which is further supported by a concurrent increase in total hemoglobin concentration (Table 12 and FIG. 4F (Subject 2, Total Hb) and FIG. 4J (Subject 2, Total Hb)). Key markers of hemolysis (reticulocyte count, indirect bilirubin, lactate dehydrogenase, and haptoglobin) displayed a trend of improvement or normalized in treated subjects (n=10, 5 male, 5 female).

TABLE 12
Hemolysis Markers for Subject 1 and Subject 2

| | Subject 1 | | | | | | | Subject 2 | | | |
|---|---|---|---|---|---|---|---|---|---|---|---|
| Laboratory | Normal | | | | | | | | | | |
| parameter | Range | BL | M1 | M1.5 | M2 | M3 | M6 | BL | M1 | M1.5 | M2 |
| Lactate | 113-226 | 360 | 232 | 247 | 222 | 192 | 161 | 378 | 167 | 156 | 162 |
| dehydrogenase | | | | | | | | | | | |
| (U/L) | | | | | | | | | | | |
| Indirect | 0.0-16.6 | 99.1 | 6.9 | 12.1 | 14 | 20.6 | 31.3 | 33.2 | 6.2 | 5.1 | 5.2 |
| bilirubin | | | | | | | | | | | |
| (μmol/L) | | | | | | | | | | | |
| Haptoglobin | 0.30-2.0 | <0.08 | 1.39 | 1.12 | 1.17 | 1.08 | 1.18 | <0.07 | 0.35 | 0.19 | 0.1 |
| (g/L) | | | | | | | | | | | |
| Reticulocytes | 0.3-2.3 | 8.2 | 6.3 | 3.4 | 2.7 | 2.2 | 1.6 | 10.3 | 7.5 | No | 1.8 |
| (%) | | | | | | | | | | result | |

BL = Baseline
M = Month

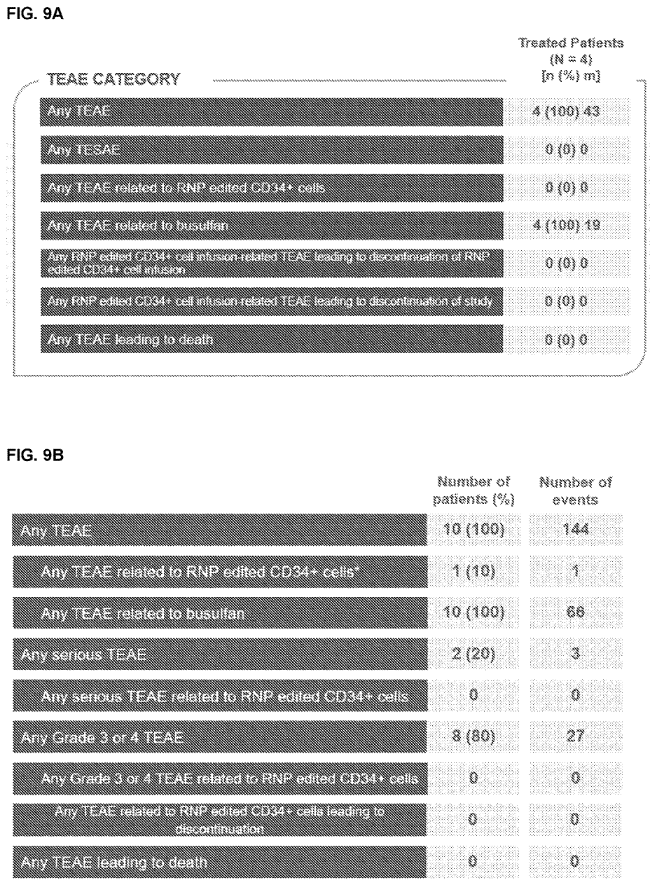

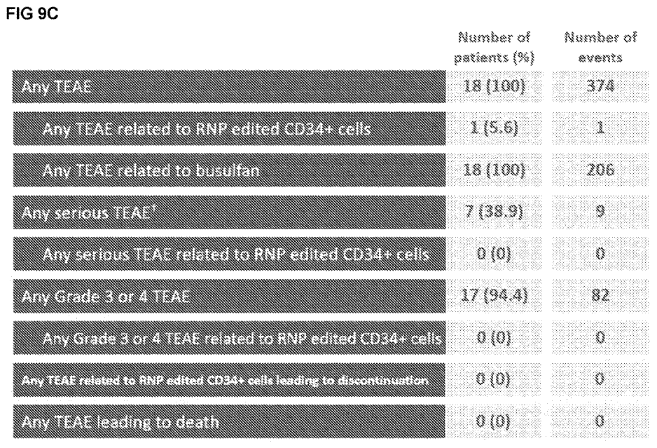

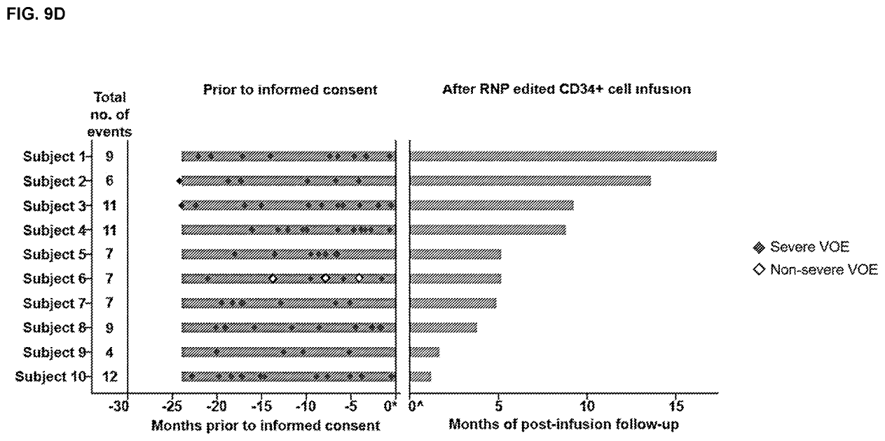

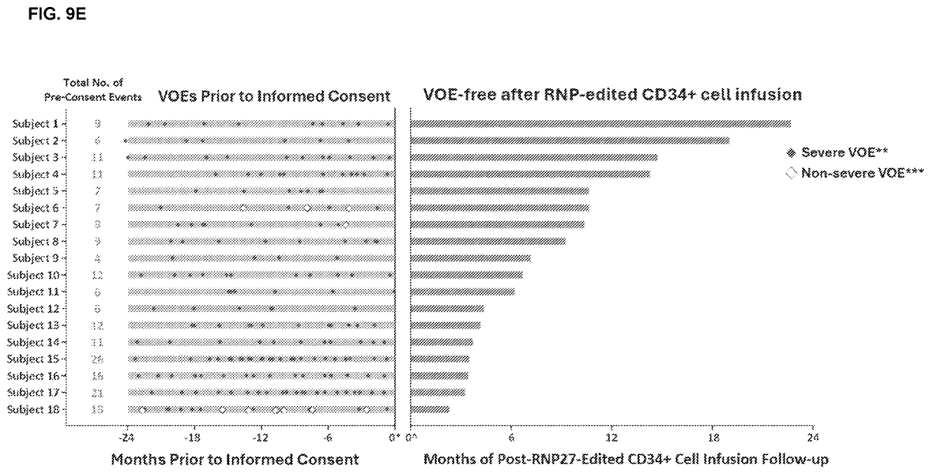

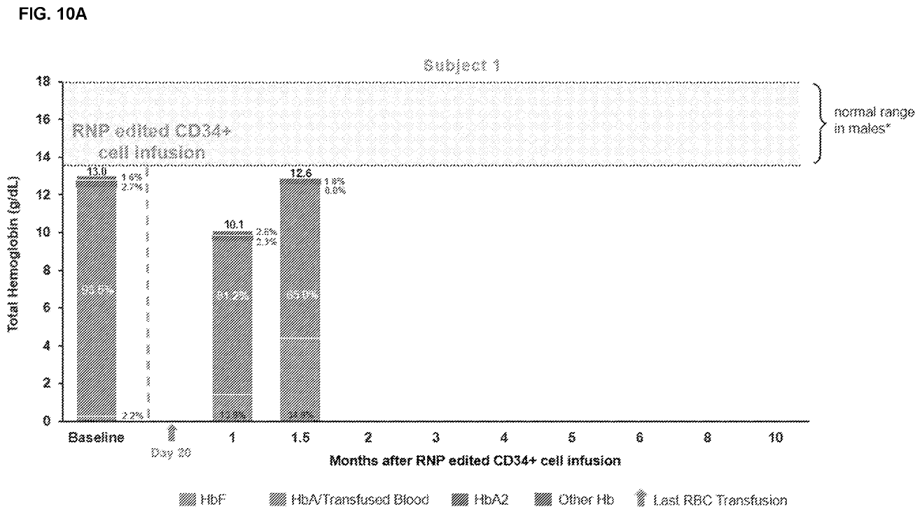

RNP27 edited CD34+ cell infusion was well tolerated by Subjects 1-10. Overall, the safety profile of RNP27 edited CD34+ cell infusion was consistent with that reported for HSCT myeloablative conditioning with busulfan (see, e.g., FIG. 9A for a prior data cut for Subjects 1-4 and FIG. 9B for the most recent data cut for Subjects 1-10). For Subjects 1-4, at a prior data cut, the majority of treatment-emergent adverse events (TEAEs) (E=26) occurred within the first 30 days after RNP27 edited CD34+ cell infusion. For Subjects 1-7, at a prior data cut, no SAEs occurred after RNP27 edited CD34+ cell treatment, and no AEs were reported as related to RNP27 edited CD34+ cell infusion. For Subjects 1-10 (FIG. 911), at a prior data cut, no serious TEAEs were reported as related to RNP27 edited CD34+ cell infusion and no TEAEs occurred after RNP27 edited CD34+ cell infusion. For Subjects 1-18 (FIG. 9C), at the most recent data cut, the safety profile of RNP27-edited CD34+ cell infusion is consistent with that of myeloablative conditioning with busulfan and HSCT. No serious TEAEs were reported as related to RNP27 edited CD34+ cell infusion.

For Subjects 1-10 who reached the Month 1 visit, no vaso-occlusive events (VOEs) were observed in subjects with SCD since infusion with RNP27 edited CD34+ cells (FIG. 9D). Compared with a mean of 4 severe VOEs/year in the 2 years before informed consent, all 10 subjects who reached the Month 1 visit have been VOE-free since infusion with RNP27 edited CD34+ cells. VOEs are the primary cause of morbidity and account for most emergency department visits and hospitalizations for patients with SCD in the United States and Europe, and as such are an important factor to address when treating patients with SCD.

In sum, Subjects 1-10 showed successful engraftment and exhibited increases in HbF (note that Subject 11 did not reach the 1 Month follow-up time point therefore n=10). Subjects 1-7 have had no VOEs since RNP27 edited CD34+ cell treatment during their respective post-infusion follow-ups versus a mean (range) of 4.2 (3.0-5.5) VOEs/year for the 2 years prior to study enrollment (n=6). For Subject 1, HbF reached >45.4% at Month 5 after RNP27 edited CD34+ cell infusion. Also for Subject 1, total hemoglobin reached 16.4 g/dL, F-cell pancellularity was 96%, and mean corpuscular HbF rose to 13.8 pg/RBC, exceeding the 10 pg/RBC threshold to suppress RBC sickling. Following infusion, Hb levels increased from a mean (range) of 10.5 (8.5-11.9) g/dL at baseline (n=5) to 14.2 (12.4-15.7) g/dL at Month 4 (n=4), reaching the normal physiological range. By Month 4, mean (range) HbF levels were 6.8 (5.7-7.6) g/dL (n=4), with Subjects 1-4 having >40% HbF and Subjects 3 and 4 having >50% HbF. Percentage of F-cells was >83% by Month 4 (n=3) and mean HbF concentration/F-cell (MCH-F/F-cell) was >10.0 pg/F-cell by Month 1 (n=4).

When Subjects 1-17 were evaluated, the subjects had a mean (standard deviation, SD) follow-up of 6.2 (5.8) months post-RNP27 edited CD34+ cell treatment; two of these subjects had >1 year follow-up. Neutrophil and platelet engraftment were achieved after a mean (SD) of 22.2 (3.8) and 25.2 (6.2) days (n=14), respectively. No subjects reported a VOE post-RNP27 edited CD34+ cell infusion, compared with a mean (SD) of 5.2 (3.0) severe VOEs/year in the 2 years before enrollment (n=17). Following RNP27 edited CD34+ cell infusion, mean (SD) Hb levels rapidly increased and were maintained at >14.4 (2.5) g/dL from Month 5 (n=8). Mean (SD) percentage of HbF was 48.0% (4.0) by Month 4 (n=7) and was sustained at >40% through the last follow-up. Percentage of F-cells and MCH-F/F-cell increased early and were maintained at >98% F-cells from Month 5 (n=8) and >15 pg/F-cell from Month 3 (n=7), respectively, which represent clinically significant targets. Key markers of hemolysis improved or normalized in all 17 subjects. The safety profile of RNP27 edited CD34+ cell treatment was consistent with myeloablative conditioning with busulfan. No AEs related to RNP27 edited CD34+ cell treatment were reported. Additionally, all 17 subjects were VOE-free post-RNP27 edited CD34+ cell infusion through the data cutoff date.

When Subjects 1-18 were evaluated, all subjects were VOE-free for up to 22.8 months post RNP27 edited CD34+ cell infusion (FIG. 9E). Subjects experienced early correction of anemia, with durable normalization of total Hb. Increases in HbF and the percentage of F-cells were sustained at >40% and >90%, respectively. Subjects also experienced improvements in markers of hemolysis.

These data demonstrate successful trilineage engraftment; a rapid and sustained increase in total Hb, including a rapid and sustained normalization of total Hb; an increase in HbF level and percentage of F-cells; improvements in key markers of hemolysis; resolution of VOEs; and a favorable safety profile in all subjects treated with autologous RNP27 edited CD34+ cells. Data from subjects with >1 month of follow up demonstrated early engraftment, a favorable safety profile, and increases in HbF levels and the percentage of F-cells.

### Example 2: Autologous CD34+ Cells Edited by RNP27 at the HBG1/HBG2 Promoter Shows Early, Promising Efficacy and Safety in a Phase 1/2 Study in Subjects with Transfusion-Dependent Beta-Thalassemia

To determine the safety, tolerability, and efficacy of treatment with autologous RNP27 edited CD34+ cells, a Phase 1/2, multicenter, open-label, single arm study is performed with subjects with transfusion-dependent beta-thalassemia (TDT). TDT is a hereditary blood disorder caused by reduced or absent production of D-globin. Clinical evidence has demonstrated that increased fetal hemoglobin (HbF) can lead to durable transfusion independence, reduced disease severity, and improved quality of life for patients with TDT. RNP27 (Table 18) comprises a gRNA comprising the sequence set forth in SEQ ID NO:237 complexed with a Cas12a protein comprising the sequence set forth in SEQ ID NO:283 (exemplary nucleotide sequence is SEQ ID NO:300). As shown in FIG. 3, RNP27 cleaves the genomic DNA in the distal CCAAT box region of γ-globin gene promoters (HBG1 and HBG2) on chromosome 11, resulting in reactivation of γ-globin expression and increased HbF production. In preclinical studies, editing of this genomic region at the HBG1 and HBG2 promoters with RNP27 in CD34+ cells from subjects with TDT led to improved erythropoiesis in vitro and erythroid progeny with increased total hemoglobin (Hb) production.

As described herein, autologous RNP27 edited CD34+ cells, administered as a one-time, autologous CD34+ cell therapy has the potential to lead to sustained, elevated levels of HbF in TDT subjects, ameliorating the disease phenotypes.

In the Phase 1/2, multicenter, open-label, single arm study, key inclusion criteria include the following: subjects are 18-35 years old with a diagnosis of TDT defined as (1) documented homozygous β-thalassemia or compound heterozygous β-thalassemia including β-thalassemia/hemoglobin E (HbE) based on historical data in medical records, and (2) history of at least 100 mL/kg/year or 10 U/year of packed red blood cell (RBC) transfusions in the 2 years prior to signing informed consent.

Key exclusion criteria include the following: available genetically-matched (10/10 HLA) related donor, previous or current malignancy or immunodeficiency disorder, and unable to tolerate stem cell therapy or receive RBC transfusion.

Primary endpoints in the study include (1) proportion of subjects achieving neutrophil engraftment (defined as demonstrating absolute neutrophil count (ANC) ≥0.5×10\hat{ }9/L post infusion for 3 consecutive measurements obtained on different days) on or by 42 days post RNP27 infusion, and (2) safety and tolerability of treatment with RNP27 edited CD34+ cells (frequency and severity of adverse events (AEs)).

In the Phase 1/2 study set forth herein in Example 2, autologous CD34⁺ hematopoietic stem and progenitor cells are collected by apheresis after mobilization with a combination of G-CSF (filgrastim) and plerixafor, and then edited at the HBG1/HBG2 promoter with RNP27.

Following myeloablative conditioning with busulfan, TDT subjects received a single infusion of autologous RNP27 edited CD34+ cells (≥3×10⁶ CD34+ cells/kg). Subjects will be monitored for a 24-month follow-up period.

Results for Subjects 1-7 of the TDT phase 1/2 study are described herein.

The demographics for Subjects 1-7 of the TDT phase 1/2 study are described in Table 13. The mean age of the seven treated subjects was 19.6 years and 57% of them were female. 43% of the subjects had the β⁰/β⁰ or β⁰/β⁰-like genotype and the rest were non-β⁰/β⁰.

TABLE 13
Demographics of Subjects 1-7

| Demographics | N = 7 |
|---|---|
| Genotype, n (%) | |
| β⁰/β⁰ or β⁰/β⁰-like* | 3 (42.9) |
| Non-β⁰/β⁰** | 4 (57.1) |
| Sex, n (%) | |
| Female | 4 (57.1) |
| Age, years, median (min, max) | 19.0 (18.0, 24.0) |
| Packed RBC transfusions, pre-study | 153.5 (52.9) |
| annual rate^{#}, mL/kg/year, mean (SD) | |

*β⁰/β⁰-like includes IVS-I-110/ IVS-I-110 (n = 1).
**Non-β⁰/β⁰ includes β⁰/β⁺ (n = 3) and β^{E}/β⁰ (n = 1).
^{#}The pre-study period is defined as the 2-year period prior to informed consent.
IVS, intervening sequence;
RBC, red blood cell;
SD, standard deviation.

The demographics for Subject 1 of the TDT phase 1/2 study are described in Table 14. Subject 1 had a non-β⁰/β⁰ genotype.

TABLE 14
Demographics of Subject 1

| Demographics | Subject 1 |
|---|---|
| Genotype | β⁰/β⁺ |
| Gender | Male |
| Age, years, median (min, max) | 18 |
| Packed RBC transfusions (average/year) | 13.5 |

Subject 1 received 27 units of packed RBCs in the 2 years prior to enrollment in the TDT phase ½ study.

The infusion and engraftment for Subjects 1-7 are shown in Table 15. As shown in Table 15, the average dose of autologous RNP27 edited CD34+ cells infused into these subjects was 6.7×10⁶ CD34+ cells/kg. At the time of data cutoff, the average follow-up duration post-autologous RNP27 edited CD34+ cell infusion was about 8.8 months. All seven subjects achieved neutrophil engraftment, with the average time to engraftment being about 23 days. All seven subjects also achieved platelet engraftment by the time of the data cut off. Average time to platelet engraftment for these 7 subjects was 38 days (Table 15). At Month 6, mean (SD) editing levels were 75.4% (5.3%) in subject peripheral blood nucleated cells (n=6) and 79.9% (9.1%) in subject bone marrow-derived CD34+ cells (n=4).

TABLE 15
Infusion and Engraftment for Subjects 1-7

| Treatment | N = 7 |
|---|---|
| No. of apheresis cycles, median (min, max) | 1.0 (1.0, 1.0) |
| Total RNP27 edited CD34+ cells (10⁶/kg), | 6.7 (5.7, 11.9) |
| median (min, max) | |
| Follow-up Duration, Months, (min, max) | 8.8 (4.7, 13.4) |
| Time to neutrophil engraftment*, days, | 23.0 (16.0, 30.0) |
| median (min, max) | |
| Time to platelet engraftment**, days, | 38.0 (24.0, 49.0) |
| median (min, max) | |

*= Three consecutive measurements with absolute neutrophil count (ANC) ≥0.5 × 10⁹/L.
**= Three consecutive measurements with platelet count ≥20 × 10⁹/L starting at least 7 days after the platelet transfusion, and 10 days after thrombopoietin (TPO).

As shown in Table 16, Subject 1 received a dose of 6.1×10⁶ RNP27 edited CD34+ cells/kg. Subject 1 experienced early successful engraftment, similar to Subjects 1-10 in the SCD phase 1/2/3 study in Example 1. The safety profile for Subject 1 was also similar to that seen with Subjects 1-10 seen with the SCD study in Example 1. At 1 month after treatment, no severe adverse events (SAEs) occurred after RNP27 edited CD34+ cell infusion and no adverse events (AEs) were reported to be related to RNP27 edited CD34+ cell infusion.

TABLE 16
Engraftment Results for Subject 1

| Treatment | Subject 1 |
|---|---|
| Total RNP27 edited CD34+ cells (106/kg) | 6.1 |
| Neutrophil engraftment (day)* | 23 |
| Platelet engraftment (day) ** | 26 |
| Follow-up Duration (Months) | 1 |

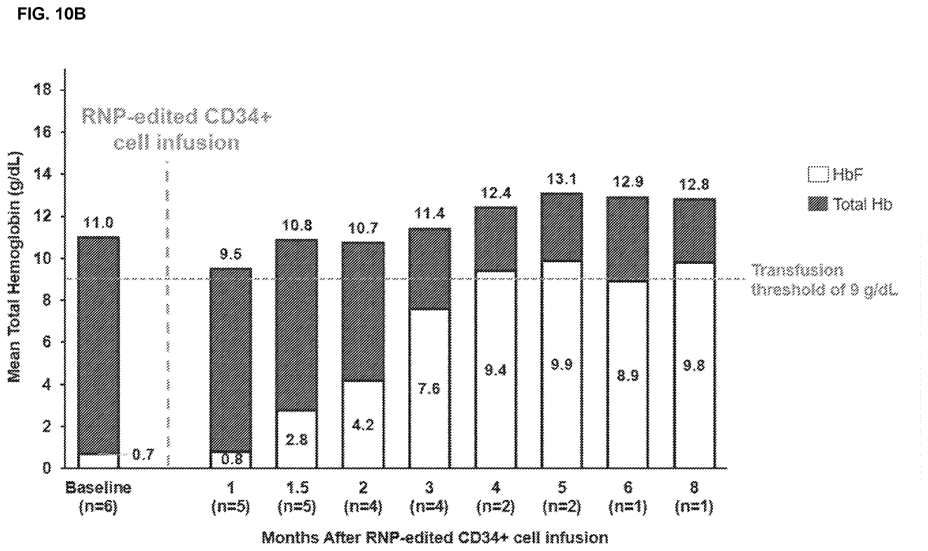

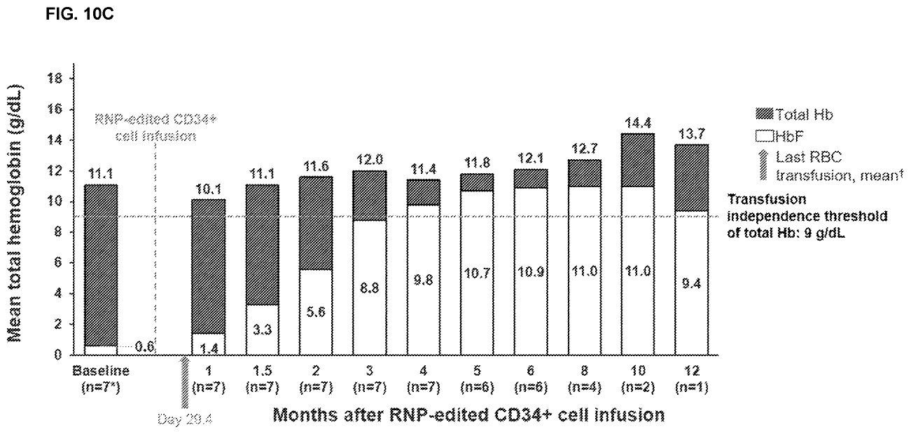

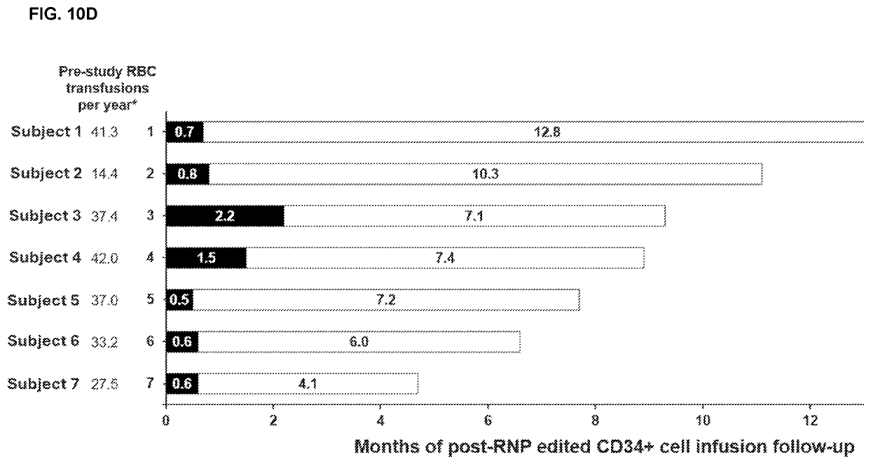

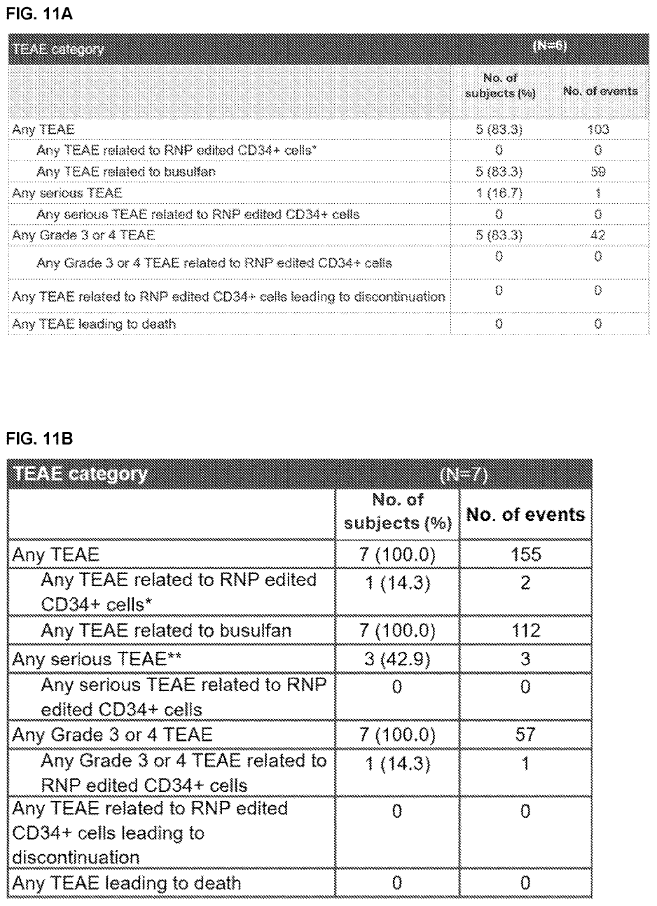

By the 1.5-month visit post-infusion with autologous RNP27 edited CD34+ cells, Subject 1 followed a similar trajectory expressing >30% HbF (>4 g/dL) as those subjects treated in the SCD phase 1/2/3 study in Example 1 (FIG. 10A). By the 3 month visit post-infusion, Subject 1 had 7.2 g/dL HbF, stopped receiving RBC transfusions 20 days after RNP27 edited CD34+ cell infusion, and remained transfusion free through the 3 month period. Subject 2 at less than 1 month post-infusion, also showed early improvements,

FIG. 10B shows results for Subjects 1-6 at 0-8 months following treatment. As shown in FIG. 10B, the five subjects with TDT treated with autologous RNP27 edited CD34+ cells for which data was available up to a maximum of 8 months show increased HbF levels with total HbF above the transfusion threshold of 9 g/dL. Following infusion with autologous RNP27 edited CD34+ cells, mean (SD) Hb levels rapidly increased to 13.1 (3.5) g/dL by Month 5 (n=2), from a mean (SD) of 11.0 (1.9) g/dL at baseline (n=6). All subjects who have >1 month follow-up (5/5) have stopped RBC transfusions after a mean of 1.2 months after infusion and remained transfusion free for a mean of 3.6 months (range of 1.8-7.5 months). Following infusion, mean total Hb level increased; all subjects maintained Hb levels above the transfusion threshold at last follow-up (FIG. 10B).

By Month 6, mean (SD) total Hb remained above the transfusion-independence threshold of 9.0 g/dL, and increased to 12.8 (1.0) g/dL (n=3). The mean (SD) HbF concentration increased early and was 8.9 (2.6) g/dL by Month 3 with pancellular distribution (n=6).

FIGS. 10C and 10D show results for Subjects 1-7 at 1-12 months following treatment. As shown in FIG. 10C, the seven subjects with TDT treated with autologous RNP27 edited CD34+ cells for which data was available up to a maximum of 12 months show increased HbF levels with total HbF above the transfusion threshold of 9 g/dL. All subjects maintained Hb levels above the transfusion threshold at last follow-up. The mean (SD) HbF concentration increased early and was 10.9 (1.5) g/dL by Month 6 (n=6). The mean (SD) percentage of F-cells was 99.2% (0.8%) by Month 6 (n=5).

As shown in FIG. 10D, after receiving the last RBC transfusion at 0.5-2.2 months post RNP27 edited CD34+ cells infusion, all seven subjects with TDT were transfusion free for a range of 4.1-12.8 months.

FIG. 11A shows results for Subjects 1-6 at 0-8 months following treatment. FIG. 11A shows that infusion with autologous RNP27 edited CD34+ cells was generally well tolerated by the 6 dosed subjects with TDT. Overall, the safety profile of RNP27 edited CD34+ cells in dosed subjects with TDT has been consistent with myeloablative conditioning with busulfan and autologous hematopoietic stem cell transplantation (n=7). No serious TEAEs related to RNP27 edited CD34+ cell infusion were reported after RNP27 edited CD34+ cell infusion through the data cutoff date. After receiving the last RBC transfusion at 0.5-2.2 months post-autologous RNP27 edited CD34+ cell infusion, all 7 subjects were transfusion independent for a range of 1.2-9.9 months.

FIG. 11B shows results for Subjects 1-7 at 1-12 months following treatment. As shown in FIG. 11B, the safety profile of RNP27 edited CD34+ cell infusion in subjects with TDT was consistent with myeloablative conditioning with busulfan and autologous HSCT. No serious TEAEs related to RNP27 edited CD34+ cell infusion were reported.

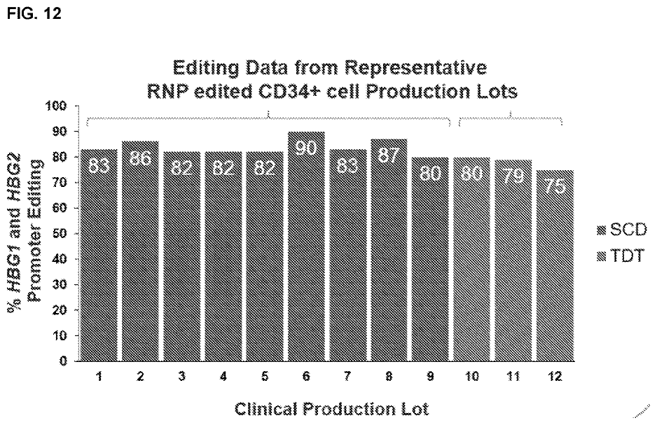

As shown in FIG. 12, the consistent levels of ex vivo editing correlated with similar clinical responses in dosed subjects for subjects in the SCD phase 1/2/3 and TDT phase 1/2 studies in Examples 1 and 2, respectively. Similar editing levels were achieved in cells from additional subjects, predicting similar and robust clinical responses and no detectable off target editing (FIG. 12).

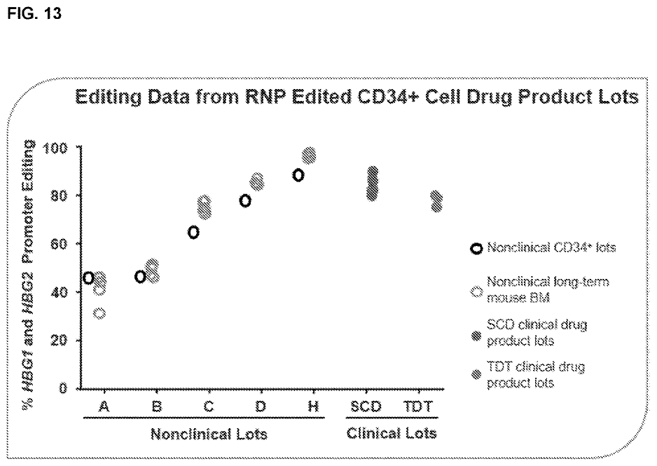

FIG. 13 demonstrates that RNP27 edited CD34+ cell drug product lots are efficiently edited. Results indicated a consistent production of RNP27 edited CD34+ cells (≥80% editing in lots from SCD subjects in Example 1 and >75% editing in lots from TDT subjects in Example 2). Based on the nonclinical data, efficient editing is predicted to lead to durable high levels of editing in subject bone marrow (BM) and sustained clinical benefits.

In sum, RNP27 edited CD34+ cells, the first investigational AsCas12a gene-edited therapy, showed promising results for gene editing of the γ-globin gene (HBG1 and HBG2) promoters to induce HbF expression in subjects with TDT. All subjects maintained Hb levels above the transfusion threshold and have been transfusion free for up to 12.8 months after RNP27 edited CD34+ cell infusion. All subjects experienced early increases in HbF, with sustained HbF levels at or near the transfusion threshold from 6 months onward. These data demonstrate successful engraftment and a safety profile that is consistent with myeloablative busulfan conditioning and autologous hematopoietic stem cell transplantation. These findings from treated subjects with TDT of longer duration build on strong clinical evidence that support the ongoing investigation of the phase 1/2 clinical trial.

### Example 3: Treatment of β-Hemoglobinopathy Using Edited Hematopoietic Stem Cells

The methods and genome editing systems disclosed herein may be used for the treatment of a β-hemoglobinopathy, such as sickle cell disease or beta-thalassemia, in a subject in need thereof. In certain embodiments, the beta-thalassemia may be transfusion-dependent beta-thalassemia (TDT). In certain embodiments, genome editing may be performed on cells derived from the subject in an autologous procedure. Correction of the subject's cells ex-vivo and reintroduction of the cells into the subject may result in increased HbF expression and treatment of the β-hemoglobinopathy.

For example, HSCs may be extracted from the bone marrow of a subject with a β-hemoglobinopathy using techniques that are well-known to skilled artisans. The HSCs may be modified using methods disclosed herein for genome editing. For example, RNPs comprised of guide RNAs (gRNA) that target one or more regions in the HBG gene complexed with an RNA-guided nuclease may be used to edit the HSCs. In certain embodiments, the RNA-guided nuclease may be a Cas12a protein. In certain embodiments, the Cas12a protein may comprise a sequence set forth in SEQ ID NOs:200, 201, 205-215, 221, 222-226, 280-283, 293-295, 335 (Cas12a polypeptide sequences). In certain embodiments, the Cas12a protein may be encoded by a sequence comprising SEQ ID NOs:216-218, 296-303, 336 (Cas12a polynucleotide sequences). For example, the Cas12a protein may comprise the sequence set forth in SEQ ID NO:283. In certain embodiments, the Cas12a protein may comprise the sequence set forth in SEQ ID NO:335. In certain embodiments, the gRNA may be a modified or unmodified gRNA. In certain embodiments, the gRNA may comprise a sequence set forth in Table 20. For example, in certain embodiments, the gRNA may comprise the sequence set forth in SEQ ID NO:237. In certain embodiments, the gRNA may comprise a targeting domain sequence set forth in Table 20. For example, in certain embodiments, the gRNA may comprise a targeting domain sequence set forth in SEQ ID NO:202. In certain embodiments, the gRNA may comprise a targeting domain sequence set forth in SEQ ID NO:320. In certain embodiments, the gRNA may comprise a DNA extension sequence set forth in Table 19. For example, in certain embodiments, the gRNA may comprise a DNA extension sequence set forth in SEQ ID NO:306. In certain embodiments, the RNP complex may comprise an RNP complex set forth in Table 18. For example, the RNP complex may include a gRNA comprising the sequence set forth in SEQ ID NO:237 and a Cas12a protein comprising the sequence set forth in SEQ ID NO:283 (RNP27, Table 18). In certain embodiments, the RNP complex may include a gRNA comprising the sequence set forth in SEQ ID NO:237 and a Cas12a protein comprising the sequence set forth in SEQ ID NO:335. In certain embodiments, modified HSCs have an increase in the frequency or level of an indel in the human HBG1 gene, HBG2 gene, or both, relative to unmodified HSCs. In certain embodiments, the modified HSCs can differentiate into erythroid cells that express an increased level of HbF. A population of the modified HSCs may be selected for reintroduction into the subject via transfusion or other methods known to skilled artisans. The population of modified HSCs for reintroduction may be selected based on, for example, increased HbF expression of the erythroid progeny of the modified HSCs or increased indel frequency of the modified HSCs. In some embodiments, any form of ablation prior to reintroduction of the cells may be used to enhance engraftment of the modified HSCs. In other embodiments, peripheral blood stem cells (PBSCs) can be extracted from a subject with a β-hemoglobinopathy using techniques that are well-known to skilled artisans (e.g., apheresis or leukapheresis) and stem cells can be removed from the PBSCs. The genome editing methods described above can be performed on the stem cells and the modified stem cells can be reintroduced into the subject as described above.

TABLE 17
Cas12a Molecules

| | Cas12a Amino Acid | Cas12a Nucleotide SEQ |
|---|---|---|
| Cas12a ID | SEQ ID NO* | ID NO* |
| His-AsCas12a-sNLS- | SEQ ID NO: 221 | SEQ ID NO: 296 |
| SNLS H800A | | |
| Cas12a-1 | SEQ ID NO: 280 | SEQ ID NO: 297 |
| Cas12a-2 | SEQ ID NO: 281 | SEQ ID NO: 298 |
| Cas12a-3 | SEQ ID NO: 282 | SEQ ID NO: 299 |
| Cas12a-4 | SEQ ID NO: 283 | SEQ ID NO: 300 |
| Cas12a-5 | SEQ ID NO: 293 | SEQ ID NO: 301 |
| Cas12a-6 | SEQ ID NO: 294 | SEQ ID NO: 302 |
| Cas12a-7 | SEQ ID NO: 295 | SEQ ID NO: 303 |

*See FIG. 14

TABLE 18
RNP complexes

| RNP Name | gRNA SEQ ID NO* | Cas12a Amino Acid SEQ ID NO** |
|---|---|---|
| RNP1 | SEQ ID NO: 219 | SEQ ID NO: 295 |
| RNP2 | SEQ ID NO: 237 | SEQ ID NO: 281 |
| RNP3 | SEQ ID NO: 284 | SEQ ID NO: 280 |
| RNP4 | SEQ ID NO: 285 | SEQ ID NO: 280 |
| RNP5 | SEQ ID NO: 286 | SEQ ID NO: 280 |
| RNP6 | SEQ ID NO: 287 | SEQ ID NO: 280 |
| RNP7 | SEQ ID NO: 254 | SEQ ID NO: 280 |
| RNP8 | SEQ ID NO: 288 | SEQ ID NO: 280 |
| RNP9 | SEQ ID NO: 267 | SEQ ID NO: 280 |
| RNP10 | SEQ ID NO: 268 | SEQ ID NO: 280 |
| RNP11 | SEQ ID NO: 269 | SEQ ID NO: 280 |
| RNP12 | SEQ ID NO: 255 | SEQ ID NO: 280 |
| RNP13 | SEQ ID NO: 270 | SEQ ID NO: 280 |
| RNP14 | SEQ ID NO: 271 | SEQ ID NO: 280 |
| RNP15 | SEQ ID NO: 274 | SEQ ID NO: 280 |
| RNP16 | SEQ ID NO: 275 | SEQ ID NO: 280 |
| RNP17 | SEQ ID NO: 276 | SEQ ID NO: 280 |
| RNP18 | SEQ ID NO: 277 | SEQ ID NO: 280 |
| RNP19 | SEQ ID NO: 234 | SEQ ID NO: 283 |
| RNP20 | SEQ ID NO: 279 | SEQ ID NO: 280 |
| RNP21 | SEQ ID NO: 237 | SEQ ID NO: 282 |
| RNP22 | SEQ ID NO: 237 | SEQ ID NO: 293 |
| RNP23 | SEQ ID NO: 237 | SEQ ID NO: 294 |
| RNP24 | SEQ ID NO: 289 | SEQ ID NO: 280 |
| RNP25 | SEQ ID NO: 290 | SEQ ID NO: 280 |
| RNP26 | SEQ ID NO: 291 | SEQ ID NO: 280 |
| RNP27 | SEQ ID NO: 237 | SEQ ID NO: 283 |
| RNP28 | SEQ ID NO: 219 | SEQ ID NO: 221 |
| RNP29 | SEQ ID NO: 227 | SEQ ID NO: 221 |
| RNP30 | SEQ ID NO: 228 | SEQ ID NO: 221 |
| RNP31 | SEQ ID NO: 229 | SEQ ID NO: 221 |
| RNP32 | SEQ ID NO: 230 | SEQ ID NO: 221 |
| RNP33 | SEQ ID NO: 231 | SEQ ID NO: 221 |
| RNP34 | SEQ ID NO: 232 | SEQ ID NO: 221 |
| RNP35 | SEQ ID NO: 233 | SEQ ID NO: 221 |
| RNP36 | SEQ ID NO: 234 | SEQ ID NO: 221 |
| RNP37 | SEQ ID NO: 235 | SEQ ID NO: 221 |
| RNP38 | SEQ ID NO: 219 | SEQ ID NO: 280 |
| RNP39 | SEQ ID NO: 232 | SEQ ID NO: 280 |
| RNP40 | SEQ ID NO: 233 | SEQ ID NO: 280 |
| RNP41 | SEQ ID NO: 238 | SEQ ID NO: 280 |
| RNP42 | SEQ ID NO: 234 | SEQ ID NO: 280 |
| RNP43 | SEQ ID NO: 239 | SEQ ID NO: 280 |
| RNP44 | SEQ ID NO: 240 | SEQ ID NO: 280 |
| RNP45 | SEQ ID NO: 241 | SEQ ID NO: 280 |
| RNP46 | SEQ ID NO: 242 | SEQ ID NO: 280 |
| RNP47 | SEQ ID NO: 243 | SEQ ID NO: 280 |
| RNP48 | SEQ ID NO: 244 | SEQ ID NO: 280 |
| RNP49 | SEQ ID NO: 245 | SEQ ID NO: 280 |
| RNP50 | SEQ ID NO: 246 | SEQ ID NO: 280 |
| RNP51 | SEQ ID NO: 237 | SEQ ID NO: 280 |
| RNP52 | SEQ ID NO: 253 | SEQ ID NO: 280 |
| RNP53 | SEQ ID NO: 254 | SEQ ID NO: 280 |
| RNP54 | SEQ ID NO: 227 | SEQ ID NO: 281 |
| RNP55 | SEQ ID NO: 227 | SEQ ID NO: 280 |
| RNP56 | SEQ ID NO: 219 | SEQ ID NO: 281 |

*See Table 20
**See Table 17

TABLE 19
gRNA 5′ Extensions

| 5′ | | |
|---|---|---|
| extension | 5′ extension sequence | |
| Sequence | | |
| ID No: | | 5′ modification |
| 328 | rCrUrUrUrU | +5 RNA |
| 329 | rArArGrArCrCrUrUrUrU | +10 RNA |
| 330 | rArUrGrUrGrUrUrUrUrUrGrUrCrArArArArGrArCrCrUrUrUrU | +25 RNA |
| 331 | rArGrGrCrCrArGrCrUrUrGrCrCrGrGrUrUrUrUrUrUrArGrUrC | +60 RNA |
| | rGrUrGrCrUrGrCrUrUrCrArUrGrUrGrUrUrUrUrUrGrUrCrArA | |
| | rArArGrArCrCrUrUrUrU | |
| 304 | CTTTT | +5 DNA |
| 305 | AAGACCTTTT | +10 DNA |
| 306 | ATGTGTTTTTGTCAAAAGACCTTTT | +25 DNA |
| 307 | AGGCCAGCTTGCCGGTTTTTTAGTCGTGCTGCTTCATGTGTTTTTGTCAA | +60 DNA |
| | AAGACCTTTT | |
| 308 | TTTTTGTCAAAAGACCTTTT | +20 DNA |
| 309 | GCTTCATGTGTTTTTGTCAAAAGACCTTTT | +30 DNA |
| 310 | GCCGGTTTTTTAGTCGTGCTGCTTCATGTGTTTTTGTCAAAAGACCTTTT | +50 DNA |
| 311 | TAGTCGTGCTGCTTCATGTGTTTTTGTCAAAAGACCTTTT | +40 DNA |
| 312 | C*C*GAAGTTTTCTTCGGTTTT | +20 DNA + 2xPS |
| 313 | T*T*TTTCCGAAGTTTTCTTCGGTTTT | +25 DNA + 2xPS |
| 314 | A*A*CGCTTTTTCCGAAGTTTTCTTCGGTTTT | +30 DNA + 2xPS |
| 315 | G*C*GTTGTTTTCAACGCTTTTTCCGAAGTTTTCTTCGGTTTT | +41 DNA + 2xPS |
| 316 | G*G*CTTCTTTTGAAGCCTTTTTGCGTTGTTTTCAACGCTTTTTCCGAAG | +62 DNA + 2xPS |
| | TTTTCTTCGGTTTT | |
| 317 | A*T*GTGTTTTTGTCAAAAGACCTTTT | +25 DNA + 2xPS |
| 318 | AAAAAAAAAAAAAAAAAAAAAAAAA | +25 A |
| 319 | TTTTTTTTTTTTTTTTTTTTTTTTT | +25 T |
| 332 | mA*mU*rGrUrGrUrUrUrUrUrGrUrCrArArArArGrArCrCrUrUrU | +25 RNA + 2xPS |
| | rU | |
| 333 | mA*mA*rArArArArArArArArArArArArArArArArArArArArArA | PolyA RNA + |
| | rA | 2xPS |
| 334 | mU*U*rUrUrUrUrUrUrUrUrUrUrUrUrUrUrUrUrUrUrUrUrUrUr | PolyU RNA + |
| | U | 2xPS |

All bases are in upper case
Lowercase “r” represents RNA, 2′-hydroxy; bases not modified by an “r” are DNA
All bases are linked via standard phosphodiester bonds except as noted:
“*” represents phosphorothioate modification
“PS” represents phosphorothioate modification

TABLE 20
Cas12a guide RNAs

| | | | | Length of | | |
|---|---|---|---|---|---|---|
| gRNA | | | | crRNA + | Length of | gRNA |
| Sequence | | | | gRNA | gRNA | Targeting |
| SEQ ID | | | | targeting | targeting | Domain |
| NO. | gRNA Sequence | 5′ mod .** | 3′ mod. | domain | domain | (RNA) |
| 219 | rUrArArUrUrUrCr | | 1 | 40 | 20 | CCUUGUC |
| | UrArCrUrCrUrUrG | | | | | AAGGCUA |
| | rUrArGrArUrCrCr | | | | | UUGGUC |
| | UrUrGrUrCrArArG | | | | | (SEQ ID |
| | rGrCrUrArUrUrGr | | | | | NO: 202) |
| | GrUrC | | | | | |
| 220 | rUrArArUrUrUrCr | | 1xPS2 | 41 | 21 | CCUUGUC |
| | UrArCrUrCrUrUrG | | OMe + | | | AAGGCUA |
| | rUrArGrArUrCrCr | | 1xOMe | | | UUGGUCA |
| | UrUrGrUrCrArArG | | | | | (SEQ ID |
| | rGrCrUrArUrUrGr | | | | | NO: 320) |
| | GrUmC/PS2/mA | | | | | |
| 227 | rUrArArUrUrUrCr | — | 1xPS2 | 40 | 20 | CCUUGUC |
| | UrArCrUrCrUrUrG | | OMe + | | | AAGGCUA |
| | rUrArGrArUrCrCr | | 1×OMe | | | UUGGUC |
| | UrUrGrUrCrArArG | | | | | (SEQ ID |
| | rGrCrUrArUrUrGr | | | | | NO: 202) |
| | GmU/PS2/mC | | | | | |
| 228 | rCrUrUrUrUrUrAr | +5 RNA | | 45 | 20 | CCUUGUC |
| | ArUrUrUrCrUrArC | | | | | AAGGCUA |
| | rUrCrUrUrGrUrAr | | | | | UUGGUC |
| | GrArUrCrCrUrUrG | | | | | (SEQ ID |
| | rUrCrArArGrGrCr | | | | | NO: 202) |
| | UrArUrUrGrGrUrC | | | | | |
| 229 | rArArGrArCrCrUr | +10 RNA | | 50 | 20 | CCUUGUC |
| | UrUrUrUrArArUrU | | | | | AAGGCUA |
| | rUrCrUrArCrUrCr | | | | | UUGGUC |
| | UrUrGrUrArGrArU | | | | | (SEQ ID |
| | rCrCrUrUrGrUrCr | | | | | NO: 202) |
| | ArArGrGrCrUrArU | | | | | |
| | rUrGrGrUrC | | | | | |
| 230 | rArUrGrUrGrUrUr | +25 RNA | | 65 | 20 | CCUUGUC |
| | UrUrUrGrUrCrArA | | | | | AAGGCUA |
| | rArArGrArCrCrUr | | | | | UUGGUC |
| | UrUrUrUrArArUrU | | | | | (SEQ ID |
| | rUrCrUrArCrUrCr | | | | | NO: 202) |
| | UrUrGrUrArGrArU | | | | | |
| | rCrCrUrUrGrUrCr | | | | | |
| | ArArGrGrCrUrArU | | | | | |
| | rUrGrGrUrC | | | | | |
| 231 | rArGrGrCrCrArGr | +60 RNA | | 100 | 20 | CCUUGUC |
| | CrUrUrGrCrCrGrG | | | | | AAGGCUA |
| | rUrUrUrUrUrUrAr | | | | | UUGGUC |
| | GrUrCrGrUrGrCrU | | | | | (SEQ ID |
| | rGrCrUrUrCrArUr | | | | | NO: 202) |
| | GrUrGrUrUrUrUrU | | | | | |
| | rGrUrCrArArArAr | | | | | |
| | GrArCrCrUrUrUrU | | | | | |
| | rUrArArUrUrUrCr | | | | | |
| | UrArCrUrCrUrUrG | | | | | |
| | rUrArGrArUrCrCr | | | | | |
| | UrUrGrUrCrArArG | | | | | |
| | rGrCrUrArUrUrGr | | | | | |
| | GrUrC | | | | | |
| 232 | CTTTTrUrArArUrU | +5 DNA | | 45 | 20 | CCUUGUC |
| | rUrCrUrArCrUrCr | | | | | AAGGCUA |
| | UrUrGrUrArGrArU | | | | | UUGGUC |
| | rCrCrUrUrGrUrCr | | | | | (SEQ ID |
| | ArArGrGrCrUrArU | | | | | NO: 202) |
| | rUrGrGrUrC | | | | | |
| 233 | AAGACCTTTTrUrAr | +10 DNA | | 50 | 20 | CCUUGUC |
| | ArUrUrUrCrUrArC | | | | | AAGGCUA |
| | rUrCrUrUrGrUrAr | | | | | UUGGUC |
| | GrArUrCrCrUrUrG | | | | | (SEQ ID |
| | rUrCrArArGrGrCr | | | | | NO: 202) |
| | UrArUrUrGrGrUrC | | | | | |
| 234 | ATGTGTTTTTGTCAA | +25 DNA | | 65 | 20 | CCUUGUC |
| | AAGACCTTTTrUrAr | | | | | AAGGCUA |
| | ArUrUrUrCrUrArC | | | | | UUGGUC |
| | rUrCrUrUrGrUrAr | | | | | (SEQ ID |
| | GrArUrCrCrUrUrG | | | | | NO: 202) |
| | rUrCrArArGrGrCr | | | | | |
| | UrArUrUrGrGrUrC | | | | | |
| 235 | AGGCCAGCTTGCCGG | +60 DNA | | 100 | 20 | CCUUGUC |
| | TTTTTTAGTCGTGCT | | | | | AAGGCUA |
| | GCTTCATGTGTTTTT | | | | | UUGGUC |
| | GTCAAAAGACCTTTT | | | | | (SEQ ID |
| | rUrArArUrUrUrCr | | | | | NO: 202) |
| | UrArCrUrCrUrUrG | | | | | |
| | rUrArGrArUrCrCr | | | | | |
| | UrUrGrUrCrArArG | | | | | |
| | rGrCrUrArUrUrGr | | | | | |
| | GrUrC | | | | | |
| 236 | ATGTGTTTTTGTCAA | +25 DNA | 1xPS2 | 66 | 21 | CCUUGUC |
| | AAGACCTTTTrUrAr | | OMe + | | | AAGGCUA |
| | ArUrUrUrCrUrArC | | 1xOMe | | | UUGGUCA |
| | rUrCrUrUrGrUrAr | | | | | (SEQ ID |
| | GrArUrCrCrUrUrG | | | | | NO: 320) |
| | rUrCrArArGrGrCr | | | | | |
| | UrArUrUrGrGrUm | | | | | |
| | C/PS2/mA | | | | | |
| 237 | ATGTGTTTTTGTCAA | +25 DNA | 1xPS | 66 | 21 | CCUUGUC |
| | AAGACCTTTTrUrAr | | OMe | | | AAGGCUA |
| | ArUrUrUrCrUrArC | | | | | UUGGUCA |
| | rUrCrUrUrGrUrAr | | | | | (SEQ ID |
| | GrArUrCrCrUrUrG | | | | | NO: 320) |
| | rUrCrArArGrGrCr | | | | | |
| | UrArUrUrGrGrUrC | | | | | |
| | *mA | | | | | |
| 238 | TTTTTGTCAAAAGAC | +20 DNA | | 60 | 20 | CCUUGUC |
| | CTTTTrUrArArUrU | | | | | AAGGCUA |
| | rUrCrUrArCrUrCr | | | | | UUGGUC |
| | UrUrGrUrArGrArU | | | | | (SEQ ID |
| | rCrCrUrUrGrUrCr | | | | | NO: 202) |
| | ArArGrGrCrUrArU | | | | | |
| | rUrGrGrUrC | | | | | |
| 239 | GCTTCATGTGTTTTT | +30 DNA | | 70 | 20 | CCUUGUC |
| | GTCAAAAGACCTTTT | | | | | AAGGCUA |
| | rUrArArUrUrUrCr | | | | | UUGGUC |
| | UrArCrUrCrUrUrG | | | | | (SEQ ID |
| | rUrArGrArUrCrCr | | | | | NO: 202) |
| | UrUrGrUrCrArArG | | | | | |
| | rGrCrUrArUrUrGr | | | | | |
| | GrUrC | | | | | |
| 240 | GCCGGTTTTTTAGTC | +50 DNA | | 90 | 20 | CCUUGUC |
| | GTGCTGCTTCATGTG | | | | | AAGGCUA |
| | TTTTTGTCAAAAGAC | | | | | UUGGUC |
| | CTTTTrUrArArUrU | | | | | (SEQ ID |
| | rUrCrUrArCrUrCr | | | | | NO: 202) |
| | UrUrGrUrArGrArU | | | | | |
| | rCrCrUrUrGrUrCr | | | | | |
| | ArArGrGrCrUrArU | | | | | |
| | rUrGrGrUrC | | | | | |
| 241 | TAGTCGTGCTGCTTC | +40 DNA | | 80 | 20 | CCUUGUC |
| | ATGTGTTTTTGTCAA | | | | | AAGGCUA |
| | AAGACCTTTTrUrAr | | | | | UUGGUC |
| | ArUrUrUrCrUrArC | | | | | (SEQ ID |
| | rUrCrUrUrGrUrAr | | | | | NO: 202) |
| | GrArUrCrCrUrUrG | | | | | |
| | rUrCrArArGrGrCr | | | | | |
| | UrArUrUrGrGrUrC | | | | | |
| 242 | C*C*GAAGTTTTCTT | +20 DNA + | | 60 | 20 | CCUUGUC |
| | CGGTTTTrUrArArU | 2xPS | | | | AAGGCUA |
| | rUrUrCrUrArCrUr | | | | | UUGGUC |
| | CrUrUrGrUrArGrA | | | | | (SEQ ID |
| | rUrCrCrUrUrGrUr | | | | | NO: 202) |
| | CrArArGrGrCrUrA | | | | | |
| | rUrUrGrGrUrC | | | | | |
| 243 | T*T*TTTCCGAAGTT | +25 DNA + | | 65 | 20 | CCUUGUC |
| | TTCTTCGGTTTTrUr | 2xPS | | | | AAGGCUA |
| | ArArUrUrUrCrUrA | | | | | UUGGUC |
| | rCrUrCrUrUrGrUr | | | | | (SEQ ID |
| | ArGrArUrCrCrUrU | | | | | NO: 202) |
| | rGrUrCrArArGrGr | | | | | |
| | CrUrArUrUrGrGrU | | | | | |
| | rC | | | | | |
| 244 | A*A*CGCTTTTTCCG | +30 DNA + | | 70 | 20 | CCUUGUC |
| | AAGTTTTCTTCGGTT | 2xPS | | | | AAGGCUA |
| | TTrUrArArUrUrUr | | | | | UUGGUC |
| | CrUrArCrUrCrUrU | | | | | (SEQ ID |
| | rGrUrArGrArUrCr | | | | | NO: 202) |
| | CrUrUrGrUrCrArA | | | | | |
| | rGrGrCrUrArUrUr | | | | | |
| | GrGrUrC | | | | | |
| 245 | G*C*GTTGTTTTCAA | +41 DNA + | | 81 | 20 | CCUUGUC |
| | CGCTTTTTCCGAAGT | 2xPS | | | | AAGGCUA |
| | TTTCTTCGGTTTTrU | | | | | UUGGUC |
| | rArArUrUrUrCrUr | | | | | (SEQ ID |
| | ArCrUrCrUrUrGrU | | | | | NO: 202) |
| | rArGrArUrCrCrUr | | | | | |
| | UrGrUrCrArArGrG | | | | | |
| | rCrUrArUrUrGrGr | | | | | |
| | UrC | | | | | |
| 246 | G*G*CTTCTTTTGAA | +62 DNA + | | 102 | 20 | CCUUGUC |
| | GCCTTTTTGCGTTGT | 2xPS | | | | AAGGCUA |
| | TTTCAACGCTTTTTC | | | | | UUGGUC |
| | CGAAGTTTTCTTCGG | | | | | (SEQ ID |
| | TTTTrUrArArUrUr | | | | | NO: 202) |
| | UrCrUrArCrUrCrU | | | | | |
| | rUrGrUrArGrArUr | | | | | |
| | CrCrUrUrGrUrCrA | | | | | |
| | rArGrGrCrUrArUr | | | | | |
| | UrGrGrUrC | | | | | |
| 247 | mUrArArUrUrUrCr | 1xOMe | | 40 | 20 | CCUUGUC |
| | UrArCrUrCrUrUrG | | | | | AAGGCUA |
| | rUrArGrArUrCrCr | | | | | UUGGUC |
| | UrUrGrUrCrArArG | | | | | (SEQ ID |
| | rGrCrUrArUrUrGr | | | | | NO: 202) |
| | GrUrC | | | | | |
| 248 | mU*rArArUrUrUrC | 1xPS OMe | | 40 | 20 | CCUUGUC |
| | rUrArCrUrCrUrUr | | | | | AAGGCUA |
| | GrUrArGrArUrCrC | | | | | UUGGUC |
| | rUrUrGrUrCrArAr | | | | | (SEQ ID |
| | GrGrCrUrArUrUrG | | | | | NO: 202) |
| | rGrUrC | | | | | |
| 249 | mUmArArUrUrUrCr | 2xOMe | | 40 | 20 | CCUUGUC |
| | UrArCrUrCrUrUrG | | | | | AAGGCUA |
| | rUrArGrArUrCrCr | | | | | UUGGUC |
| | UrUrGrUrCrArArG | | | | | (SEQ ID |
| | rGrCrUrArUrUrGr | | | | | NO: 202) |
| | GrUrC | | | | | |
| 250 | mU*mA*rArUrUrUr | 2xPS OMe | | 40 | 20 | CCUUGUC |
| | CrUrArCrUrCrUrU | | | | | AAGGCUA |
| | rGrUrArGrArUrCr | | | | | UUGGUC |
| | CrUrUrGrUrCrArA | | | | | (SEQ ID |
| | rGrGrCrUrArUrUr | | | | | NO: 202) |
| | GrGrUrC | | | | | |
| 251 | mUmAmArUrUrUrCr | 3xOMe | | 40 | 20 | CCUUGUC |
| | UrArCrUrCrUrUrG | | | | | AAGGCUA |
| | rUrArGrArUrCrCr | | | | | UUGGUC |
| | UrUrGrUrCrArArG | | | | | (SEQ ID |
| | rGrCrUrArUrUrGr | | | | | NO: 202) |
| | GrUrC | | | | | |
| 252 | mU*mA*mA*rUrUrU | 3xPS OMe | | 40 | 20 | CCUUGUC |
| | rCrUrArCrUrCrUr | | | | | AAGGCUA |
| | UrGrUrArGrArUrC | | | | | UUGGUC |
| | rCrUrUrGrUrCrAr | | | | | (SEQ ID |
| | ArGrGrCrUrArUrU | | | | | NO: 202) |
| | rGrGrUrC | | | | | |
| 253 | A*T*GTGTTTTTGTC | +25 DNA + | | 65 | 20 | CCUUGUC |
| | AAAAGACCTTTTrUr | 2xPS | | | | AAGGCUA |
| | ArArUrUrUrCrUrA | | | | | UUGGUC |
| | rCrUrCrUrUrGrUr | | | | | (SEQ ID |
| | ArGrArUrCrCrUrU | | | | | NO: 202) |
| | rGrUrCrArArGrGr | | | | | |
| | CrUrArUrUrGrGrU | | | | | |
| | rC | | | | | |
| 254 | A*T*GTGTTTTTGTC | +25 DNA + | 1 xPS | 66 | 21 | CCUUGUC |
| | AAAAGACCTTTTrUr | 2xPS | OMe | | | AAGGCUA |
| | ArArUrUrUrCrUrA | | | | | UUGGUCA |
| | rCrUrCrUrUrGrUr | | | | | (SEQ ID |
| | ArGrArUrCrCrUrU | | | | | NO: 320) |
| | rGrUrCrArArGrGr | | | | | |
| | CrUrArUrUrGrGrU | | | | | |
| | rC*mA | | | | | |
| 255 | rUrArArUrUrUrCr | | 1xPS | 41 | 21 | CCUUGUC |
| | UrArCrUrCrUrUrG | | OMe | | | AAGGCUA |
| | rUrArGrArUrCrCr | | | | | UUGGUCA |
| | UrUrGrUrCrArArG | | | | | (SEQ ID |
| | rGrCrUrArUrUrGr | | | | | NO: 320) |
| | GrUrC*mA | | | | | |
| 256 | rUrArArUrUrUrCr | | 1xPS | 42 | 21 | CCUUGUC |
| | UrArCrUrCrUrUrG | | OMe + | | | AAGGCUA |
| | rUrArGrArUrCrCr | | rU | | | UUGGUCA |
| | UrUrGrUrCrArArG | | | | | (SEQ ID |
| | rGrCrUrArUrUrGr | | | | | NO: 320) |
| | GrUrCmA*rU | | | | | |
| 257 | rUrArArUrUrUrCr | | rU | 41 | 20 | CCUUGUC |
| | UrArCrUrCrUrUrG | | | | | AAGGCUA |
| | rUrArGrArUrCrCr | | | | | UUGGUC |
| | UrUrGrUrCrArArG | | | | | (SEQ ID |
| | rGrCrUrArUrUrGr | | | | | NO: 202) |
| | GrUrCrU | | | | | |
| 258 | rUrArArUrUrUrCr | | rU | 42 | 21 | CCUUGUC |
| | UrArCrUrCrUrUrG | | | | | AAGGCUA |
| | rUrArGrArUrCrCr | | | | | UUGGUCA |
| | UrUrGrUrCrArArG | | | | | (SEQ ID |
| | rGrCrUrArUrUrGr | | | | | NO: 320) |
| | GrUrCrArU | | | | | |
| 259 | rUrArArUrUrUrCr | | rU | 44 | 23 | CCUUGUC |
| | UrArCrUrCrUrUrG | | | | | AAGGCUA |
| | rUrArGrArUrCrCr | | | | | UUGGUCA |
| | UrUrGrUrCrArArG | | | | | AG (SEQ ID |
| | rGrCrUrArUrUrGr | | | | | NO: 322) |
| | GrUrCrArArGrU | | | | | |
| 260 | A*T*GTGTTTTTGTC | +25 DNA | rU | 66 | 20 | CCUUGUC |
| | AAAAGACCTTTTrUr | + 2xPS | | | | AAGGCUA |
| | ArArUrUrUrCrUrA | | | | | UUGGUC |
| | rCrUrCrUrUrGrUr | | | | | (SEQ ID |
| | ArGrArUrCrCrUrU | | | | | NO: 202) |
| | rGrUrCrArArGrGr | | | | | |
| | CrUrArUrUrGrGrU | | | | | |
| | rCrU | | | | | |
| 261 | A*T*GTGTTTTTGTC | +25 DNA | rU | 69 | 23 | CCUUGUC |
| | AAAAGACCTTTTrUr | + 2xPS | | | | AAGGCUA |
| | ArArUrUrUrCrUrA | | | | | UUGGUCA |
| | rCrUrCrUrUrGrUr | | | | | AG (SEQ ID |
| | ArGrArUrCrCrUrU | | | | | NO: 322) |
| | rGrUrCrArArGrGr | | | | | |
| | CrUrArUrUrGrGrU | | | | | |
| | rCrArArGrU | | | | | |
| 262 | rUrArArUrUrUrCr | | | 41 | 21 | CCUUGUC |
| | UrArCrUrCrUrUrG | | | | | AAGGCUA |
| | rUrArGrArUrCrCr | | | | | UUGGUCA |
| | UrUrGrUrCrArArG | | | | | (SEQ ID |
| | rGrCrUrArUrUrGr | | | | | NO: 320) |
| | GrUrCrA | | | | | |
| 263 | rUrArArUrUrUrCr | | | 43 | 23 | CCUUGUC |
| | UrArCrUrCrUrUrG | | | | | AAGGCUA |
| | rUrArGrArUrCrCr | | | | | UUGGUCA |
| | UrUrGrUrCrArArG | | | | | AG (SEQ ID |
| | rGrCrUrArUrUrGr | | | | | NO: 322) |
| | GrUrCrArArG | | | | | |
| 264 | A*T*GTGTTTTTGTC | +25 DNA | | 68 | 23 | CCUUGUC |
| | AAAAGACCTTTTrUr | + 2xPS | | | | AAGGCUA |
| | ArArUrUrUrCrUrA | | | | | UUGGUCA |
| | rCrUrCrUrUrGrUr | | | | | AG (SEQ ID |
| | ArGrArUrCrCrUrU | | | | | NO: 322) |
| | rGrUrCrArArGrGr | | | | | |
| | CrUrArUrUrGrGrU | | | | | |
| | rCrArArG | | | | | |
| 265 | rUrArArUrUrUrCr | | 4xrU | 44 | 20 | CCUUGUC |
| | UrArCrUrCrUrUrG | | | | | AAGGCUA |
| | rUrArGrArUrCrCr | | | | | UUGGUC |
| | UrUrGrUrCrArArG | | | | | (SEQ ID |
| | rGrCrUrArUrUrGr | | | | | NO: 202) |
| | GrUrCrUrUrUrU | | | | | |
| 266 | rUrArArUrUrUrCr | | 3xPS | 44 | 20 | CCUUGUC |
| | UrArCrUrCrUrUrG | | OMe U | | | AAGGCUA |
| | rUrArGrArUrCrCr | | + rU | | | UUGGUC |
| | UrUrGrUrCrArArG | | | | | (SEQ ID |
| | rGrCrUrArUrUrGr | | | | | NO: 202) |
| | GrUrCmU*mU*mU*r | | | | | |
| | U | | | | | |
| 267 | A*T*GTGTTTTTGTC | +25 DNA | 4xrU | 69 | 20 | CCUUGUC |
| | AAAAGACCTTTTrUr | + 2xPS | | | | AAGGCUA |
| | ArArUrUrUrCrUrA | | | | | UUGGUC |
| | rCrUrCrUrUrGrUr | | | | | (SEQ ID |
| | ArGrArUrCrCrUrU | | | | | NO: 202) |
| | rGrUrCrArArGrGr | | | | | |
| | CrUrArUrUrGrGrU | | | | | |
| | rCrUrUrUrU | | | | | |
| 268 | AAAAAAAAAAAAAAA | +25 A | 1xPS | 66 | 21 | CCUUGUC |
| | AAAAAAAAAArUrAr | | OMe | | | AAGGCUA |
| | ArUrUrUrCrUrArC | | | | | UUGGUCA |
| | rUrCrUrUrGrUrAr | | | | | (SEQ ID |
| | GrArUrCrCrUrUrG | | | | | NO: 320) |
| | rUrCrArArGrGrCr | | | | | |
| | UrArUrUrGrGrUrC | | | | | |
| | *mA | | | | | |
| 269 | TTTTTTTTTTTTTTT | +25 T | 1xPS | 66 | 21 | CCUUGUC |
| | TTTTTTTTTTrUrAr | | OMe | | | AAGGCUA |
| | ArUrUrUrCrUrArC | | | | | UUGGUCA |
| | rUrCrUrUrGrUrAr | | | | | (SEQ ID |
| | GrArUrCrCrUrUrG | | | | | NO: 320) |
| | rUrCrArArGrGrCr | | | | | |
| | UrArUrUrGrGrUrC | | | | | |
| | *mA | | | | | |
| 270 | C*C*GAAGTTTTCTT | +20 DNA | 1xPS | 61 | 21 | CCUUGUC |
| | CGGTTTTrUrArArU | + 2xPS | OMe | | | AAGGCUA |
| | rUrUrCrUrArCrUr | | | | | UUGGUCA |
| | CrUrUrGrUrArGrA | | | | | (SEQ ID |
| | rUrCrCrUrUrGrUr | | | | | NO: 320) |
| | CrArArGrGrCrUrA | | | | | |
| | rUrUrGrGrUrC*mA | | | | | |
| 271 | rUrArArUrUrUrCr | | 1xPS | 41 | 21 | AGACAGA |
| | UrArCrUrCrUrUrG | | OMe | | | UAUUUGC |
| | rUrArGrArUrArGr | | | | | AUUGAGA |
| | ArCrArGrArUrArU | | | | | (SEQ ID |
| | rUrUrGrCrArUrUr | | | | | NO: 323) |
| | GrArG*mA | | | | | |
| 272 | ATGTGTTTTTGTCAA | +25 DNA | 1xPS | 66 | 21 | AGACAGA |
| | AAGACCTTTTrUrAr | | OMe | | | UAUUUGC |
| | ArUrUrUrCrUrArC | | | | | AUUGAGA |
| | rUrCrUrUrGrUrAr | | | | | (SEQ ID |
| | GrArUrArGrArCrA | | | | | NO: 323) |
| | rGrArUrArUrUrUr | | | | | |
| | GrCrArUrUrGrArG | | | | | |
| | *mA | | | | | |
| 273 | C*C*GAAGTTTTCTT | +20 DNA + | 1xPS | 61 | 21 | AGACAGA |
| | CGGTTTTrUrArArU | 2xPS | OMe | | | UAUUUGC |
| | rUrUrCrUrArCrUr | | | | | AUUGAGA |
| | CrUrUrGrUrArGrA | | | | | (SEQ ID |
| | rUrArGrArCrArGr | | | | | NO: 323) |
| | ArUrArUrUrUrGrC | | | | | |
| | rArUrUGrArG*mA | | | | | |
| 274 | rUrArArUrUrUrCr | | 1xPS | 41 | 21 | CAUUGAG |
| | UrArCrUrCrUrUrG | | OMe | | | AUAGUGU |
| | rUrArGrArUrCrAr | | | | | GGGGAAG |
| | UrUrGrArGrArUrA | | | | | |
| | rGrUrGrUrGrGrGr | | | | | (SEQ ID |
| | GrArA*mG | | | | | NO: 324) |
| 275 | ATGTGTTTTTGTCAA | +25 DNA | 1xPS | 66 | 21 | CAUUGAG |
| | AAGACCTTTTrUrAr | | OMe | | | AUAGUGU |
| | ArUrUrUrCrUrArC | | | | | GGGGAAG |
| | rUrCrUrUrGrUrAr | | | | | (SEQ ID |
| | GrArUrCrArUrUrG | | | | | NO: 324) |
| | rArGrArUrArGrUr | | | | | |
| | GrUrGrGrGrGrArA | | | | | |
| | *mG | | | | | |
| 276 | C*C*GAAGTTTTCTT | +20 DNA + | 1xPS | 61 | 21 | CAUUGAG |
| | CGGTTTTrUrArArU | 2xPS | OMe | | | AUAGUGU |
| | rUrUrCrUrArCrUr | | | | | GGGGAAG |
| | CrUrUrGrUrArGrA | | | | | (SEQ ID |
| | rUrCrArUrUrGrAr | | | | | NO: 324) |
| | GrArUrArGrUrGrU | | | | | |
| | rGrGrGrGrArA*mG | | | | | |
| 277 | rUrArArUrUrUrCr | | 1xPS | 41 | 21 | CUUCUCC |
| | UrArCrUrCrUrUrG | | OMe | | | CAUCAUA |
| | rUrArGrArUrCrUr | | | | | GAGGAUA |
| | UrCrUrCrCrCrArU | | | | | (SEQ ID |
| | rCrArUrArGrArGr | | | | | NO: 325) |
| | GrArU*mA | | | | | |
| 278 | ATGTGTTTTTGTCAA | +25 DNA | 1xPS | 66 | 21 | CUUCUCC |
| | AAGACCTTTTrUrAr | | OMe | | | CAUCAUA |
| | ArUrUrUrCrUrArC | | | | | GAGGAUA |
| | rUrCrUrUrGrUrAr | | | | | (SEQ ID |
| | GrArUrCrUrUrCrU | | | | | NO: 325) |
| | rCrCrCrArUrCrAr | | | | | |
| | UrArGrArGrGrArU | | | | | |
| | *mA | | | | | |
| 279 | C*C*GAAGTTTTCTT | +20 DNA + | 1xPS | 61 | 21 | CUUCUCC |
| | CGGTTTTrUrArArU | 2xPS | OMe | | | CAUCAUA |
| | rUrUrCrUrArCrUr | | | | | GAGGAUA |
| | CrUrUrGrUrArGrA | | | | | (SEQ ID |
| | rUrCrUrUrCrUrCr | | | | | NO: 325) |
| | CrCrArUrCrArUrA | | | | | |
| | rGrArGrGrArU*mA | | | | | |
| 284 | A*T*GTGTTTTTGTC | +25 DNA + | 1xPS | 67 | 21 | CCUUGUC |
| | AAAAGACCTTTTrUr | 2xPS | OMe + | | | AAGGCUA |
| | ArArUrUrUrCrUrA | | rU | | | UUGGUCA |
| | rCrUrCrUrUrGrUr | | | | | (SEQ ID |
| | ArGrArUrCrCrUrU | | | | | NO: 320) |
| | rGrUrCrArArGrGr | | | | | |
| | CrUrArUrUrGrGrU | | | | | |
| | rCmA*rU | | | | | |
| 285 | A*T*GTGTTTTTGTC | +25 DNA + | 2×PS | 68 | 22 | CCUUGUC |
| | AAAAGACCTTTTrUr | 2xPS | OMe + | | | AAGGCUA |
| | ArArUrUrUrCrUrA | | rU | | | UUGGUCA |
| | rCrUrCrUrUrGrUr | | | | | A (SEQ ID |
| | ArGrArUrCrCrUrU | | | | | NO: 321) |
| | rGrUrCrArArGrGr | | | | | |
| | CrUrArUrUrGrGrU | | | | | |
| | rCmA*mA*rU | | | | | |
| 286 | A*T*GTGTTTTTGTC | +25 DNA + | 1xPS | 66 | 20 | CCUUGUC |
| | AAAAGACCTTTTrUr | 2xPS | OMe + | | | AAGGCUA |
| | ArArUrUrUrCrUrA | | rU | | | UUGGUC |
| | rCrUrCrUrUrGrUr | | | | | (SEQ ID |
| | ArGrArUrCrCrUrU | | | | | NO: 202) |
| | rGrUrCrArArGrGr | | | | | |
| | CrUrArUrUrGrGrU | | | | | |
| | mC*rU | | | | | |
| 287 | A*T*GTGTTTTTGTC | +25 DNA + | 1xPS | 65 | 20 | CCUUGUC |
| | AAAAGACCTTTTrUr | 2xPS | OMe | | | AAGGCUA |
| | ArArUrUrUrCrUrA | | | | | UUGGUC |
| | rCrUrCrUrUrGrUr | | | | | (SEQ ID |
| | ArGrArUrCrCrUrU | | | | | NO: 202) |
| | rGrUrCrArArGrGr | | | | | |
| | CrUrArUrUrGrGrU | | | | | |
| | *mC | | | | | |
| 288 | A*T*GTGTTTTTGTC | +25 DNA + | 2xPS | 67 | 22 | CCUUGUC |
| | AAAAGACCTTTTrUr | 2xPS | OMe | | | AAGGCUA |
| | ArArUrUrUrCrUrA | | | | | UUGGUCA |
| | rCrUrCrUrUrGrUr | | | | | A (SEQ ID |
| | ArGrArUrCrCrUrU | | | | | NO: 321) |
| | rGrUrCrArArGrGr | | | | | |
| | CrUrArUrUrGrGrU | | | | | |
| | rC*mA*mA | | | | | |
| 289 | mA*mU*rGrUrGrUr | +25 RNA + | 1xPS | 66 | 21 | CCUUGUC |
| | UrUrUrUrGrUrCrA | 2xPS | OMe | | | AAGGCUA |
| | rArArArGrArCrCr | | | | | UUGGUCA |
| | UrUrUrUrUrArArU | | | | | (SEQ ID |
| | rUrUrCrUrArCrUr | | | | | NO: 320) |
| | CrUrUrGrUrArGrA | | | | | |
| | rUrCrCrUrUrGrUr | | | | | |
| | CrArArGrGrCrUrA | | | | | |
| | rUrUrGrGrUrC*mA | | | | | |
| 290 | mA*mA*rArArArAr | PolyA | 1xPS | 66 | 21 | CCUUGUC |
| | ArArArArArArArA | RNA + | OMe | | | AAGGCUA |
| | rArArArArArArAr | 2xPS | | | | UUGGUCA |
| | ArArArArUrArArU | | | | | (SEQ ID |
| | rUrUrCrUrArCrUr | | | | | NO: 320) |
| | CrUrUrGrUrArGrA | | | | | |
| | rUrCrCrUrUrGrUr | | | | | |
| | CrArArGrGrCrUrA | | | | | |
| | rUrUrGrGrUrC*mA | | | | | |
| 291 | mU*mU*rUrUrUrUr | PolyU | 1xPS | 66 | 21 | CCUUGUC |
| | UrUrUrUrUrUrUrU | RNA + | OMe | | | AAGGCUA |
| | rUrUrUrUrUrUrUr | 2xPS | | | | UUGGUCA |
| | UrUrUrUrUrArArU | | | | | (SEQ ID |
| | rUrUrCrUrArCrUr | | | | | NO: 320) |
| | CrUrUrGrUrArGrA | | | | | |
| | rUrCrCrUrUrGrUr | | | | | |
| | CrArArGrGrCrUrA | | | | | |
| | rUrUrGrGrUrC*mA | | | | | |

All bases are in upper case
Lowercase “r” represents RNA, 2′-hydroxy; bases not modified by an “r” are DNA
All bases are linked via standard phosphodiester bonds except as noted:
“*” represents phosphorothioate modification
“PS” represents phosphorothioate modification
“PS2” represents phosphorodithioate modification
“OMe” represents a 2′-o-methyl modification
“m” represents a 2′-o-methyl modification
**Table 19 provides a listing of the sequences of the gRNA 5′ extensions

### SEQUENCES

Genome editing system components according to the present disclosure (including without limitation, RNA-guided nucleases, guide RNAs, donor template nucleic acids, nucleic acids encoding nucleases or guide RNAs, and portions or fragments of any of the foregoing), are exemplified by the nucleotide and amino acid sequences presented in the Sequence Listing. The sequences presented in the Sequence Listing are not intended to be limiting, but rather illustrative of certain principles of genome editing systems and their component parts, which, in combination with the instant disclosure, will inform those of skill in the art about additional implementations and modifications that are within the scope of this disclosure.

### INCORPORATION BY REFERENCE

All publications, patents, and patent applications mentioned herein are hereby incorporated by reference in their entirety as if each individual publication, patent or patent application was specifically and individually indicated to be incorporated by reference. In case of conflict, the present application, including any definitions herein, will control.

### EQUIVALENTS

Those skilled in the art will recognize, or be able to ascertain using no more than routine experimentation, many equivalents to the specific embodiments described herein. Such equivalents are intended to be encompassed by the following claims.

### REFERENCES

Ahern et al., Br J Haematol 25(4):437-444 (1973)
Akinbami Hemoglobin 40:64-65 (2016)
Aliyu et al. Am J Hematol 83:63-70 (2008)
Anders et al. Nature 513(7519):569-573 (2014)
Angastiniotis & Modell Ann N Y Acad Sci 850:251-269 (1998)
Bae et al. Bioinformatics 30(10):1473-1475 (2014)
Barbosa et al. Braz J Med Bio Res 43(8):705-711 (2010)
Bauer et al. Nat. Med. 25(5):776-783 (2019)
Bernaudin et al. Blood 110:2749-2756 (2007)
Bothmer et al. CRISPR J 3(3):177-187 (2020)
Bouva Hematologica 91(1):129-132 (2006)
Briner et al. Mol Cell 56(2):333-339 (2014)
Brousseau Am J Hematol 85(1):77-78 (2010)
Caldecott Nat Rev Genet 9(8):619-631 (2008)
Canvers et al. Nature 527(12):192-197 (2015)
Chang et al. Mol Ther Methods Clin Dev 4:137-148 (2017)
Chassanidis Ann Hematol 88(6):549-555 (2009)
Chylinski et al. RNA Biol 10(5):726-737 (2013)
Cong et al. Science 399(6121):819-823 (2013)
Costa et al., Cad Saude Publica 18(5):1469-1471 (2002)
Davis & Maizels 2 Proc Natl Acad Sci USA 111(10):E924-932 (2014)
Fine et al. Sci Rep. 5:10777 (2015)
Frit et al. DNA Repair (Amst) 17:81-97 (2014)
Fu et al. Nat Biotechnol 32:279-284 (2014)
Gao et al. Nat Biotechnol.: 35(8):789-792 (2017)
Giarratana et al. Nat Biotechnol. 23(1):69-74 (2005)
Giarratana et al. Blood: 118, 5071-5079 (2011)
Giannoukos et al. BMC Genomics 19(1):212 (2018)
Guilinger et al. Nat Biotechnol 32:577-582 (2014)
Heigwer et al. Nat Methods 11(2):122-123 (2014)
Hsu et al. Nat Biotechnol 31(9):827-832 (2013)
Iyama & Wilson DNA Repair (Amst) 12(8):620-636 (2013)
Jiang et al. Nat Biotechnol 31(3):233-239 (2013)
Jiao B et al. Blood Adv 2022009202 (2023)
Jinek et al. Science 337(6096):816-821 (2012)
Jinek et al. Science 343(6176):1247997 (2014)
Kato G J et al. Nat Rev Dis Primers 4: 18010 (2018)
Kleinstiver et al. Nature 523(7561):481-485 (2015a)
Kleinstiver et al. Nat Biotechnol 33(12):1293-1298 (2015b)
Kleinstiver et al. Nature 529(7587):490-495 (2016)
Kleinstiver et al. Nat Biotechnol 37(3):276-282 (2019)
Komor et al. Nature 533(7603):420-424 (2016)
Kosicki et al. Nat Biotechnol 36(8): 765-771 (2018)
Lee et al. Nano Lett 12(12):6322-6327 (2012)
Lewis “Medical-Surgical Nursing: Assessment and Management of Clinical Problems” (2014)
Li Cell Res 18(1):85-98 (2008)
Makarova et al. Nat Rev Microbiol 9(6):467-477 (2011)
Mali et al. Science 339(6121):823-826 (2013)
Mantovani et al. Nucleic Acids Res 16(16):7783-7797 (1988)
Masala Methods Enzymol. 231:21-44 (1994)
Marteijn et al. Nat Rev Mol Cell Biol 15(7):465-481 (2014)
Martyn et al., Biochim Biophys Acta 1860(5):525-536 (2017)
Métais et al. Blood Adv. 3(21):3379-92 (2019)
Nishimasu et al. Cell 156(5):935-949 (2014)
Nishimasu et al. Cell 162:1113-1126 (2015)
Notta et al. Science 333(6039):218-21 (2011)
Pausch et al. Science 369(6501):333-337 (2020)
Ran & Hsu Cell 154(6):1380-1389 (2013)
Richardson et al. Nat Biotechnol 34:339-344 (2016)
Swarts et al. May 22:e1481. doi: 10.1002/wrna.1481. Epub ahead of print. PMID: 29790280 (2018)
Shmakov et al. Molecular Cell 60(3):385-397 (2015)
Steinberg et al. Blood. 123(4):481-485 (2014)
Sternberg et al. Nature 507(7490):62-67 (2014)
Strohkendl et al. Mol. Cell 71:816-824 (2018)
Superti-Furga et al. EMBO J 7(10):3099-3107 (1988)
Thein Hum Mol Genet 18(R2):R216-223 (2009)
Thorpe et al. Br J Haematol. 87(1):125-132 (1994)
Tsai et al. Nat Biotechnol 34(5): 483 (2016)
Waber et al. Blood 67(2):551-554 (1986)
Walter et al. NEJM 335:369-76 (1996)
Wang et al. Cell 153(4):910-918 (2013)
Wastnedge E et al. J Glob Health; 8 (2): 021103 (2018)
Weber et al. Sci Adv. 6(7):eaay9392 (2020)
Williams T N et al. Annu Rev Genomics Hum Genet; 19: 113-147 (2018)
Wu et al. Nat. Med. 25(5): 776-83 (2019)
Xiao et al. Bioinformatics 30(8):1180-1182 (2014)
Xu et al. Genes Dev 24(8):783-798 (2010)
Yamano et al. Cell 165(4): 949-962 (2016)
Zetsche et al. Nat Biotechnol 33(2):139-42 (2015)

1. A method of inducing expression of fetal hemoglobin (HbF) in a population of cells (e.g., CD34+ hematopoietic stem and progenitor cells (HSPCs) and/or red blood cells (RBCs), in a subject suffering from a β-hemoglobinopathy, the method comprising:
administering to the subject a population of modified cells comprising a plurality of modified CD34+ or hematopoietic stem cells comprising an indel in an HBG promoter,
thereby inducing expression of HbF in the population of cells (e.g., HSPCs and/or RBCs), wherein HbF as a percentage of total hemoglobin (% HbF) in the subject is about 5%, 10%, 15%, 20%, 25%, 30%, 35%, 40%, 45%, 50%, 55%, 60%, 65%, 70%, 75%, or 80% HbF.

2. The method of claim 1, wherein the % HbF is from about 10% to about 30%, from about 20% to about 40%, from about 30% to about 50%, from about 40% to about 60%, from about 50% to about 70%, from about 60% to about 80%, from about 10% to about 20%, from about 20% to about 30%, from about 30% to about 40%, from about 40% to about 50%, from about 50% to about 60%, from about 60% to about 70%, from about 70% to about 80% HbF, or a range defined by any of the two preceding values.

3. The method of claim 1 or 2, wherein a concentration of total hemoglobin in the subject is about 10 g/dL, 11 g/dL, 12 g/dL, 13 g/dL, 14 g/dL, 15 g/dL, 16 g/dL, 17 g/dL, 18 g/dL, 19 g/dL, or 20 g/dL.

4. The method of claim 1 or 2, wherein a concentration of total hemoglobin in the subject is from about 10.0 to about 20.0 g/dL, from about 13.6 to about 18.0 g/dL (for males), from about 12.0 to about 16.0 g/dL (for females), or a range defined by any of the two preceding values.

5. The method of any one of claims 1-4, wherein a Hemoglobin F concentration in the subject is about 1 g/dL, 2 g/dL, 3 g/dL, 4 g/dL, 5 g/dL, 6 g/dL, 7 g/dL, 8 g/dL, 9 g/dL, 10 g/dL, 11 g/dL, 12 g/dL, 13 g/dL, 14 g/dL, 15 g/dL, 16 g/dL, 17 g/dL, 18 g/dL, 19 g/dL, or 20 g/dL.

6. The method of any one of claims 1-4, wherein a Hemoglobin F concentration in the subject is from about 1 g/dL to about 20 g/dL, or a range defined by any of the two preceding values.

7. The method of any one of claims 1-6, wherein a percentage of F-cells among circulating RBCs in the subject is about 5%, 10%, 15%, 20%, 25%, 30%, 35%, 40%, 45%, 50%, 55%, 60%, 65%, 70%, 75%, 80%, 85%, 90%, 95%, 100% F-cells.

8. The method of any one of claims 1-6, wherein a percentage of F-cells among circulating RBCs in the subject is from about 50% to about 100%, from about 60% to about 100%, from about 70% to about 100%, from about 80% to about 100%, from about 50% to about 60%, from about 60% to about 70%, from about 70% to about 80%, from about 80% to about 90%, from about 90% to about 100%, or a range defined by any of the two preceding values.

9. The method of any one of claims 1-8, wherein a mean corpuscular HbF (pg/RBC) in the subject is about 1.0 pg/RBC, 2.0 pg/RBC, 3.0 pg/RBC, 4.0 pg/RBC, 5.0 pg/RBC, 6.0 pg/RBC, 7.0 pg/RBC, 8.0 pg/RBC, 9.0 pg/RBC, 10.0 pg/RBC, 11.0 pg/RBC, 12.0 pg/RBC, 13.0 pg/RBC, 14.0 pg/RBC 15.0 pg/RBC, 16.0 pg/RBC, 17.0 pg/RBC, 18.0 pg/RBC, 19.0 pg/RBC, 20.0 pg/RBC, 25.0 pg/RBC, or 30.0 pg/RBC.

10. The method of any one of claims 1-8, wherein a mean corpuscular HbF (pg/RBC) in the subject is from about 1.0 pg/RBC to about 5.00 pg/RBC, from about 5.0 pg/RBC to about 10.00 pg/RBC, from about 10.0 pg/RBC to about 15.00 pg/RBC, from about 15.0 pg/RBC to about 20.00 pg/RBC, from about 20.0 pg/RBC to about 25.00 pg/RBC, from about 25.0 pg/RBC to about 30.00 pg/RBC, or a range defined by any of the two preceding values.

11. The method of any one of claims 1-8, wherein a mean corpuscular HbF (pg/RBC) in the subject is ≥10.0 pg/RBC.

12. The method of any one of claims 1-11, wherein a mean proportion of HbF as a percentage of total hemoglobin in the subject is about 5%, 10%, 15%, 20%, 25%, 30%, 35%, 40%, 45%, 50%, 55%, 60%, 65%, 70%, 75%, 80%, 85%, 90%, 95%.

13. The method of any one of claims 1-12, wherein a mean proportion of HbS as a percentage of total hemoglobin in the subject is about 5%, 10%, 15%, 20%, 25%, 30%, 35%, 40%, 45%, 50%, 55%, 60%, 65%, 70%, 75%, 80%, 85%, 90%, 95%.

14. The method of any one of claims 1-13, wherein a mean proportion of HbA as a percentage of total hemoglobin in the subject is about 1%, 2%, 3%, 4%, 5%, 10%, 15%, 20%, 25%, 30%, 35%, 40%, 45%, 50%, 55%, 60%, 65%, 70%, 75%, 80%, 85%, 90%, 95%.

15. The method of any one of claims 1-14, wherein a mean proportion of HbA2 as a percentage of total hemoglobin in the subject is about 1%, 2%, 3%, 4%, 5%.

16. The method of any one of claims 1-15, wherein a lactate dehydrogenase (U/L) in the subject is about 110 U/L, 120 U/L, 130 U/L, 140 U/L, 150 U/L, 160 U/L, 170 U/L, 180 U/L, 190 U/L, 200 U/L, 210 U/L, 220 U/L, or 230 U/L.

17. The method of any one of claims 11-15, wherein a lactate dehydrogenase (U/L) in the subject is from about 110 U/L to about 230 U/L, or a range defined by any of the two preceding values.

18. The method of any one of claims 1-17, wherein an indirect bilirubin (μmol/L) in the subject is about 0.0 μmol/L, 1 μmol/L, 2 μmol/L, 3 mol/L, 4 μmol/L, 5 μmol/L, 6 mol/L, 7 μmol/L, 8 μmol/L, 9 μmol/L, 10 μmol/L, 11 μmol/L, 12 μmol/L, 13 μmol/L, 14 μmol/L, 15 μmol/L, 16 μmol/L, or 17 μmol/L.

19. The method of any one of claims 1-17, wherein an indirect bilirubin (μmol/L) in the subject is from about 0.0 μmol/L to about 16.6 μmol/L, or a range defined by the two preceding values.

20. The method of any one of claims 1-19, wherein a haptoglobin (g/L) in the subject is about 0.3 g/L, 0.4 g/L, 0.5 g/L, 0.6 g/L, 0.7 g/L, 0.8 g/L, 0.9 g/L, 1.0 g/L, 1.1 g/L, 1.2 g/L, 1.3 g/L, 1.4 g/L, 1.5 g/L, 1.6 g/L, 1.7 g/L, 1.8 g/L, 1.9 g/L, or 2.0 g/L.

21. The method of any one of claims 1-19, wherein a haptoglobin (g/L) in the subject is from about 0.3 g/L to about 2.0 g/L or a range defined by the two preceding values.

22. The method of any one of claims 1-21, wherein a reticulocyte count (%) in the subject is about 0.3%, 0.4%, 0.5%, 0.6%, 0.7%, 0.8%, 0.9%, 1.0%, 1.1%, 1.2%, 1.3%, 1.4%, 1.5%, 1.6%, 1.7%, 1.8%, 1.9%, 2.0%, 2.1%, 2.2%, or 2.3%.

23. The method of any one of claims 1-21, wherein a reticulocyte count (%) in the subject is from about 0.3% to about 2.3% or a range defined by the two preceding values.

24. The method of any one of claims 1-23, wherein the subject undergoes myeloablative conditioning with busulfan prior to administering the population of modified cells.

25. The method of any one of claim 1-24, wherein administering the population of modified cells comprises a single infusion of the modified population of cells.

26. The method of any one of claims 1-25, wherein the population of modified cells is about ≥1×10⁶ cells/kg, ≥2×10⁶ cells/kg, ≥3×10⁶ cells/kg, ≥4×10⁶ cells/kg, ≥5×10⁶ cells/kg, ≥6×10⁶ cells/kg, ≥7×10⁶ cells/kg, ≥8×10⁶ cells/kg, ≥9×10⁶ cells/kg, ≥10×10⁶ cells/kg, ≥11×10⁶ cells/kg, ≥12×10⁶ cells/kg, ≥13×10⁶ cells/kg, ≥14×10⁶ cells/kg, ≥15×10⁶ cells/kg, ≥16×10⁶ cells/kg, ≥17×10⁶ cells/kg, ≥18×10⁶ cells/kg, ≥19×10⁶ cells/kg, ≥20×10⁶ cells/kg, ≥21×10⁶ cells/kg, ≥22×10⁶ cells/kg, ≥23×10⁶ cells/kg, ≥24×10⁶ cells/kg, ≥25×10⁶ cells/kg, 1×10⁶ cells/kg to 25×10⁶ cells/kg, 1×10⁶ cells/kg to 5×10⁶ cells/kg, 3×10⁶ cells/kg to 20×10⁶ cells/kg, 3×10⁶ cells/kg to 15×10⁶ cells/kg, 3×10⁶ cells/kg to 10×10⁶ cells/kg, 3×10⁶ cells/kg to 5×10⁶ cells/kg, 5×10⁶ cells/kg to 10×10⁶ cells/kg, 10×10⁶ cells/kg to 15×10⁶ cells/kg, 15×10⁶ cells/kg to 20×10⁶ cells/kg, or 20×10⁶ cells/kg to 25×10⁶ cells/kg.

27. The method of any one of claims 1-26, wherein the % HbF is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

28. The method of any one of claims 3-27, wherein the concentration of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

29. The method of any one of claims 5-28, wherein the Hemoglobin F concentration is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

30. The method of any one of claims 7-29, wherein the percentage of F-cells is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

31. The method of any one of claims 9-30, wherein the mean corpuscular HbF (pg/RBC) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

32. The method of any one of claims 12-31, wherein the mean proportion of HbF as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

33. The method of any one of claims 13-32, wherein the mean proportion of HbS as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

34. The method of any one of claims 14-33, wherein the mean proportion of HbA as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

35. The method of any one of claims 15-34, wherein the mean proportion of HbA2 as a percentage of total hemoglobin is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

36. The method of any one of claims 16-35, wherein the lactate dehydrogenase (U/L) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

37. The method of any one of claims 18-36, wherein the indirect bilirubin (mol/L) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

38. The method of any one of claims 20-37, wherein the haptoglobin (g/L) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

39. The method of any one of claims 22-38, wherein the reticulocyte count (%) is determined 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

40. The method of any one of claims 1-39, wherein normalization of total hemoglobin in the subject occurs by at least 1 month, 1.5 months, 2 months, 3 months, 4 months, 5 months after administering the population of modified cells to the subject.

41. The method of any one of claims 1-40, wherein the subject experiences no vaso-occlusive events (VOEs) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

42. The method of any one of claims 1-40, wherein the subject experiences no severe VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

43. The method of any one of claims 1-40, wherein the subject experiences fewer than 2, 3, 4, 5, 6, 7, 8, 9, 10, 11, 12, 13, 14, 15, 16, 17, 18, 19, or 20 VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

44. The method of any one of claims 1-40, wherein the subject experiences fewer than 2, 3, 4, 5, 6, 7, 8, 9, or 10 severe VOEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

45. The method of any one of claims 1-44, wherein the subject experiences fewer than 1, 2, 3, 4, 5, 10, 15, 20, 25, 30, 35, 40, 45, 50, 55, 60, 65 treatment emergent adverse events (TEAEs) 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

46. The method of any one of claims 1-44, wherein the subject may experience fewer than 1, 2, 3, 4, 5, 6, 7, 8, 9, or 10 serious TEAEs 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

47. The method of any one of claims 1-46, wherein the subject is red blood cell (RBC) transfusion free 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

48. The method of any one of claims 1-47, wherein the subject is suffering from severe sickle cell disease.

49. The method of any one of claims 1-47, wherein the subject is suffering from transfusion-dependent beta-thalassemia (TDT).

50. The method of any one of claims 1-49, wherein an RNP complex comprising a guide RNA (gRNA) and a Cas12a protein is delivered to a population of unmodified cells comprising a plurality of unmodified CD34+ or hematopoietic stem cells from the subject to generate the population of modified cells.

51. The method of claim 50, wherein the gRNA comprises a targeting domain comprising SEQ ID NO:202 or SEQ ID NO:320.

52. The method of claim 50 or 51, wherein the gRNA comprises a 5′ end and a 3′ end, a DNA extension at the 5′ end, and a 2′-O-methyl and phosphorothioate modification at the 3′ end.

53. The method of claim 52, wherein the DNA extension comprises a sequence selected from the group consisting of SEQ ID NOs:304-319.

54. The method of any one of claims 50-53, wherein the Cas12a protein comprises a sequence selected from the group consisting of SEQ ID NO:200, 201, 205-215, 221, 222-226, 280-283, 293-295, and 335.

55. The method of any one of claims 50-53, wherein the Cas12a protein is encoded by a sequence comprising a sequence selected from the group consisting of SEQ ID NOs:216-218, 296-303, and 336.

56. The method of any one of claims 1-55, wherein the indel in the HBG gene promoter is in a CCAAT box target region.

57. A method of producing a modified population of peripheral blood nucleated cells in a subject, the method comprising:
administering to the subject a population of modified cells comprising a plurality of modified CD34+ or hematopoictic stem cells comprising an indel in an HBG promoter,
thereby producing the modified population of peripheral blood nucleated cells, wherein the modified population of peripheral blood nucleated cells as a percentage of the total population of peripheral blood nucleated cells is at least 50%, 55%, 60%, 65%, 70%, 75%, 80%, 85%, 90% or 95%.

58. The method of claim 57, wherein the subject undergoes myeloablative conditioning with busulfan prior to administering the population of modified cells.

59. The method of claim 57 or 58, wherein the subject has a single homozygous mutation in the HBB gene, c.17A>T (HbS mutation).

60. The method of any one of claims 57-59, wherein the subject is suffering from severe sickle cell disease.

61. The method of any one of claims 57-59, wherein the subject is suffering from transfusion-dependent beta-thalassemia (TDT).

62. A method of generating a population of F-cells in a subject, the method comprising:
administering to the subject a population of modified cells comprising a plurality of modified CD34+ or hematopoietic stem cells comprising an indel in an HBG promoter,
wherein the percentage of F-cells among circulating red blood cells six months after said administering is at least 90%.

63. The method of claim 62, wherein mean corpuscular HbF per F-cell in the population of F-cells is at least 18 pg.

64. The method of claim 53 or 54, wherein the subject exhibits no vaso-occlusive events (VOEs) within six months after said administering.

65. The method of any one of claims 62 to 64, wherein the subject exhibits no VOEs within one year after said administering.

66. The method of any one of claims 62 to 65, wherein the subject experiences fewer than 2, 3, 4, 5, 6, 7, 8, 9, or 10 severe VOEs, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.

67. The method of any one of claims 62 to 66, wherein the subject is red blood cell (RBC) transfusion free 1, 2, 3, 4, 5, 6, 7, 8, 9, 10, 11, 12, 13, 14, 15, 16, 17, 18, 19, 20, 21, 22, 23, 24, 25, 26, 27, 28, 29, 30 months after administering the population of modified cells to the subject.
